(19) United States
(12) Patent Application Publication
(10) Pub. No.: US 2025/0154173 A1
(43) Pub. Date: May 15, 2025

(54) RIPK1 INHIBITORS AND METHODS OF USE

(71) Applicant: Merck Sharp & Dohme LLC, Rahway, NJ (US)

(72) Inventors: Barbara Pio, West Orange, NJ (US); Phieng Siliphaivanh, Newton, MA (US); Abdelghani Abe Achab, Melrose, MA (US); Yaroslav Boyko, Brookline, MA (US); Yi-Heng Chen, Whippany, NJ (US); Erin F. DiMauro, Cambridge, MA (US); Jenny Lorena Rico Duque, Canton, MA (US); Xavier Fradera, Jamaica Plain, MA (US); Anandan Palani, Needham, MA (US); Meredith M. Rickard, Chestnut Hill, MA (US); Jing Su, Scotch Plains, NJ (US); Brandon A. Vara, Melrose, MA (US)

(73) Assignee: Merck Sharp & Dohme LLC, Rahway, NJ (US)

(21) Appl. No.: 18/932,819

(22) Filed: Oct. 31, 2024

Related U.S. Application Data
(60) Provisional application No. 63/595,482, filed on Nov. 2, 2023.

(51) Int. Cl. C07D 519/00; A61K 31/4188; A61K 31/4439; A61K 31/497; A61K 31/506; A61K 31/53; C07D 498/10

(52) U.S. Cl. CPC C07D 519/00; A61K 31/4188; A61K 31/4439; A61K 31/497; A61K 31/506; A61K 31/53; C07D 498/10

(57) ABSTRACT

Described herein are compounds of Formula I

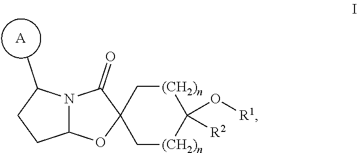

or a pharmaceutically acceptable salt thereof. The compounds of Formula I act as RIPK1 inhibitors and can be useful in preventing, treating or acting as a remedial agent for RIPK1-related diseases.

### CROSS-REFERENCE TO RELATED APPLICATIONS

The application claims the benefit of priority to U.S. Provisional Application No. 63/595,482, filed Nov. 2, 2023, the contents of each of which are incorporated herein by reference in their entirety.

### FIELD

Disclosed herein are novel RIPK1 inhibitors. The RIPK1 inhibitors described herein can be useful in preventing, treating or acting as a remedial agent for RIPK1-related diseases.

### BACKGROUND

Receptor-interacting protein-1 kinase (RIPK1) belongs to the family serine/threonine protein kinase involved in innate immune signaling. RIPK1 has emerged as a promising therapeutic target for the treatment of a wide range of human neurodegenerative, autoimmune, and inflammatory diseases. This is supported by extensive studies which have demonstrated that RIPK1 is a key mediator of apoptotic and necrotic cell death as well as inflammatory pathways.

For example, RIPK1 inhibition has been found to be useful as a treatment of acute kidney injury (AKI), a destructive clinical condition induced by multiple insults including ischemic reperfusion, nephrotoxic drugs and sepsis. It has been found that RIPK1-mediated necroptosis plays an important role in AKI and a RIPK1 inhibitor may serve as a promising clinical candidate for AKI treatment. Wang J N, Liu M M, Wang F, Wei B, Yang Q, Cai Y T, Chen X, Liu X Q, Jiang L, Li C, Hu X W, Yu J T, Ma T T, Jin J, Wu Y G, Li J, Meng X M, RIPK1 Inhibitor Cpd-71 Attenuates Renal Dysfunction in Cisplatin-Treated Mice via Attenuating Necroptosis, Inflammation and Oxidative Stress. Clin Sci (Lond). 2019 Jul. 25; 133(14):1609-1627.

Additionally, human genetic evidence has linked the dysregulation of RIPK1 to the pathogenesis of amyotrophic lateral sclerosis (ALS), Alzheimer's disease and multiple sclerosis as well as other inflammatory and neurodegenerative diseases. Alexei Degterev, Dimitry Ofengeim, and Junying Yuan, Targeting RIPK1 for the treatment of human diseases, PNAS, May 14, 2019, 116 (20), 9714-9722; Ito Y, Ofengeim D, Najafov A, Das S, Saberi S, Li Y, et al., RIPK1 mediates axonal degeneration by promoting inflammation and necroptosis in ALS, Science, 2016, 353:603-8; Caccamo A, Branca C, Piras I S, Ferreira E, Huentelman M J, Liang W S, et al., Necroptosis activation in Alzheimer's disease, Nat Neurosci, 2017, 20:1236-46; Ofengeim D, Ito Y, Najafov A, Zhang Y, Shan B, DeWitt J P, et al., Activation of necroptosis in multiple sclerosis, Cell Rep., 2015, 10:1836-49.

It also has been demonstrated that necroptosis is a delayed component of ischemic neuronal injury, thus RIPK1 inhibition may also play a promising role as a treatment for stroke. Degterev A, et al., Chemical inhibitor of nonapoptotic cell death with therapeutic potential for ischemic brain injury, Nat Chem Biol 2005, 1(2):112-119.

Therefore, there is a need for inhibitors of RIPK1 that offer high selectivity which can penetrate the blood-brain barrier, thus offering the possibility to target neuroinflammation and cell death which drive various neurologic conditions including Alzheimer's disease, ALS, and multiple sclerosis as well as acute neurological diseases such as stroke and traumatic brain injuries.

### SUMMARY

Described herein are compounds of Formula I:

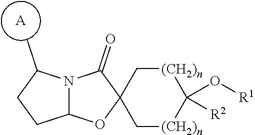

or a pharmaceutically acceptable salt thereof, wherein A, R1, R2, and n are described below.

The compounds described herein are RIPK1 inhibitors, which can be useful in the prevention, treatment or amelioration of neurodegenerative, autoimmune, inflammatory diseases and other RIPK1-related diseases.

Also described herein are methods of treating neurodegenerative, autoimmune, and inflammatory diseases comprising administering to a patient in need thereof a compound described herein, or a pharmaceutically acceptable salt thereof.

Also described herein are uses of a compound described herein, or a pharmaceutically acceptable salt thereof, to treat neurodegenerative, autoimmune, and inflammatory diseases in a patient in need thereof.

Also described herein are pharmaceutical compositions comprising a compound described herein, or a pharmaceutically acceptable salt thereof, and a pharmaceutically acceptable carrier.

Also described herein are pharmaceutical compositions comprising a compound described herein and a pharmaceutically acceptable carrier.

Also described herein are methods of treating neurodegenerative, autoimmune, and inflammatory diseases comprising administering to a patient in need thereof a compound described herein, or a pharmaceutically acceptable salt thereof, and at least one additional therapeutic agent.

Also described herein are uses of a compound described herein, or a pharmaceutically acceptable salt thereof, in combination with at least one additional agent, to treat neurodegenerative, autoimmune, and inflammatory diseases in a patient in need thereof.

Also described herein are pharmaceutical compositions comprising a compound described herein, or a pharmaceutically acceptable salt thereof, at least one additional therapeutic agent and a pharmaceutically acceptable carrier.

Also described herein are pharmaceutical compositions comprising a compound described herein, at least one additional therapeutic agent and a pharmaceutically acceptable carrier.

The summary of the technology described above is non-limiting and other features and advantages of the technology will be apparent from the following detailed description, and from the claims.

### DETAILED DESCRIPTION

Described herein are compounds of Formula I:

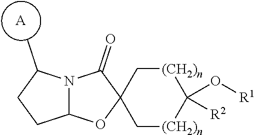

or a pharmaceutically acceptable salt thereof, wherein:

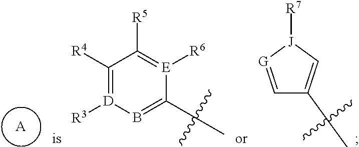

R¹ is —(R⁷)m-R⁸
R² is H or (C1-C6) alkyl;
R³ is H or halogen;
R⁴ is H or halogen;
R⁵ is H or halogen;
R⁶ is H or halogen;
R⁷ is (C1-C6)alkyl or (C1-C6)carbonyl;
R⁸ is
   a) an aryl optionally substituted by up to 3 halogen, O(C₁-C₆)alkyl, O(C₃-C₆)cycloalkyl, (C₃-C₈)cycloalkyl optionally substituted by a nitrile, O—(C₁-C₆)alkyl-O—CH₃, C(O)NH₂, (C₁-C₆)alkyl optionally substituted by up to 3 halogen, S(O)₂—CH₃, (C₁-C₆)nitrile, ((C₁-C₆)alkyl)ₚ-(C₅-C₆)heteroaryl having up to 3 heteroatoms selected from N, O, and S and further optionally substituted by (C₁-C₆)alkyl which is optionally substituted by up to 3 halogen;
   b) a heteroaryl having up to 5 heteroatoms selected from N, O, and S and optionally substituted by up to 3 halogen, O(C₁-C₆)alkyl, O(C₁-C₆)alkyl-F, S(C₁-C₆)alkyl, C(O)NH₂, (C₁-C₆)alkyl optionally substituted by up to 3 halogen, (C₁-C₆)nitrile, (C₃-C₈)cycloalkyl optionally substituted by a nitrile, ((C₁-C₆)alkyl)ₚ-aryl, ((C₁-C₆)alkyl)ₚ-(C₅-C₆)heterocycle having up to 3 heteroatoms selected from N, O, and S, ((C₁-C₆)alkyl)ₚ-(C₅-C₆)heteroaryl having up to 3 heteroatoms selected from N, O, and S optionally substituted by (C₁-C₆)alkyl, ((C₁-C₆)alkyl)ₚ-(C₅-C₆)cycloalkyl optionally substituted by (C₅-C₆)heterocycle having up to 3 heteroatoms selected from N, O, and S and further optionally substituted by (C₁-C₆)alkyl which is optionally substituted by up to 3 halogen, O—(C₅-C₆)heterocycle having up to 3 heteroatoms selected from N, O, and S, (C₅-C₆)heteroaryl having up to 3 heteroatoms selected from N, O, and S and further optionally substituted by (C₁-C₆)alkyl which is optionally substituted by up to 3 halogen; or
   c) a cycloalkyl;
B is CH or N;
D is C or N, provided that if D is N then R³ is absent;
E is C or N, provided that if E is N then R⁶ is absent;
G is CH or N;
J is CH or N;
m is 0 or 1;
n is 0 or 1; and
p is 0 or 1.

In certain embodiments of the present invention, A is

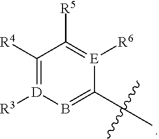

In some embodiments of the present invention, B is CH and D, and E are C.

In particular embodiments of the present invention, D and E are both N.

In further embodiments of the present invention, E is N.

In still further embodiments of the present invention, R⁴ and R⁶ are both F.

In some embodiments of the present invention, R⁵ is F.

In other embodiments of the present invention, R² is H.

In other particular embodiments of the present invention, R⁷ is (C₁-C₆) alkyl and m is 1.

In further embodiments of the present invention, m is 0.

In some embodiments of the present invention, R⁸ is an aryl. In particular embodiments of the present invention, R⁸ is an aryl substituted by up to 3 halogen. In additional embodiments of the present invention, R⁸ is an aryl substituted by O(C₁-C₆)alkyl. In certain embodiments of the present invention, R⁸ is an aryl substituted by (C₃-C₈)cycloalkyl optionally substituted by a nitrile, In further embodiments of the present invention, R⁸ is an aryl substituted by O(C₃-C₆)cycloalkyl. In still further embodiments of the present invention, R⁸ is an aryl substituted by O—(C₁-C₆)alkyl-O—CH₃. In additional embodiments of the present invention, R⁸ is an aryl substituted by C(O)NH₂. In further embodiments of the present invention, R⁸ is an aryl substituted by (C₁-C₆)alkyl optionally substituted by up to 3 halogen. In still further embodiments of the present invention, R⁸ is an aryl substituted by S(O)₂—CH₃. In additional embodiments of the present invention, R⁸ is an aryl substituted by (C₁-C₆)nitrile. In particular embodiments of the present invention, R⁸ is an aryl substituted by ((C₁-C₆)alkyl)ₚ-(C₅-C₆)heteroaryl having up to 3 heteroatoms selected from N, O, and S. In further particular embodiments of the present invention, R⁸ is an aryl substituted by ((C₁-C₆)alkyl)ₚ-(C₅-C₆)heteroaryl having up to 3 heteroatoms selected from N, O, and S further substituted by (C₁-C₆)alkyl. In additional embodiments of the present invention, R⁸ is an aryl substituted by ((C₁-C₆)alkyl)ₚ-(C₅-C₆)heteroaryl having up to 3 heteroatoms selected from N, O, and S further substituted by (C₁-C₆)alkyl which is further substituted by up to 3 halogen.

In some embodiments of the present invention, R⁸ is a heteroaryl having up to 5 heteroatoms selected from N, O and S. In other particular embodiments of the present invention, R⁸ is a heteroaryl having up to 5 heteroatoms selected from N, O and S and wherein the heteroaryl is substituted by up to 3 halogen. In additional embodiments of the present invention, R⁸ is a heteroaryl having up to 5 heteroatoms selected from N, O and S and wherein the heteroaryl is substituted by O(C₁-C₆)alkyl. In certain embodiments of the present invention, R⁸ is a heteroaryl having up to 5 heteroatoms selected from N, O and S and wherein the heteroaryl is substituted by ((C₁-C₆)alkyl)ₚ-aryl. In some embodiments of the present invention, R⁸ is a heteroaryl having up to 5 heteroatoms selected from N, O and S and wherein the heteroaryl is substituted by ((C₁-C₆)alkyl)ₚ-(C₅-C₆)heterocycle having up to 3 heteroatoms selected from N, O, and S. In further embodiments of the present invention, R⁸ is a heteroaryl having up to 5 heteroatoms selected from N, O and S and wherein the heteroaryl is substituted by O(C₁-C₆)alkyl-F. In still further embodiments of the present invention, R⁸ is a heteroaryl having up to 5 heteroatoms selected from N, O and S and wherein the heteroaryl is substituted by S(C₁-C₆)alkyl. In additional embodiments of the present invention, R⁸ is a heteroaryl having up to 5 heteroatoms selected from N, O and S and wherein the heteroaryl is substituted by C(O)NH₂. In further embodiments of the present invention, R⁸ is a heteroaryl having up to 5 heteroatoms selected from N, O and S and wherein the heteroaryl is substituted by (C₁-C₆)alkyl. In still further embodiments of the present invention, R⁸ is a heteroaryl having up to 5 heteroatoms selected from N, O and S and wherein the heteroaryl is substituted by (C₁-C₆)alkyl which is further substituted by up to 3 halogen. In additional embodiments of the present invention, R⁸ is a heteroaryl having up to 5 heteroatoms selected from N, O and S and wherein the heteroaryl is substituted by (C₁-C₆)nitrile. In further embodiments of the present invention, R⁸ is a heteroaryl having up to 5 heteroatoms selected from N, O and S and wherein the heteroaryl is substituted by (C₃-C₈)cycloalkyl. In still further embodiments of the present invention, R⁸ is a heteroaryl having up to 5 heteroatoms selected from N, O and S and wherein the heteroaryl is substituted by (C₃-C₈)cycloalkyl which is further substituted by a nitrile. In additional embodiments of the present invention, R⁸ is a heteroaryl having up to 5 heteroatoms selected from N, O and S and wherein the heteroaryl is substituted by ((C₁-C₆)alkyl)ₚ-(C₅-C₆)heteroaryl having up to 3 heteroatoms selected from N, O, and S. In certain embodiments of the present invention, R⁸ is a heteroaryl having up to 5 heteroatoms selected from N, O and S and wherein the heteroaryl is substituted by ((C₁-C₆)alkyl)ₚ-(C₅-C₆)heteroaryl having up to 3 heteroatoms selected from N, O, and S which is further substituted by (C₁-C₆)alkyl. In further embodiments of the present invention, R⁸ is a heteroaryl having up to 5 heteroatoms selected from N, O and S and wherein the heteroaryl is substituted by ((C₁-C₆)alkyl)ₚ-(C₅-C₆)cycloalkyl. In still further embodiments of the present invention, R⁸ is a heteroaryl having up to 5 heteroatoms selected from N, O and S and wherein the heteroaryl is substituted by ((C₁-C₆)alkyl)ₚ-(C₅-C₆)cycloalkyl which is substituted by (C₅-C₆)heterocycle having up to 3 heteroatoms selected from N, O, and S. In particular embodiments of the present invention, R⁸ is a heteroaryl having up to 5 heteroatoms selected from N, O and S and wherein the heteroaryl is substituted by ((C₁-C₆)alkyl)ₚ-(C₅-C₆)cycloalkyl which is substituted by (C₅-C₆)heterocycle having up to 3 heteroatoms selected from N, O, and S which is substituted by (C₁-C₆)alkyl. In more particular embodiments of the present invention, R⁸ is a heteroaryl having up to 5 heteroatoms selected from N, O and S and wherein the heteroaryl is substituted by ((C₁-C₆)alkyl)ₚ-(C₅-C₆)cycloalkyl which is substituted by (C₅-C₆)heterocycle having up to 3 heteroatoms selected from N, O, and S which is substituted by (C₁-C₆)alkyl which is substituted by up to 3 halogen. In additional embodiments of the present invention, R¹ is a heteroaryl having up to 5 heteroatoms selected from N, O, and S and wherein the heteroaryl is substituted by O—(C₅-C₆)heterocycle having up to 3 heteroatoms selected from N, O, and S. In certain embodiments of the present invention, R⁸ is a heteroaryl having up to 5 heteroatoms selected from N, O, and S and wherein the heteroaryl is substituted by a (C₅-C₆) heteroaryl having up to 3 heteroatoms selected from N, O, and S. In other embodiments of the present invention, R¹ is a heteroaryl having up to 5 heteroatoms selected from N, O, and S and wherein the heteroaryl is substituted by a (C₅-C₆) heteroaryl having up to 3 heteroatoms selected from N, O, and S which is further substituted by (C₁-C₆) alkyl. In additional embodiments of the present invention, R⁸ is a heteroaryl having up to 5 heteroatoms selected from N, O, and S and wherein the heteroaryl is substituted by a (C₅-C₆) heteroaryl having up to 3 heteroatoms selected from N, O, and S which is further substituted by (C₁-C₆) alkyl which is further substituted by up to 3 halogen.

In some embodiments of the present invention, R⁸ is a cycloalkyl.

In particular embodiments of the present invention, n is 0.

In other particular embodiments of the present invention, n is 1.

In particular embodiments of the present invention, p is 0.

In other particular embodiments of the present invention, p is 1.

In specific embodiments of the present invention, the compound of formula I is selected from:
((1r),4R,5′S,7a′R)-4-(benzyloxy)-5′-phenyltetrahydro-3′H-spiro[cyclohexane-1,2′-pyrrolo[2,1-b]oxazol]-3′-one;
((1s),4R,5′S,7a′R)-4-(benzyloxy)-5′-phenyltetrahydro-3′H-spiro[cyclohexane-1,2′-pyrrolo[2,1-b]oxazol]-3′-one;
(5′S,7a′R)-3-(benzyloxy)-5′-phenyltetrahydro-3′H-spiro[cyclobutane-1,2′-pyrrolo[2,1-b]oxazol]-3′-one;
((1r),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one;
((1s),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one;
(1s,3R,5′S,7a′R)-3-(benzyloxy)-5′-(5-fluoropyridin-2-yl)tetrahydro-3′H-spiro[cyclobutane-1,2′-pyrrolo[2,1-b]oxazol]-3′-one;
((1r),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one;
((1s),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one;
cis 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carbonitrile;
trans 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carboxamide;
cis 6-((((1r),3R,5′(S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
trans 6-((((1s),3R,5′(S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
cis 6-((((1r),3R,5′(R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
trans 6-((((1s),3R,5′(R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
(1r,3R,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one;
3-(benzyloxy)-5′-(pyrazin-2-yl)tetrahydro-3′H-spiro[cyclobutane-1,2′-pyrrolo[2,1-b]oxazol]-3′-one;
(1s,3S,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one;
2-fluoro-4-(((1r,3R,5′S,7a′R)-3′-oxo-5′-phenyltetrahydro-3′H-spiro[cyclobutane-1,2′-pyrrolo[2,1-b]oxazol]-3-yl)oxy)benzonitrile;
5-{[(5′S,7a′R)-3′-oxo-5′-phenyltetrahydro-3′H-spiro[cyclobutane-1,2′-pyrrolo[2,1-b][1,3]oxazol]-3-yl]oxy}pyridine-2-carbonitrile;
4-fluoro-3-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-5′-phenyl-3-{[2-(trifluoromethyl)pyridin-4-yl]oxy}tetrahydro-3′H-spiro[cyclobutane-1,2′-pyrrolo[2,1-b][1,3]oxazol]-3′-one;
3-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-3-[(5-methoxypyridin-3-yl)oxy]-5′-phenyltetrahydro-3′H-spiro[cyclobutane-1,2′-pyrrolo[2,1-b][1,3]oxazol]-3′-one;
(5′S,7a′R)-3-[(3-fluoropyridin-4-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-{[5-(trifluoromethyl)pyridin-3-yl]oxy}tetrahydro-3′H-spiro[cyclobutane-1,2′-pyrrolo[2,1-b][1,3]oxazol]-3′-one;
4-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-3-(3-fluorophenoxy)-5′-phenyltetrahydro-3′H-spiro[cyclobutane-1,2′-pyrrolo[2,1-b][1,3]oxazol]-3′-one;
(5′S,7a′R)-3-[(2-chloropyridin-4-yl)oxy]-5′-phenyltetrahydro-3′H-spiro[cyclobutane-1,2′-pyrrolo[2,1-b][1,3]oxazol]-3′-one;
(5′S,7a′R)-3-[(1,2-benzothiazol-4-yl)oxy]-5′-phenyltetrahydro-3′H-spiro[cyclobutane-1,2′-pyrrolo[2,1-b][1,3]oxazol]-3′-one;
(5′S,7a′R)-3-(4-fluoro-3-methylphenoxy)-5′-phenyltetrahydro-3′H-spiro[cyclobutane-1,2′-pyrrolo[2,1-b][1,3]oxazol]-3′-one;
(5′S,7a′R)-3-[(1,2-benzothiazol-5-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-[3-(trifluoromethyl)phenoxy]tetrahydro-3′H-spiro[cyclobutane-1,2′-pyrrolo[2,1-b][1,3]oxazol]-3′-one;
(5′S,7a′R)-3-[3-(1,3,4-oxadiazol-2-yl)phenoxy]-5′-phenyltetrahydro-3′H-spiro[cyclobutane-1,2′-pyrrolo[2,1-b][1,3]oxazol]-3′-one;
(5′S,7a′R)-3-[(5-chloropyridin-3-yl)oxy]-5′-phenyltetrahydro-3′H-spiro[cyclobutane-1,2′-pyrrolo[2,1-b][1,3]oxazol]-3′-one;
(5′S,7a′R)-3-(4-fluoro-3-methoxyphenoxy)-5′-phenyltetrahydro-3′H-spiro[cyclobutane-1,2′-pyrrolo[2,1-b][1,3]oxazol]-3′-one;
(5′S,7a′R)-3-[(1,2-benzoxazol-7-yl)oxy]-5′-phenyltetrahydro-3′H-spiro[cyclobutane-1,2′-pyrrolo[2,1-b][1,3]oxazol]-3′-one;
(5′S,7a′R)-3-[(imidazo[1,2-c]pyrimidin-5-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-[(pyrrolo[1,2-b]pyridazin-4-yl)oxy]tetrahydro-3′H-spiro[cyclobutane-1,2′-pyrrolo[2,1-b][1,3]oxazol]-3′-one;
(1r,3R,5′S,7a′R)-5′-(4-fluorophenyl)-3-(pyrrolo[1,2-b]pyridazin-4-yloxy)tetrahydro-3′H-spiro[cyclobutane-1,2′-pyrrolo[2,1-b]oxazol]-3′-one;
(5′S,7a′R)-5′-phenyl-3-[(pyrrolo[1,2-d][1,2,4]triazin-4-yl)oxy]tetrahydro-3′H-spiro[cyclobutane-1,2′-pyrrolo[2,1-b][1,3]oxazol]-3′-one;
6-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrimidine-4-carbonitrile;
6-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
(5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one;
(1s,3S,5′S,7a′R)-5′-(3,5-difluorophenyl)-3-((3-fluoropyrazolo[1,5-a]pyrimidin-7-yl)oxy)tetrahydro-3′H-spiro[cyclobutane-1,2′-pyrrolo[2,1-b]oxazol]-3′-one;
(1r,3R,5′S,7a′R)-5′-(3,5-difluorophenyl)-3-((3-fluoropyrazolo[1,5-a]pyrimidin-7-yl)oxy)tetrahydro-3′H-spiro[cyclobutane-1,2′-pyrrolo[2,1-b]oxazol]-3′-one;
(5′S,7a′R)-5′-(3,5-difluorophenyl)-3-{[6-(1H-pyrazol-1-yl)pyrimidin-4-yl]oxy}tetrahydro-3′H-spiro[cyclobutane-1,2′-pyrrolo[2,1-b][1,3]oxazol]-3′-one;
(5′S,7a′R)-5′-(3,5-difluorophenyl)-3-[(2-methyl[1,3]thiazolo[5,4-d]pyrimidin-7-yl)oxy]tetrahydro-3′H-spiro[cyclobutane-1,2′-pyrrolo[2,1-b][1,3]oxazol]-3′-one;
(5′S,7a′R)-5′-(3,5-difluorophenyl)-3-{[6-(1H-pyrazol-1-yl)pyrimidin-4-yl]oxy}tetrahydro-3′H-spiro[cyclobutane-1,2′-pyrrolo[2,1-b][1,3]oxazol]-3′-one;
(1s,3S,5′S,7a′R)-5′-(3,5-difluorophenyl)-3-((6-(4-methyl-1H-imidazol-1-yl)pyrimidin-4-yl)oxy)tetrahydro-3′H-spiro[cyclobutane-1,2′-pyrrolo[2,1-b]oxazol]-3′-one;
(1s,3S,5′S,7a′R)-5′-(3,5-difluorophenyl)-3-((6-fluoropyrimidin-4-yl)oxy)tetrahydro-3′H-spiro[cyclobutane-1,2′-pyrrolo[2,1-b]oxazol]-3′-one;
(5′S,7a′R)-5′-(3,5-difluorophenyl)-3-{[6-(4-methyl-1H-imidazol-1-yl)pyrimidin-4-yl]oxy}tetrahydro-3′H-spiro[cyclobutane-1,2′-pyrrolo[2,1-b][1,3]oxazol]-3′-one;
6-(((1s,3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
(1s,3S,5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one;
3-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyridine-2-carbonitrile;
5-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyridine-3-carbonitrile;
6-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrazine-2-carbonitrile;
6-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-4-carbonitrile;
4-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-2-carbonitrile;
2-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-4-carbonitrile;
2-{[(1r,3R,5′S,7a′R)-5′-(3,5-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
(1r,3R,5′S,7a′R)-5′-(3,5-difluorophenyl)-3-[(2-methylpyrrolo[2,1-f][1,2,4]triazin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(thieno[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(furo[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(6-methylfuro[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(6-methylthieno[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(4-fluorophenyl)-3-[(imidazo[1,2-c]pyrimidin-5-yl)oxy]tetrahydro-3′H-spiro[cyclobutane-1,2′-pyrrolo[2,1-b][1,3]oxazol]-3′-one;
(1s,3S,5′S,7a′R)-5′-(3,5-difluorophenyl)-3-((5-fluoropyridin-2-yl)oxy)tetrahydro-3′H-spiro[cyclobutane-1,2′-pyrrolo[2,1-b]oxazol]-3′-one;
(5′S,7a′R)-3-[(2-chloropyridin-3-yl)oxy]-5′-(3,5-difluorophenyl)tetrahydro-3′H-spiro[cyclobutane-1,2′-pyrrolo[2,1-b][1,3]oxazol]-3′-one;
(5′S,7a′R)-5′-(3,5-difluorophenyl)-3-[(5-fluoropyridin-2-yl)oxy]tetrahydro-3′H-spiro[cyclobutane-1,2′-pyrrolo[2,1-b][1,3]oxazol]-3′-one;
cis (5′S,7a′R)-5′-(3,5-difluorophenyl)-3-[(3-fluoropyridin-2-yl)oxy]tetrahydro-3′H-spiro[cyclobutane-1,2′-pyrrolo[2,1-b][1,3]oxazol]-3′-one;
trans (5′S,7a′R)-5′-(3,5-difluorophenyl)-3-[(3-fluoropyridin-2-yl)oxy]tetrahydro-3′H-spiro[cyclobutane-1,2′-pyrrolo[2,1-b][1,3]oxazol]-3′-one;
cis 6-{[(5′S,7a′R)-5′-(2,3-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
trans 6-{[(5′S,7a′R)-5′-(2,3-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
6-(((5′S,7a′R)-3′-oxo-5′-(pyrazin-2-yl)tetrahydro-3′H-spiro[cyclobutane-1,2′-pyrrolo[2,1-b]oxazol]-3-yl)oxy)pyrimidine-4-carbonitrile;
6-{[(5′S,7a′R)-5′-(2-fluorophenyl)-3′-oxotetrahydro-3′H-spiro[cyclobutane-1,2′-pyrrolo[2,1-b][1,3]oxazol]-3-yl]oxy}pyrimidine-4-carbonitrile;
6-(((5′S,7a′R)-5′-(3-cyano-5-fluorophenyl)-3′-oxotetrahydro-3′H-spiro[cyclobutane-1,2′-pyrrolo[2,1-b]oxazol]-3-yl)oxy)pyrimidine-4-carbonitrile;
(1s,3S,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
(1r,3R,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
6-{[(5′S,7a′R)-5′-(5-fluoropyridin-2-yl)-3′-oxotetrahydro-3′H-spiro[cyclobutane-1,2′-pyrrolo[2,1-b][1,3]oxazol]-3-yl]oxy}pyrimidine-4-carbonitrile;
(1r,3R,5′S,7a′R)-5′-(4-fluorophenyl)-3-[(5-fluoropyridin-3-yl)oxy]tetrahydro-3′H-spiro[cyclobutane-1,2′-pyrrolo[2,1-b][1,3]oxazol]-3′-one;
5-{[(1r,3R,5′S,7a′R)-5′-(4-fluorophenyl)-3′-oxotetrahydro-3′H-spiro[cyclobutane-1,2′-pyrrolo[2,1-b][1,3]oxazol]-3-yl]oxy}pyridine-3-carbonitrile;
6-(((5′R,7a'S)-3′-oxo-5′-(pyrazin-2-yl)tetrahydro-3′H-spiro[cyclobutane-1,2′-pyrrolo[2,1-b]oxazol]-3-yl)oxy)pyrimidine-4-carbonitrile;
(1s,3R,5′R,7a'S)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
(5′S,7a′R)-5′-phenyl-4-[(pyrazin-2-yl)oxy]tetrahydro-3′H-spiro[cyclohexane-1,2′-pyrrolo[2,1-b][1,3]oxazol]-3′-one;
(5′S,7a′R)-5′-(3,5-difluorophenyl)-3-((6-(5-methyl-1,3,4-oxadiazol-2-yl)pyrimidin-4-yl)oxy)tetrahydro-3′H-spiro[cyclobutane-1,2′-pyrrolo[2,1-b]oxazol]-3′-one;
(5′S,7a′R)-3-((6-(1-(difluoromethyl)-1H-pyrazol-4-yl)pyrimidin-4-yl)oxy)-5′-(3,5-difluorophenyl)tetrahydro-3′H-spiro[cyclobutane-1,2′-pyrrolo[2,1-b]oxazol]-3′-one;
(5′S,7a′R)-5′-(3,5-difluorophenyl)-3-((6-(1-methyl-1H-pyrazol-5-yl)pyrimidin-4-yl)oxy)tetrahydro-3′H-spiro[cyclobutane-1,2′-pyrrolo[2,1-b]oxazol]-3′-one;
(5′S,7a′R)-3-((6-(4-(difluoromethyl)-1H-pyrazol-1-yl)pyrimidin-4-yl)oxy)-5′-(3,5-difluorophenyl)tetrahydro-3′H-spiro[cyclobutane-1,2′-pyrrolo[2,1-b]oxazol]-3′-one;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinamide;
(1r,3R,5′S,7a′R)-3-((4-(2H-tetrazol-5-yl)pyridin-2-yl)oxy)-5′-(3,5-difluorophenyl)tetrahydro-3′H-spiro[cyclobutane-1,2′-pyrrolo[2,1-b]oxazol]-3′-one;
2-(((1r,3R,5′S,7a′R)-3′-oxo-5′-phenyltetrahydro-3′H-spiro[cyclobutane-1,2′-pyrrolo[2,1-b]oxazol]-3-yl)oxy)isonicotinonitrile;
2-(((1r,3R,5′S,7a′R)-3′-oxo-5′-phenyltetrahydro-3′H-spiro[cyclobutane-1,2′-pyrrolo[2,1-b]oxazol]-3-yl)oxy)isonicotinamide;
2-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)isonicotinamide;
(1r,3R,5′S,7a′R)-3-((4-(2H-tetrazol-5-yl)pyridin-2-yl)oxy)-5′-(4-fluorophenyl)tetrahydro-3′H-spiro[cyclobutane-1,2′-pyrrolo[2,1-b]oxazol]-3′-one;
(1r,3R,5′S,7a′R)-3-((7-bromopyrrolo[2,1-f][1,2,4]triazin-4-yl)oxy)-5′-(3,5-difluorophenyl)tetrahydro-3′H-spiro[cyclobutane-1,2′-pyrrolo[2,1-b]oxazol]-3′-one;
4-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile;
4-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile;
cis (1R,3R,5′S,7a′R)-5′-(3,5-difluorophenyl)-3-((R)-1-(5-fluoropyridin-2-yl)ethoxy)tetrahydro-3′H-spiro[cyclobutane-1,2′-pyrrolo[2,1-b]oxazol]-3′-one;
trans (1R,3R,5′S,7a′R)-5′-(3,5-difluorophenyl)-3-((S)-1-(5-fluoropyridin-2-yl)ethoxy)tetrahydro-3′H-spiro[cyclobutane-1,2′-pyrrolo[2,1-b]oxazol]-3′-one;
(1R,3R,5′S,7a′R)-5′-(3,5-difluorophenyl)-3-((R)-1-(3-fluoropyridin-2-yl)ethoxy)tetrahydro-3′H-spiro[cyclobutane-1,2′-pyrrolo[2,1-b]oxazol]-3′-one;
(1S,3S,5′S,7a′R)-5′-(3,5-difluorophenyl)-3-((S)-1-(3-fluoropyridin-2-yl)ethoxy)tetrahydro-3′H-spiro[cyclobutane-1,2′-pyrrolo[2,1-b]oxazol]-3′-one;
(1R,3S,5′S,7a′R)-5′-(3,5-difluorophenyl)-3-((s)-1-(3-fluoropyridin-2-yl)ethoxy)tetrahydro-3′H-spiro[cyclobutane-1,2′-pyrrolo[2,1-b]oxazol]-3′-one;
(1r,3R,5′S,7a′R)-5′-(3,5-difluorophenyl)-3′-oxotetrahydro-3′H-spiro[cyclobutane-1,2′-pyrrolo[2,1-b]oxazol]-3-yl picolinate;
(1r,3R,5′S,7a′R)-5′-(3,5-difluorophenyl)-3′-oxotetrahydro-3′H-spiro[cyclobutane-1,2′-pyrrolo[2,1-b]oxazol]-3-yl benzoate;
((1S),3R,5′S,7a′R)-5′-(3,5-difluorophenyl)-3-((S)-1-phenylethoxy)tetrahydro-3′H-spiro[cyclobutane-1,2′-pyrrolo[2,1-b]oxazol]-3′-one;
((1R),3R,5′S,7a′R)-5′-(3,5-difluorophenyl)-3-((S)-1-phenylethoxy)tetrahydro-3′H-spiro[cyclobutane-1,2′-pyrrolo[2,1-b]oxazol]-3′-one;
((1S),3R,5′S,7a′R)-5′-(3,5-difluorophenyl)-3-((R)-1-phenylethoxy)tetrahydro-3′H-spiro[cyclobutane-1,2′-pyrrolo[2,1-b]oxazol]-3′-one;
((1R),3R,5′S,7a′R)-5′-(3,5-difluorophenyl)-3-((R)-1-phenylethoxy)tetrahydro-3′H-spiro[cyclobutane-1,2′-pyrrolo[2,1-b]oxazol]-3′-one;
((1S),3R,5′S,7a′R)-5′-(3,5-difluorophenyl)-3-phenoxytetrahydro-3′H-spiro[cyclobutane-1,2′-pyrrolo[2,1-b]oxazol]-3′-one;
((1R),3R,5′S,7a′R)-5′-(3,5-difluorophenyl)-3-phenoxytetrahydro-3′H-spiro[cyclobutane-1,2′-pyrrolo[2,1-b]oxazol]-3′-one;
(1R),(5′S,7a′R)-5′-(5-fluoropyridin-3-yl)-4-phenoxytetrahydro-3′H-spiro[cyclohexane-1,2′-pyrrolo[2,1-b]oxazol]-3′-one;
(1S), (5′S,7a′R)-5′-(5-fluoropyridin-3-yl)-4-phenoxytetrahydro-3′H-spiro[cyclohexane-1,2′-pyrrolo[2,1-b]oxazol]-3′-one;
2-((((1S),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile;
2-((((1R),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile;
(5′S,7a′R)-5′-(4-fluorophenyl)-3-[(imidazo[1,2-a]pyrazin-8-yl)oxy]tetrahydro-3′H-spiro[cyclobutane-1,2′-pyrrolo[2,1-b][1,3]oxazol]-3′-one;
(5′S,7a′R)-5′-(4-fluorophenyl)-3-[([1,2,4]triazolo[1,5-a]pyrazin-8-yl)oxy]tetrahydro-3′H-spiro[cyclobutane-1,2′-pyrrolo[2,1-b][1,3]oxazol]-3′-one;
(5′S,7a′R)-5′-(4-fluorophenyl)-3-[(pyridin-3-yl)methoxy]tetrahydro-3′H-spiro[cyclobutane-1,2′-pyrrolo[2,1-b][1,3]oxazol]-3′-one;
(5′S,7a′R)-5′-(4-fluorophenyl)-3-[(pyridin-4-yl)methoxy]tetrahydro-3′H-spiro[cyclobutane-1,2′-pyrrolo[2,1-b][1,3]oxazol]-3′-one;
(5′S,7a′R)-5′-(3-fluoropyridin-2-yl)-3-[(pyrrolo[2,1-f][1,2,4]triazin-4-yl)oxy]tetrahydro-3′H-spiro[cyclobutane-1,2′-pyrrolo[2,1-b][1,3]oxazol]-3′-one;
(1r,3R,5′S,7a′R)-5′-(1-methyl-1H-pyrazol-3-yl)-3-[(pyrrolo[2,1-f][1,2,4]triazin-4-yl)oxy]tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-[(pyrazin-2-yl)oxy]tetrahydro-3′H-spiro[cyclobutane-1,2′-pyrrolo[2,1-b][1,3]oxazol]-3′-one;
(1S,3S,5′S,7a′R)-5′-(pyrazin-2-yl)-3-(pyridin-3-ylmethoxy)tetrahydro-3′H-spiro[cyclobutane-1,2′-pyrrolo[2,1-b]oxazol]-3′-one;
(5′S)-5′-(pyrazin-2-yl)-3-[([1,2,4]triazolo[4,3-a]pyrimidin-6-yl)methoxy]tetrahydro-3′H-spiro[cyclobutane-1,2′-pyrrolo[2,1-b][1,3]oxazol]-3′-one;
(5′S)-3-[(1-cyclopropyl-1H-pyrazol-4-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(pyrazolo[1,5-a]pyrimidin-3-yl)methoxy]tetrahydro-3′H-spiro[cyclobutane-1,2′-pyrrolo[2,1-b][1,3]oxazol]-3′-one;
(5′S)-3-[(1-methyl-1H-pyrazol-4-yl)methoxy]-5′-(pyrazin-2-yl)tetrahydro-3′H-spiro[cyclobutane-1,2′-pyrrolo[2,1-b][1,3]oxazol]-3′-one;
(5′S,7a′R)-3-{[1-(bicyclo[1.1.1]pentan-1-yl)-1H-pyrazol-4-yl]methoxy}-5′-(pyrazin-2-yl)tetrahydro-3′H-spiro[cyclobutane-1,2′-pyrrolo[2,1-b][1,3]oxazol]-3′-one;
(5′S)-3-[(1-phenyl-1H-pyrazol-4-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(2-ethoxypyrimidin-5-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-({3-[2-(4H-1,2,4-triazol-4-yl)ethyl]phenyl}methoxy)tetrahydro-3′H-spiro[cyclobutane-1,2′-pyrrolo[2,1-b][1,3]oxazol]-3′-one;
[3-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]acetonitrile;
(5′S)-3-{[5-(difluoromethyl)pyridin-2-yl]methoxy}-5′-(pyrazin-2-yl)tetrahydro-3′H-spiro[cyclobutane-1,2′-pyrrolo[2,1-b][1,3]oxazol]-3′-one;
(5′S)-3-[(5-benzyl-1,3,4-thiadiazol-2-yl)methoxy]-5′-(pyrazin-2-yl)tetrahydro-3′H-spiro[cyclobutane-1,2′-pyrrolo[2,1-b][1,3]oxazol]-3′-one;
(5′S)-3-({6-[(butan-2-yl)oxy]pyridin-2-yl}methoxy)-5′-(pyrazin-2-yl)tetrahydro-3′H-spiro[cyclobutane-1,2′-pyrrolo[2,1-b][1,3]oxazol]-3′-one;
5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)quinoline-8-carbonitrile;
(5′S)-3-{[5-(4-methyl-1H-pyrazol-1-yl)pyrazin-2-yl]methoxy}-5′-(pyrazin-2-yl)tetrahydro-3′H-spiro[cyclobutane-1,2′-pyrrolo[2,1-b][1,3]oxazol]-3′-one;
(5′S)-3-{[3-(methylsulfanyl)[1,2,4]triazolo[4,3-a]pyridin-6-yl]methoxy}-5′-(pyrazin-2-yl)tetrahydro-3′H-spiro[cyclobutane-1,2′-pyrrolo[2,1-b][1,3]oxazol]-3′-one;
(5′S)-3-{[4-(methylsulfonyl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(thieno[2,3-b]pyridin-3-yl)methoxy]tetrahydro-3′H-spiro[cyclobutane-1,2′-pyrrolo[2,1-b][1,3]oxazol]-3′-one;
(5′S)-3-[(imidazo[1,2-a]pyridin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(thieno[2,3-d]pyrimidin-6-yl)methoxy]tetrahydro-3′H-spiro[cyclobutane-1,2′-pyrrolo[2,1-b][1,3]oxazol]-3′-one;
(5′S)-3-[(4-fluoroisoquinolin-5-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(2-cyclopropylimidazo[1,2-a]pyridin-6-yl)methoxy]-5′-(pyrazin-2-yl)tetrahydro-3′H-spiro[cyclobutane-1,2′-pyrrolo[2,1-b][1,3]oxazol]-3′-one;
(1s,3S,5′S,7a′R)-3-[(1,3-benzothiazol-5-yl)methoxy]-5′-(pyrazin-2-yl)tetrahydro-3′H-spiro[cyclobutane-1,2′-pyrrolo[2,1-b][1,3]oxazol]-3′-one;
(5′S)-3-{[2-(morpholin-4-yl)pyrimidin-5-yl]methoxy}-5′-(pyrazin-2-yl)tetrahydro-3′H-spiro[cyclobutane-1,2′-pyrrolo[2,1-b][1,3]oxazol]-3′-one;
(5′S)-3-({4-[(1H-imidazol-1-yl)methyl]phenyl}methoxy)-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-({3-[(1H-1,2,3-triazol-1-yl)methyl]phenyl}methoxy)tetrahydro-3′H-spiro[cyclobutane-1,2′-pyrrolo[2,1-b][1,3]oxazol]-3′-one;
(5′S)-3-{[5-(2-fluoroethoxy)pyridin-3-yl]methoxy}-5′-(pyrazin-2-yl)tetrahydro-3′H-spiro[cyclobutane-1,2′-pyrrolo[2,1-b][1,3]oxazol]-3′-one;
1-[5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridin-2-yl]cyclobutane-1-carbonitrile;
1-[3-chloro-4-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]cyclopropane-1-carbonitrile;
(5′S)-3-{[4-(1,2-oxazol-3-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(pyrazolo[1,5-a]pyrimidin-7-yl)methoxy]tetrahydro-3′H-spiro[cyclobutane-1,2′-pyrrolo[2,1-b][1,3]oxazol]-3′-one;
(5′S)-3-[(2-methoxy-1,7-naphthyridin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-3-{[5-chloro-2-(1,3-oxazol-5-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-[(5-methoxyimidazo[1,2-a]pyridin-7-yl)methoxy]-5′-(pyrazin-2-yl)tetrahydro-3′H-spiro[cyclobutane-1,2′-pyrrolo[2,1-b][1,3]oxazol]-3′-one;
(5′S)-3-{[6-(morpholin-4-yl)pyridin-3-yl]methoxy}-5′-(pyrazin-2-yl)tetrahydro-3′H-spiro[cyclobutane-1,2′-pyrrolo[2,1-b][1,3]oxazol]-3′-one;
(5′S)-3-[(imidazo[1,2-a]pyridin-5-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(isoquinolin-6-yl)methoxy]-5′-(pyrazin-2-yl)tetrahydro-3′H-spiro[cyclobutane-1,2′-pyrrolo[2,1-b][1,3]oxazol]-3′-one;
[2-methoxy-5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]acetonitrile;
(5′S)-3-{[4-(1H-imidazol-1-yl)pyridin-2-yl]methoxy}-5′-(pyrazin-2-yl)tetrahydro-3′H-spiro[cyclobutane-1,2′-pyrrolo[2,1-b][1,3]oxazol]-3′-one;
4-(cyclobutyloxy)-2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)benzonitrile;
(5′S)-3-[(2-methoxy-8-methylquinolin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(5,6,7,8-tetrahydroquinolin-3-yl)methoxy]tetrahydro-3′H-spiro[cyclobutane-1,2′-pyrrolo[2,1-b][1,3]oxazol]-3′-one;
(5′S)-5′-(pyrazin-2-yl)-3-[(thieno[3,2-b]pyridin-2-yl)methoxy]tetrahydro-3′H-spiro[cyclobutane-1,2′-pyrrolo[2,1-b][1,3]oxazol]-3′-one;
(5′S)-3-{[6-(2-fluoroethoxy)pyridazin-3-yl]methoxy}-5′-(pyrazin-2-yl)tetrahydro-3′H-spiro[cyclobutane-1,2′-pyrrolo[2,1-b][1,3]oxazol]-3′-one;
3-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridine-4-carbonitrile;
(5′S)-3-[(imidazo[1,2-a]pyrimidin-6-yl)methoxy]-5′-(pyrazin-2-yl)tetrahydro-3′H-spiro[cyclobutane-1,2′-pyrrolo[2,1-b][1,3]oxazol]-3′-one;
3-[4-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]propanenitrile;
(5′S)-5′-(pyrazin-2-yl)-3-{[2-(trifluoromethyl)-1,3-benzothiazol-6-yl]methoxy}tetrahydro-3′H-spiro[cyclobutane-1,2′-pyrrolo[2,1-b][1,3]oxazol]-3′-one;
[3-chloro-5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridin-2-yl]acetonitrile;
(5′S)-3-{[4-(2-methoxyethoxy)phenyl]methoxy}-5′-(pyrazin-2-yl)tetrahydro-3′H-spiro[cyclobutane-1,2′-pyrrolo[2,1-b][1,3]oxazol]-3′-one;
3-fluoro-2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)benzonitrile;
(5′S)-5′-(pyrazin-2-yl)-3-{[1-(pyridin-4-yl)-1H-pyrazol-4-yl]methoxy}tetrahydro-3′H-spiro[cyclobutane-1,2′-pyrrolo[2,1-b][1,3]oxazol]-3′-one;
(5′S)-5′-(pyrazin-2-yl)-3-{[2-(1H-pyrazol-1-yl)phenyl]methoxy}tetrahydro-3′H-spiro[cyclobutane-1,2′-pyrrolo[2,1-b][1,3]oxazol]-3′-one;
(5′S)-5′-(pyrazin-2-yl)-3-{[2-(trifluoromethyl)imidazo[1,2-a]pyridin-6-yl]methoxy}tetrahydro-3′H-spiro[cyclobutane-1,2′-pyrrolo[2,1-b][1,3]oxazol]-3′-one;
2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)furan-3-carbonitrile;
(5′S)-3-{[4-(5-ethyl-1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-({6-[(oxan-4-yl)oxy]pyridin-3-yl}methoxy)-5′-(pyrazin-2-yl)tetrahydro-3′H-spiro[cyclobutane-1,2′-pyrrolo[2,1-b][1,3]oxazol]-3′-one;
(5′S,7a′R)-3-{[4-(1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-[(1,2-benzoxazol-5-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(pyrazolo[1,5-a]pyrimidin-7-yl)methoxy]tetrahydro-3′H-spiro[cyclobutane-1,2′-pyrrolo[2,1-b][1,3]oxazol]-3′-one;
(5′S)-3-[(1,3-benzothiazol-5-yl)methoxy]-5′-(pyrazin-2-yl)tetrahydro-3′H-spiro[cyclobutane-1,2′-pyrrolo[2,1-b][1,3]oxazol]-3′-one;
[3-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]acetonitrile;
(5′S)-3-{[5-(difluoromethyl)pyridin-2-yl]methoxy}-5′-(pyrazin-2-yl)tetrahydro-3′H-spiro[cyclobutane-1,2′-pyrrolo[2,1-b][1,3]oxazol]-3′-one;
(5′S)-3-[(5-benzyl-1,3,4-thiadiazol-2-yl)methoxy]-5′-(pyrazin-2-yl)tetrahydro-3′H-spiro[cyclobutane-1,2′-pyrrolo[2,1-b][1,3]oxazol]-3′-one;
5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)quinoline-8-carbonitrile;
(5′S,7a′R)-3-({6-[(butan-2-yl)oxy]pyridin-2-yl}methoxy)-5′-(pyrazin-2-yl)tetrahydro-3′H-spiro[cyclobutane-1,2′-pyrrolo[2,1-b][1,3]oxazol]-3′-one;
(5′S)-3-{[4-(methylsulfonyl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(thieno[2,3-b]pyridin-3-yl)methoxy]tetrahydro-3′H-spiro[cyclobutane-1,2′-pyrrolo[2,1-b][1,3]oxazol]-3′-one;
(5′S)-3-[(imidazo[1,2-a]pyridin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(6-methoxypyridin-3-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(2-cyclopropyl-1,3-thiazol-4-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(4-fluoroisoquinolin-5-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(2-cyclopropylimidazo[1,2-a]pyridin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-3-({4-[(1H-imidazol-1-yl)methyl]phenyl}methoxy)-5′-(pyrazin-2-yl)tetrahydro-3′H-spiro[cyclobutane-1,2′-pyrrolo[2,1-b][1,3]oxazol]-3′-one;
5-methoxy-6-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridine-2-carbonitrile;
(5′S)-5′-(pyrazin-2-yl)-3-({3-[(1H-1,2,3-triazol-1-yl)methyl]phenyl}methoxy)tetrahydro-3′H-spiro[cyclobutane-1,2′-pyrrolo[2,1-b][1,3]oxazol]-3′-one;
(5′S)-3-{[5-(2-fluoroethoxy)pyridin-3-yl]methoxy}-5′-(pyrazin-2-yl)tetrahydro-3′H-spiro[cyclobutane-1,2′-pyrrolo[2,1-b][1,3]oxazol]-3′-one;
1-[5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridin-2-yl]cyclobutane-1-carbonitrile;
1-[3-chloro-4-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]cyclopropane-1-carbonitrile;
(5′S)-3-{[4-(1,2-oxazol-3-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-[(2-methoxy-1,7-naphthyridin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-3-{[5-chloro-2-(1,3-oxazol-5-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-[(5-methoxyimidazo[1,2-a]pyridin-7-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(imidazo[1,2-a]pyridin-5-yl)methoxy]-5′-(pyrazin-2-yl)tetrahydro-3′H-spiro[cyclobutane-1,2′-pyrrolo[2,1-b][1,3]oxazol]-3′-one;
(5′S)-3-[(isoquinolin-6-yl)methoxy]-5′-(pyrazin-2-yl)tetrahydro-3′H-spiro[cyclobutane-1,2′-pyrrolo[2,1-b][1,3]oxazol]-3′-one;
[2-methoxy-5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]acetonitrile;
(5′S)-3-{[4-(1H-imidazol-1-yl)pyridin-2-yl]methoxy}-5′-(pyrazin-2-yl)tetrahydro-3′H-spiro[cyclobutane-1,2′-pyrrolo[2,1-b][1,3]oxazol]-3′-one;
4-(cyclobutyloxy)-2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)benzonitrile;
(5′S)-3-[(2-methoxy-8-methylquinolin-6-yl)methoxy]-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-[(5,6,7,8-tetrahydroquinolin-3-yl)methoxy]tetrahydro-3′H-spiro[cyclobutane-1,2′-pyrrolo[2,1-b][1,3]oxazol]-3′-one;
(5′S)-5′-(pyrazin-2-yl)-3-[(thieno[3,2-b]pyridin-2-yl)methoxy]tetrahydro-3′H-spiro[cyclobutane-1,2′-pyrrolo[2,1-b][1,3]oxazol]-3′-one;
(5′S)-3-[(2,3-dihydro-1-benzofuran-5-yl)methoxy]-5′-(pyrazin-2-yl)tetrahydro-3′H-spiro[cyclobutane-1,2′-pyrrolo[2,1-b][1,3]oxazol]-3′-one;
3-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridine-4-carbonitrile;
(5′S)-3-[(imidazo[1,2-a]pyrimidin-6-yl)methoxy]-5′-(pyrazin-2-yl)tetrahydro-3′H-spiro[cyclobutane-1,2′-pyrrolo[2,1-b][1,3]oxazol]-3′-one;
3-[4-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]propanenitrile;
(5′S)-5′-(pyrazin-2-yl)-3-{[6-(pyrrolidin-1-yl)pyridin-2-yl]methoxy}tetrahydro-3′H-spiro[cyclobutane-1,2′-pyrrolo[2,1-b][1,3]oxazol]-3′-one;
(5′S)-5′-(pyrazin-2-yl)-3-{[2-(trifluoromethyl)-1,3-benzothiazol-6-yl]methoxy}tetrahydro-3′H-spiro[cyclobutane-1,2′-pyrrolo[2,1-b][1,3]oxazol]-3′-one;
[3-chloro-5-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)pyridin-2-yl]acetonitrile;
(5′S)-3-{[4-(2-methoxyethoxy)phenyl]methoxy}-5′-(pyrazin-2-yl)tetrahydro-3′H-spiro[cyclobutane-1,2′-pyrrolo[2,1-b][1,3]oxazol]-3′-one;
3-fluoro-2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)benzonitrile;
(5′S)-5′-(pyrazin-2-yl)-3-{[1-(pyridin-4-yl)-1H-pyrazol-4-yl]methoxy}tetrahydro-3′H-spiro[cyclobutane-1,2′-pyrrolo[2,1-b][1,3]oxazol]-3′-one;
(5′S)-5′-(pyrazin-2-yl)-3-{[2-(1H-pyrazol-1-yl)phenyl]methoxy}tetrahydro-3′H-spiro[cyclobutane-1,2′-pyrrolo[2,1-b][1,3]oxazol]-3′-one;
(5′S)-5′-(pyrazin-2-yl)-3-{[2-(trifluoromethyl)imidazo[1,2-a]pyridin-6-yl]methoxy}tetrahydro-3′H-spiro[cyclobutane-1,2′-pyrrolo[2,1-b][1,3]oxazol]-3′-one;
2-({[(5′S)-3′-oxo-5′-(pyrazin-2-yl)tetrahydro-3′H-spiro[cyclobutane-1,2′-pyrrolo[2,1-b][1,3]oxazol]-3-yl]oxy}methyl)furan-3-carbonitrile;
(5′S)-3-{[4-(5-ethyl-1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S)-3-({6-[(oxan-4-yl)oxy]pyridin-3-yl}methoxy)-5′-(pyrazin-2-yl)tetrahydro-3′H-spiro[cyclobutane-1,2′-pyrrolo[2,1-b][1,3]oxazol]-3′-one;
(5′S)-5′-(pyrazin-2-yl)-3-(thieno[2,3-d]pyrimidin-6-ylmethoxy)tetrahydro-3′H-spiro[cyclobutane-1,2′-pyrrolo[2,1-b]oxazol]-3′-one;
(5′S)-3-((2,3-dihydrobenzofuran-5-yl)methoxy)-5′-(pyrazin-2-yl)tetrahydro-3′H-spiro[cyclobutane-1,2′-pyrrolo[2,1-b]oxazol]-3′-one;
(5′S,7a′R)-5′-(pyrazin-2-yl)-3-(pyridin-3-yloxy)tetrahydro-3′H-spiro[cyclobutane-1,2′-pyrrolo[2,1-b]oxazol]-3′-one;
(5′S,7a′R)-5′-(1-methyl-1H-pyrazol-3-yl)-3-(3-(1-methyl-1H-pyrazol-5-yl)phenoxy)tetrahydro-3′H-spiro[cyclobutane-1,2′-pyrrolo[2,1-b]oxazol]-3′-one;
3-{[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}benzonitrile;
3-fluoro-5-{[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}benzonitrile;
(5′S,7a′R)-3-(3,4-difluorophenoxy)-5′-(pyrazin-2-yl)tetrahydro-3′H-spiro[cyclobutane-1,2′-pyrrolo[2,1-b][1,3]oxazol]-3′-one;
3-{[(1s,3S,5′S,7a′R)-5′-(1-methyl-1H-pyrazol-3-yl)-3′-oxotetrahydro-3′H-spiro[cyclobutane-1,2′-pyrrolo[2,1-b][1,3]oxazol]-3-yl]oxy}benzonitrile;
(5′S,7a′R)-5′-(1-methyl-1H-pyrazol-3-yl)-3-[(6-methylpyridin-3-yl)oxy]tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-[(pyridin-3-yl)oxy]tetrahydro-3′H-spiro[cyclobutane-1,2′-pyrrolo[2,1-b][1,3]oxazol]-3′-one;
(5′S,7a′R)-3-[(5-chloropyridin-3-yl)oxy]-5′-(1-methyl-1H-pyrazol-3-yl)tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-[3-(1-methyl-1H-pyrazol-5-yl)phenoxy]tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-[(pyridin-3-yl)oxy]tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-[(6-methylpyridin-3-yl)oxy]tetrahydro-3′H-spiro[cyclobutane-1,2′-pyrrolo[2,1-b][1,3]oxazol]-3′-one;
(5′S,7a′R)-5′-(1-methyl-1H-pyrazol-3-yl)-3-(3-(1-methyl-1H-pyrazol-5-yl)phenoxy)tetrahydro-3′H-spiro[cyclobutane-1,2′-pyrrolo[2,1-b]oxazol]-3′-one;
(5′S,7a′R)-5′-(pyrazin-2-yl)-3-[(pyridin-2-yl)methoxy]tetrahydro-3′H-spiro[cyclobutane-1,2′-pyrrolo[2,1-b][1,3]oxazol]-3′-one;
(5′S,7a′R)-3-[(2-methyl-2H-indazol-5-yl)methoxy]-5′-(pyrazin-2-yl)tetrahydro-3′H-spiro[cyclobutane-1,2′-pyrrolo[2,1-b][1,3]oxazol]-3′-one;
(5′S,7a′R)-5′-(pyrazin-2-yl)-3-[([1,2,4]triazolo[4,3-a]pyrimidin-5-yl)methoxy]tetrahydro-3′H-spiro[cyclobutane-1,2′-pyrrolo[2,1-b][1,3]oxazol]-3′-one;
(5′S,7a′R)-3-[(2-methylpyridin-3-yl)methoxy]-5′-(pyrazin-2-yl)tetrahydro-3′H-spiro[cyclobutane-1,2′-pyrrolo[2,1-b][1,3]oxazol]-3′-one;
(5′S,7a′R)-5′-(pyrazin-2-yl)-3-{[2-(trifluoromethyl)pyridin-3-yl]methoxy}tetrahydro-3′H-spiro[cyclobutane-1,2′-pyrrolo[2,1-b][1,3]oxazol]-3′-one;
(5′S,7a′R)-3-[(1,3-benzothiazol-5-yl)methoxy]-5′-(pyrazin-2-yl)tetrahydro-3′H-spiro[cyclobutane-1,2′-pyrrolo[2,1-b][1,3]oxazol]-3′-one;
1-[4-({[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}methyl)phenyl]cyclopropane-1-carbonitrile;
(5′S,7a′R)-3-[(5-chloropyridin-3-yl)oxy]-5′-(1-methyl-1H-pyrazol-3-yl)tetrahydro-3′H-spiro[cyclobutane-1,2′-pyrrolo[2,1-b][1,3]oxazol]-3′-one;
cis (1s,3S,5′S,7'S,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one;
trans (1s,3S,5′S,7′R,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one;
5′-(5-fluoropyridin-2-yl)-3-[(pyrrolo[2,1-f][1,2,4]triazin-4-yl)oxy]tetrahydro-3′H-spiro[cyclobutane-1,2′-pyrrolo[2,1-b][1,3]oxazol]-3′-one;
(5′S,7a′R)-5′-(pyrazin-2-yl)-3-[(pyrazin-2-yl)oxy]tetrahydro-3′H-spiro[cyclobutane-1,2′-pyrrolo[2,1-b][1,3]oxazol]-3′-one;
(5′S,7a′R)-3-(2,5-difluorophenoxy)-5′-(pyrazin-2-yl)tetrahydro-3′H-spiro[cyclobutane-1,2′-pyrrolo[2,1-b][1,3]oxazol]-3′-one;
3-((((5′S,7a′R)-3′-oxo-5′-(pyrazin-2-yl)tetrahydro-3′H-spiro[cyclobutane-1,2′-pyrrolo[2,1-b]oxazol]-3-yl)oxy)methyl)benzonitrile;
(5′S,7a′R)-3-(benzo[d]isoxazol-6-ylmethoxy)-5′-(pyrazin-2-yl)tetrahydro-3′H-spiro[cyclobutane-1,2′-pyrrolo[2,1-b]oxazol]-3′-one;
cis (5′S,7a′R)-3-[3-(1-methyl-1H-pyrazol-5-yl)phenoxy]-5′-(pyrazin-2-yl)tetrahydro-3′H-spiro[cyclobutane-1,2′-pyrrolo[2,1-b][1,3]oxazol]-3′-one;
trans (5′S,7a′R)-3-[3-(1-methyl-1H-pyrazol-5-yl)phenoxy]-5′-(pyrazin-2-yl)tetrahydro-3′H-spiro[cyclobutane-1,2′-pyrrolo[2,1-b][1,3]oxazol]-3′-one;
3-{[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}benzonitrile;
(5′S,7a′R)-3-phenoxy-5′-(pyrazin-2-yl)tetrahydro-3′H-spiro[cyclobutane-1,2′-pyrrolo[2,1-b][1,3]oxazol]-3′-one;
(5′S,7a′R)-3-[(5-chloropyridin-3-yl)oxy]-5′-(pyrazin-2-yl)tetrahydro-3′H-spiro[cyclobutane-1,2′-pyrrolo[2,1-b][1,3]oxazol]-3′-one;
(5′S,7a′R)-5′-(pyrazin-2-yl)-3-[(thieno[2,3-b]pyridin-3-yl)methoxy]tetrahydro-3′H-spiro[cyclobutane-1,2′-pyrrolo[2,1-b][1,3]oxazol]-3′-one;
(5′S,7a′R)-3-{[6-(1-methyl-1H-pyrazol-5-yl)pyrimidin-4-yl]oxy}-5′-(pyrazin-2-yl)tetrahydro-3′H-spiro[cyclobutane-1,2′-pyrrolo[2,1-b][1,3]oxazol]-3′-one;
(5′S,7a′R)-5′-(pyrazin-2-yl)-3-[(pyridin-3-yl)oxy]tetrahydro-3′H-spiro[cyclobutane-1,2′-pyrrolo[2,1-b][1,3]oxazol]-3′-one;
(5′S,7a′R)-3-(3,4-difluorophenoxy)-5′-(pyrazin-2-yl)tetrahydro-3′H-spiro[cyclobutane-1,2′-pyrrolo[2,1-b][1,3]oxazol]-3′-one;
(1S,3S,5′S,7a′R)-3-((2-chlorobenzyl)oxy)-5′-(pyrazin-2-yl)tetrahydro-3′H-spiro[cyclobutane-1,2′-pyrrolo[2,1-b]oxazol]-3′-one;
3-fluoro-5-{[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}benzonitrile;
4-((((5′S,7a′R)-3′-oxo-5′-(pyrazin-2-yl)tetrahydro-3′H-spiro[cyclobutane-1,2′-pyrrolo[2,1-b]oxazol]-3-yl)oxy)methyl)benzonitrile;
(5′S,7a′R)-3-((5-chloro-3-methylpyridin-2-yl)methoxy)-5′-(pyrazin-2-yl)tetrahydro-3′H-spiro[cyclobutane-1,2′-pyrrolo[2,1-b]oxazol]-3′-one;
(5′S,7a′R)-5′-(pyrazin-2-yl)-3-(thiophen-2-ylmethoxy)tetrahydro-3′H-spiro[cyclobutane-1,2′-pyrrolo[2,1-b]oxazol]-3′-one;
(5′S,7a′R)-3-[(3-fluorophenyl)methoxy]-5′-(pyrazin-2-yl)tetrahydro-3′H-spiro[cyclobutane-1,2′-pyrrolo[2,1-b][1,3]oxazol]-3′-one;
(5′S,7a′R)-3-[(3-chlorophenyl)methoxy]-5′-(pyrazin-2-yl)tetrahydro-3′H-spiro[cyclobutane-1,2′-pyrrolo[2,1-b][1,3]oxazol]-3′-one;
4-({[(5′S,7a′R)-3′-oxo-5′-(pyrazin-2-yl)tetrahydro-3′H-spiro[cyclobutane-1,2′-pyrrolo[2,1-b][1,3]oxazol]-3-yl]oxy}methyl)benzonitrile;
(5′S,7a′R)-3-[(3-fluorophenyl)methoxy]-5′-(pyrazin-2-yl)tetrahydro-3′H-spiro[cyclobutane-1,2′-pyrrolo[2,1-b][1,3]oxazol]-3′-one;
(5′S,7a′R)-3-{[4-(5-methyl-1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one;
(5′S,7a′R)-3-(cyclobutylmethoxy)-5′-(pyrazin-2-yl)tetrahydro-3′H-spiro[cyclobutane-1,2′-pyrrolo[2,1-b]oxazol]-3′-one;
cis (5′S,7a′R)-3-(cyclopentylmethoxy)-5′-(pyrazin-2-yl)tetrahydro-3′H-spiro[cyclobutane-1,2′-pyrrolo[2,1-b]oxazol]-3′-one; and
trans (5′S,7a′R)-3-(cyclopentylmethoxy)-5′-(pyrazin-2-yl)tetrahydro-3′H-spiro[cyclobutane-1,2′-pyrrolo[2,1-b]oxazol]-3′-one.

In more specific embodiments of the present invention, the compound of formula I is selected from:

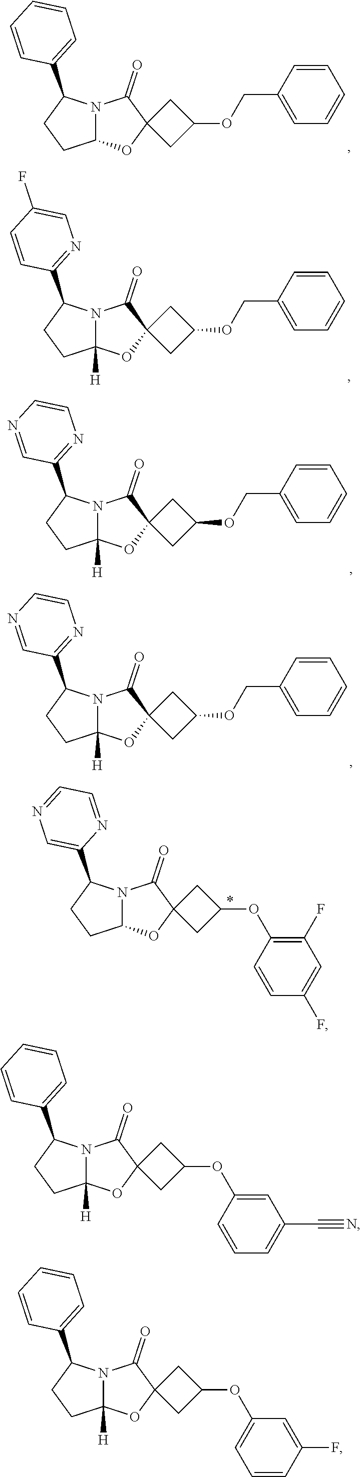

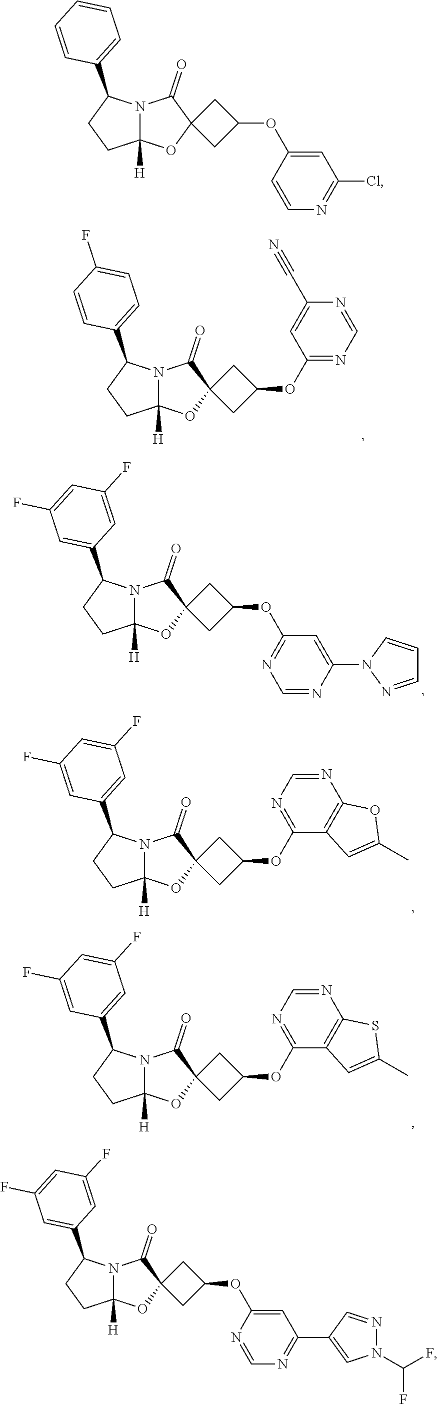

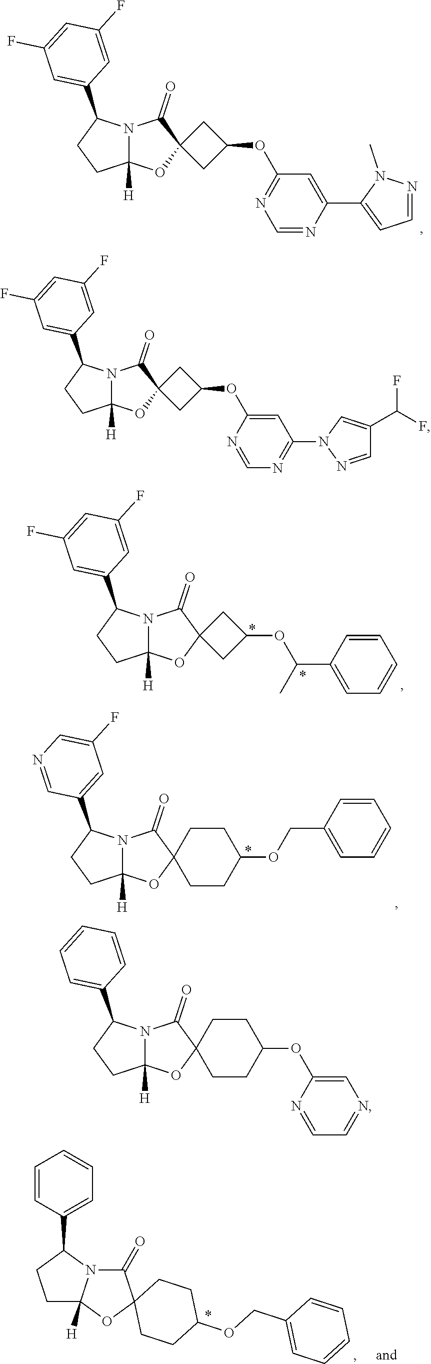

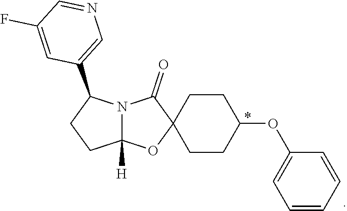 [SMILES: O=C1N2[C@@H](CC[C@H]2c2cncc(F)c2)OC12CCC(Oc1ccccc1)CC2]

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

The term “halogen” includes fluorine, chlorine, bromine or iodine.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl.

The term “C₃-C₁₀cycloalkyl” encompasses bridged, saturated or unsaturated cycloalkyl groups having 3 to 10 carbons. “Cycloalkyl” also includes non-aromatic rings as well as monocyclic, non-aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Examples described by structure include,

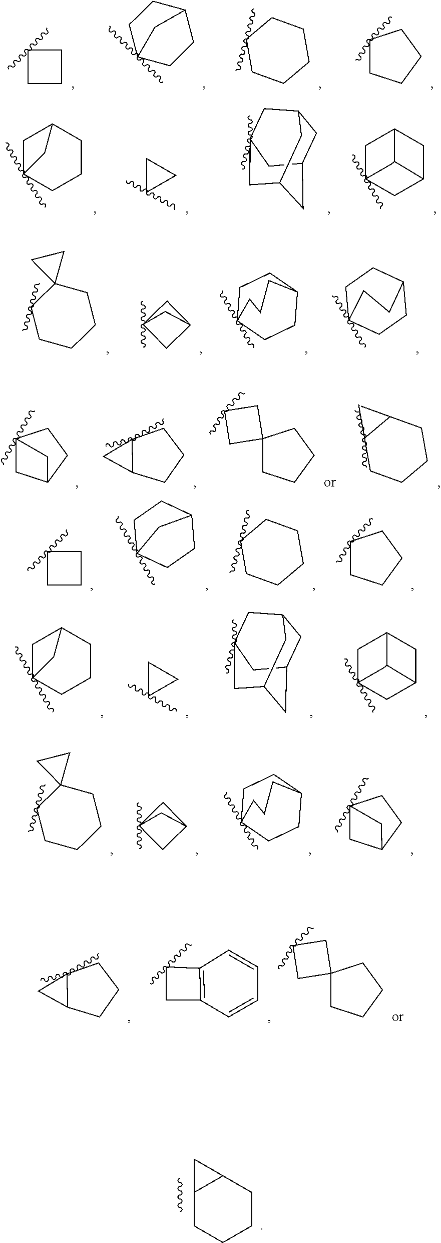

The term “heteroaryl” means a monocyclic or multicyclic, including bicyclic, aromatic heterocycloalkyl that contains at least one ring heteroatom selected from O, S and N. Examples of heteroaryl groups include pyridyl (pyridinyl), oxazolyl, azabenzothiazole, benzothiazole, imidazolyl, triazolyl, furyl, triazinyl, thienyl, pyrimidyl, pyrazinyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like.

The term “heterocycloalkyl” means mono- or bicyclic or bridged partially unsaturated and saturated rings containing at least one heteroatom selected from N, S and O, each of said rings having from 3 to 10 atoms in which the point of attachment may be carbon or nitrogen.

Examples include azetidine, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or n-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include,

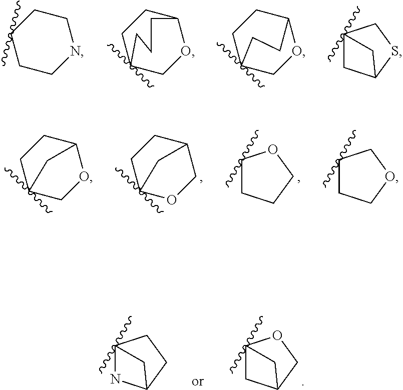

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, n-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, n-ethylmorpholine, n-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, preferably a human patient, receiving or about to receive medical treatment.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. A ³H, ¹¹C, ¹⁸F labeled compound may be used for PET or SPECT or other imaging studies.

Isotopically-enriched compounds can be prepared without undue experimentation by conventional techniques well known to those skilled in the art or by processes analogous to those described in the Schemes and Examples herein using appropriate isotopically-enriched reagents or Intermediates.

It should be noted that chemically unstable compounds are excluded from the embodiments contained herein.

### Methods of Treatment

The compounds described herein may be particularly useful for the prevention, treatment or amelioration of RIPK1-mediated diseases or disorders. Such RIPK1-mediated diseases or disorders are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFL D), kidney damage/injury (nephritis, renal transplant, surgery, administration of nephrotoxic drugs e.g., cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CV A, stroke), myocardial infarction (Ml), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the Formulae described herein, or a pharmaceutically acceptable salt thereof, may be particularly useful for the treatment of the following RIPK1-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH) autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g., cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (Ml), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the Formulae described herein, or a pharmaceutically acceptable salt thereof, may be useful for the treatment of glaucoma.

The compounds of the Formulae described herein, or a pharmaceutically acceptable salt thereof, may be particularly useful for treatment of pancreatic ductal adenocarcinoma, hepatocellular carcinoma, mesothelioma, or melanoma.

The compounds of the Formulae described herein, or a pharmaceutically acceptable salt thereof, may be particularly useful for the treatment of the following RIPK1-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen.

The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g., cisplatin.

The compounds of this invention may be particularly useful for amelioration of solid organ tissue (particularly kidney, liver, and heart and/or lung) injury or damage following transplant or the administration of nephrotoxic drugs or substances e.g., cisplatin. It will be understood that amelioration of such tissue damage may be achieved where possible, by pre-treatment with a compound of the Formulae described herein, or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of the Formulae described herein, or a pharmaceutically acceptable salt thereof, during transplant surgery.

Amelioration of such tissue damage may also be achieved by short-term treatment of a patient with a compound of the Formulae described herein, or a pharmaceutically acceptable salt thereof, after transplant surgery.

In one embodiment, the compounds of the Formulae described herein, or a pharmaceutically acceptable salt thereof, may be useful for the treatment of retinal detachment, macular degeneration, and retinitis pigmentosa.

In another embodiment, the compounds of the Formulae described herein, or a pharmaceutically acceptable salt thereof, may be useful for the treatment of multiple sclerosis.

In one embodiment, the compounds of the Formulae described herein, or a pharmaceutically acceptable salt thereof, may be useful for the treatment of traumatic brain injury.

In another embodiment, the compounds of the Formulae described herein, or a pharmaceutically acceptable salt thereof, may be useful for the treatment of Huntington's Disease or Niemann-Pick disease.

In another embodiment, the compounds of the Formulae described herein, or a pharmaceutically acceptable salt thereof, may be useful for the treatment of amyotrophic lateral sclerosis (ALS), progressive supranuclear palsy (PSP), and Alzheimer's disease.

In another embodiment, the compounds of the Formulae described herein, or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease, Alzheimer's Disease, amyotrophic lateral sclerosis, and Niemann-Pick disease may concern, more specifically, the amelioration of organ injury or damage sustained as a result of these diseases/disorders. For example, the compounds described herein may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury, Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of the Formulae described herein, or a pharmaceutically acceptable salt thereof, may be useful for the treatment of psoriasis, rheumatoid arthritis, and ulcerative and colitis.

In another embodiment, the compounds of the Formulae described herein, or a pharmaceutically acceptable salt thereof, may be useful for the treatment of lupus, inflammatory bowel disease (IBD), Crohn's disease, and ulcerative colitis.

In another embodiment, the compounds of the Formulae described herein, or a pharmaceutically acceptable salt thereof, may be useful for the treatment of cerebrovascular accident (CVA, stroke), Huntington's disease, Alzheimer's disease, amyotrophic lateral sclerosis (ALS), traumatic brain injury, multiple sclerosis, Gaucher disease, Niemann-Pick disease, and spinal cord injury.

In another embodiment, the compounds of the Formulae described herein, or a pharmaceutically acceptable salt thereof, may be useful for the treatment of amyotrophic lateral sclerosis (ALS).

In another embodiment, the compounds of the Formulae described herein, or a pharmaceutically acceptable salt thereof, may be useful for the treatment of multiple sclerosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the Formulae described herein, or a pharmaceutically acceptable salt thereof, may be useful for the treatment of pancreatic ductal adenocarcinoma (PDAC).

In another embodiment, the compounds of the Formulae described herein, or a pharmaceutically acceptable salt thereof, may be useful for the treatment of intracerebral hemorrhage and subarachnoid hemorrhage.

In another embodiment, the compounds of the Formulae described herein, or a pharmaceutically acceptable salt thereof, may be useful for the treatment of type II diabetes and obesity.

In another embodiment, the compounds of the Formulae described herein, or a pharmaceutically acceptable salt thereof, may be useful for the treatment of atherosclerosis.

In another embodiment, the compounds of the Formulae described herein, or a pharmaceutically acceptable salt thereof, may be useful for the treatment of vasculitis.

In another embodiment, the compounds of the Formulae described herein, or a pharmaceutically acceptable salt thereof, may be useful for the treatment of dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis.

In another embodiment, the compounds of the Formulae described herein, or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the Formulae described herein, or pharmaceutically acceptable salt thereof, may be useful for the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of the Formulae described herein, or a pharmaceutically acceptable salt thereof, may be particularly useful for the treatment of the RIPK1-mediated, cancer-related diseases or disorders. Gong et. al.., The role of necroptosis in cancer biology and therapy, Molecular Cancer (2019) 18:100. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use.

Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9 by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

### EXAMPLES

The following examples are meant to be illustrative and should not be construed as further limiting. The contents of the figures and all references, patents, and published patent applications cited throughout this application are expressly incorporated herein by reference.

### Abbreviations

The abbreviations used herein have the following tabulated meanings. Abbreviations not tabulated below have their meanings as commonly used unless specifically stated otherwise.

| | |
|---|---|
| ACN | acetonitrile |
| AcOH | acetic acid |
| CH₂Cl₂ | dichloromethane |
| ClCH₂CH₂Cl | 1,2-dichloroethane |
| Cu(OAc)₂ | copper(II) acetate |
| DCM | dichloromethane |
| DIEA | diisopropylethylamine |
| DMA | dimethylacetamide |
| DMF | dimethylformamide |
| DMPU | N,N′-Dimethylpropyleneurea |
| DMSO | dimethylsulfoxide |
| EI | electron ionization |
| EtOAc | ethyl acetate |
| EtOH | ethanol |
| H₂O | Water |
| HCl | Hydrochloric acid |
| ¹H NMR | proton nuclear magnetic resonance |
| HPLC | high performance liquid chromatography |
| Ir(ppy)₃ | Tris(2-phenylpyridine)iridium(III) |
| IPA | isopropyl alcohol |
| i-PrMgCl•LiCl | Isopropylmagnesium chloride lithium chloride |
| K₂CO₃ | potassium carbonate |
| LC/MS | liquid chromatography coupled to mass spectrometer |
| LDA | Lithium diisopropylamide |
| LiHMDS/LHMDS | Lithium bis(trimethylsilyl)amide |
| LiOH | lithium hydroxide |
| MgSO₄ | magnesium sulfate |
| MeCN | acetonitrile |
| MeOH | methanol |
| MHz | megahertz |
| MS | mass spectrum |
| Ms-Cl | methanesulfonyl chloride (mesyl chloride) |
| MTBE | methyl tert-butyl ether |
| NaHCO₃ | sodium bicarbonate |
| Na₂SO₄ | sodium sulfate |
| NaOH | sodium hydroxide |
| NaBH₄ | sodium borohydride |
| NaBH₃CN | sodium cyanoborohydride |
| NH₄Cl | ammonium chloride |
| NH₄HCO₃ | ammonium bicabonate |
| NH₄OH | ammonium hydroxide |
| NMR | nuclear magnetic resonance |
| o/n | Overnight |
| PE | etroleum ether |
| RT | room temperature |
| Selectfluor | 1-chloromethyl-4-fluoro-1,4- |
| | diazoniabicyclo[2.2.2]octane |
| | bis(tetrafluoroborate) |
| SFC | Supercritical fluid chromatography |
| SM | Starting material |
| SnAr | Nucleophilic Aromatic Substitution |
| TEA | Triethylamine |
| TFA | trifluoroacetic acid |
| THF | tetrahydrofuran |
| TLC | thin layer chromatography |
| TsOH | toluenesulfonic acid |

### General Synthetic Schemes

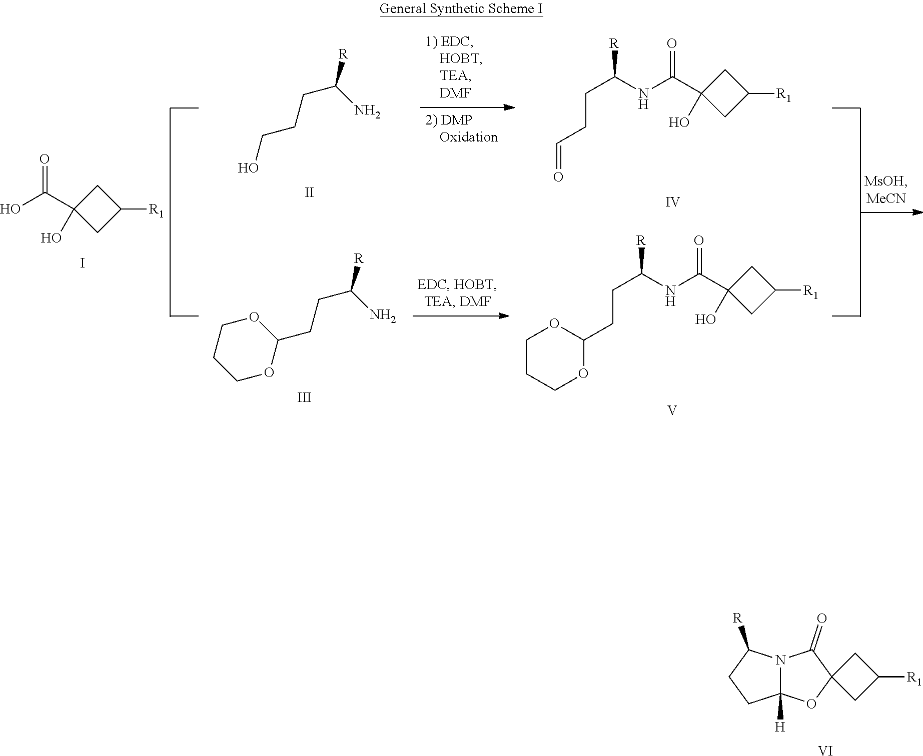

Beginning with hydroxy acids I, chiral amino alcohols II or amino acetals III can be coupled to provide either an aldehyde IV after oxidation or a protected aldehyde V directly. Both IV and V are primed for ring closing cyclization under mild acidic conditions. Diastereomeric mixture can be separated using chiral SFC chromatography or chiral hydroxy acids can also be employed to afford the desired (5′S,7a′R) enantiomer. R can be aromatic, heteroaromatic or alkyl while R¹ can be a variety of functional groups or versatile handles to allow for further functionalization of the ring system.

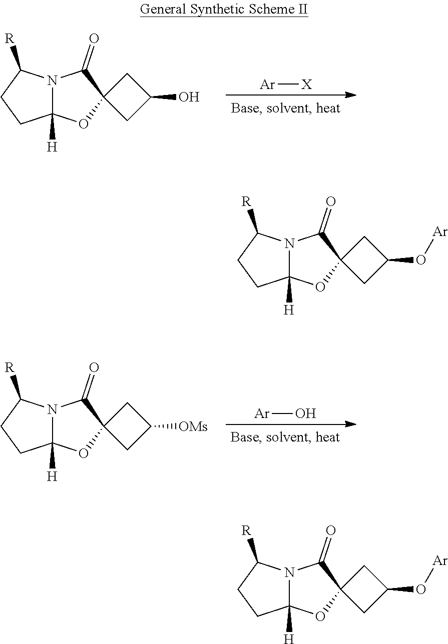

For instances where the functional handle on the spirocycle is an alcohol installation of aromatic and heteroaromatic components can be largely conducted via either traditional SnAr conditions with Ar—X in polar aprotic solvents, such as DMA or DMF, base is added, and the mixtures are heated until completion. In cases where SnAr chemistry is not viable, conversion of the alcohol to a leaving group for use in SN2 reactions with Ar—OH. Both approaches can be implemented into library format.

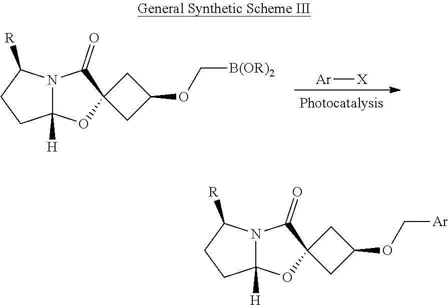

Broad functionalization of benzyl ether spirocycles can be achieved via the spirocycle boronate and photoredox coupling to Ar—X.

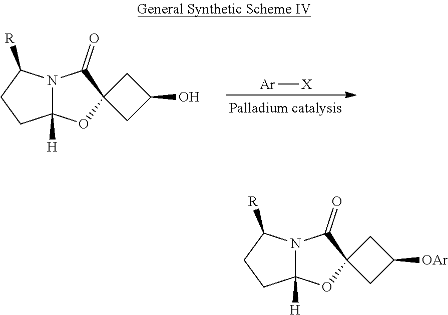

Palladium-catalyzed cross-coupling between alcohol and aryl halide can be used to access aryl ethers.

### Synthesis of Common Intermediates

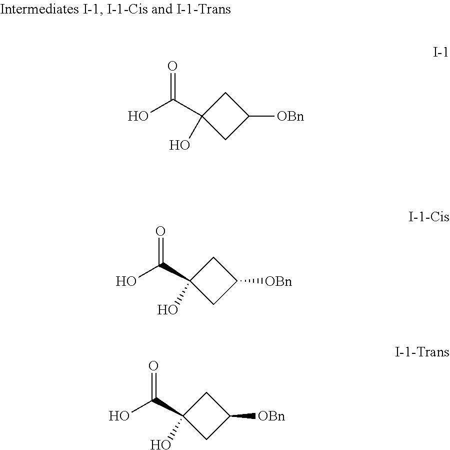 [SMILES: O=C(O)C1(O)CC(OCc2ccccc2)C1 ; O=C(O)[C@]1(O)C[C@@H](OCc2ccccc2)C1 ; O=C(O)[C@]1(O)C[C@H](OCc2ccccc2)C1]

### 3-(benzyloxy)-1-hydroxycyclobutane-1-carboxylic acid (I-1) and (1s,3s)-3-(benzyloxy)-1-hydroxycyclobutane-1-carboxylic acid (I-1-Cis) and (1r,3r)-3-(benzyloxy)-1-hydroxycyclobutane-1-carboxylic acid (I-1-Trans)

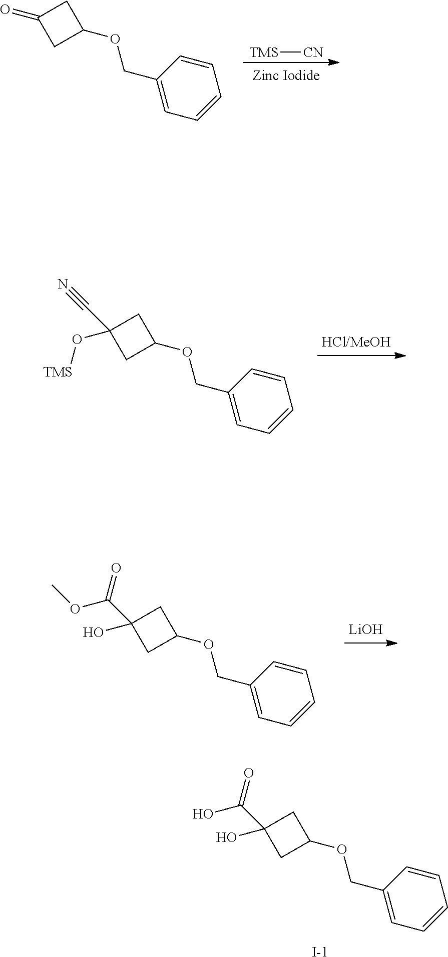

### Step 1. 3-(benzyloxy)-1-((trimethylsilyl)oxy)cyclobutane-1-carbonitrile

To a solution of 3-(benzyloxy)cyclobutan-1-one (40 g, 227 mmol) and TMS-CN (59 g, 595 mmol) in DCM (300 ml) was added zinc iodide (2 g, 6.27 mmol) at 0° C. and the resulting mixture was stirred at 20° C. for 12 h. TLC showed new spots were found. The reaction mixture was directly concentrated and the residue was purified by flash silica gel chromatography (ISCO®; 120 g SepaFlash® Silica Flash Column, eluent of 5% ethyl acetate/pet. ether gradient) to give 3-(benzyloxy)-1-((trimethylsilyl)oxy)cyclobutane-1-carbonitrile as colorless oil. ¹H NMR (400 MHz, CDCl₃-d) δ 7.29-7.39 (m, 5H), 4.45 (s, 2H), 3.90-4.03 (m, 1H), 3.01 (ddd, J=2.8, 6.8, 9.6 Hz, 2H), 2.29-2.41 (m, 2H), 0.22-0.26 (m, 9H).

### Step 2. Methyl 3-(benzyloxy)-1-hydroxycyclobutane-1-carboxylate

A solution of 3-(benzyloxy)-1-((trimethylsilyl)oxy)cyclobutane-1-carbonitrile (46 g, 167 mmol) in HCl/MeOH (4M) (150 ml) and MeOH (150 ml) was stirred at 60° C. for 2 h. TLC and LCMS showed the starting material was consumed and desired compound was found. The mixture was directly concentrated to give methyl 3-(benzyloxy)-1-hydroxycyclobutane-1-carboxylate as white solid, which was used in the next step without further purification. MS (ESI): m/z 237 [M+H]⁺.

### Step 3. 3-(Benzyloxy)-1-hydroxycyclobutane-1-carboxylic acid (racemic) (I-1)

To a solution of methyl 3-(benzyloxy)-1-hydroxycyclobutane-1-carboxylate (40 g, 169 mmol) in MeOH (300 ml) was added LiOH·H₂O (254 ml, 508 mmol). The reaction was stirred at 20° C. for 2 h. LCMS showed the starting material was consumed and desired compound was found. The reaction solution was concentrated. The residue was dissolved in water (100 mL) and extracted with EtOAc (200 mL). The aqueous phase was acidified with HCl (3 N) to pH-2, extracted with EtOAc (200 ml×3). The combined organic layers were dried over Na₂SO₄, filtered and the filtrate was concentrated in vacuo to give 3-(benzyloxy)-1-hydroxycyclobutane-1-carboxylic acid as colorless oil, which was used in the next step without further purification. MS (ESI): m/z 223 [M+H]⁺. ¹H NMR (400 MHz, MeOD-d₄) δ 7.21-7.41 (m, 5H), 4.42-4.46 (m, 2H), 3.99-4.35 (m, 1H), 2.49-2.88 (m, 2H), 2.15-2.38 (m, 2H)

### Step 4. I-1-Cis and I-1-Trans

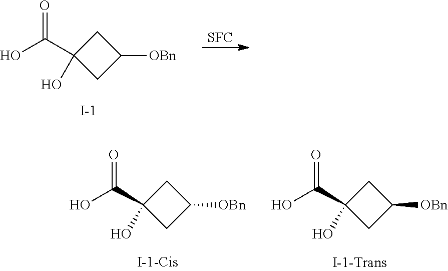

The 3-(benzyloxy)-1-hydroxycyclobutane-1-carboxylic acid (90 g, 405 mmol) was resolved by Chiral-SFC (Column DAICEL CHIRALPAK IG (250 mm×50 mm, 10 m) Condition n-Heptane-EtOH (0.1% NH₃·H₂O)) to give (1s,3s)-3-(benzyloxy)-1-hydroxycyclobutane-1-carboxylic acid (I-1-Cis) (Rₜ=2.7, ee=100%) as a yellow solid and (1r,3r)-3-(benzyloxy)-1-hydroxycyclobutane-1-carboxylic acid (I-1-Trans) (Rₜ=2.9, ee=100%) as a yellow oil. MS (ESI): m/z 262 [M+H]+I-1-Cis (Peak 1)¹H NMR (400 MHz, CD₃OD-d₄) δ 7.21-7.37 (m, 5H), 4.44 (s, 2H), 4.09 (t, J=6.8 Hz, 1H), 2.70-2.79 (m, 2H), 2.09-2.17 (m, 2H) I-1-Trans (Peak 2) ¹H NMR (400 MHz, CD₃OD-d₄) δ 7.26-7.36 (m, 5H), 4.45 (s, 2H), 4.34 (t, J=7.2 Hz, 1H), 2.53-2.61 (m, 2H), 2.31 (ddd, J=2.8, 7.2, 9.6 Hz, 2H)

Compounds presented in Table 1 were prepared in accordance with the synthetic routes in Intermediate I-1, using procedures analogous to those described above.

TABLE 1

| Intermediate | Structure | IUPAC Name |
|---|---|---|
| I-2 | 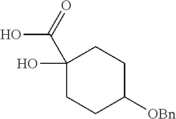 [SMILES: O=C(O)C1(O)CCC(OCc2ccccc2)CC1] | 4-(benzyloxy)-1- hydroxycyclohexane-1- carboxylic acid |

### Intermediate I-3

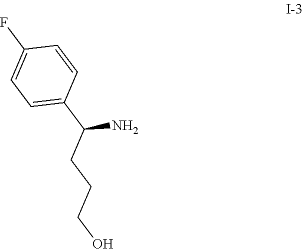 [SMILES: N[C@@H](CCCO)c1ccc(F)cc1]

### (S)-4-Amino-4-(4-fluorophenyl)butan-1-ol (I-3)

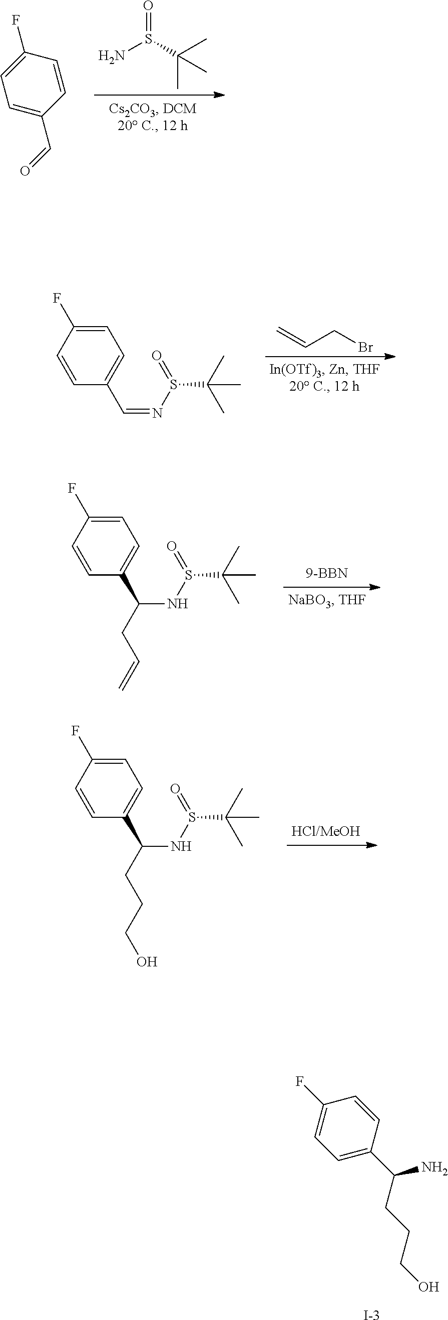

### Step 1. (R,Z)—N-(4-fluorobenzylidene)-2-methylpropane-2-sulfinamide

To a stirred mixture of 4-fluorobenzaldehyde (25 g, 201 mmol) in DCM (500 mL) was added (R)-2-methylpropane-2-sulfinamide (29.3 g, 242 mmol) and Cs₂CO₃ (98 g, 302 mmol) and the mixture was stirred at 20° C. for 12 h. LCMS showed the desired product was found. The mixture was filtered. The mixture was added by water (100 mL), extracted with DCM (100 mL×3). The combined organic layers were dried over Na₂SO₄, filtered and concentrated.

The crude was purified by flash silica gel chromatography (ISCO®; 330 g SepaFlash® Silica Flash Column, eluent of 30% EtOAc/Pet.ether gradient) to give (R,Z)—N-(4-fluorobenzylidene)-2-methylpropane-2-sulfinamide as yellow oil. MS (ESI) m/z 228 [M+H]⁺. ¹H NMR (CDCl₃-d, 400 MHz): δ 8.56 (s, 1H), 7.84-7.91 (m, 2H), 7.14-7.21 (m, 2H), 1.27 (s, 9H).

### Step 2. (R)—N—((S)-1-(4-fluorophenyl)but-3-en-1-yl)-2-methylpropane-2-sulfinamide

To a stirred solution of (R,Z)—N-(4-fluorobenzylidene)-2-methylpropane-2-sulfinamide (35 g, 154 mmol), indium(III) trifluoromethanesulfonate (95 g, 169 mmol) in THF (500 mL) was added Zn (20.470 g, 313 mmol) and 3-bromoprop-1-ene (17.30 mL, 200 mmol) at 0° C. Then the mixture was stirred at 20° C. for 16 h under N₂. LCMS and TLC (Pet. ether/EtOAc=2:1) shows the desired mass was detected. The mixture was quenched with brine (200 mL) and extracted with ethyl acetate (200 mL×3). The combined organic fractions were dried over anhydrous Na₂SO₄, filtered and the solvent was evaporated under reduced pressure. The residue was purified by flash silica gel chromatography (ISCO®; Agela® Flash Column Silica-CS (220 g), eluent of 0-50% Ethyl acetate/Petroleum ether gradient) to give (R)—N—((S)-1-(4-fluorophenyl)but-3-en-1-yl)-2-methylpropane-2-sulfinamide as a yellow solid. MS (ESI) m/z 270 [M+H]⁺. ¹H NMR (CDCl₃-d, 400 MHz): δ 7.28-7.33 (m, 2H), 6.99-7.08 (m, 2H), 5.66-5.78 (m, 1H), 5.17-5.22 (m, 1H), 5.15 (s, 1H), 4.52 (br t, J=6.4 Hz, 1H), 3.79-4.00 (m, 1H), 2.54-2.63 (m, 1H), 2.44-2.54 (m, 1H), 1.19-1.24 (m, 9H).

### Step 3. (R)—N—((S)-1-(4-fluorophenyl)-4-hydroxybutyl)-2-methylpropane-2-sulfinamide

To a solution of (R)—N—((S)-1-(4-fluorophenyl)but-3-en-1-yl)-2-methylpropane-2-sulfinamide (28 g, 104 mmol) in THF (300 mL) was added 9-BBN (416 mL, 208 mmol) (0.5 M in THF) dropwise at −20° C. under N₂, after the addition was finished, the reaction was stirred at 20° C. for 16 h. To the reaction mixture was added sodium perborate tetrahydrate (128 g, 832 mmol) with water (100 mL) and stirred for another 3 h. LCMS showed the starting material was consumed and desired compound was found. The reaction solution was extracted with EtOAc (200 mL×3), washed with brine (100 mL), dried over Na₂SO₄, filtered and concentrated. The residue was purified by flash silica gel chromatography (ISCO®; 220 g SepaFlash® Silica Flash Column, eluent of 100% EtOAc/Pet.ether gradient) to give (R)—N—((S)-1-(4-fluorophenyl)-4-hydroxybutyl)-2-methylpropane-2-sulfinamide as yellow oil. MS (ESI) m/z 288 [M+H]⁺. ¹H NMR (CDCl₃-d, 400 MHz): δ 7.27-7.30 (m, 2H), 7.00-7.06 (m, 2H), 4.41 (br t, J=7.2 Hz, 1H), 3.58-3.69 (m, 2H), 1.86-1.94 (m, 2H), 1.46-1.55 (m, 2H), 1.16-1.22 (m, 9H).

### Step 4. (S)-4-Amino-4-(4-fluorophenyl)butan-1-ol (I-3)

To a solution of (R)—N—((S)-1-(4-fluorophenyl)-4-hydroxybutyl)-2-methylpropane-2-sulfinamide (15 g, 52.2 mmol) in MeOH (150 mL) was added HCl/MeOH (4 M) (80 mL). Then the mixture was stirred at 25° C. for 12 h. LCMS show the desired product mass formed. On completion, the mixture was concentrated in vacuo to give (S)-4-amino-4-(4-fluorophenyl)butan-1-ol as yellow oil. MS (ESI) m/z 184 [M+H]+

Compounds presented in Table 2 were prepared in accordance with the synthetic routes in Intermediate I-3, using procedures analogous to those described above.

TABLE 2

| Intermediate | Structure | IUPAC Name |
|---|---|---|
| I-4 | 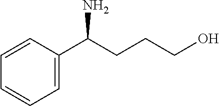 [SMILES: N[C@@H](CCCO)c1ccccc1] | (S)-4-amino-4-phenylbutan-1-ol |
| I-5 | 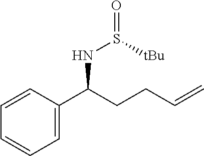 [SMILES: C=CCC[C@H](N[S@](=O)C(C)(C)C)c1ccccc1] | (R)-2-methyl-N-((S)-1-phenylpent-4- en-1-yl)propane-2-sulfinamide |
| I-6 | 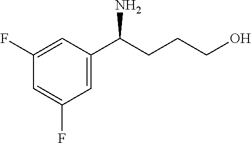 [SMILES: N[C@@H](CCCO)c1cc(F)cc(F)c1] | (S)-4-amino-4-(3,5- difluorophenyl)butan-1-ol |
| I-7 | 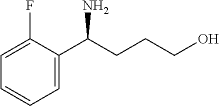 [SMILES: N[C@@H](CCCO)c1ccccc1F] | (S)-4-amino-4-(2- fluorophenyl)butan-1-ol |
| I-8 | 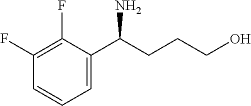 [SMILES: N[C@@H](CCCO)c1cccc(F)c1F] | (S)-4-amino-4-(2,3- difluorophenyl)butan-1-ol |
| I-9 | 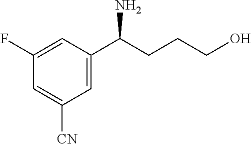 [SMILES: N#Cc1cc(F)cc([C@@H](N)CCCO)c1] | (S)-4-amino-4-(3,5- difluorophenyl)butan-1-ol |
| I-10 | 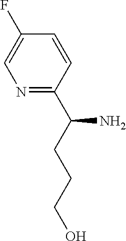 [SMILES: N[C@@H](CCCO)c1ccc(F)cn1] | (S)-4-amino-4-(5-fluoropyridin-2- yl)butan-1-ol |
| I-11 | 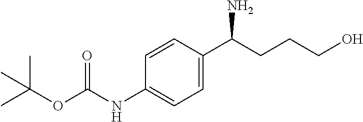 [SMILES: CC(C)(C)OC(=O)Nc1ccc([C@@H](N)CCCO)cc1] | tert-butyl (S)-(4-(1-amino-4- hydroxybutyl)phenyl)carbamate |
| I-12 | 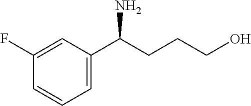 [SMILES: N[C@@H](CCCO)c1cccc(F)c1] | (S)-4-amino-4-(3- fluorophenyl)butan-1-ol |
| I-13 | 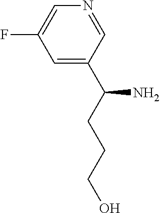 [SMILES: N[C@@H](CCCO)c1cncc(F)c1] | (S)-4-amino-4-(5-fluoropyridin-3- yl)butan-1-ol |

### Intermediate I-14

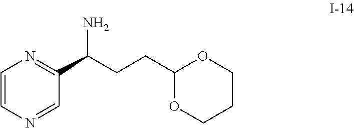 [SMILES: N[C@@H](CCC1OCCCO1)c1cnccn1]

### (S)-3-(1,3-dioxan-2-yl)-1-(pyrazin-2-yl)propan-1-amine

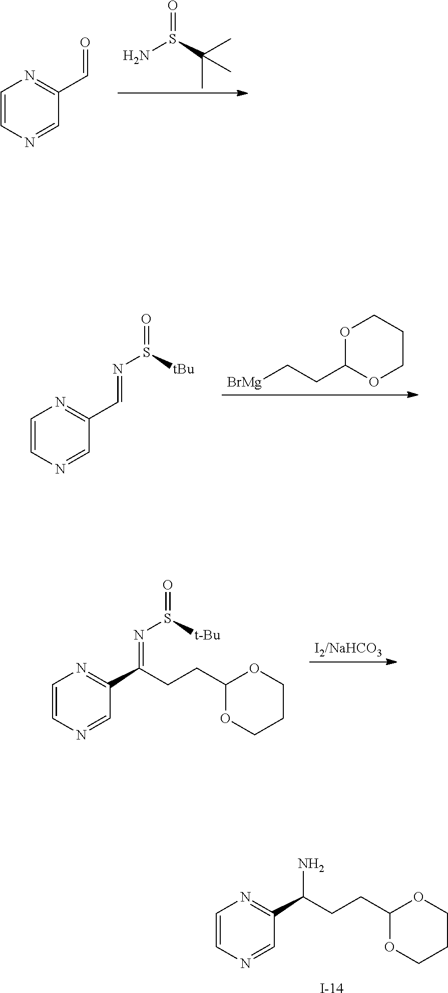

### Step 1. (S)-2-methyl-N-(pyrazin-2-ylmethylene)propane-2-sulfinamide

To a solution of pyrazine-2-carbaldehyde (10 g, 93 mmol) in DCM (300 ml) was added (S)-2-methylpropane-2-sulfinamide (13.4 g, 111 mmol) and Cs₂CO₃ (90 g, 278 mmol) at 25° C. and the mixture was stirred at 25° C. for 3 h. The reaction mixture was filtered and the filtrate was concentrated in vacuum to get the brown oil. The oil was purified by column chromatography (SiO₂, 330 g ISCO column eluting with 10-70% EtOAc/Hex) to obtain (S)-2-methyl-N-(pyrazin-2-ylmethylene)propane-2-sulfinamide. MS (ESI) m/z: 212 [M+H]⁺. ¹H NMR (CDCl₃-d) δ: 9.27 (s, 1H), 8.77 (s, 1H), 8.75-8.71 (m, 1H), 8.69 (d, J=2.1 Hz, 1H), 1.33 (s, 9H)

### Step 2. (S)—N—((S)-3-(1,3-dioxan-2-yl)-1-(pyrazin-2-yl)propyl)-2-methylpropane-2-sulfinamide

A 200-ml two-neck RBF equipped with 25-ml addition funnel was charged with (S)-2-methyl-N-(pyrazin-2-ylmethylene)propane-2-sulfinamide (5.0 g, 23.7 mmol) in 2-MeTHF (118 ml) under inert atmosphere. The solution was cooled to −78° C. and treated with a 0.5 M solution of ((2-(1,3-dioxan-2-yl)ethyl)magnesium bromide (49.7 ml, 24.9 mmol) in tetrahydrofuran dropwise over 10 min. The reaction mixture was warmed to 0° C. over 4 h. The reaction mixture was quenched with aqueous ammonium chloride and brought to RT. The resulting mixture was transferred into separatory funnel with ethyl acetate and the organic layer was cut and subsequently washed with brine. The aqueous phase was backwashed with ethyl acetate twice. The combined organic phases were dried over Na₂SO₄ and concentrated. The product was purified on ISCO Gold—12 g silica gel column—0-100% [3:1 EtOAc:EtOH]/Hexanes to obtain (S)—N—((S)-3-(1,3-dioxan-2-yl)-1-(pyrazin-2-yl)propyl)-2-methylpropane-2-sulfinamide was isolated as an orange oil.

### Step 3: (S)-3-(1,3-dioxan-2-yl)-1-(pyrazin-2-yl)propan-1-amine (I-14)

(S)—N—((S)-3-(1,3-dioxan-2-yl)-1-(pyrazin-2-yl)propyl)-2-methylpropane-2-sulfinamide (1081 mg, 3.3 mmol) in 2-Me THF (17.6 ml)/water (4.4 ml) was treated with iodine (419 mg, 1.650 mmol) at RT for 20 hours. LCMS indicated no SM remained. The reaction was quenched with the addition of 2.5 ml of ammonium hydroxide solution. After stirring for 2 hours the aqueous layer was removed and the THF was decanted into a recovery flask and concentrated. The aqueous layer was azeotroped to remove water and the residue was taken up in acetonitrile and filtered into the first flask and the combined organics were concentrated to give (S)-3-(1,3-dioxan-2-yl)-1-(pyrazin-2-yl)propan-1-amine as a red oil which was used without further purification. MS (ESI) m/z: 224 [M+H]⁺.

Compounds presented in Table 3 were prepared in accordance with the synthetic routes in Intermediate I-14, using procedures analogous to those described above.

TABLE 3

| Intermediate | Structure | IUPAC Name |
|---|---|---|
| I-15 | 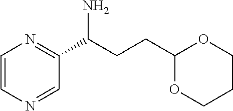 | (R)-3-(1,3-dioxan-2-yl)-1-(pyrazin-2- yl)propan-1-amine |
| I-16 | 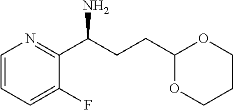 [SMILES: N[C@@H](CCC1OCCCO1)c1ncccc1F] | (S)-4-amino-4-phenylbutan-1-ol |
| I-17 | 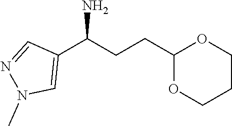 [SMILES: Cn1cc([C@@H](N)CCC2OCCCO2)cn1] | (S)-3-(1,3-dioxan-2-yl)-1-(1-methyl-1H- pyrazol-4-yl)propan-1-amine |

### Intermediates I-18, I-18-Trans and I-18-Cis

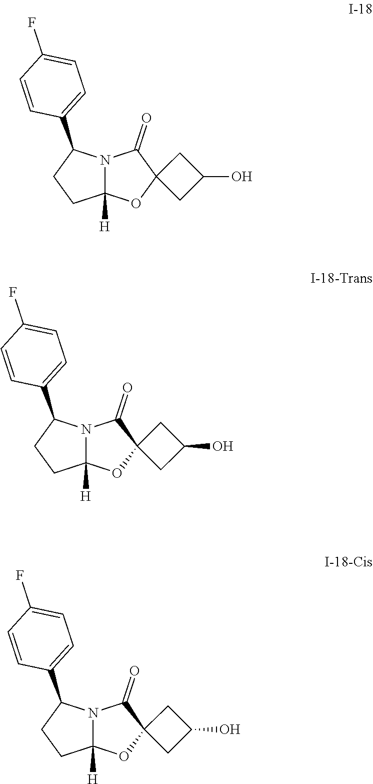 [SMILES: O=C1N2[C@@H](CC[C@H]2c2ccc(F)cc2)OC12CC(O)C2 ; O=C1N2[C@@H](CC[C@H]2c2ccc(F)cc2)O[C@]12C[C@H](O)C2 ; O=C1N2[C@@H](CC[C@H]2c2ccc(F)cc2)O[C@]12C[C@@H](O)C2]

### (5′S,7a′R)-5′-(4-fluorophenyl)-3-hydroxytetrahydro-3′H-spiro[cyclobutane-1,2′-pyrrolo[2,1-b]oxazol]-3′-one (I-18) and (1r,3R,5′S,7a′R)-5′-(4-fluorophenyl)-3-hydroxytetrahydro-3′H-spiro[cyclobutane-1,2′-pyrrolo[2,1-b]oxazol]-3′-one (I-18-Trans) and (1s,3S,5′S,7a′R)-5′-(4-fluorophenyl)-3-hydroxytetrahydro-3′H-spiro[cyclobutane-1,2′-pyrrolo[2,1-b]oxazol]-3′-one (I-18-Cis)

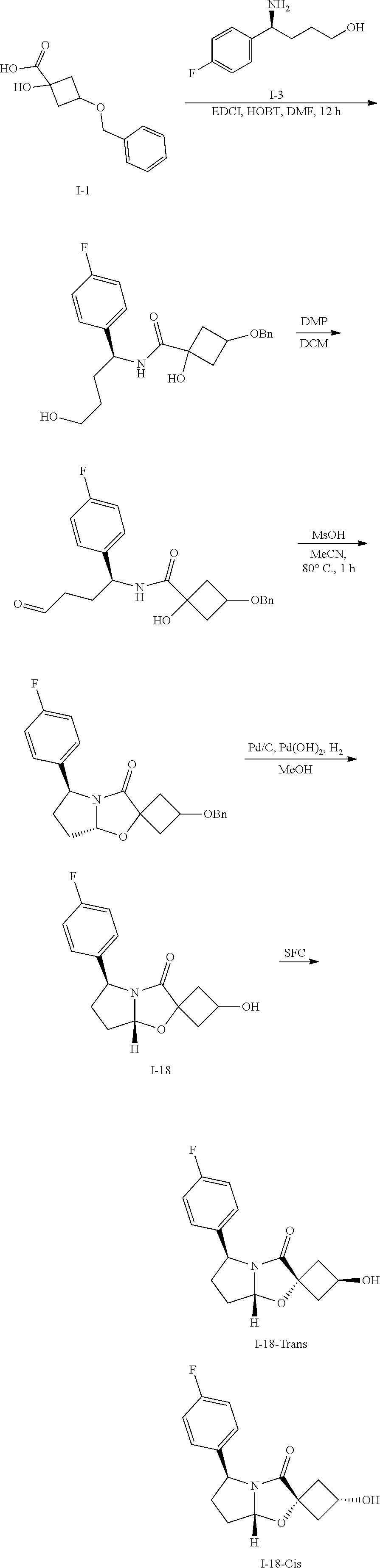

### Step 1. (S)-3-(benzyloxy)-N-(1-(4-fluorophenyl)-4-hydroxybutyl)-1-hydroxycyclobutane-1-carboxamide

To a stirred mixture of 3-(benzyloxy)-1-hydroxycyclobutane-1-carboxylic acid (8.0 g, 36 mmol) in DMF (150 mL) was added to TEA (22.82 mL, 164 mmol), HOBT (5.5 g, 36.0 mmol), EDCI (6.9 g, 36 mmol) and (S)-4-amino-4-(4-fluorophenyl)butan-1-ol (6.0 g, 33 mmol). Then the mixture was stirred at 20° C. for 12 h. LCMS showed desired product. Water (200 mL) was added and the mixture was extracted with EtOAc (3×100 mL). The combined organic fraction were concentrated washed with brine (2×100 mL), dried over Na₂SO₄, filtered and the solvent was evaporated under reduced pressure. The crude was purified by flash silica gel chromatography (ISCO®; 80 g SepaFlash® Silica Flash Column, eluent of 10% EtOAc/Pet.ether gradient) to give (S)-3-(benzyloxy)-N-(1-(4-fluorophenyl)-4-hydroxybutyl)-1-hydroxycyclobutane-1-carboxamide as yellow oil MS (ESI): m/z 388 [M+H]⁺. ¹H NMR (CDCl₃-d, 400 MHz): δ 7.27-7.42 (m, 4H), 7.23-7.26 (m, 1H), 6.89-7.08 (m, 3H), 4.83-4.98 (m, 1H), 3.51-3.71 (m, 2H), 2.38-2.85 (m, 2H), 2.07-2.17 (m, 1H), 1.82-1.95 (m, 2H), 1.34-1.74 (m, 6H).

### Step 2. (S)-3-(benzyloxy)-N-(1-(4-fluorophenyl)-4-oxobutyl)-1-hydroxycyclobutane-1-carboxamide

To a solution of (S)-3-(benzyloxy)-N-(1-(4-fluorophenyl)-4-hydroxybutyl)-1-hydroxycyclobutane-1-carboxamide (6 g, 15.49 mmol) in DCM (100 mL) was added DMP (7.88 g, 18.58 mmol) at 0° C. The resulting mixture was stirred at 20° C. for 1 h. LCMS showed the starting material was consumed and desired compound was found. The reaction was added sat. NaHCO₃ (70 mL) and extracted with DCM (2×50 mL). The combined organic layers were dried over Na₂SO₄, filtered and concentrated to give crude (S)-3-(benzyloxy)-N-(1-(4-fluorophenyl)-4-oxobutyl)-1-hydroxycyclobutane-1-carboxamide as yellow oil. MS (ESI): m/z 386 [M+H]+

### Step 3. (5′S,7a′R)-3-(benzyloxy)-5′-(4-fluorophenyl)tetrahydro-3′H-spiro[cyclobutane-1,2′-pyrrolo[2,1-b]oxazol]-3′-one

A mixture of (S)-3-(benzyloxy)-N-(1-(4-fluorophenyl)-4-oxobutyl)-1-hydroxycyclobutane-1-carboxamide (6 g, 15.57 mmol) and MsOH (2.022 mL, 31.1 mmol) in MeCN (100 mL) was stirred at 80° C. for 2 h to give yellow mixture. LCMS showed desired MS was detected. The mixture was concentrated in vacuo and purified by flash silica gel chromatography (ISCO®; Agela® Flash Column Silica-CS (80 g), Eluent of 20% Ethyl acetate/Petroleum ether gradient) (5′S,7a′R)-3-(benzyloxy)-5′-(4-fluorophenyl)tetrahydro-3′H-spiro[cyclobutane-1,2′-pyrrolo[2,1-b]oxazol]-3′-one as yellow solid. MS (ESI): m/z 368 [M+H]⁺. ¹H NMR (CDCl₃-d, 400 MHz): δ 7.28-7.37 (m, 5H), 7.19-7.25 (m, 2H), 6.99-7.06 (m, 2H), 5.55-5.66 (m, 1H), 4.98 (q, J=8.0 Hz, 1H), 4.44-4.49 (m, 2H), 4.10-4.38 (m, 1H), 2.54-2.93 (m, 3H), 2.30-2.49 (m, 2H), 2.15-2.26 (m, 1H), 1.93-2.04 (m, 1H), 1.63-1.76 (m, 1H).

### Step 4. (5′S,7a′R)-5′-(4-fluorophenyl)-3-hydroxytetrahydro-3′H-spiro[cyclobutane-1,2′-pyrrolo[2,1-b]oxazol]-3′-one (I-18)

To a solution of (5′S,7a′R)-3-(benzyloxy)-5′-(4-fluorophenyl)tetrahydro-3′H-spiro[cyclobutane-1,2′-pyrrolo[2,1-b]oxazol]-3′-one (2.8 g, 7.62 mmol) in MeOH (60 mL) was added Pd(OH)₂ (0.535 g, 0.762 mmol) and Pd/C (0.405 g, 0.762 mmol) at 20° C. The resulting mixture was stirred at 40° C. for 16 h under H₂. TLC showed new compound. The reaction solution was filtered and concentrated in vacuo. The crude product was purified by column chromatography on silica gel eluted with 100% EtOA and 20% EtOH/EtOAc to give (5′S,7a′R)-5′-(4-fluorophenyl)-3-hydroxytetrahydro-3′H-spiro[cyclobutane-1,2′-pyrrolo[2,1-b]oxazol]-3′-one as a yellow solid. MS (ESI): m/z 278 [M+H]⁺. ¹H NMR (CD₃OD-d4, 400 MHz): δ 7.23-7.38 (m, 2H), 6.99-7.14 (m, 2H), 5.65-5.76 (m, 1H), 4.91 (br t, J=8.0 Hz, 1H), 4.07-4.49 (m, 1H), 2.78-2.78 (m, 1H), 2.54-2.88 (m, 2H), 2.31-2.48 (m, 1H), 2.15-2.30 (m, 2H), 1.89-2.02 (m, 1H), 1.57-1.69 (m, 1H).

### Step 5. (1r,3R,5′S,7a′R)-5′-(4-fluorophenyl)-3-hydroxytetrahydro-3′H-spiro[cyclobutane-1,2′-pyrrolo[2,1-b]oxazol]-3′-one (I-18-Trans) and (1s,3S,5′S,7a′R)-5′-(4-fluorophenyl)-3-hydroxytetrahydro-3′H-spiro[cyclobutane-1,2′-pyrrolo[2,1-b]oxazol]-3′-one (I-18-Cis)

(5′S,7a′R)-5′-(4-fluorophenyl)-3-hydroxytetrahydro-3′H-spiro[cyclobutane-1,2′-pyrrolo[2,1-b]oxazol]-3′-one (1.8 g, 6.33 mmol) was separated by SFC Column DAICEL CHIRALCEL IG eluting with 0.1% NH₃H₂O EtOH to give (5′S,7a′R)-5′-(4-fluorophenyl)-3-hydroxytetrahydro-3′H-spiro[cyclobutane-1,2′-pyrrolo[2,1-b]oxazol]-3′-one and (5′S,7a′R)-5′-(4-fluorophenyl)-3-hydroxytetrahydro-33H-spiro[cyclobutane-1,2′-pyrrolo[2,1-b]oxazol]-3′-one both as light yellow solid.

I-18-Trans (Peak 1): ¹H NMR (CD₃OD-d₄, 400 MHz): δ 7.26-7.33 (m, 2H), 7.02-7.10 (m, 2H), 5.70 (dd, J=7.2, 5.2 Hz, 1H), 4.90-4.95 (m, 1H), 4.44 (t, J=7.2 Hz, 1H), 2.68-2.76 (m, 1H), 2.61-2.68 (m, 1H), 2.55-2.61 (m, 1H), 2.44 (br d, J=6.0 Hz, 1H), 2.26 (br dd, J=12.4, 1.2 Hz, 2H), 1.92-2.03 (m, 1H), 1.63-1.74 (m, 1H).

I-18-Cis (Peak 2): ¹H NMR (CD₃OD-d₄, 400 MHz): δ 7.27-7.33 (m, 2H), 7.03-7.10 (m, 2H), 5.71 (dd, J=7.6, 4.8 Hz, 1H), 4.89-4.94 (m, 1H), 4.13 (quin, J=7.2 Hz, 1H), 2.79-2.87 (m, 2H), 2.66 (dtd, J=13.2, 7.6, 2.4 Hz, 1H), 2.31-2.39 (m, 1H), 2.17-2.25 (m, 2H), 1.95 (dddd, J=13.2, 11.6, 8.0, 6.4 Hz, 1H), 1.63 (tt, J=12.0, 7.6 Hz, 1H).

Compounds presented in Table 4 were prepared in accordance with the synthetic routes in Intermediate I-18, using procedures analogous to those described above.

TABLE 4

| Intermediate | Structure | IUPAC Name |
|---|---|---|
| I-19 | 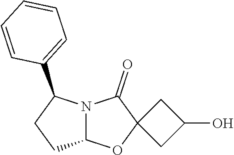 [SMILES: O=C1N2[C@@H](CC[C@H]2c2ccccc2)OC12CC(O)C2] | (5′S,7a′R)-3-hydroxy-5′-phenyltetrahydro- 3′H-spiro[cyclobutane-1,2′-pyrrolo[2,1- b]oxazol]-3′-one |
| I-19-Cis | 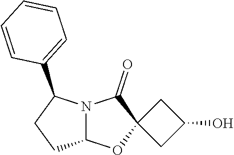 [SMILES: O=C1N2[C@@H](CC[C@H]2c2ccccc2)O[C@]12C[C@@H](O)C2] | (1s,3S,5′S,7a′R)-3-hydroxy-5′- phenyltetrahydro-3′-spiro[cyclobutane- 1,2′-pyrrolo[2,1-b]oxazol]-3′-one |
| I-19-Trans | 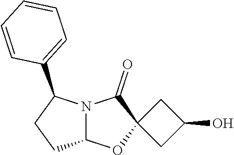 [SMILES: O=C1N2[C@@H](CC[C@H]2c2ccccc2)O[C@]12C[C@H](O)C2] | (1r,3R,5′S,7a′R)-3-hydroxy-5′- phenyltetrahydro-3′H-spiro[cyclobutane- 1,2′-pyrrolo[2,1-b]oxazol]-3′-one |
| I-20 | 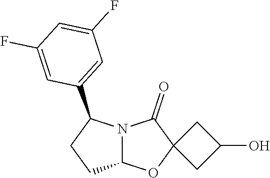 [SMILES: O=C1N2[C@@H](CC[C@H]2c2cc(F)cc(F)c2)OC12CC(O)C2] | (5′S,7a′R)-5′-(3,5-difluorophenyl)-3- hydroxytetrahydro-3′H-spiro[cyclobutane- 1,2′-pyrrolo[2,1-b]oxazol]-3′-one |
| I-20-Cis | 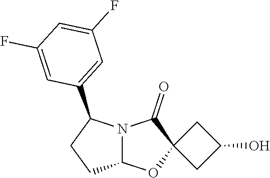 [SMILES: O=C1N2[C@@H](CC[C@H]2c2cc(F)cc(F)c2)O[C@]12C[C@@H](O)C2] | (1s,3S,5′S,7a′R)-5′-(3,5-difluorophenyl)-3- hydroxytetrahydro-3′H-spiro[cyclobutane- 1,2′-pyrrolo[2,1-b]oxazol]-3′-one |
| I-20-Trans | 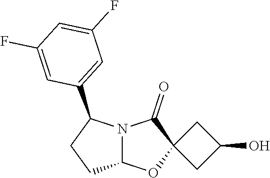 [SMILES: O=C1N2[C@@H](CC[C@H]2c2cc(F)cc(F)c2)O[C@]12C[C@H](O)C2] | (1r,3R,5′S,7a′R)-5′-(3,5-difluorophenyl)-3- hydroxytetrahydro-3′H-spiro[cyclobutane- 1,2′-pyrrolo[2,1-b]oxazol]-3′-one |
| I-21 | 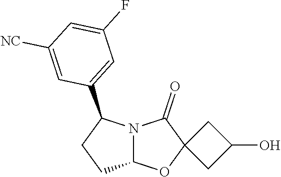 [SMILES: N#Cc1cc(F)cc([C@@H]2CC[C@H]3OC4(CC(O)C4)C(=O)N32)c1] | 3-fluoro-5-((5′S,7a′R)-3-hydroxy-3′- oxotetrahydro-3′H-spiro[cyclobutane-1,2′- pyrrolo[2,1-b]oxazol]-5′-yl)benzonitrile |
| I-22 | 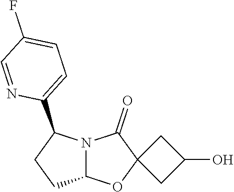 [SMILES: O=C1N2[C@@H](CC[C@H]2c2ccc(F)cn2)OC12CC(O)C2] | (5′S,7a′R)-5′-(5-fluoropyridin-2-yl)-3- hydroxytetrahydro-3′H-spiro[cyclobutane- 1,2′-pyrrolo[2,1-b]oxazol]-3′-one |
| I-23 | 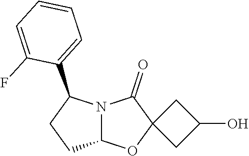 [SMILES: O=C1N2[C@@H](CC[C@H]2c2ccccc2F)OC12CC(O)C2] | (5′S,7a′R)-5′-(2-fluorophenyl)-3- hydroxytetrahydro-3′H-spiro[cyclobutane- 1,2′-pyrrolo[2,1-b]oxazol]-3′-one |
| I-24 | 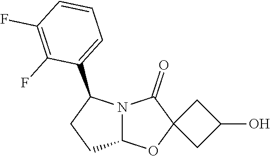 [SMILES: O=C1N2[C@@H](CC[C@H]2c2cccc(F)c2F)OC12CC(O)C2] | (5′S,7a′R)-5′-(2,3-difluorophenyl)-3- hydroxytetrahydro-3′H-spiro[cyclobutane- 1,2′-pyrrolo[2,1-b]oxazol]-3′-one |
| I-25 | 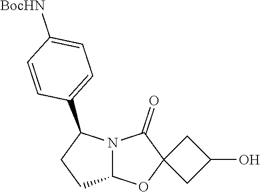 [SMILES: CC(C)(C)OC(=O)Nc1ccc([C@@H]2CC[C@H]3OC4(CC(O)C4)C(=O)N32)cc1] | tert-butyl (4-((5′S,7a′R)-3-hydroxy-3′- oxotetrahydro-3′H-spiro[cyclobutane-1,2′- pyrrolo[2,1-b]oxazol]-5′- yl)phenyl)carbamate |
| I-26 | 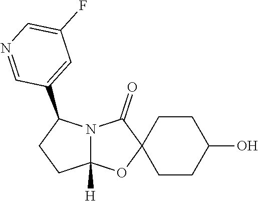 [SMILES: O=C1N2[C@@H](CC[C@H]2c2cncc(F)c2)OC12CCC(O)CC2] | (5′S,7a′R)-5′-(5-fluoropyridin-3-yl)-4- hydroxytetrahydro-3′H-spiro[cyclohexane- 1,2′-pyrrolo[2,1-b]oxazol]-3′-one |
| I-27 | 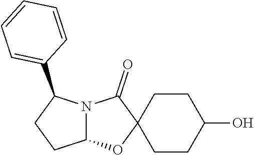 | (5′S,7a′R)-4-hydroxy-5′-phenyltetrahydro- 3′H-spiro[cyclohexane-1,2′-pyrrolo[2,1- b]oxazol]-3′-one |

### Intermediate I-28

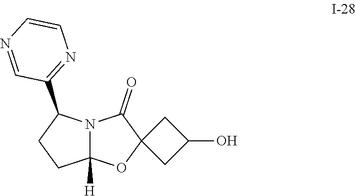 [SMILES: O=C1N2[C@@H](CC[C@H]2c2cnccn2)OC12CC(O)C2]

### (5′S,7a′R)-3-hydroxy-5′-(pyrazin-2-yl)tetrahydro-3′H-spiro[cyclobutane-1,2′-pyrrolo[2,1-b]oxazol]-3′-one (I-28)

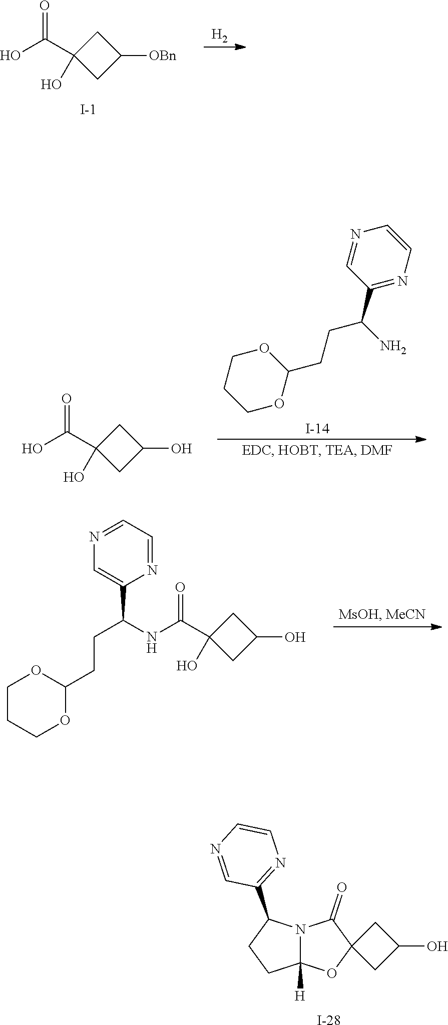

### Step 1. 1,3-dihydroxycyclobutane-1-carboxylic acid

To a solution of (1R,3R)-3-(benzyloxy)-1-hydroxycyclobutane-1-carboxylic acid (5.6 g, 25.2 mmol) in MeOH (100 mL) was added Pd(OH)₂/C (1.8 g, 2.56 mmol) and Pd/C (1.4 g, 2.63 mmol) at 20° C. The resulting mixture was stirred at 40° C. (50 psi) for 16 h under H₂. The reaction completion was detected by TLC (Pet. ether/EtOAc 1:1). The mixture was filtered and evaporated under reduced pressure to give 1,3-dihydroxycyclobutane-1-carboxylic acid as a yellow solid. ¹H NMR (400 MHz, CD₃OD-d₄) δ 4.19 (quin, J=7.12 Hz, 1H), 2.83 (ddd, J=2.68, 7.21, 9.78 Hz, 2H), 2.14 (ddd, J=2.68, 7.30, 9.69 Hz, 2H)

### Step 2. (S)—N-(3-(1,3-dioxan-2-yl)-1-(pyrazin-2-yl)propyl)-1,3-dihydroxycyclobutane-1-carboxamide

To a solution of (S)-3-(1,3-dioxan-2-yl)-1-(pyrazin-2-yl)propan-1-amine (357 mg, 1.6 mmol) in DMF (10.7 ml) was added 1,3-dihydroxycyclobutane-1-carboxylic acid (254 mg, 1.920 mmol), TEA (669 μl, 4.80 mmol), HOBT (368 mg, 2.400 mmol), EDC (460 mg, 2.400 mmol) and the resulting mixture was stirred at 20° C. for 12 h. LCMS showed the desired compound was found. The mixture was filtered the mixture and the filtrate was purified by Prep-HPLC (Prep HPLC condition: Preparative HPLC on 245 g Gold ISOC RP (C18) column using the mobile phase A-B: water (0.05% TFA)-ACN (0.05% TFA) Gradient: 5-35%) to give (S)—N-(3-(1,3-dioxan-2-yl)-1-(pyrazin-2-yl)propyl)-1,3-dihydroxycyclobutane-1-carboxamide as yellow oil. MS (ESI): m/z 338[M+H]⁺.

### Step 3. (5′S,7a′R)-3-hydroxy-5′-(pyrazin-2-yl)tetrahydro-3′H-spiro[cyclobutane-1,2′-pyrrolo[2,1-b]oxazol]-3′-one (I-28)

To a solution of (S)—N-(3-(1,3-dioxan-2-yl)-1-(pyrazin-2-yl)propyl)-1,3-dihydroxycyclobutane-N-carboxamide (467 mg, 1.384 mmol) in MeCN (6921 μl) was added MsOH (270 μl, 4.15 mmol) and the resulting mixture was stirred at 50° C. for 2 h. LCMS showed the starting material was consumed and desired compound was found. The reaction was filtered and then concentrated to provide crude residue which was purified by MPLC: ISCO 24 g Gold column eluting with [13:1 EtOAc:EtOH]/Hexanes (gradient 50-100%) to obtain (5′S, 7a′R)-3-hydroxy-5′-(pyrazin-2-yl)tetrahydro-3′H-spiro[cyclobutane-,2′-pyrrolo[2,1-b]oxazol]-3′-one MS (ESI): m/z 262 [M+H]⁺.

Compounds presented in Table 5 below were prepared using procedure analogous to the one described for Intermediate I-28 using appropriately chosen intermediates.

TABLE 5

| Intermediate | Structure | IUPAC Name |
|---|---|---|
| I-29-Cis | 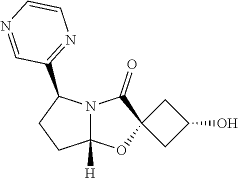 [SMILES: O=C1N2[C@@H](CC[C@H]2c2cnccn2)O[C@]12C[C@@H](O)C2] | (1s,3S,5′S,7a′R)-3-hydroxy-5′-(pyrazin-2- yl)tetrahydro-3′H-spiro[cyclobutane-1,2′- pyrrolo[2,1-b]oxazol]-3′-one |
| I-30-Trans | 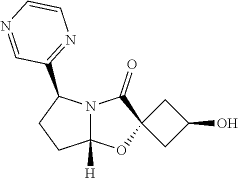 [SMILES: O=C1N2[C@@H](CC[C@H]2c2cnccn2)O[C@]12C[C@H](O)C2] | (1r,3R,5′S,7a′R)-3-hydroxy-5′-(pyrazin-2- yl)tetrahydro-3′H-spiro[cyclobutane-1,2′- pyrrolo[2,1-b]oxazol]-3′-one |
| I-31-Cis | 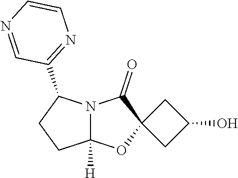 [SMILES: O=C1N2[C@H](CC[C@@H]2c2cnccn2)O[C@]12C[C@@H](O)C2] | (1s,3R,5′R,7a′S)-3-hydroxy-5′-(pyrazin-2- yl)tetrahydro-3′H-spiro[cyclobutane-1,2′- pyrrolo[2,1-b]oxazol]-3′-one |
| I-32-Trans | 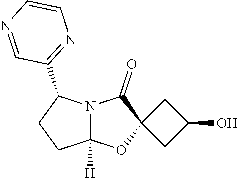 [SMILES: O=C1N2[C@H](CC[C@@H]2c2cnccn2)O[C@]12C[C@H](O)C2] | (1r,3S,5′R,7a′S)-3-hydroxy-5′-(pyrazin-2- yl)tetrahydro-3′H-spiro[cyclobutane-1,2′- pyrrolo[2,1-b]oxazol]-3′-one |
| I-33 | 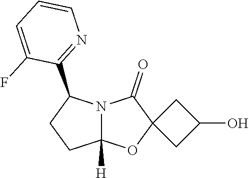 [SMILES: O=C1N2[C@@H](CC[C@H]2c2ncccc2F)OC12CC(O)C2] | (5′S,7a′R)-5′-(3-fluoropyridin-2-yl)-3- hydroxy-7a′-methyltetrahydro-3′H- spiro[cyclobutane-1,2′-pyrrolo[2,1- b]oxazol]-3′-one |
| I-34-Cis | 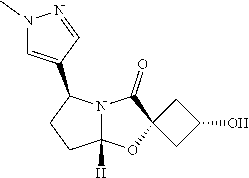 [SMILES: Cn1cc([C@@H]2CC[C@H]3O[C@]4(C[C@@H](O)C4)C(=O)N32)cn1] | (1s,3S,5′S,7a′R)-3-hydroxy-5′-(1-methyl- 1H-pyrazol-4-yl)tetrahydro-3′H- spiro[cyclobutane-1,2′-pyrrolo[2,1- b]oxazol]-3′-one |
| I-35-Trans | 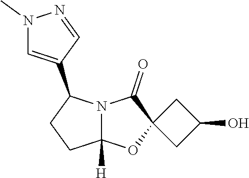 [SMILES: Cn1cc([C@@H]2CC[C@H]3O[C@]4(C[C@H](O)C4)C(=O)N32)cn1] | (1r,3R,5′S,7a′R)-3-hydroxy-5′-(1-methyl- 1H-pyrazol-4-yl)tetrahydro-3′H- spiro[cyclobutane-1,2′-pyrrolo[2,1- b]oxazol]-3′-one |

### Intermediate I-36

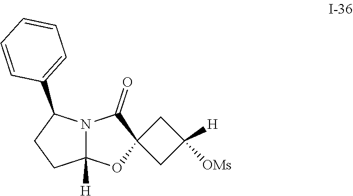 [SMILES: CS(=O)(=O)O[C@H]1C[C@]2(C1)O[C@@H]1CC[C@@H](c3ccccc3)N1C2=O]

### (1S,3S,5′S,7a′R)-3′-oxo-5′-phenyltetrahydro-3′H-spiro[cyclobutane-1,2′-pyrrolo[2,1-b]oxazol]-3-yl methanesulfonate

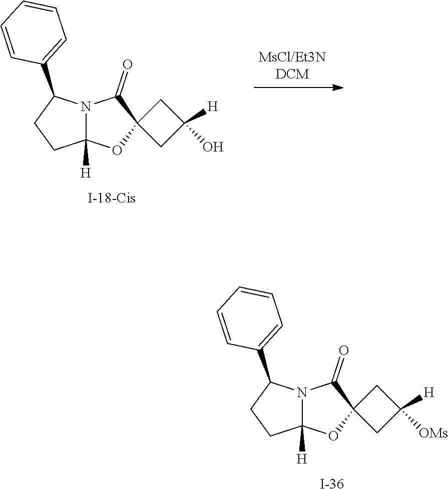

A solution (1S,3S,5′S,7a′R)-3-hydroxy-5′-phenyltetrahydro-3′H-spiro[cyclobutane-1,2′-pyrrolo[2,1-b]oxazol]-3′-one (120 mg, 0.463 mmol) and triethylamine (129 μl, 0.926 mmol) in DCM (3085 μl) was cooled to 0° C. where Ms-Cl (54.1 μl, 0.694 mmol) was added. The reaction was allowed to warm to RT and after 30 minutes the reaction was quenched with saturated sodium bicarbonate solution and extracted with DCM. The organic phase was washed with water followed by brine and then dried over sodium sulfate, filtered and concentrated. Purification via ISCO Silica 24 g Gold column eluting with 10-60% [EtOAc/EtOH (3/1)]/Hexanes to obtain (1S,3S,5′S,7a′R)-3′-oxo-5′-phenyltetrahydro-3′H-spiro[cyclobutane-1,2′-pyrrolo[2,1-b]oxazol]-3-yl methanesulfonate. MS (ESI) m/z [M+H]⁺ calc'd for C₁₆H₁₉INO₅S: 337, found: 337.

### Intermediate I-37

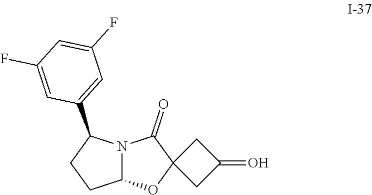 [SMILES: O=C1CC2(C1)O[C@@H]1CC[C@@H](c3cc(F)cc(F)c3)N1C2=O]

### (5′S,7a′R)-5′-(3,5-difluorophenyl)tetrahydro-3′H-spiro[cyclobutane-1,2′-pyrrolo[2,1-b]oxazole]-3,3′-dione

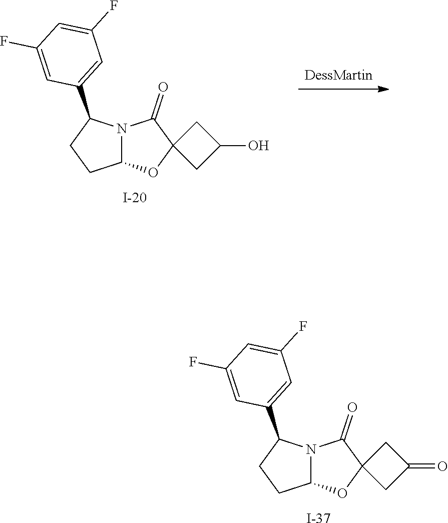

To a solution of (5′S,7a′R)-5′-(3,5-difluorophenyl)-3-hydroxytetrahydro-3′H-spiro[cyclobutane-1,2′-pyrrolo[2,1-b]oxazol]-3′-one (1 g, 3.39 mmol) in DCM (20 ml) was added DMP (2.155 g, 5.08 mmol) at 0° C. and the resulting mixture was stirred at 20° C. for 12 h. LCMS showed the starting material was consumed and desired compound was found. The reaction solution was added sat·NaHCO₃ (20 mL) and extracted with DCM (10 mL×3). The combined organic layers were washed with brine (5 mL), dried over Na2SO4, filtered and concentrated. The residue was purified by flash silica gel chromatography (ISCO®; 12 g SepaFlash® Silica Flash Column, eluent of 23% ethyl acetate/pet. ether gradient) to give (5′S,7a′R)-5′-(3,5-difluorophenyl)tetrahydro-3′H-spiro[cyclobutane-1,2′-pyrrolo[2,1-b]oxazole]-3,3′-dione as colorless oil. MS (ESI) m/z [M+H]⁺ calc'd for C₁₅H₁₃F₂NO₃: 294, found: 294.

### Intermediate I-38-Cis

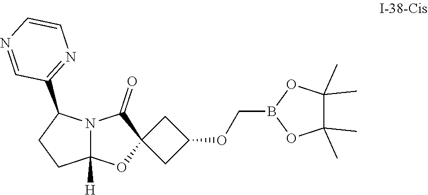 [SMILES: CC1(C)OB(CO[C@H]2C[C@]3(C2)O[C@@H]2CC[C@@H](c4cnccn4)N2C3=O)OC1(C)C]

### (1s,3S,5′S,7a′R)-5′-(pyrazin-2-yl)-3-((4,4,5,5-tetramethyl-1,3,2-dioxaborolan-2-yl)methoxy)tetrahydro-3′H-spiro[cyclobutane-1,2′-pyrrolo[2,1-b]oxazol]-3′-one (I-38-Cis)

A microwave vial was charged with (1S,3S,5′S,7a′R)-3-hydroxy-5′-(pyrazin-2-yl)tetrahydro-3′H-spiro[cyclobutane-1,2′-pyrrolo[2,1-b]oxazol]-3′-one (I-29-Cis) (40 mg, 0.153 mmol) sodium hydride (4.90 mg, 0.184 mmol) and potassium fluoride (26.7 mg, 0.459 mmol) inside of glovebox. The vial was sealed. Dry THF was added under an inert atmosphere. The solution was aged for 30 min until hydrogen evolution had completely ceased. Then 2-(bromomethyl)-4,4,5,5-tetramethyl-1,3,2-dioxaborolane (34.9 μl, 0.199 mmol) was added neat. Active bubbling was observed. After an additional 10 min, the vial was irradiated in the microwave for 40 min at 130° C. The suspension was filtered through celite affording crude (1s,3S,5′S,7a′R)-5′-(pyrazin-2-yl)-3-((4,4,5,5-tetramethyl-1,3,2-dioxaborolan-2-yl)methoxy)tetrahydro-3′H-spiro[cyclobutane-1,2′-pyrrolo[2,1-b]oxazol]-3′-one as brown oil. The material was used immediately without further purification. MS (ESI) m/z [M-pinacol+H₂O+H]+ calc'd for C₁₄H₁₈BN₃O₅: 320, found: 320.

The compounds presented in Table 6 below were prepared using a procedure analogous to one described for I-38-Cis with the appropriately chosen intermediate from Table 5.

TABLE 6

| Intermediate | Structure | IUPAC Name |
|---|---|---|
| I-39-Trans | 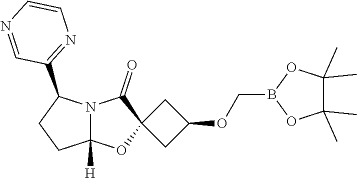 [SMILES: CC1(C)OB(CO[C@H]2C[C@@]3(C2)O[C@@H]2CC[C@@H](c4cnccn4)N2C3=O)OC1(C)C] | (1r,3R,5′S,7a′R)-5′-(pyrazin-2-yl)-3- ((4,4,5,5-tetramethyl-1,3,2- dioxaborolan-2- yl)methoxy)tetrahydro-3′H- spiro[cyclobutane-1,2′-pyrrolo[2,1- b]oxazol]-3′-one |

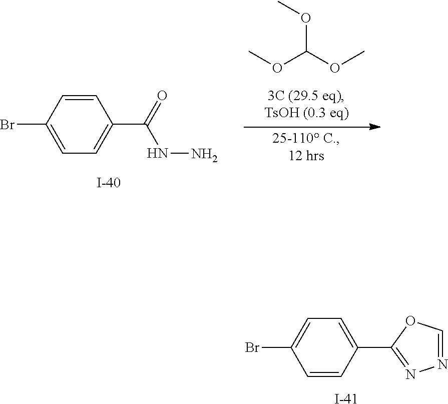

In a 3.00 L three neck round bottom flask with magnetic stirrer was added 4-bromobenzohydrazide (90.0 g, 418 mmol, 1.00 eq) and trimethoxymethane (1310 g, 12.35 mol, 29.5 eq) followed by TsOH (21.6 g, 125 mmol, 0.30 eq) at 25° C. The reaction was stirred at inner temperature 110° C. for 12 hrs under N₂ atmosphere. The reaction was then concentrated under vacuum to give a residue. It was purified by column chromatography (SiO₂, Petroleum ether/Ethyl acetate=10/1 to 1/1). 2-(4-bromophenyl)-1,3,4-oxadiazole (70.0 g, 311.05 mmol, 74.32% yield). MS (ESI): m/z 224.9, 226.9 [M+H]⁺. ¹H NMR: (400 MHz, DMSO-d6) δ=7.82 (d, J=8.8 Hz, 2H), 7.95 (d, J=8.8 Hz, 2H), 9.36 (s, 1H).

### Example 1.1 and 1.2

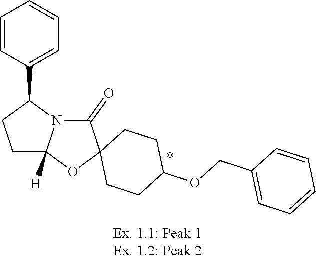 [SMILES: O=C1N2[C@@H](CC[C@H]2c2ccccc2)OC12CCC(OCc1ccccc1)CC2]

### ((1r or s),4R,5′S,7a′R)-4-(benzyloxy)-5′-phenyltetrahydro-3′H-spiro[cyclohexane-1,2′-pyrrolo[2,1-b]oxazol]-3′-one and ((1s or r),4R,5′S,7a′R)-4-(benzyloxy)-5′-phenyltetrahydro-3′H-spiro[cyclohexane-1,2′-pyrrolo[2,1-b]oxazol]-3′-one

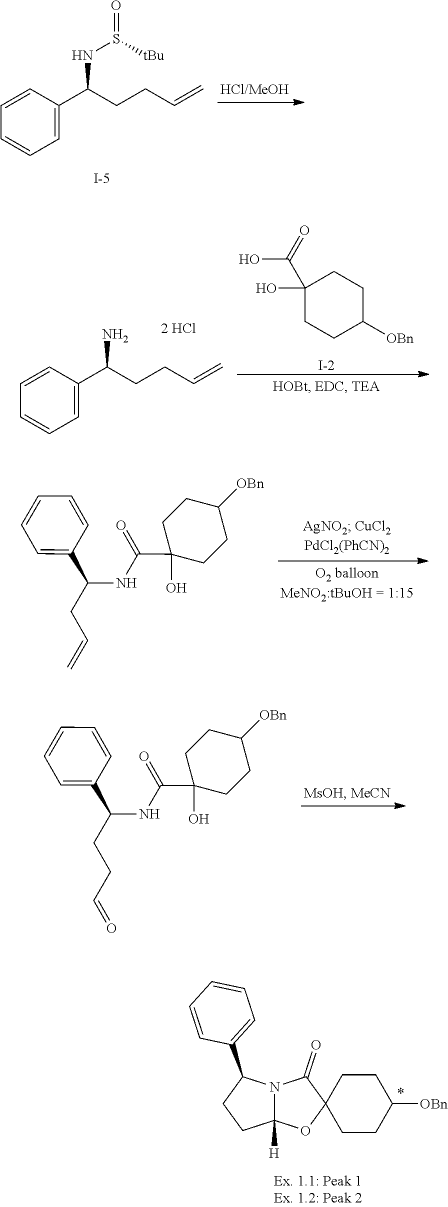

### Step 1. (S)-1-phenylbut-3-en-1-amine dihydrochloride

A mixture of (R)-2-methyl-N—((S)-1-phenylbut-3-en-1-yl)propane-2-sulfinamide (1.07 g, 4.26 mmol, I-5) in MeOH (20 ml) was added HCl/MeOH (4M) (5 ml) and the resulting mixture was stirred at 20° C. for 12 h. LCMS showed the starting material was consumed and desired compound was found. The reaction was directly concentrated to give (S)-1-phenylbut-3-en-1-amine dihydrochloride as a light-yellow solid, which was used to next step without further purification. MS (ESI): m/z 148.3 [M+H]⁺. ¹H NMR (400 MHz, CDCl₃-d) δ 8.08 (br s, 2H), 7.45 (br d, J=7.09 Hz, 2H), 7.28-7.37 (m, 3H), 5.43-5.63 (m, 1H), 4.96-5.16 (m, 2H), 4.29 (br s, 1H), 2.68-2.95 (m, 2H)

### Step 2. (S)-4-(benzyloxy)-1-hydroxy-N-(1-phenylbut-3-en-1-yl)cyclohexane-1-carboxamide

To a solution of 4-(benzyloxy)-1-hydroxycyclohexane-1-carboxylic acid (0.3 g, 1.2 mmol, I-2) in DMF (6 mL) was added TEA (0.668 mL, 4.79 mmol), HOBT (0.275 g, 1.80 mmol), EDC (0.35 g, 1.80 mmol) and (S)-1-phenylbut-3-en-1-amine dihydrochloride (0.31 g, 1.32 mmol) at 20° C. The resulting mixture was stirred at 20° C. After 16 h, the mixture was added to H₂O (20 mL) and then the mixture was extracted with EtOAc (10 mL×3). The combined organic layers were dried over Na₂SO₄, filtered and concentrated. The residue was purified by flash silica gel chromatography (ISCO®; 4 g SepaFlash® Silica Flash Column, eluent of 18% ethyl acetate/pet. ether gradient) to give (S)-4-(benzyloxy)-1-hydroxy-N-(1-phenylbut-3-en-1-yl)cyclohexane-1-carboxamide as yellow oil. MS (ESI): m/z 380 [M+H]⁺. ¹H NMR (400 MHz, CDCl₃-d) δ 7.28-7.32 (m, 5H), 7.14-7.26 (m, 5H), 5.56-5.74 (m, 1H), 4.94-5.08 (m, 3H), 4.53 (s, 2H), 3.30-3.46 (m, 1H), 2.58-2.60 (m, 1H), 2.46-2.60 (m, 1H), 1.83-2.01 (m, 4H), 1.45-1.71 (m, 4H).

### Step 3. (S)-4-(benzyloxy)-1-hydroxy-N-(4-oxo-1-phenylbutyl)cyclohexane-1-carboxamide

To a stirred mixture of silver nitrate (0.019 g, 0.13 mmol), copper(II) chloride (0.026 g, 0.19 mmol), bis(benzonitrile)palladium chloride (0.073 g, 0.19 mmol) under O₂ at 20° C. for 5 min, was added (S)-4-(benzyloxy)-1-hydroxy-N-(1-phenylbut-3-en-1-yl)cyclohexane-1-carboxamide (0.6 g, 1.58 mmol) in t-BuOH (20 mL) and MeNO₂ (1.3 mL). The mixture was stirred at 20° C. under O₂ for 16 h. The reaction was then concentrated in vacuum to give crude product (S)-4-(benzyloxy)-1-hydroxy-N-(4-oxo-1-phenylbutyl)cyclohexane-1-carboxamide which was used in the next step directly. MS (ESI): m/z 396. [M+H]⁺.

### Step 4. (5′S,7a′R)-4-(benzyloxy)-5′-phenyltetrahydro-3′H-spiro[cyclohexane-1,2′-pyrrolo[2,1-b]oxazol]-3′-one

To a stirred mixture of (S)-4-(benzyloxy)-1-hydroxy-N-(4-oxo-1-phenylbutyl)cyclohexane-1-carboxamide (0.6 g, 1.5 mmol) in CH₃CN (8 mL) was added methanesulfonic acid (0.44 g, 4.6 mmol) at 20° C. The mixture was stirred at 80° C. for 2 h. The reaction was then concentrated to give crude product which was purified by prep-HPLC (Column Boston Green ODS 150 mm×30 mm×5 m; Condition water (TFA)-ACN) to give two diastereomers. MS (ESI): m/z 378 [M+H]⁺

Ex. 1.1 (Peak 1): ¹H NMR (500 MHz, CD₃OD-d₄) δ 7.30-7.39 (m, 6H), 7.21-7.29 (m, 4H), 5.75 (dd, J=5.0, 7.0 Hz, 1H), 4.95 (t, J=7.5 Hz, 1H), 4.58 (s, 2H), 3.48 (tt, J=3.5, 10.0 Hz, 1H), 2.67 (dtd, J=5.5, 8.0, 13.5 Hz, 1H), 2.21-2.28 (m, 1H), 2.15 (qd, J=3.5, 13.5 Hz, 1H), 1.95-2.10 (m, 3H), 1.57-1.88 (m, 6H).

Ex. 1.2 (Peak 2): ¹H NMR (500 MHz, CD₃OD-d₄) δ 7.20-7.44 (m, 10H), 5.75 (dd, J=5.5, 7.5 Hz, 1H), 4.96 (t, J=8.0 Hz, 1H), 4.53 (s, 2H), 3.71 (br s, 1H), 2.67 (dtd, J=5.5, 8.0, 13.5 Hz, 1H), 2.16-2.28 (m, 2H), 1.93-2.05 (m, 4H), 1.83-1.92 (m, 2H), 1.66-1.81 (m, 2H), 1.51 (br d, J=13.5 Hz, 1H).

Each compound presented in Table 7 below were prepared using procedure analogous to one described for Example 1.1/1.2 with appropriately chosen intermediates.

TABLE 7

| | | | Exact Mass |
|---|---|---|---|
| Example | Structure | IUPAC Name | [M + H]+ |
| 1.3 | 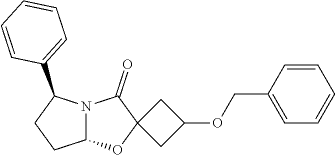 | (5′S,7a′R)-3- (benzyloxy)-5′- phenyltetrahydro-3′H- spiro[cyclobutane-1,2′- pyrrolo[2,1-b]oxazol]- 3′-one | Calc'd 380, found 380 |

### Example 2.1 and 2.2

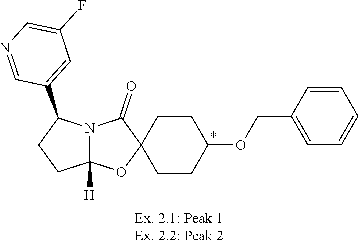 [SMILES: O=C1N2[C@@H](CC[C@H]2c2cncc(F)c2)OC12CCC(OCc1ccccc1)CC2]

### ((1r or s),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one and ((1s or r),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one

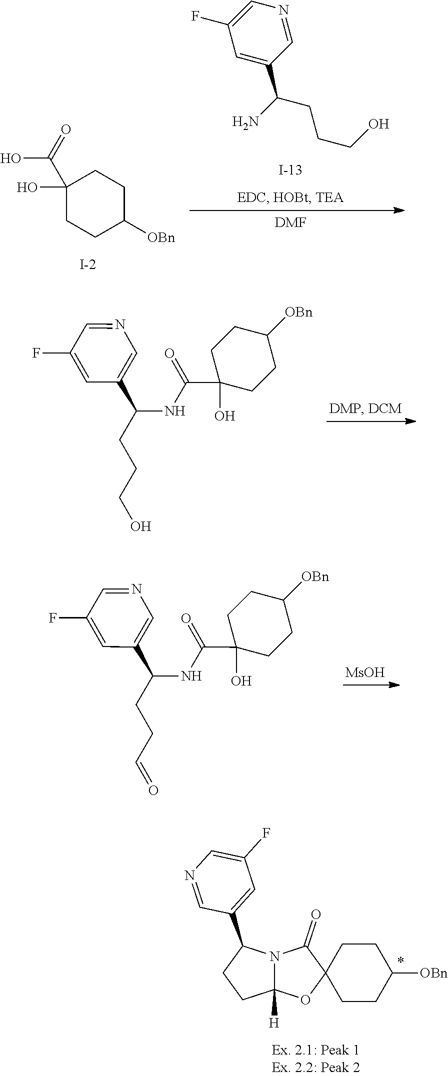

### Step 1. (S)-4-(benzyloxy)-N-(1-(5-fluoropyridin-3-yl)-4-hydroxybutyl)-1-hydroxycyclohexane-1-carboxamide

To a solution of 4-(benzyloxy)-1-hydroxycyclohexane-1-carboxylic acid (1 g, 4.00 mmol, I-2) in DMF (20 mL) was added TEA (2.23 mL, 16 mmol), HOBT (0.92 g, 5.99 mmol), EDC (1.15 g, 5.99 mmol) and (S)-4-amino-4-(5-fluoropyridin-3-yl)butan-1-ol (0.88 g, 4.79 mmol, I-13) at 20° C. The resulting mixture was stirred at 20° C. for 16 h. The mixture was added to H₂O (40 mL) and then extracted with EtOAc (20 mL×3). The combined organic layers were washed with brine (40 mL), dried over Na₂SO₄, filtered and concentrated. The residue was purified by flash silica gel chromatography (ISCO®; 12 g SepaFlash® Silica Flash Column, eluent of 5% MeOH/DCM) to give (S)-4-(benzyloxy)-N-(1-(5-fluoropyridin-3-yl)-4-hydroxybutyl)-1-hydroxycyclohexane-1-carboxamide as yellow oil. MS (ESI): m/z 417 [M+H]⁺. ¹H NMR (500 MHz, CDCl₃-d) δ 8.33-8.44 (m, 2H), 7.28-7.46 (m, 6H), 4.94-5.04 (m, 1H), 4.45-4.60 (m, 2H), 3.39-3.80 (m, 3H), 1.85-2.05 (m, 7H), 1.65-1.82 (m, 2H), 1.51-1.63 (m, 4H).

### Step 2. (S)-4-(benzyloxy)-N-(1-(5-fluoropyridin-3-yl)-4-oxobutyl)-1-hydroxycyclohexane-1-carboxamide

To a solution of (S)-4-(benzyloxy)-N-(1-(5-fluoropyridin-3-yl)-4-hydroxybutyl)-1-hydroxycyclohexane-1-carboxamide (1.6 g, 3.7 mmol), pyridine (0.295 mL, 3.65 mmol) in DCM (25 mL) was added DMP (3.10 g, 7.30 mmol) at 0° C. and the resulting mixture was stirred at 20° C. After 16 h. sat. NaHCO₃ (40 mL) was added and the reaction extracted with DCM (30 mL×2). The combined organic layers were dried over Na₂SO₄, filtered and concentrated to give (S)-4-(benzyloxy)-N-(1-(5-fluoropyridin-3-yl)-4-oxobutyl)-1-hydroxycyclohexane-1-carboxamide as colorless oil, which was used in the next step without further purification. MS (ESI): m/z 415 [M+H]⁺.

### Step 3. (5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one

To a mixture of (S)-4-(benzyloxy)-N-(1-(5-fluoropyridin-3-yl)-4-oxobutyl)-1-hydroxycyclohexane-1-carboxamide (1.5 g, 3.6 mmol) in acetonitrile (20 mL) was added methanesulfonic acid (0.678 mL, 10.86 mmol) and the resulting mixture was stirred at 80° C. for 2 h. The reaction was then cooled, filtered and the filtrate was purified by Prep-HPLC (water (TFA)-ACN) to give two diastereomers. Ex. 2.1 (Peak 1) both as colorless oil. Peak 2, was further purified by prep-HPLC (NH₃H₂O+NH₄HCO₃)-ACN to give Ex. 2.2 as colorless oil. MS (ESI): m/z 397 [M+H]⁺

Ex. 2.1 (Peak 1): ¹H NMR (400 MHz, CD₃OD-d₄) δ 8.37-8.50 (m, 2H), 7.69 (br d, J=9.2 Hz, 1H), 7.22-7.38 (m, 5H), 5.77 (dd, J=5.2, 7.6 Hz, 1H), 5.04 (t, J=8.0 Hz, 1H), 4.58 (s, 2H), 3.44-3.54 (m, 1H), 2.70-2.83 (m, 1H), 2.25-2.36 (m, 1H), 2.17-2.24 (m, 1H), 2.00-2.12 (m, 3H), 1.59-1.87 (m, 6H).

Ex. 2.2 (Peak 2): ¹H NMR (400 MHz, CD₃OD-d₄) δ 8.38 (d, J=2.0 Hz, 2H), 7.57-7.67 (m, 1H), 7.20-7.44 (m, 5H), 5.77 (dd, J=4.8, 7.2 Hz, 1H), 5.03 (t, J=8.0 Hz, 1H), 4.53 (s, 2H), 3.71 (br s, 1H), 2.70-2.80 (m, 1H), 2.15-2.32 (m, 2H), 1.94-2.07 (m, 4H), 1.86-1.92 (m, 2H), 1.70-1.81 (m, 2H), 1.53 (br d, J=13.2 Hz, 1H).

### Example 3.1

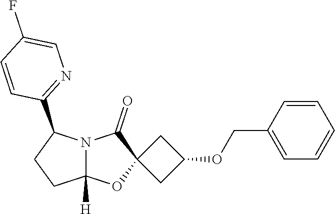 [SMILES: O=C1N2[C@@H](CC[C@H]2c2ccc(F)cn2)O[C@]12C[C@@H](OCc1ccccc1)C2]

### (1s,3R,5′S,7a′R)-3-(benzyloxy)-5′-(5-fluoropyridin-2-yl)tetrahydro-3′H-spiro[cyclobutane-1,2′-pyrrolo[2,1-b]oxazol]-3′-one

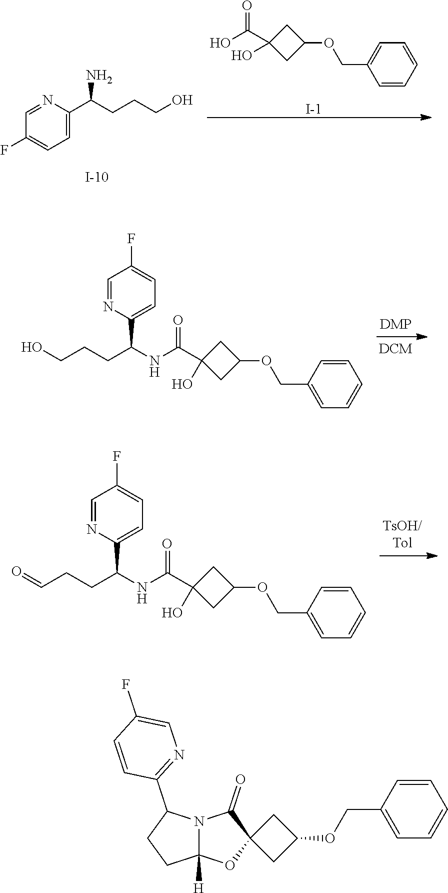

### Step 1. (S)-3-(benzyloxy)-N-(1-(5-fluoropyridin-2-yl)-4-hydroxybutyl)-1-hydroxycyclobutane-1-carboxamide

To a stirred mixture of (S)-4-amino-4-(5-fluoropyridin-2-yl)butan-1-ol (1.5 g, 8.14 mmol, I-10) in DMF (40 ml) was added to TEA (1.14 ml, 8.14 mmol), 3-(benzyloxy)-1-hydroxycyclobutane-1-carboxylic acid (2.71 g, 12.2 mmol, I-1), HOBT (1.87 g, 12.2 mmol), EDC (3.12 g, 16.3 mmol). Then the mixture was stirred at 20° C. After 12 h, water (300 mL) was added, and the mixture was extracted with EtOAc (3×300 mL). The combined organic layers were concentrated, washed with brine (2×200 mL), dried over Na₂SO₄, filtered and the solvent was evaporated under reduced pressure. The crude was purified by flash silica gel chromatography (ISCO®; 220 g SepaFlash® Silica Flash Column, eluent of 10% EtOAc/Pet.ether gradient) to give (S)-3-(benzyloxy)-N-(1-(5-fluoropyridin-2-yl)-4-hydroxybutyl)-1-hydroxycyclobutane-1-carboxamide as yellow oil. MS (ESI) m/z 389 [M+H]⁺. ¹H NMR (400 MHz, CDCl₃-d) δ ppm 1.45-1.57 (m, 2H) 1.80-1.92 (m, 2H) 2.09 (dd, J=12.1, 6.3 Hz, 1H) 2.17 (dd, J=12.1, 6.38 Hz, 1H) 2.49-2.58 (m, 1H) 2.74-2.81 (m, 1H) 2.84 (s, 1H) 2.91 (s, 2H) 3.56-3.63 (m, 2H) 4.04-4.12 (m, 1H) 4.36-4.42 (m, 2H) 5.00-5.10 (m, 1H) 5.26 (s, 1H) 7.22-7.26 (m, 2H) 7.27-7.30 (m, 2H) 7.31-7.38 (m, 2H) 7.89 (d, J=8.5 Hz, 1H) 8.39 (d, J=2.74 Hz, 1H).

### Step 2. (S)-3-(benzyloxy)-N-(1-(5-fluoropyridin-2-yl)-4-oxobutyl)-1-hydroxycyclobutane-1-carboxamide

To a solution of (S)-3-(benzyloxy)-N-(1-(5-fluoropyridin-2-yl)-4-hydroxybutyl)-1-hydroxycyclobutane-1-carboxamide (500 mg, 1.29 mmol) in DCM (8 ml) was added DMP (819 mg, 1.93 mmol) at 0° C. and the resulting mixture was stirred at 20° C. After 1 h. the reaction was added to sat·NaHCO₃ (15 mL) and extracted with DCM (10 mL×2), and washed with brine (10 mL). The combined organic layers were dried over Na₂SO₄, filtered and concentrated to afford (S)-3-(benzyloxy)-N-(1-(5-fluoropyridin-2-yl)-4-oxobutyl)-1-hydroxycyclobutane-1-carboxamide as yellow oil, which was used in the next step directly. MS (ESI): m/z 387 [M+H]⁺

### Step 3. (5′S,7a′R)-3-(benzyloxy)-5′-(5-fluoropyridin-2-yl)tetrahydro-3′H-spiro[cyclobutane-1,2′-pyrrolo[2,1-b]oxazol]-3′-one

To a solution of (S)-3-(benzyloxy)-N-(1-(5-fluoropyridin-2-yl)-4-oxobutyl)-1-hydroxycyclobutane-1-carboxamide (450 mg, 1.17 mmol) in toluene (8 ml) was added p-toluenesulfonic acid monohydrate (55.4 mg, 0.29 mmol) and the resulting mixture was stirred at 80° C. for 12 h. The mixture was then quenched with water (20 mL) and extracted with EtOAc (20 mL×3). The combined organic layers were washed with NaSO₄ (20 mL) and brine (20 mL), dried over Na₂SO₄, filtered and the solvent was evaporated under reduced pressure and the residue was purified by flash silica gel chromatography (ISCO®; Agela® Flash Column Silica-CS (1 g), Eluent of 0-5% Ethyl acetate/Petroleum ether) to give the two diastereomers of (5′S,7a′R)-3-(benzyloxy)-5′-(5-fluoropyridin-2-yl)tetrahydro-3′H-spiro[cyclobutane-1,2′-pyrrolo[2,1-b]oxazol]-3′-one. MS (ESI): m/z 369 [M+H]⁺ Ex. 3.1 (Cis): ¹H NMR (500 MHz, CDCl₃-d) δ 8.50 (d, J=2.5 Hz, 1H), 7.48 (dt, J=2.5, 8.20 Hz, 1H), 7.38 (dd, J=4.5, 8.5 Hz, 1H), 7.28 (d, J=1.5 Hz, 1H), 7.19-7.27 (m, 2H), 5.61 (dd, J=5.0, 7.5 Hz, 1H), 5.01 (t, J=7.5 Hz, 1H), 4.39 (s, 2H), 3.98 (t, J=6.5 Hz, 1H), 2.74-2.87 (m, 2H), 2.64 (dtd, J=2.5, 8.0, 13.5 Hz, 1H), 2.38 (dd, J=7.0, 11.0 Hz, 1H), 2.28 (dd, J=7.0, 11.83 Hz, 1H), 2.17-2.24 (m, 1H), 2.04-2.15 (m, 1H), 1.57-1.70 (m, 1H).

### Example 4.1 and 4.2

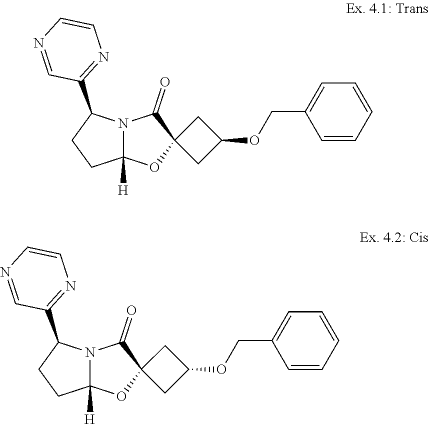 [SMILES: O=C1N2[C@@H](CC[C@H]2c2cnccn2)O[C@]12C[C@H](OCc1ccccc1)C2 ; O=C1N2[C@@H](CC[C@H]2c2cnccn2)O[C@]12C[C@@H](OCc1ccccc1)C2]

### ((1r),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one (Ex. 4.1 Trans) and ((1s),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one (Ex. 4.1 Cis)

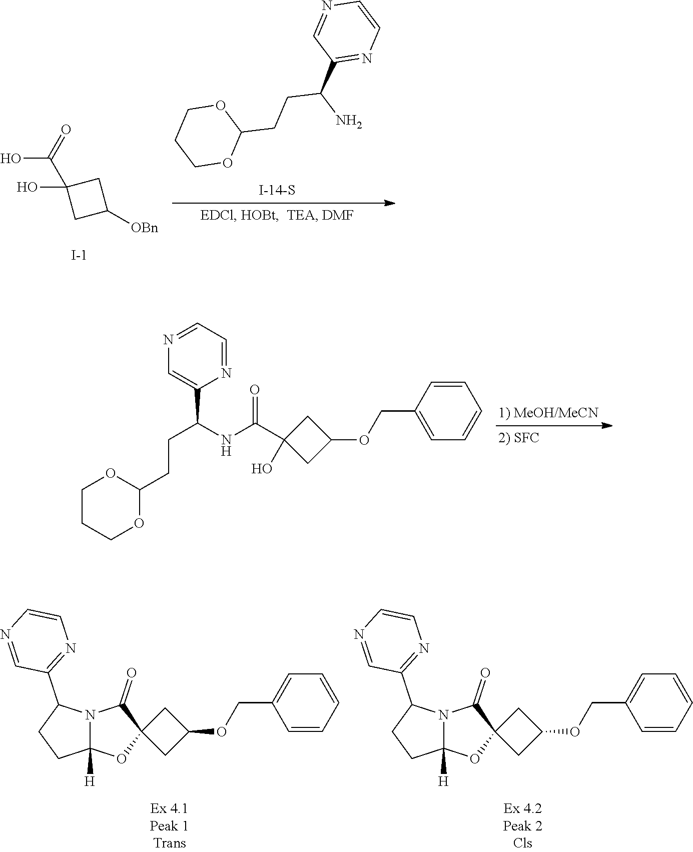 [SMILES: O=C(O)C1(O)CC(OCc2ccccc2)C1 ; N[C@@H](CCC1OCCCO1)c1cnccn1 ; [I-] ; O=C(N[C@@H](CCC1OCCCO1)c1cnccn1)C1(O)CC(OCc2ccccc2)C1 ; O=C1N2C(c3cnccn3)CC[C@H]2O[C@]12C[C@H](OCc1ccccc1)C2 ; O=C1N2C(c3cnccn3)CC[C@H]2O[C@]12C[C@@H](OCc1ccccc1)C2]

### Step 1. N-(3-(1,3-dioxan-2-yl)-1-(pyrazin-2-yl)propyl)-3-(benzyloxy)-1-hydroxycyclobutane-1-carboxamide

To a solution of 3-(1,3-dioxan-2-yl)-1-(pyrazin-2-yl)propan-1-amine (700 mg, 3.14 mmol, I-14-S) in DMF (20 mL) was added 3-(benzyloxy)-1-hydroxycyclobutane-1-carboxylic acid (836 mg, 3.76 mmol, I-1), TEA (1.31 mL, 9.41 mmol), HOBT (720 mg, 4.70 mmol), EDCI (902 mg, 4.70 mmol) and the resulting mixture was stirred at 20° C. for 6 h. The mixture was then added to water (50 mL) and extracted with EtOAc (20 mL×3). The combined organic fractions were washed with brine (20 mL), dried over Na₂SO₄, filtered and concentrated under reduced pressure. The residue was purified by flash silica gel chromatography (ISCO®; Agela® Flash Column Silica-CS (20 g), Eluent of 0-10% Ethyl acetate/Petroleum ether gradient) to give N-(3-(1,3-dioxan-2-yl)-1-(pyrazin-2-yl)propyl)-3-(benzyloxy)-1-hydroxycyclobutane-1-carboxamide as yellow oil. MS (ESI): m/z 428 [M+H]⁺

### Step 2. 3-(benzyloxy)-5′-(pyrazin-2-yl)tetrahydro-3′H-spiro[cyclobutane-1,2′-pyrrolo[2,1-b]oxazol]-3′-one

To a solution of N-(3-(1,3-dioxan-2-yl)-1-(pyrazin-2-yl)propyl)-3-(benzyloxy)-1-hydroxycyclobutane-1-carboxamide (800 mg, 1.87 mmol) in MeCN (15 mL) was added MsOH (0.122 mL, 1.87 mmol) and the resulting mixture was stirred at 80° C. for 2 h. The reaction mixture was filtered and concentrated and the residue was purified by flash silica gel chromatography (ISCO®; Agela® Flash Column Silica-CS (12 g), Eluent of 0-10% Ethyl acetate/Petroleum ether gradient) to give crude 3-(benzyloxy)-5′-(pyrazin-2-yl)tetrahydro-3′H-spiro[cyclobutane-1,2′-pyrrolo[2,1-b]oxazol]-3′-one. The mixture of diastereomers (cis/trans) for (5′S,7a′R)-3-(benzyloxy)-5′-(pyrazin-2-yl)tetrahydro-3′H-spiro[cyclobutane-1,2′-pyrrolo[2,1-b]oxazol]-3′-one (420 mg, 1.195 mmol) was separated by SFC (Column DAICEL CHIRALPAK AD (250 mm×30 mm, 10 m) Condition 0.1% NH₃·H₂O EtOH) to give the two diastereomers of (5′S,7a′R)-3-(benzyloxy)-5′-(pyrazin-2-yl)tetrahydro-3′H-spiro[cyclobutane-1,2′-pyrrolo[2,1-b]oxazol]-3′-one. Ex. 4.1 (Peak 1/Trans) ee=92%, t_{R}=1.99 min. and Ex. 4.2 (Peak 2/Cis) ee=98.7%, t_{R}=2.7 min. both as colorless oil. MS (ESI): m/z 352 [M+H]⁺

Ex. 4.1 (Peak 1/trans): ¹H NMR (CDCl₃-d, 400 MHz): δ 8.67 (d, J=1.2 Hz, 1H), 8.49-8.56 (m, 2H), 7.27-7.38 (m, 5H), 5.63 (t, J=5.6 Hz, 1H), 5.08-5.16 (m, 1H), 4.42-4.51 (m, 2H), 4.33 (quin, J=7.2 Hz, 1H), 2.80 (dd, J=13.2, 7.6 Hz, 1H), 2.61-2.68 (m, 1H), 2.53-2.60 (m, 1H), 2.39-2.47 (m, 2H), 2.24-2.34 (m, 2H), 1.70-1.81 (m, 1H)

Ex. 4.2 (Peak 2/cis): ¹H NMR (CDCl₃-d, 400 MHz): δ 8.67 (s, 1H), 8.49-8.57 (m, 2H), 7.28-7.38 (m, 5H), 5.64-5.71 (m, 1H), 5.05-5.15 (m, 1H), 4.46 (s, 2H), 4.07 (m, 1H), 2.79-2.94 (m, 2H), 2.56-2.66 (m, 1H), 2.43 (dd, J=11.6, 7.2 Hz, 1H), 2.24-2.38 (m, 3H), 1.67-1.77 (m, 1H)

### Example 5.1 and 5.2

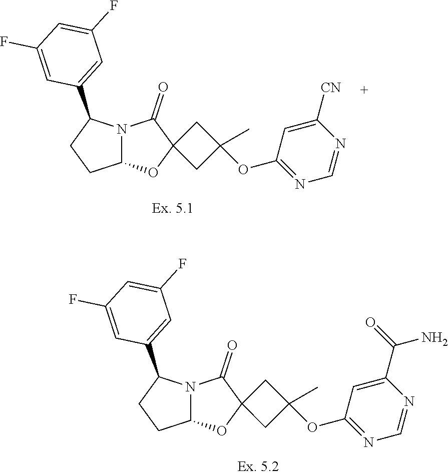

### 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carbonitrile (Ex. 5.1) and 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carboxamide (Ex. 5.2)

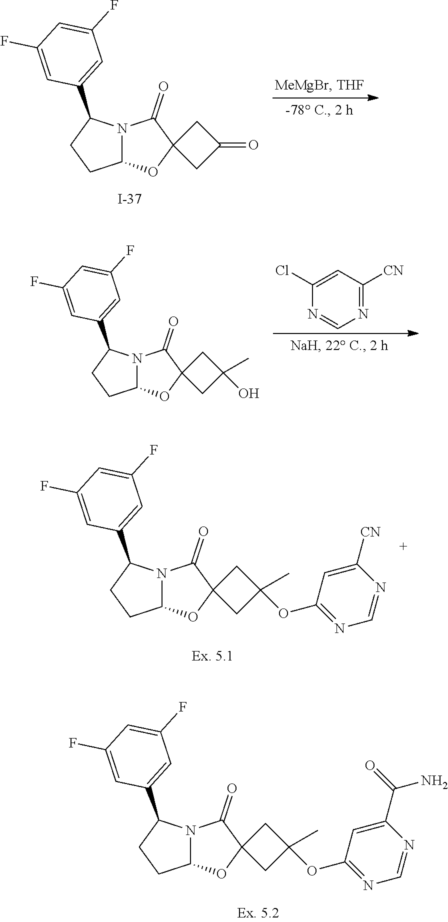

### Step 1. (5′S,7a′R)-5′-(3,5-difluorophenyl)-3-hydroxy-3-methyltetrahydro-3′H-spiro[cyclobutane-1,2′-pyrrolo[2,1-b]oxazol]-3′-one

To a solution of (5′S,7a′R)-5′-(3,5-difluorophenyl)tetrahydro-3′H-spiro[cyclobutane-1,2′-pyrrolo[2,1-b]oxazole]-3,3′-dione (100 mg, 0.34 mmol, I-37) in THF (2 mL) was added methylmagnesium bromide (0.23 mL, 0.68 mmol) at −78° C. and the reaction was stirred at −78° C. for 2 h. The mixture was then added to sat. NH₄Cl (5 mL) and extracted with EtOAc (2×3 mL). The combined organic layers were dried over Na₂SO₄, filtered and concentrated. The residue was purified by preparative HPLC (Boston Uni C18 [150 mm×40 mm×5 μm]; 35-65% Water [0.01% TFA]/MeCN) to give (5′S,7a′R)-5′-(3,5-difluorophenyl)-3-hydroxy-3-methyltetrahydro-3′H-spiro[cyclobutane-1,2′-pyrrolo[2,1-b]oxazol]-3′-one as white solid. MS (ESI) m/z [M+H]⁺ calc'd for C₁₆H₁₇F₂NO₃: 310, found: 310.

### Step 2. 6-(((5'S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carbonitrile and 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carboxamide

To a solution of (5′S,7a′R)-5′-(3,5-difluorophenyl)-3-hydroxy-3-methyltetrahydro-3′H-spiro[cyclobutane-1,2′-pyrrolo[2,1-b]oxazol]-3′-one (10 mg, 0.032 mmol) in THF (1 mL) was added NaH (2.59 mg, 0.065 mmol) at 0° C. The mixture was stirred 0° C. for 5 min. Then 6-chloropyrimidine-4-carbonitrile (6.77 mg, 0.048 mmol) was added. The reaction was stirred at room temperature for 2 h. The mixture was filtered and the filtrate was concentrated to give a residue which was purified by preparative HPLC (C18; 50-80% water [0.05% NH₃H₂O+10 mM NH₄HCO₃]/MeCN) to give 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carbonitrile (Ex. 5.1) and 6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carboxamide (Ex. 5.2).

Ex. 5.1: ¹H NMR (400 MHz, CDCl₃) δ 8.78 (d, J=1.2 Hz, 1H), 7.05 (d, J=1.2 Hz, 1H), 6.59-6.88 (m, 3H), 5.62 (dd, J=5.6 Hz, 1H), 4.96 (t, J=7.6 Hz, 1H), 3.20 (br d, J=13.6 Hz, 1H), 2.88-2.97 (m, 1H), 2.73-2.81 (m, 1H), 2.51-2.65 (m, 2H), 2.19-2.28 (m, 1H), 1.93-2.03 (m, 1H), 1.86 (s, 3H), 1.69-1.78 (m, 1H). MS (ESI) m/z [M+H]⁺ calc'd for C₂₁H₁₈F₂N₄O₃: 413, found: 413.

Ex. 5.2: ¹H NMR (400 MHz, CDCl₃) δ 8.70 (d, J=1.2 Hz, 1H), 7.73 (br s, 1H), 7.47 (d, J=1.6 Hz, 1H), 6.61-6.83 (m, 3H), 5.54-5.70 (m, 2H), 4.96 (t, J=7.6 Hz, 1H), 3.22 (br d, J=13.2 Hz, 1H), 2.94 (br d, J=13.6 Hz, 1H), 2.72-2.82 (m, 1H), 2.51-2.65 (m, 2H), 2.18-2.30 (m, 1H), 1.93-2.02 (m, 1H), 1.86 (s, 3H), 1.70-1.78 (m, 1H). MS (ESI) m/z [M+H]⁺ calc'd for C₂₁H₂₀F₂N₄O₄: 431, found: 431.

### Example 6.1, 6.2, 6.3, 6.4

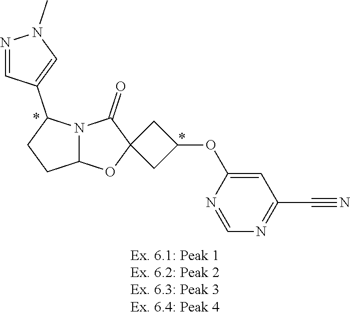 [SMILES: Cn1cc(C2CCC3OC4(CC(Oc5cc(C#N)ncn5)C4)C(=O)N32)cn1]

### 6-((((1r or s),3R,5′(S or R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile and 6-((((1s or r),3R,5′(S or R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile and 6-((((1r or s),3R,5′(R or S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile and 6-((((1s or r),3R,5′(R or S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile

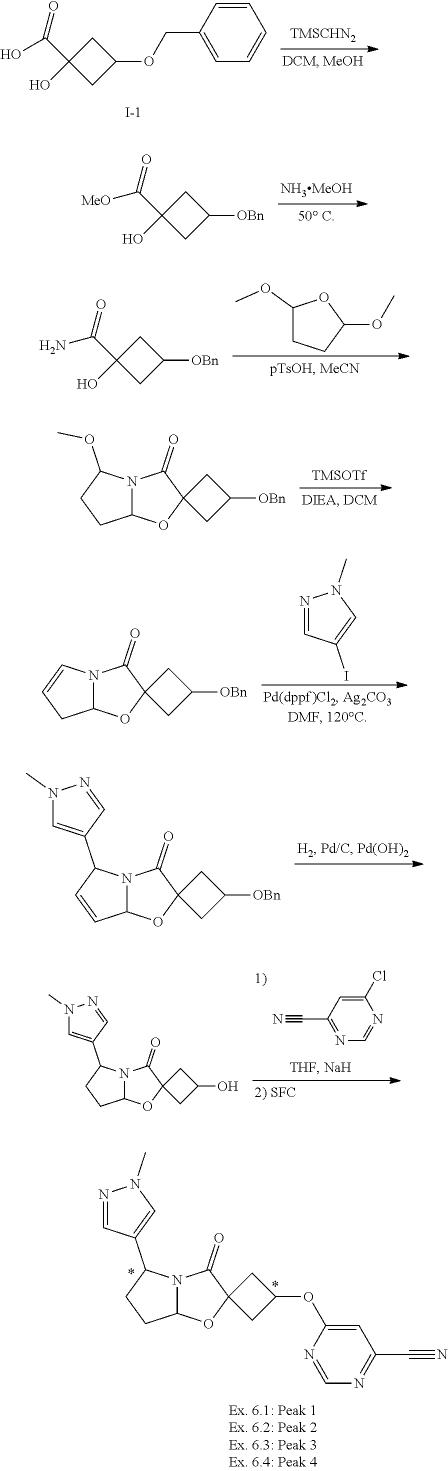

### Step 1. Methyl-3-(benzyloxy)-1-hydroxycyclobutane-1-carboxylate

To a mixture of 3-(benzyloxy)-1-hydroxycyclobutane-1-carboxylic acid (6.0 g, 27.0 mmol) in DCM (100 mL) and MeOH (30 mL) was added TMS-diazomethane (27.0 mL, 54.0 mmol) at 0° C. The mixture was stirred at 20° C. for 1 h. The mixture was then concentrated to give methyl 3-(benzyloxy)-1-hydroxycyclobutane-1-carboxylate which was used without further purification. ¹H NMR (CDCl₃-d, 400 MHz): δ 7.27-7.38 (m, 5H), 4.46 (d, J=1.2 Hz, 2H), 3.99-4.44 (m, 1H), 3.76-3.89 (m, 3H), 2.83 (ddd, J=10.0, 6.8, 3.2 Hz, 1H), 2.58-2.67 (m, 1H), 2.33-2.47 (m, 2H).

### Step 2. 3-(benzyloxy)-1-hydroxycyclobutane-1-carboxamide

A solution of methyl 3-(benzyloxy)-1-hydroxycyclobutane-1-carboxylate (6.2 g, 26.2 mmol) in NH₃-MeOH (7 M) (100 mL) was stirred at 50° C. for 12 h. After completion, the mixture was concentrated in vacuo to give crude 3-(benzyloxy)-1-hydroxycyclobutane-1-carboxamide which was used without further purification. MS (ESI): m/z 221 [M+H]⁺

### Step 3. 3-(benzyloxy)-5′-methoxytetrahydro-3′H-spiro[cyclobutane-1,2′-pyrrolo[2,1-b]oxazol]-3′-one

To a solution of 3-(benzyloxy)-1-hydroxycyclobutane-1-carboxamide (4.5 g, 20.3 mmol) in MeCN (100 mL) was added 2,5-dimethoxytetrahydrofuran (3.36 g, 25.4 mmol), 4-methylbenzenesulfonic acid hydrate (0.387 g, 2.03 mmol) and the resulting mixture was stirred at 40° C. for 12 h. The mixture was quenched with sat. aq. NaHCO₃ (40 mL) and extracted with EtOAc (20 mL×3). The combined organic fractions were washed with brine (20 mL), dried over Na₂SO₄, filtered and concentrated. The residue was purified by flash silica gel chromatography (ISCO®; Agela® Flash Column Silica-CS (4 g), Eluent of 0-10% Ethyl acetate/Petroleum ether gradient) to give 3-(benzyloxy)-5′-methoxytetrahydro-3′H-spiro[cyclobutane-1,2′-pyrrolo[2,1-b]oxazol]-3′-one. MS (ESI): m/z 303 [M+H]⁺. ¹H NMR (CDCl₃-d, 400 MHz): δ 7.28-7.36 (m, 5H), 5.46-5.58 (m, 1H), 5.00-5.08 (m, 1H), 4.44-4.48 (m, 2H), 4.00-4.35 (m, 1H), 3.41 (d, J=1.2 Hz, 3H), 2.78-2.84 (m, 1H), 2.57-2.77 (m, 1H), 2.37-2.44 (m, 1H), 2.36 (s, 3H), 1.88-1.99 (m, 1H), 1.61-1.72 (m, 1H).

### Step 4. 3-(benzyloxy)-7′,7a′-dihydro-3′H-spiro[cyclobutane-1,2′-pyrrolo[2,1-b]oxazol]-3′-one

To a DCM (60 mL) solution of the 3-(benzyloxy)-5′-methoxytetrahydro-3′H-spiro[cyclobutane-1,2′-pyrrolo[2,1-b]oxazol]-3′-one (3.0 g, 9.9 mmol) and DIPEA (3.27 mL, 19.8 mmol) was slowly added TMSOTf (2.54 mL, 14.8 mmol) at 0° C. The mixture was then stirred at 20° C. for 3 h. The reaction was quenched by the addition of water (50 mL) and extracted with DCM (50 mL×3). The combined organic fractions were washed with brine (50 mL×2), dried over Na₂SO₄, filtered and concentrated in vacuo. The residue was purified by flash silica gel chromatography (ISCO®; Agela® Flash Column Silica-CS (12 g), Eluent of 0-10% Ethyl acetate/Petroleum ether gradient) to give 3-(benzyloxy)-7′,7a′-dihydro-3′H-spiro[cyclobutane-1,2′-pyrrolo[2,1-b]oxazol]-3′-one. MS (ESI): m/z 272 [M+H]⁺. ¹H NMR (CDCl₃-d, 400 MHz): δ 7.34-7.36 (m, 4H), 7.29-7.32 (m, 1H), 6.46 (td, J=4.0, 2.0 Hz, 1H), 5.80-5.95 (m, 1H), 5.33-5.38 (m, 1H), 4.46 (d, J=3.6 Hz, 2H), 4.27-4.36 (m, 1H), 2.56-2.72 (m, 3H), 2.28-2.46 (m, 3H).

### Step 5. 3-(benzyloxy)-5′-(1-methyl-1H-pyrazol-4-yl)-5′,7a′-dihydro-3′H-spiro[cyclobutane-1,2′-pyrrolo[2,1-b]oxazol]-3′-one

To a solution of 3-(benzyloxy)-7′,7a′-dihydro-3′H-spiro[cyclobutane-1,2′-pyrrolo[2,1-b]oxazol]-3′-one (200 mg, 0.74 mmol) in DMF (6 mL) was added 4-iodo-1-methyl-1H-pyrazole (307 mg, 1.47 mmol), Ag₂CO₃ (407 mg, 1.47 mmol), Pd(dppf)Cl₂ (108 mg, 0.15 mmol) and the resulting mixture was stirred at 120° C. under N₂ for 12 h. The mixture was quenched with water (10 mL) and extracted with EtOAc (10 mL×3). The combined organic fractions were washed with brine (10 mL×2), dried over Na₂SO₄, filtered and concentrated. The residue was purified by prep. HPLC (Column Welch Xtimate C18 150×25 mm×5 m; Condition water (0.01% TFA)-MeCN) to give 3-(benzyloxy)-5′-(1-methyl-1H-pyrazol-4-yl)-5′,7a′-dihydro-3′H-spiro[cyclobutane-1,2′-pyrrolo[2,1-b]oxazol]-3′-one. MS (ESI): m/z 352 [M+H].

### Step 6. 3-hydroxy-5′-(1-methyl-1H-pyrazol-4-yl)tetrahydro-3′H-spiro[cyclobutane-1, 2′-pyrrolo[2, 1-b]oxazol]-3′-one

To a solution of 3-(benzyloxy)-5′-(1-methyl-1H-pyrazol-4-yl)-5′,7a′-dihydro-3′H-spiro[cyclobutane-1,2′-pyrrolo[2,1-b]oxazol]-3′-one (60 mg, 0.17 mmol) in MeOH (5 mL) was added Pd(OH)₂/C (11.99 mg, 0.017 mmol) and Pd/C (9.09 mg, 0.017 mmol) at 20° C. The resulting mixture was stirred at 40° C. for 16 h under H₂. The mixture was filtered and evaporated under reduced pressure to give 3-hydroxy-5′-(1-methyl-1H-pyrazol-4-yl)tetrahydro-3′H-spiro[cyclobutane-1, 2′-pyrrolo[2, 1-b]oxazol]-3′-one. MS (ESI): m/z 264 [M+H]⁺

### Step 7. 6-((5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile

To a solution of 3-hydroxy-5′-(1-methyl-1H-pyrazol-4-yl)tetrahydro-3′H-spiro[cyclobutane-1, 2′-pyrrolo[2, 1-b]oxazol]-3′-one (30 mg, 0.114 mmol) in THF (1 mL) was added NaH (9 mg, 0.23 mmol) at 0° C. and stirred for 10 min. Then 6-chloropyrimidine-4-carbonitrile (234 mg, 0.17 mmol) was added and the resulting mixture was stirred at 20° C. for 2 h. To the reaction mixture was then added MeCN (1 mL), filtered, concentrated in vacuo and purified by prep. reversed-phase HPLC (Column Welch Xtimate C18 150×25 mm×5 m; Condition water (10 mM-NH₄—HCO₃)-MeCN) to give 6-((5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile. MS (ESI): m/z 367 [M+H]⁺.

### Step 8. Chiral resolution of diastereomers of 6-((5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile

6-((5′-(1-methyl-1H-pyrazol-4-yl)-3′-oxotetrahydro-3′H-spiro[cyclobutane-1, 2′-pyrrolo[2, 1-b]oxazol]-3-yl)oxy)pyrimidine-4-carbonitrile (15 mg, 0.041 mmol) was purified by prep. SFC (Column Phenomenex-Cellulose-2 (250 mm×50 mm, 10 m); Condition 0.1% NH₃H₂O EtOH) to give four diastereomers of 6-(((5′S,7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile as a white solids. MS (ESI): m/z 367 [M+H]⁺.

Ex. 6.1 Peak 1 (RT=2.91 min, ee=99%) first trans isomer: ¹H NMR (400 MHz, CDCl₃-d): δ 8.81 (d, J=0.8 Hz, 1H), 7.39 (d, J=9.6 Hz, 2H), 7.11 (d, J=0.8 Hz, 1H), 5.43-5.62 (m, 2H), 4.97 (t, J=7.2 Hz, 1H), 3.80-3.92 (m, 3H), 2.87-3.01 (m, 2H), 2.67-2.76 (m, 1H), 2.62 (m, 1H), 2.47-2.57 (m, 1H), 2.22-2.30 (m, 1H), 2.08-2.17 (m, 1H), 1.71-1.78 (m, 1H).

Ex. 6.2 Peak 2 (RT=4.19 min, ee=92%) first cis isomer: ¹H NMR (400 MHz, CDCl₃-d): δ 8.81 (s, 1H), 7.39 (d, J=10.0 Hz, 2H), 7.08 (s, 1H), 5.57 (dd, J=6.8, 5.2 Hz, 1H), 5.17-5.34 (m, 1H), 4.98 (t, J=7.6 Hz, 1H), 3.88 (s, 3H), 3.05-3.23 (m, 2H), 2.46-2.60 (m, 3H), 2.21-2.29 (m, 1H), 2.07-2.15 (m, 1H), 1.68-1.72 (m, 1H).

Ex. 6.3 Peak 3 (RT=5.00 min, ee=95%) second trans isomer: ¹H NMR (400 MHz, CDCl₃-d): δ 8.81 (d, J=1.2 Hz, 1H), 7.39 (d, J=9.6 Hz, 2H), 7.11 (d, J=1.1 Hz, 1H), 5.42-5.60 (m, 2H), 4.97 (t, J=7.2 Hz, 1H), 3.80-3.95 (m, 3H), 2.88-3.00 (m, 2H), 2.68-2.75 (m, 1H), 2.62 (dd, J=13.2, 6.8 Hz, 1H), 2.48-2.56 (m, 1H), 2.22-2.30 (m, 1H), 2.07-2.16 (m, 1H), 1.67-1.78 (m, 1H).

Ex. 6.4 Peak 4 (RT=5.62 min, ee=79%) second cis isomer: ¹H NMR (400 MHz, CDCl₃-d): δ 8.81 (d, J=1.2 Hz, 1H), 7.39 (d, J=10.0 Hz, 2H), 7.08 (d, J=1.2 Hz, 1H), 5.57 (dd, J=6.8, 5.2 Hz, 1H), 5.25 (quin, J=7.2 Hz, 1H), 4.98 (t, J=7.6 Hz, 1H), 3.88 (s, 3H), 3.04-3.23 (m, 2H), 2.45-2.64 (m, 3H), 2.20-2.31 (m, 1H), 2.05-2.18 (m, 1H), 1.67-1.74 (m, 1H).

### Example 7.1 and 7.2

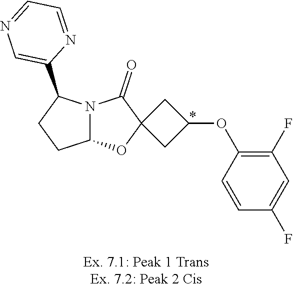

### (1r,3R,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one (Ex. 7.1)

### (1s,3S,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one (Ex. 7.2)

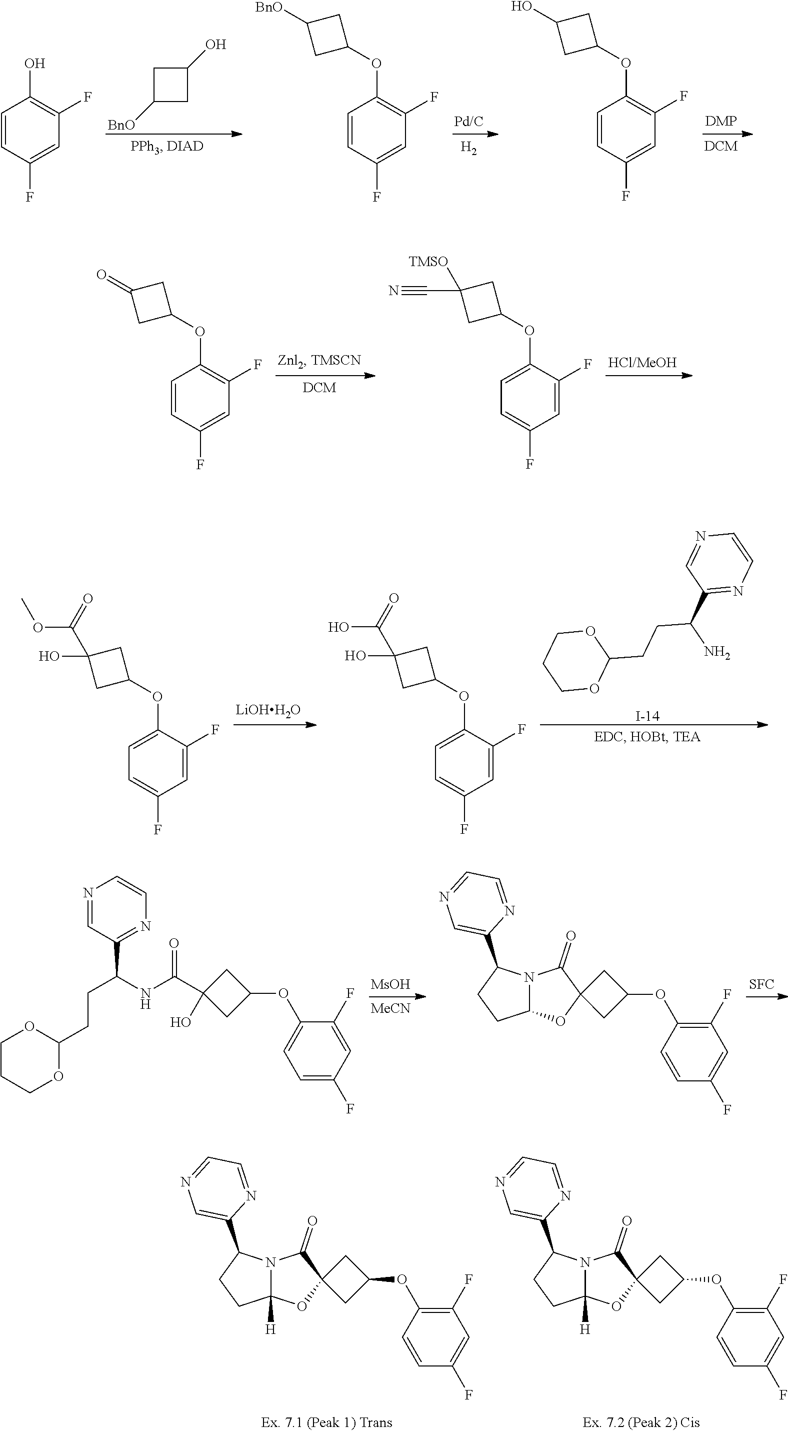

### Step 1. 1-(3-(benzyloxy)cyclobutoxy)-2,4-difluorobenzene

To a solution of 3-(benzyloxy)cyclobutan-1-ol (822 mg, 4.61 mmol), 2,4-difluorophenol (500 mg, 3.84 mmol), Ph₃P (1.21 g, 4.6 mmol) in THF (15 mL) was added DIAD (0.874 mL, 4.50 mmol) at 0° C. under N₂. The reaction mixture was stirred at 25° C. for 12 h. The reaction mixture was directly purified by flash silica gel chromatography (ISCO®; 20 g SepaFlash® Silica Flash Column, eluent of 3% ethyl acetate/pet. ether gradient) to give 1-(3-(benzyloxy)cyclobutoxy)-2,4-difluorobenzene. ¹H NMR (400 MHz, CD₃OD-d₄) δ 7.24-7.38 (m, 5H), 6.79-7.00 (m, 3H), 4.83-4.86 (m, 1H), 4.45 (s, 2H), 4.34 (m, 1H), 2.33-2.55 (m, 4H).

### Step 2. 3-(2,4-difluorophenoxy)cyclobutan-1-ol

A solution of 1-(3-(benzyloxy)cyclobutoxy)-2,4-difluorobenzene (750 mg, 2.58 mmol) in MeOH (15 mL) was added Pd/C (275 mg, 0.26 mmol) (10% in activated carbon) and dihydroxypalladium (181 mg, 0.258 mmol) at 20° C. under H₂ (15 psi). The resulting mixture was stirred at 40° C. for 12 h. The reaction mixture was filtered and the filtrate was concentrated to give 3-(2,4-difluorophenoxy)cyclobutan-1-ol which was used without further purification. ¹H NMR (400 MHz, CD₃OD-d₄) δ 6.80-7.01 (m, 3H), 4.82-4.85 (m, 1H), 4.51 (tt, J=5.2, 7.2 Hz, 1H), 2.41-2.49 (m, 2H), 2.30-2.39 (m, 2H)

### Step 3. 3-(2,4-difluorophenoxy)cyclobutan-1-one

To a solution of 3-(2,4-difluorophenoxy)cyclobutan-1-ol (500 mg, 2.5 mmol) in DCM (15 mL) was added DMP (1589 mg, 3.75 mmol) at 0° C. The reaction mixture was stirred at 25° C. for 2 h. The reaction was added to sat. NaHCO₃ (10 mL) and extracted with DCM (5 mL×2). The combined organic layers were washed with brine (5 mL), dried over Na₂SO₄, filtered and concentrated. The residue was purified by flash silica gel chromatography (ISCO®; 4 g SepaFlash® Silica Flash Column, eluent of 10% ethyl acetate/pet. ether gradient) to give 3-(2,4-difluorophenoxy)cyclobutan-1-one. ¹H NMR (400 MHz, CDCl₃-d) δ 6.76-6.96 (m, 3H), 5.00 (m, 1H), 3.43-3.54 (m, 2H), 3.30-3.41 (m, 2H)

### Step 4. 3-(2,4-difluorophenoxy)-1-((trimethylsilyl)oxy)cyclobutane-1-carbonitrile

To a solution of 3-(2,4-difluorophenoxy)cyclobutan-1-one (500 mg, 2.5 mmol), TMSCN (751 mg, 7.6 mmol) in DCM (15 mL) was added zinc(II) iodide (24 mg, 0.08 mmol) at 0° C. The reaction mixture was stirred at 25° C. for 12 h under N₂. The reaction mixture was directly concentrated, and the residue was purified by flash silica gel chromatography (ISCO®; 4 g SepaFlash® Silica Flash Column, eluent of 4% ethyl acetate/pet. ether gradient) to give 3-(2,4-difluorophenoxy)-1-((trimethylsilyl)oxy)cyclobutane-1-carbonitrile. MS (ESI): m/z 298 [M+H]⁺. ¹H NMR (400 MHz, CDCl₃-d) δ 6.85-6.93 (m, 1H), 6.74-6.84 (m, 2H), 4.48-4.91 (m, 1H), 2.85-3.28 (m, 2H), 2.56-2.84 (m, 2H), 0.25-0.29 (m, 9H).

### Step 5. 3-(2,4-difluorophenoxy)-1-hydroxycyclobutane-1-carboxylate

A solution of 3-(2,4-difluorophenoxy)-1-((trimethylsilyl)oxy)cyclobutane-1-carbonitrile (300 mg, 1.009 mmol) in MeOH (4 mL) was added HCl/MeOH (4M) (4 mL) and the reaction mixture was stirred at 60° C. for 2 h. The mixture was directly concentrated to give methyl 3-(2,4-difluorophenoxy)-1-hydroxycyclobutane-1-carboxylate which was used to next step without further purification. MS (ESI): m/z 258 [M+H]⁺

### Step 6. 3-(2,4-difluorophenoxy)-1-hydroxycyclobutane-1-carboxylic acid

To a solution of methyl 3-(2,4-difluorophenoxy)-1-hydroxycyclobutane-1-carboxylate (240 mg, 0.93 mmol) in MeOH (5 mL) and Water (0.5 mL) was added LiOH·H₂O (117 mg, 2.8 mmol). The reaction was stirred at 25° C. for 4 h, after which the reaction mixture was concentrated and the residue was dissolved in water (10 mL) and extracted with EtOAc (1×3 mL). The aqueous phase was acidified with 2 N HCl (0.5 mL) to pH-4, extracted with EtOAc (5 mL×3). The combined organic layers were dried over Na₂SO₄, filtered and the filtrate was concentrated in vacuo to give 3-(2,4-difluorophenoxy)-1-hydroxycyclobutane-1-carboxylic acid which was used in the next step without further purification. ¹H NMR (400 MHz, CDCl₃-d) δ 6.67-6.97 (m, 3H), 4.61-5.06 (m, 1H), 2.88-3.16 (m, 2H), 2.58-2.75 (m, 2H)

### Step 7. N-(3-(1,3-dioxan-2-yl)-1-(pyrazin-2-yl)propyl)-3-(2,4-difluorophenoxy)-1-hydroxycyclobutane-1-carboxamide

To a solution of 3-(1,3-dioxan-2-yl)-1-(pyrazin-2-yl)propan-1-amine (80 mg, 0.36 mmol, I-14) in DMF (5 mL) was added 3-(2,4-difluorophenoxy)-1-hydroxycyclobutane-1-carboxylic acid (80 mg, 0.33 mmol), TEA (0.150 mL, 1.08 mmol), HOBT (82.0 mg, 0.537 mmol), EDC (103 mg, 0.54 mmol) and the resulting mixture was stirred at 20° C. for 3 h. The mixture was added to water (30 mL) and extracted with EtOAc (5 mL×3). The combined organic layers were washed with brine (10 mL), dried over Na₂SO₄, filtered and concentrated to give N-(3-(1,3-dioxan-2-yl)-1-(pyrazin-2-yl)propyl)-3-(2,4-difluorophenoxy)-1-hydroxycyclobutane-1-carboxamide which was used to next step without further purification. MS (ESI): m/z 450 [M+H]⁺.

### Step 8. 3-(2,4-difluorophenoxy)-5′-(pyrazin-2-yl)tetrahydro-3′H-spiro[cyclobutane-1,2′-pyrrolo[2,1-b]oxazol]-3′-one

To a solution of N-(3-(1,3-dioxan-2-yl)-1-(pyrazin-2-yl)propyl)-3-(2,4-difluorophenoxy)-1-hydroxycyclobutane-1-carboxamide (100 mg, 0.22 mmol) in MeCN (4 mL) was added MsOH (0.029 mL, 0.45 mmol) and the resulting mixture was stirred at 80° C. for 2 The reaction was filtered and the filtrate was purified by Prep-HPLC (Prep HPLC condition: Preparative HPLC on EB instrument fitted with Boston Prime C18 150×40 mm×5 μm using the mobile phase A-B: water (0.1% TFA)-ACN, Gradient: 45-65%) to give 3-(2,4-difluorophenoxy)-5′-(pyrazin-2-yl)tetrahydro-3′H-spiro[cyclobutane-1,2′-pyrrolo[2,1-b]oxazol]-3′-one. MS (ESI): m/z 374 [M+H]⁺

### Step 9. Resolution of Diastereomers

The 3-(2,4-difluorophenoxy)-5′-(pyrazin-2-yl)tetrahydro-3′H-spiro[cyclobutane-1,2′-pyrrolo[2,1-b]oxazol]-3′-one (60 mg, 0.161 mmol) was resolved by Chiral-SFC (Instrument SFC-21 Method Column DAICEL CHIRALPAK AD-H (250 mm×30 mm, 5 m) 0.1% NH₃H₂O ETOH) to give the two diastereomers of 3-(2,4-difluorophenoxy)-5′-(pyrazin-2-yl)tetrahydro-3′H-spiro[cyclobutane-1,2′-pyrrolo[2,1-b]oxazol]-3′-one Ex. 7.1 (Peak 1, trans) (Rt=1.17 min, ee=100%) and Ex. 7.2 (Peak 2, cis) (Rt=1.78 min, ee=100%).

Ex. 7.1 (Peak 1, Trans): ¹H NMR (400 MHz, CDCl₃-d) δ 8.68 (s, 1H), 8.53 (m, 2H), 6.71-6.92 (m, 3H), 5.65-5.72 (m, 12H), 5.14 (dd, J=6.0, 7.6 Hz, 1H), 4.90 (m, 1H), 3.01 (dd, J=7.6, 13.6 Hz, 1H), 2.86 (td, J=6.4, 13.2 Hz, 1H), 2.55-2.71 (m, 3H), 2.26-2.38 (m, 2H), 1.72-1.86 (m, 1H)

Ex. 7.2 (Peak 2, Cis): ¹H NMR (400 MHz, CDCl₃-d) δ 8.67 (d, J=0.8 Hz, 1H), 8.49-8.56 (m, 2H), 6.68-6.92 (m, 3H), 5.70 (dd, J=4.8, 7.2 Hz, 1H), 5.05-5.16 (m, 1H), 4.60 (m, 1H), 2.97-3.13 (m, 2H), 2.52-2.69 (m, 3H), 2.24-2.38 (m, 2H), 1.64-1.79 (m, 1H)

### Example 8.1

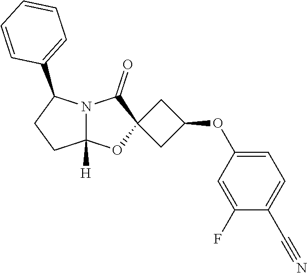 [SMILES: N#Cc1ccc(O[C@H]2C[C@@]3(C2)O[C@@H]2CC[C@@H](c4ccccc4)N2C3=O)cc1F]

### 2-fluoro-4-(((1r,3R,5′S,7a′R)-3′-oxo-5′-phenyltetrahydro-3′H-spiro[cyclobutane-1,2′-pyrrolo[2,1-b]oxazol]-3-yl)oxy)benzonitrile

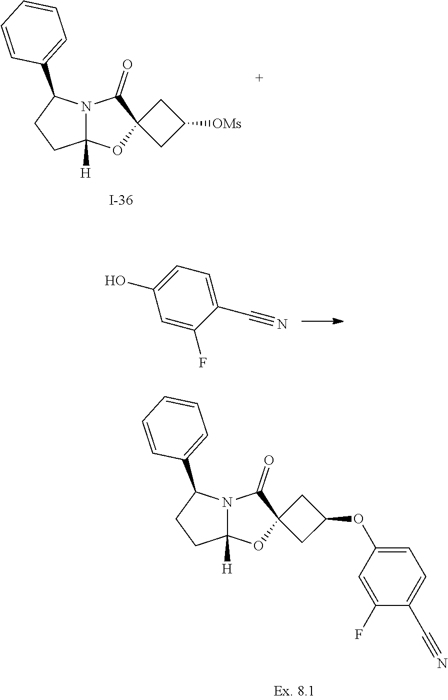

### 2-fluoro-4-(((1r,3R,5′S,7a′R)-3′-oxo-5′-phenyltetrahydro-3′H-spiro[cyclobutane-1,2′-pyrrolo[2,1-b]oxazol]-3-yl)oxy)benzonitrile

Microscale library preparation: A 0.2M solution of (1s,3S,5′S,7a′R)-3′-oxo-5′-phenyltetrahydro-3′H-spiro[cyclobutane-1,2′-pyrrolo[2,1-b]oxazol]-3-yl methanesulfonate (I-36) (150 mg in 2.1 ml of DMSO) was prepared and then 12 μl (0.0025 mmol) dosed into a reaction well containing cesium carbonate (1.6 mg, 0.005 mmol). A separate 0.35M DMSO solution of 2-fluoro-4-hydroxybenzonitrile was prepared and subsequently dosed into the reaction well (12.5 μl, 0.0044 mmol). The reaction well was sealed and heated to 80° C. overnight. After cooling, the reaction was diluted to 0.100 ml, filtered and purified via RP-HPLC (Method: TFA modified, 35% to 70% ACN in H₂O) to give 2-fluoro-4-(((1r,3R,5′S,7a′R)-3′-oxo-5′-phenyltetrahydro-3′H-spiro[cyclobutane-1,2′-pyrrolo[2,1-b]oxazol]-3-yl)oxy)benzonitrile. MS (ESI) m/z [M+H]⁺ calc'd for C₂₂H₁₉FN₂O₃: 378, found: 378.

The compounds presented in Table 8 below were prepared using procedure analogous to one described for Example 8.1 with appropriately chosen commercially aromatic alcohols. All products are in the trans configuration unless otherwise noted.

TABLE 8

| | | | Exact Mass |
|---|---|---|---|
| Example | Structure | IUPAC Name | [M + H]+ |
| 8.2 | 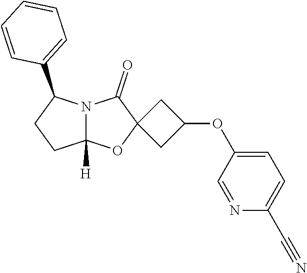 [SMILES: N#Cc1ccc(OC2CC3(C2)O[C@@H]2CC[C@@H](c4ccccc4)N2C3=O)cn1] | 5-{[(5′S,7a′R)-3′-oxo-5′- phenyltetrahydro-3′H- spiro[cyclobutane-1,2′-pyrrolo[2,1- b][1,3]oxazol]-3-yl]oxy}pyridine- 2-carbonitrile | Calc'd 362, found 362 |
| 8.3 | 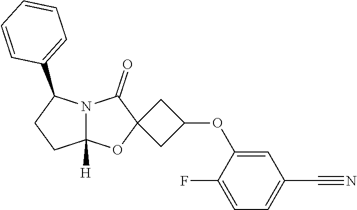 [SMILES: N#Cc1ccc(F)c(OC2CC3(C2)O[C@@H]2CC[C@@H](c4ccccc4)N2C3=O)c1] | 4-fluoro-3-{[(5′S,7a′R)-3′-oxo-5′- phenyltetrahydro-3′H- spiro[cyclobutane-1,2′-pyrrolo[2,1- b][1,3]oxazol]-3- yl]oxy}benzonitrile | Calc'd 379, found 379 |
| 8.4 | 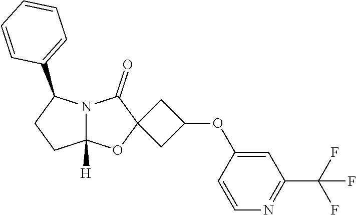 [SMILES: O=C1N2[C@@H](CC[C@H]2c2ccccc2)OC12CC(Oc1ccnc(C(F)(F)F)c1)C2] | (5′S,7a′R)-5′-phenyl-3-{[2- (trifluoromethyl)pyridin-4- yl]oxy}tetrahydro-3′H- spiro[cyclobutane-1,2′-pyrrolo[2,1- b][1,3]oxazol]-3′-one | Calc'd 405, found 405 |
| 8.5 | 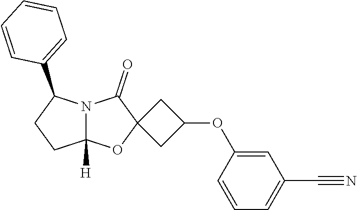 [SMILES: N#Cc1cccc(OC2CC3(C2)O[C@@H]2CC[C@@H](c4ccccc4)N2C3=O)c1] | 3-{[(5′S,7a′R)-3′-oxo-5′- phenyltetrahydro-3′H- spiro[cyclobutane-1,2′-pyrrolo[2,1- b][1,3]oxazol]-3- yl]oxy}benzonitrile | Calc'd 361, found 361 |
| 8.6 | 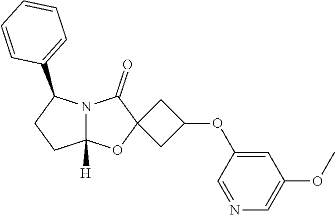 [SMILES: COc1cncc(OC2CC3(C2)O[C@@H]2CC[C@@H](c4ccccc4)N2C3=O)c1] | (5′S,7a′R)-3-[(5-methoxypyridin-3- yl)oxy]-5′-phenyltetrahydro-3′H- spiro[cyclobutane-1,2′-pyrrolo[2,1- b][1,3]oxazol]-3′-one | Calc'd 367, found 367 |
| 8.7 | 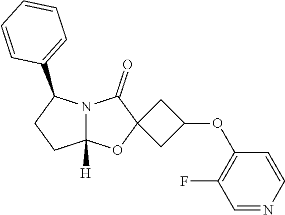 [SMILES: O=C1N2[C@@H](CC[C@H]2c2ccccc2)OC12CC(Oc1ccncc1F)C2] | (5′S,7a′R)-3-[(3-fluoropyridin-4- yl)oxy]-5′-phenyltetrahydro-3′H- spiro[cyclobutane-1,2′-pyrrolo[2,1- b][1,3]oxazol]-3′-one | Calc'd 355, found 355 |
| 8.8 | 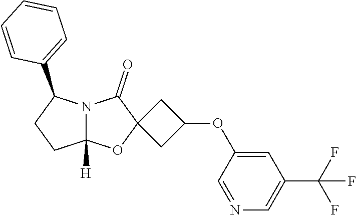 [SMILES: O=C1N2[C@@H](CC[C@H]2c2ccccc2)OC12CC(Oc1cncc(C(F)(F)F)c1)C2] | (5′S,7a′R)-5′-phenyl-3-{[5- (trifluoromethyl)pyridin-3- yl]oxy}tetrahydro-3′H- spiro[cyclobutane-1,2′-pyrrolo[2,1- b][1,3]oxazol]-3′-one | Calc'd 405, found 405 |
| 8.9 | 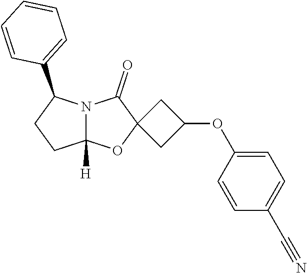 [SMILES: N#Cc1ccc(OC2CC3(C2)O[C@@H]2CC[C@@H](c4ccccc4)N2C3=O)cc1] | 4-{[5′S,7a′R)-3′-oxo-5′- phenyltetrahydro-3′H- spiro[cyclobutane-1,2′-pyrrolo[2,1- b][1,3]oxazol]-3- yl]oxy}benzonitrile | Calc'd 361, found 361 |
| 8.10 | 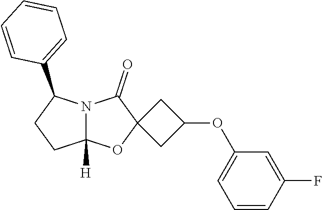 [SMILES: O=C1N2[C@@H](CC[C@H]2c2ccccc2)OC12CC(Oc1cccc(F)c1)C2] | (5′S,7a′R)-3-(3-fluorophenoxy)-5′- phenyltetrahydro-3′H- spiro[cyclobutane-1,2′-pyrrolo[2,1- b][1,3]oxazol]-3′-one | Calc'd 354, found 354 |
| 8.11 | 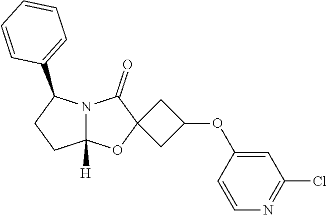 [SMILES: O=C1N2[C@@H](CC[C@H]2c2ccccc2)OC12CC(Oc1ccnc(Cl)c1)C2] | (5′S,7a′R)-3-[(2-chloropyridin-4- yl)oxy]-5′-phenyltetrahydro-3′H- spiro[cyclobutane-1,2′-pyrrolo[2,1- b][1,3]oxazol]-3′-onee | Calc'd 371, found 371 |
| 8.12 | 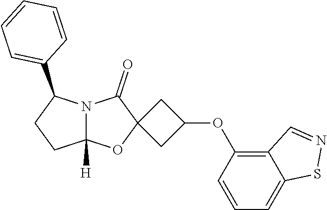 [SMILES: O=C1N2[C@@H](CC[C@H]2c2ccccc2)OC12CC(Oc1cccc3sncc13)C2] | (5′S,7a′R)-3-[(1,2-benzothiazol-4- yl)oxy]-5′-phenyltetrahydro-3′H- spiro[cyclobutane-1,2′-pyrrolo[2,1- b][1,3]oxazol]-3′-one | Calc'd 393, found 393 |
| 8.13 | 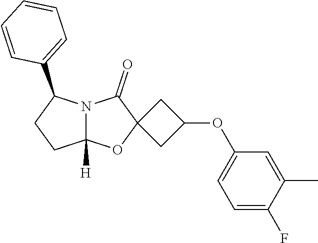 [SMILES: Cc1cc(OC2CC3(C2)O[C@@H]2CC[C@@H](c4ccccc4)N2C3=O)ccc1F] | (5′S,7a′R)-3-(4-fluoro-3- methylphenoxy)-5′- phenyltetrahydro-3′H- spiro[cyclobutane-1,2′-pyrrolo[2,1- b][1,3]oxazol]-3′-one | Calc'd 368, found 368 |
| 8.14 | 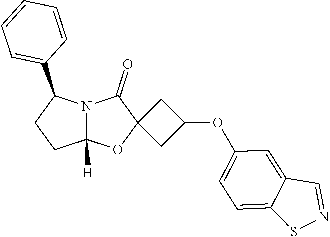 [SMILES: O=C1N2[C@@H](CC[C@H]2c2ccccc2)OC12CC(Oc1ccc3sncc3c1)C2] | (5′S,7a′R)-3-[(1,2-benzothiazol-5- yl)oxy]-5′-phenyltetrahydro-3′H- spiro[cyclobutane-1,2′-pyrrolo[2,1- b][1,3]oxazol]-3′-one | Calc'd 393, found 393 |
| 8.15 | 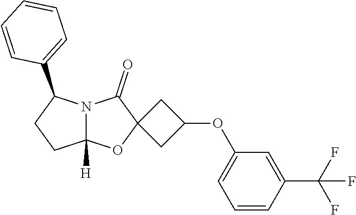 [SMILES: O=C1N2[C@@H](CC[C@H]2c2ccccc2)OC12CC(Oc1cccc(C(F)(F)F)c1)C2] | (5′S,7a′R)-5′-phenyl-3-[3- (trifluoromethyl)phenoxy]tetrahydro- 3′H-spiro[cyclobutane-1,2′- pyrrolo[2,1-b][1,3]oxazol]-3′-one | Calc'd 404, found 404 |
| 8.16 | 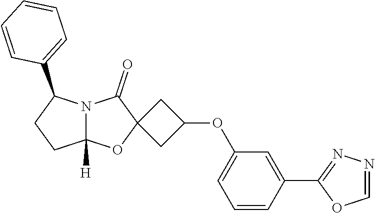 [SMILES: O=C1N2[C@@H](CC[C@H]2c2ccccc2)OC12CC(Oc1cccc(-c3nnco3)c1)C2] | (5′S,7a′R)-3-[3-(1,3,4-oxadiazol-2- yl)phenoxy]-5′-phenyltetrahydro- 3′H-spiro[cyclobutane-1,2′- pyrrolo[2,1-b][1,3]oxazol]-3′-one | Calc'd 404, found 404 |
| 8.17 | 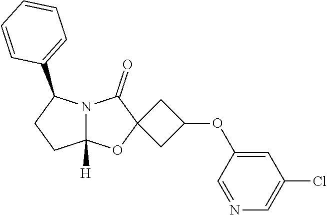 [SMILES: O=C1N2[C@@H](CC[C@H]2c2ccccc2)OC12CC(Oc1cncc(Cl)c1)C2] | (5′S,7a′R)-3-[(5-chloropyridin-3- yl)oxy-5′-phenyltetrahydro-3′H- spiro[cyclobutane-1,2′-pyrrolo[2,1- b][1,3]oxazol]-3′-one | Calc'd 371, found 371 |
| 8.18 | 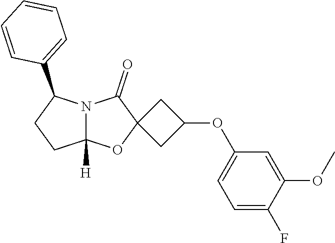 [SMILES: COc1cc(OC2CC3(C2)O[C@@H]2CC[C@@H](c4ccccc4)N2C3=O)ccc1F] | (5′S,7a′R)-3-(4-fluoro-3- methoxyphenoxy)-5′- phenyltetrahydro-3′H- spiro[cyclobutane-1,2′-pyrrolo[2,1- b][1,3]oxazol]-3′-one | Calc'd 384, found 384 |
| 8.19 | 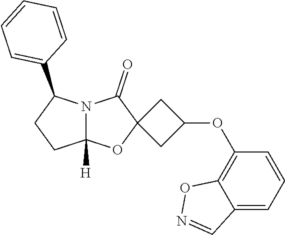 [SMILES: O=C1N2[C@@H](CC[C@H]2c2ccccc2)OC12CC(Oc1cccc3cnoc13)C2] | (5′S,7a′R)-3-[1,2-benzoxazol-7- yl)oxy]-5′-phenyltetrahydro-3′H- spiro[cyclobutane-1,2′-pyrrolo[2,1- b][1,3]oxazol]-3′-one | Calc'd 377, found 377 |
| 8.20 | 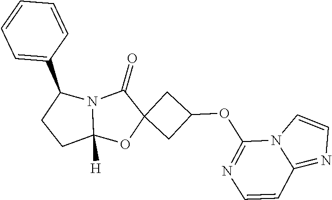 [SMILES: O=C1N2[C@@H](CC[C@H]2c2ccccc2)OC12CC(Oc1nccc3nccn13)C2] | (5′S,7a′R)-3-[(imidazo[1,2- c]pyrimidin-5-yl)oxy]-5′- phenyltetrahydro-3′H- spiro[cyclobutane-1,2′-pyrrolo[2,1- b][1,3]oxazol]-3′-one | Calc'd 377, found 377 |
| 8.21 | 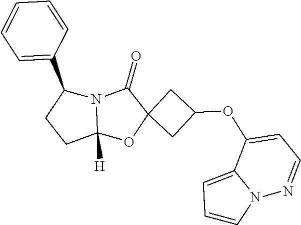 [SMILES: O=C1N2[C@@H](CC[C@H]2c2ccccc2)OC12CC(Oc1ccnn3cccc13)C2] | (5′S,7a′R)-5′-phenyl-3- [(pyrrolo[1,2-b]pyridazin-4- yl)oxy]tetrahydro-3′H- spiro[cyclobutane-1,2′-pyrrolo[2,1- b][1,3]oxazol]-3′-one | Calc'd 376, found 376 |
| 8.22 | 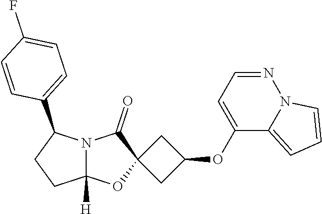 [SMILES: O=C1N2[C@@H](CC[C@H]2c2ccc(F)cc2)O[C@]12C[C@H](Oc1ccnn3cccc13)C2] | (1r,3R,5′S,7a′R)-5′-(4- fluorophenyl)-3-(pyrrolo[1,2- b]pyridazin-4-yloxy)tetrahydro- 3′H-spiro[cyclobutane-1,2′- pyrrolo[2,1-b]oxazol]-3′-one | Calc'd 393, found 394 |
| 8.23 | 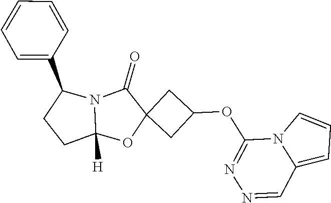 [SMILES: O=C1N2[C@@H](CC[C@H]2c2ccccc2)OC12CC(Oc1nncc3cccn13)C2] | (5′S,7a′R)-5′-phenyl-3- [(pyrrolo[1,2-d][1,2,4]triazin-4- yl)oxy]tetrahydro-3′H- spiro[cyclobutane-1,2′-pyrrolo[2,1- b][1,3]oxazol]-3′-one | Calc'd 377, found 377 |

### Example 9.1

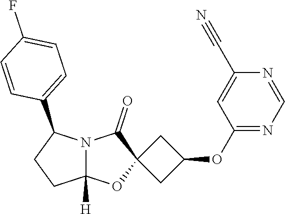 [SMILES: N#Cc1cc(O[C@H]2C[C@@]3(C2)O[C@@H]2CC[C@@H](c4ccc(F)cc4)N2C3=O)ncn1]

### 6-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrimidine-4-carbonitrile

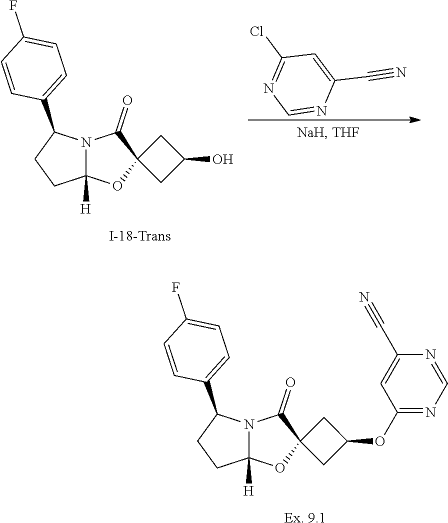

### 6-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrimidine-4-carbonitrile

To a solution of (5′S,7a′R)-5′-(4-fluorophenyl)-3-hydroxytetrahydro-3′H-spiro[cyclobutane-1,2′-pyrrolo[2,1-b]oxazol]-3′-one (I-18-Trans) (15 mg, 0.054 mmol) in THF (1 mL) was added NaH (3.2 mg, 0.081 mmol) at 0° C. and stirred for 20 min. Then 6-chloropyrimidine-4-carbonitrile (11.3 mg, 0.081 mmol) was added and the resulting mixture was stirred at 20° C. for 2 h. The reaction mixture was quenched by the addition of water (1 mL) and extracted with EtOAc (3×1 mL). The organic layer was concentrated and purified by prep. HPLC (Column Welch Xtimate C18 150×25 mm×5 m: Condition water (10 mM-NH₄HCO₃)-ACN) to give 6-(((5′S,7a′R)-5′-(4-fluorophenyl)-3′-oxotetrahydro-3′H-spiro[cyclobutane-1,2′-pyrrolo[2,1-b]oxazol]-3-yl)oxy)pyrimidine-4-carbonitrile as colorless oil. MS (ESI): m/z 403 [M+Na]⁺. ¹H NMR (CDCl₃-d, 400 MHz): δ 8.82 (d, J=1.0 Hz, 1H), 7.21-7.25 (m, 2H), 7.11 (d, J=1.0 Hz, 1H), 7.00-7.07 (m, 2H), 5.64 (dd, J=6.5, 5.3 Hz, 1H), 5.53 (m, 1H), 5.00 (t, J=7.5 Hz, 1H), 2.92-3.02 (m, 2H), 2.65-2.78 (m, 2H), 2.56-2.64 (m, 1H), 2.21-2.29 (m, 1H), 2.03 (m, 1H), 1.69-1.79 (m, 1H).

### Example 9.2 and Example 9.3

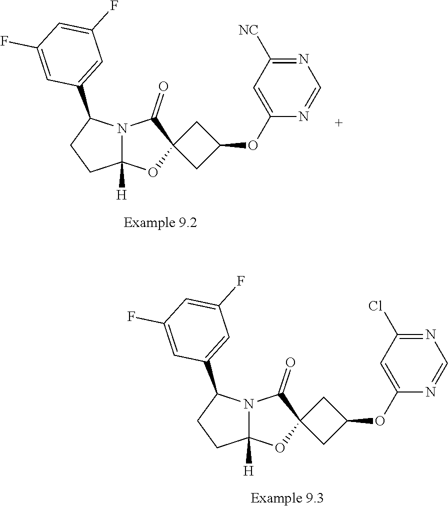 [SMILES: N#Cc1cc(O[C@H]2C[C@@]3(C2)O[C@@H]2CC[C@@H](c4cc(F)cc(F)c4)N2C3=O)ncn1 ; O=C1N2[C@@H](CC[C@H]2c2cc(F)cc(F)c2)O[C@]12C[C@H](Oc1cc(Cl)ncn1)C2]

### 6-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile

### (5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one

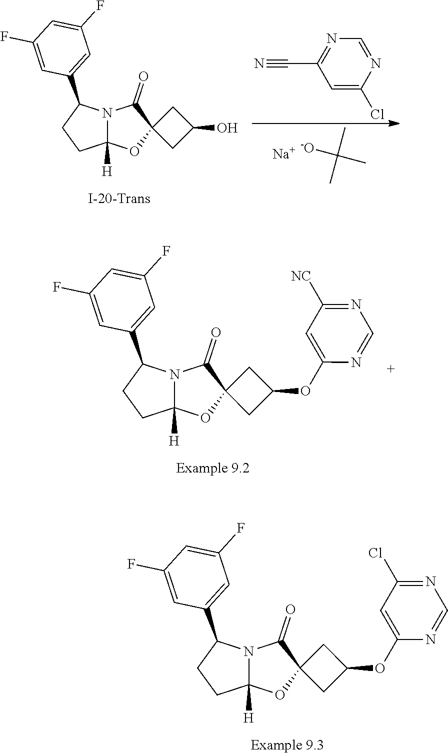

(5′S,7a'S)-5′-(3,5-difluorophenyl)-3-hydroxytetrahydro-3′H-spiro[cyclobutane-1,2′-pyrrolo[2,1-b]oxazol]-3′-one (15 mg, 0.05 mmol) (I-20-Trans), DMSO (1 mL), and 6-chloropyrimidine-4-carbonitrile (14.1 mg, 0.102 mmol) were purged with nitrogen for 1 min followed by addition of sodium tert-butoxide (0.051 mL, 0.102 mmol). The resulting reaction was heated at 90° C. in a sealed reaction vessel overnight. The reaction crude was purified by reverse phase chromatography eluting with acetonitrile/water+0.05% TFA. The obtained solid was further purified by preparative TLC (silica gel) developed with EtOAc/hexanes mixture (20% ethyl acetate v/v) to give 6-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile (Ex. 9.2) MS (ESI) m/z [M+H]⁺: 399. ¹H NMR (500 MHz, CDCl₃-d) δ 8.84 (s, 1H), 7.13 (s, 1H), 6.80 (d, J=6.1 Hz, 2H), 6.74 (t, J=8.6 Hz, 1H), 5.64 (s, 1H), 5.59-5.52 (m, 1H), 5.00 (t, J=6.9 Hz, 1H), 3.05-2.96 (m, 2H), 2.80-2.69 (m, 2H), 2.68-2.59 (m, 1H), 2.31-2.23 (m, 1H), 2.04 (d, J=18.7 Hz, 1H), 1.78 (d, J=10.1 Hz, 1H), and (5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one (Ex. 9.3). MS (ESI) m/z [M+H]⁺: 408, ¹H NMR (500 MHz, CDCl₃-d) δ 8.58 (s, 1H), 6.80 (d, J=7.4 Hz, 3H), 6.73 (t, J=8.6 Hz, 1H), 5.64 (s, 1H), 5.56-5.48 (m, 1H), 4.99 (d, J=7.1 Hz, 1H), 2.98 (s, 2H), 2.77-2.66 (m, 2H), 2.66-2.58 (m, 1H), 2.25 (s, 1H), 2.01 (s, 1H), 1.76 (s, 1H).

The compounds presented in Table 9 below were prepared using procedure analogous to one described for Example 9.1, 9.2 or 9.3 with the appropriately chosen intermediate from Table 4 along with appropriate solvent, base and commercially available or known electrophiles. All products are drawn in either cis or trans geometry unless unknown, where the order of elution is noted as Peak 1 or Peak 2.

TABLE 9

| | | | Exact Mass |
|---|---|---|---|
| Example | Structure | IUPAC Name | [M + H]+ |
| 9.4 | 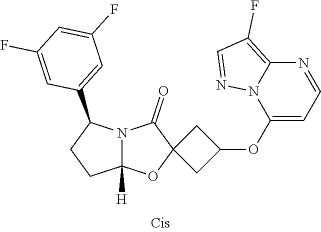 [SMILES: O=C1N2[C@@H](CC[C@H]2c2cc(F)cc(F)c2)OC12CC(Oc1ccnc3c(F)cnn13)C2] | (1s,3S,5′S,7a′R)-5′-(3,5- difluorophenyl)-3-((3- fluoropyrazolo[1,5-a]pyrimidin-7- yl)oxy)tetrahydro-3′H- spiro[cyclobutane-1,2′-pyrrolo[2,1- b]oxazol]-3′-one | Calc'd 431, found 431 |
| 9.5 | 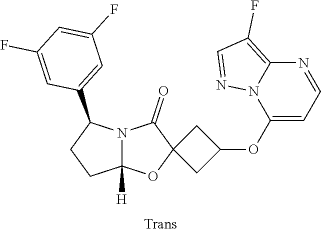 [SMILES: O=C1N2[C@@H](CC[C@H]2c2cc(F)cc(F)c2)OC12CC(Oc1ccnc3c(F)cnn13)C2] | (1r,3R,5′S,7a′R)-5′-(3,5- difluorophenyl)-3-((3- fluoropyrazolo[1,5-a]pyrimidin-7- yl)oxy)tetrahydro-3′H- spiro[cyclobutane-1,2′-pyrrolo[2,1- b]oxazol]-3′-one | Calc'd 431, found 431 |
| 9.6 | 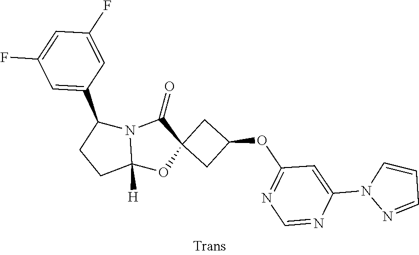 [SMILES: O=C1N2[C@@H](CC[C@H]2c2cc(F)cc(F)c2)O[C@]12C[C@H](Oc1cc(-n3cccn3)ncn1)C2] | (5′S,7a′R)-5′-(3,5-difluorophenyl)-3- {[6-(1H-pyrazol-1-yl)pyrimidin-4- yl]oxy}tetrahydro-3′H- spiro[cyclobutane-1,2′-pyrrolo[2,1- b][1,3]oxazol]-3′-one | Calc'd 440, found 440 |
| 9.7 | 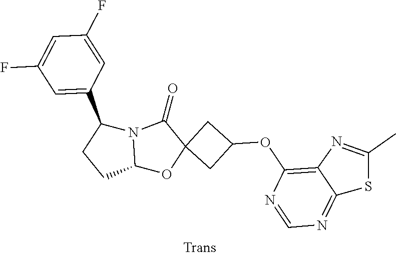 [SMILES: Cc1nc2c(OC3CC4(C3)O[C@@H]3CC[C@@H](c5cc(F)cc(F)c5)N3C4=O)ncnc2s1] | (5′S,7a′R)-5′-(3,5-difluorophenyl)-3- [(2-methyl[1,3]thiazolo[5,4- d]pyrimidin-7-yl)oxy]tetrahydro- 3′H-spiro[cyclobutane-1,2′- pyrrolo[2,1-b][1,3]oxazol]-3′-one | Calc'd 445, found 445 |
| 9.8 | 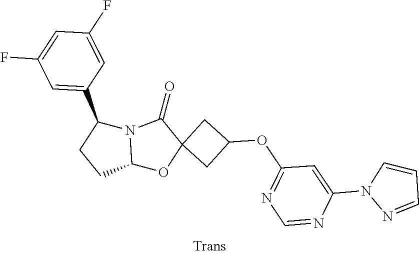 [SMILES: O=C1N2[C@@H](CC[C@H]2c2cc(F)cc(F)c2)OC12CC(Oc1cc(-n3cccn3)ncn1)C2] | (5′S,7a′R)-5′-(3,5-difluorophenyl)-3- {[6-(1H-pyrazol-1-yl)pyrimidin-4- yl]oxy}tetrahydro-3′H- spiro[cyclobutane-1,2′-pyrrolo[2,1- b][1,3]oxazol]-3′-one | Calc'd 440, found 440 |
| 9.9 | 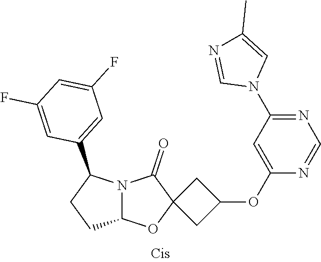 [SMILES: Cc1cn(-c2cc(OC3CC4(C3)O[C@@H]3CC[C@@H](c5cc(F)cc(F)c5)N3C4=O)ncn2)cn1] | (1s,3S,5′S,7a′R)-5′-(3,5- difluorophenyl)-3-((6-(4-methyl-1H- imidazol-1-yl)pyrimidin-4- yl)oxy)tetrahydro-3′H- spiro[cyclobutane-1,2′-pyrrolo[2,1- b]oxazol]-3′-one | Calc'd 454, found 454 |
| 9.10 | 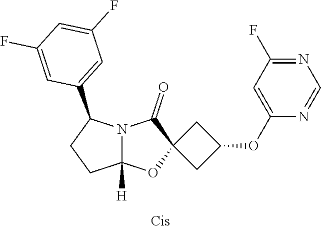 [SMILES: O=C1N2[C@@H](CC[C@H]2c2cc(F)cc(F)c2)O[C@]12C[C@@H](Oc1cc(F)ncn1)C2] | (1s,3S,5′S,7a′R)-5′-(3,5- difluorophenyl)-3-((6- fluoropyrimidin-4- yl)oxy)tetrahydro-3′H- spiro[cyclobutane-1,2′-pyrrolo[2,1- b]oxazol]-3′-one | Calc'd 392, found 392 |
| 9.11 | 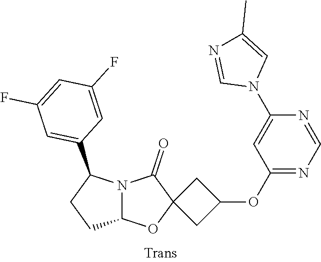 [SMILES: Cc1cn(-c2cc(OC3CC4(C3)O[C@@H]3CC[C@@H](c5cc(F)cc(F)c5)N3C4=O)ncn2)cn1] | (5′S,7a′R)-5′-(3,5-difluorophenyl)-3- {[6-(4-methyl-1H-imidazol-1- yl)pyrimidin-4-yl]oxy}tetrahydro- 3′H-spiro[cyclobutane-1,2′- pyrrolo[2,1-b][1,3]oxazol]-3′-one | Calc'd 454, found 454 |
| 9.12 | 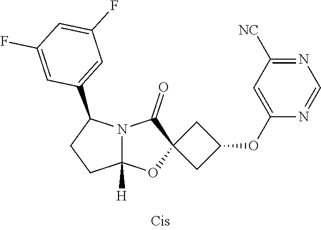 [SMILES: N#Cc1cc(O[C@H]2C[C@]3(C2)O[C@@H]2CC[C@@H](c4cc(F)cc(F)c4)N2C3=O)ncn1] | 6-(((1s,3S,5′S,7a′R)-5′-(3,5- difluorophenyl)-3′-oxotetrahydro- 3′H-spiro[cyclobutane-1,2′- pyrrolo[2,1-b]oxazol]-3- yl)oxy)pyrimidine-4-carbonitrile | Calc'd 399, found 399 |
| 9.13 | 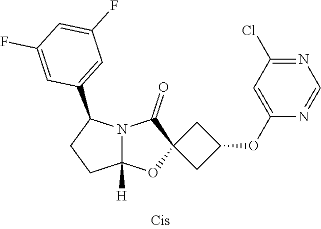 [SMILES: O=C1N2[C@@H](CC[C@H]2c2cc(F)cc(F)c2)O[C@]12C[C@@H](Oc1cc(Cl)ncn1)C2] | (1s,3S,5′S,7a′R)-3-((6- chloropyrimidin-4-yl)oxy)-5′-(3,5- difluorophenyl)tetrahydro-3′H- spiro[cyclobutane-1,2′-pyrrolo[2,1- b]oxazol]-3′-one | Calc'd 408, found 408 |
| 9.14 | 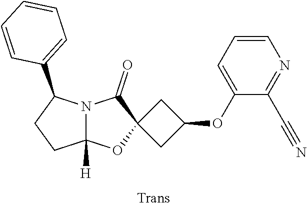 [SMILES: N#Cc1ncccc1O[C@H]1C[C@@]2(C1)O[C@@H]1CC[C@@H](c3ccccc3)N1C2=O] | 3-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyridine-2- carbonitrile | Calc'd 362, found 362 |
| 9.15 | 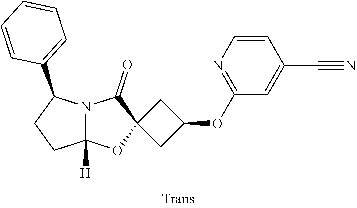 [SMILES: N#Cc1ccnc(O[C@H]2C[C@@]3(C2)O[C@@H]2CC[C@@H](c4ccccc4)N2C3=O)c1] | 5-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyridine-3- carbonitrile | Calc'd 362, found 362 |
| 9.16 | 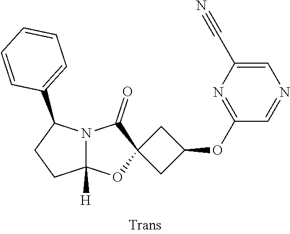 [SMILES: N#Cc1cncc(O[C@H]2C[C@@]3(C2)O[C@@H]2CC[C@@H](c4ccccc4)N2C3=O)n1] | 6-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyrazine-2- carbonitrile | Calc'd 363, found 363 |
| 9.17 | 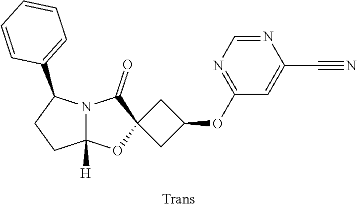 [SMILES: N#Cc1cc(O[C@H]2C[C@@]3(C2)O[C@@H]2CC[C@@H](c4ccccc4)N2C3=O)ncn1] | 6-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyrimidine- 4-carbonitrile | Calc'd 363, found 363 |
| 9.18 | 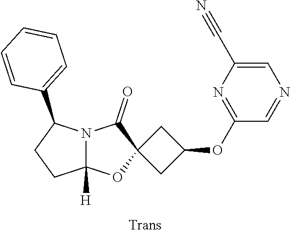 [SMILES: N#Cc1cncc(O[C@H]2C[C@@]3(C2)O[C@@H]2CC[C@@H](c4ccccc4)N2C3=O)n1] | 4-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyrimidine- 2-carbonitrile | Calc'd 363, found 363 |
| 9.19 | 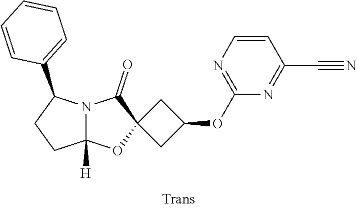 [SMILES: N#Cc1ccnc(O[C@H]2C[C@@]3(C2)O[C@@H]2CC[C@@H](c4ccccc4)N2C3=O)n1] | 2-{[(1r,3R,5′S,7a′R)-3′-oxo-5′- phenyltetrahydro-3′H- spiro[cyclobutane-1,2′-pyrrolo[2,1- b][1,3]oxazol]-3-yl]oxy}pyrimidine- 4-carbonitrile | Calc'd 363, found 363 |
| 9.20 | 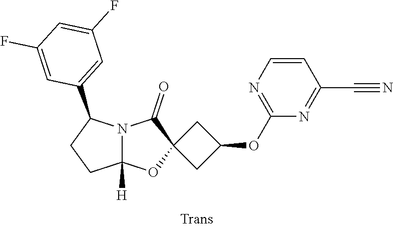 [SMILES: N#Cc1ccnc(O[C@H]2C[C@@]3(C2)O[C@@H]2CC[C@@H](c4cc(F)cc(F)c4)N2C3=O)n1] | 2-{[(1r,3R,5′S,7a′R)-5′-(3,5- difluorophenyl)-3′-oxotetrahydro- 3′H-spiro[cyclobutane-1,2′- pyrrolo[2,1-b][1,3]oxazol]-3- yl]oxy}pyrimidine-4-carbonitrile | Calc'd 399, found 399 |
| 9.21 | 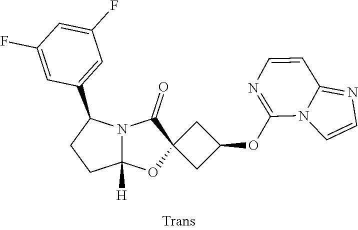 [SMILES: O=C1N2[C@@H](CC[C@H]2c2cc(F)cc(F)c2)O[C@]12C[C@H](Oc1nccc3nccn13)C2] | (1r,3R,5′S,7a′R)-5′-(3,5- difluorophenyl)-3-[(imidazo[1,2- c]pyrimidin-5-yl)oxy]tetrahydro- 3′H-spiro[cyclobutane-1,2′- pyrrolo[2,1-b][1,3]oxazol]-3′-one | Calc'd 413, found 413 |
| 9.22 | 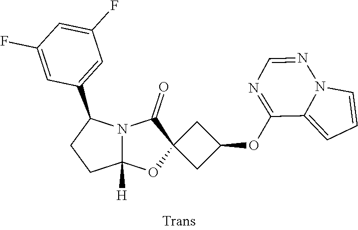 [SMILES: O=C1N2[C@@H](CC[C@H]2c2cc(F)cc(F)c2)O[C@]12C[C@H](Oc1ncnn3cccc13)C2] | (1r,3R,5′S,7a′R)-5′-(3,5- difluorophenyl)-3-[(pyrrolo[2,1- f][1,2,4]triazin-4-yl)oxy]tetrahydro- 3′H-spiro[cyclobutane-1,2′- pyrrolo[2,1-b][1,3]oxazol]-3′-one | Calc'd 413, found 413 |
| 9.23 | 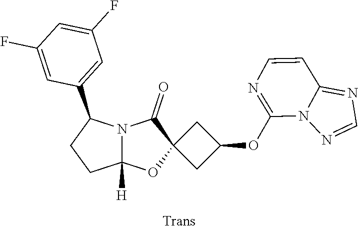 [SMILES: O=C1N2[C@@H](CC[C@H]2c2cc(F)cc(F)c2)O[C@]12C[C@H](Oc1nccc3ncnn13)C2] | (1r,3R,5′S,7a′R)-5′-(3,5- difluorophenyl)-3- [([1,2,4]triazolo[1,5-c]pyrimidin-5- yl)oxy]tetrahydro-3′H- spiro[cyclobutane-1,2′-pyrrolo[2,1- b][1,3]oxazol]-3′-one | Calc'd 414, found 414 |
| 9.24 | 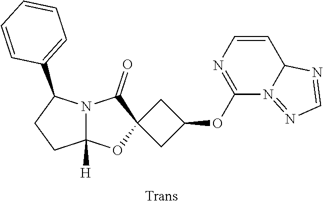 | (1r,3R,5′S,7a′R)-5′-phenyl-3- [(pyrrolo[2,1-f][1,2,4]triazin-4- yl)oxy]tetrahydro-3′H- spiro[cyclobutane-1,2′-pyrrolo[2,1- b][1,3]oxazol]-3′-one | Calc'd 377, found 377 |
| 9.25 | 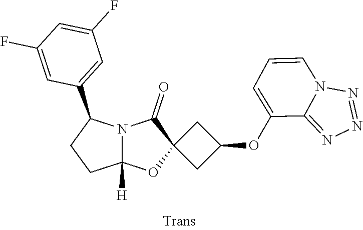 [SMILES: O=C1N2[C@@H](CC[C@H]2c2cc(F)cc(F)c2)O[C@]12C[C@H](Oc1cccn3nnnc13)C2] | (1r,3R,5′S,7a′R)-5′-(3,5- difluorophenyl)-3-[(tetrazolo[1,5- a]pyridin-8-yl)oxy]tetrahydro-3′H- spiro[cyclobutane-1,2′-pyrrolo[2,1- b][1,3]oxazol]-3′-one | Calc'd 414, found 414 |
| 9.26 | 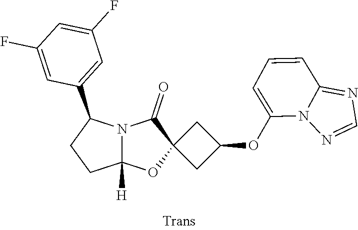 [SMILES: O=C1N2[C@@H](CC[C@H]2c2cc(F)cc(F)c2)O[C@]12C[C@H](Oc1cccc3ncnn13)C2] | (1r,3R,5′S,7a′R)-5′-(3,5- difluorophenyl)-3- [([1,2,4]triazolo[1,5-a]pyridin-5- yl)oxy]tetrahydro-3′H- spiro[cyclobutane-1,2′-pyrrolo[2,1- b][1,3]oxazol]-3′-one | Calc'd 413, found 413 |
| 9.27 | 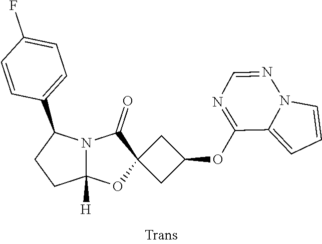 [SMILES: O=C1N2[C@@H](CC[C@H]2c2ccc(F)cc2)O[C@]12C[C@H](Oc1ncnn3cccc13)C2] | (1r,3R,5′S,7a′R)-5′-(4-fluorophenyl)- 3-[(pyrrolo[2,1-f][1,2,4]triazin-4- yl)oxy]tetrahydro-3′H- spiro[cyclobutane-1,2′-pyrrolo[2,1- b][1,3]oxazol]-3′-one | Calc'd 395, found 395 |
| 9.28 | 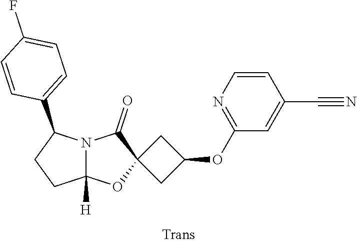 [SMILES: N#Cc1ccnc(O[C@H]2C[C@@]3(C2)O[C@@H]2CC[C@@H](c4ccc(F)cc4)N2C3=O)c1] | 2-{[(1r,3R,5′S,7a′R)-5′-(4- fluorophenyl)-3′-oxotetrahydro-3′H- spiro[cyclobutane-1,2′-pyrrolo[2,1-b][1,3] oxazol]-3-yl]oxy}pyridine-4- carbonitrile | Calc'd 380, found 380 |
| 9.29 | 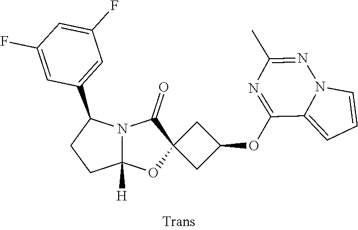 [SMILES: Cc1nc(O[C@H]2C[C@@]3(C2)O[C@@H]2CC[C@@H](c4cc(F)cc(F)c4)N2C3=O)c2cccn2n1] | (1r,3R,5′S,7a′R)-5′-(3,5- difluorophenyl)-3-[(2- methylpyrrolo[2,1-f][1,2,4]triazin-4- yl)oxy]tetrahydro-3′H- spiro[cyclobutane-1,2′-pyrrolo[2,1- b][1,3]oxazol]-3′-one | Calc'd 427, found 427 |
| 9.30 | 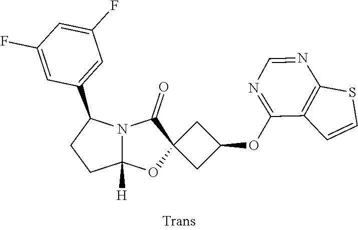 [SMILES: O=C1N2[C@@H](CC[C@H]2c2cc(F)cc(F)c2)O[C@]12C[C@H](Oc1ncnc3sccc13)C2] | (1r,3R,5′S,7a′R)-5′-(3,5- difluorophenyl)-3-[(thieno[2,3- d]pyrimidin-4-yl)oxy]tetrahydro- 3′H-spiro[cyclobutane-1,2′- pyrrolo[2,1-b][1,3]oxazol]-3′-one | Calc'd 430, found 430 |
| 9.31 | 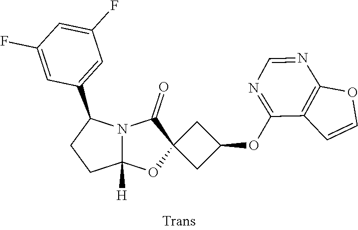 [SMILES: O=C1N2[C@@H](CC[C@H]2c2cc(F)cc(F)c2)O[C@]12C[C@H](Oc1ncnc3occc13)C2] | (1r,3R,5′S,7a′R)-5′-(3,5- difluorophenyl)-3-[(furo[2,3- d]pyrimidin-4-yl)oxy]tetrahydro- 3′H-spiro[cyclobutane-1,2′- pyrrolo[2,1-b][1,3]oxazol]-3′-one | Calc'd 414, found 414 |
| 9.32 | 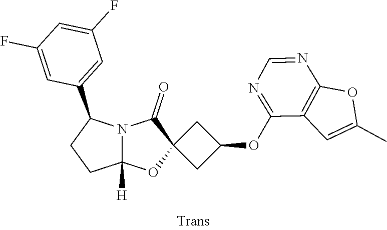 [SMILES: Cc1cc2c(O[C@H]3C[C@@]4(C3)O[C@@H]3CC[C@@H](c5cc(F)cc(F)c5)N3C4=O)ncnc2o1] | (1r,3R,5′S,7a′R)-5′-(3,5- difluorophenyl)-3-[(6- methylfuro[2,3-d]pyrimidin-4- yl)oxy]tetrahydro-3′H- spiro[cyclobutane-1,2′-pyrrolo[2,1- b][1,3]oxazol]-3′-one | Calc'd 428, found 428 |
| 9.33 | 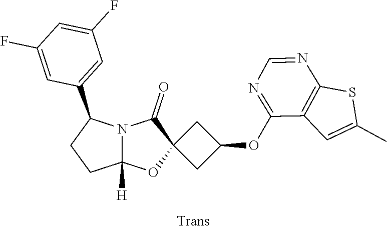 [SMILES: Cc1cc2c(O[C@H]3C[C@@]4(C3)O[C@@H]3CC[C@@H](c5cc(F)cc(F)c5)N3C4=O)ncnc2s1] | (1r,3R,5′S,7a′R)-5′-(3,5- difluorophenyl)-3-[(6- methylthieno[2,3-d]pyrimidin-4- yl)oxy]tetrahydro-3′H- spiro[cyclobutane-1,2′-pyrrolo[2,1- b][1,3]oxazol]-3′-one | Calc'd 444, found 444 |
| 9.34 | 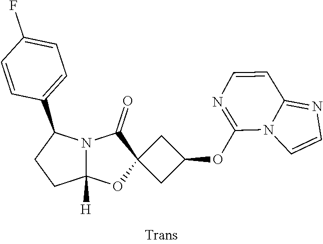 [SMILES: O=C1N2[C@@H](CC[C@H]2c2ccc(F)cc2)O[C@]12C[C@H](Oc1nccc3nccn13)C2] | (1r,3R,5′S,7a′R)-5′-(4-fluorophenyl)- 3-[(imidazo[1,2-c]pyrimidin-5- yl)oxy]tetrahydro-3′H- spiro[cyclobutane-1,2′-pyrrolo[2,1- b][1,3]oxazol]-3′-one | Calc'd 395, found 395 |
| 9.35 | 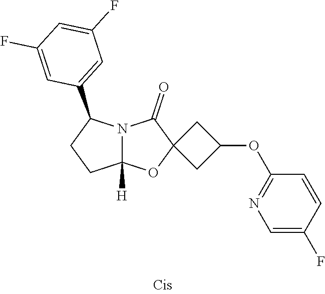 [SMILES: O=C1N2[C@@H](CC[C@H]2c2cc(F)cc(F)c2)OC12CC(Oc1ccc(F)cn1)C2] | (1s,3S,5′S,7a′R)-5′-(3,5- difluorophenyl)-3-((5-fluoropyridin- 2-yl)oxy)tetrahydro-3′H- spiro[cyclobutane-1,2′-pyrrolo[2,1- b]oxazol]-3′-one | Calc'd 391, found 391 |
| 9.36 | 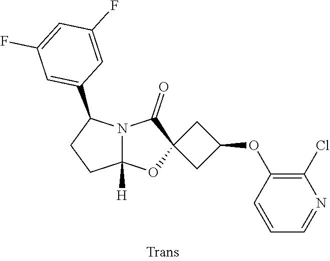 [SMILES: O=C1N2[C@@H](CC[C@H]2c2cc(F)cc(F)c2)O[C@]12C[C@H](Oc1cccnc1Cl)C2] | (5′S,7a′R)-3-[(2-chloropyridin-3- yl)oxy]-5′-(3,5- difluorophenyl)tetrahydro-3′H- spiro[cyclobutane-1,2′-pyrrolo[2,1- b][1,3]oxazol]-3′-one | Calc'd 407, found 407 |
| 9.37 | 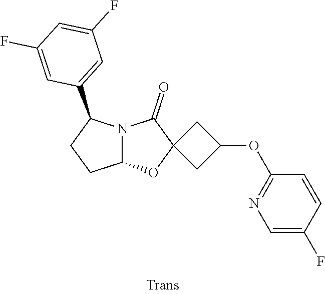 | (5′S,7a′R)-5′-(3,5-difluorophenyl)-3- [(5-fluoropyridin-2- yl)oxy]tetrahydro-3′H- spiro[cyclobutane-1,2′-pyrrolo[2,1- b][1,3]oxazol]-3′-one | Calc'd 391, found 391 |
| 9.38 | 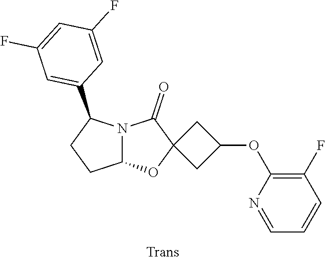 | (5′S,7a′R)-5′-(3,5-difluorophenyl)-3- [(3-fluoropyridin-2- yl)oxy]tetrahydro-3′H- spiro[cyclobutane-1,2′-pyrrolo[2,1- b][1,3]oxazol]-3′-one | Calc'd 391, found 391 |
| 9.39 | 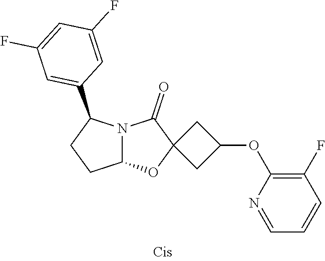 | (5′S,7a′R)-5′-(3,5-difluorophenyl)-3- [(3-fluoropyridin-2- yl)oxy]tetrahydro-3′H- spiro[cyclobutane-1,2′-pyrrolo[2,1- b][1,3]oxazol]-3′-one | Calc'd 391, found 391 |
| 9.40 | 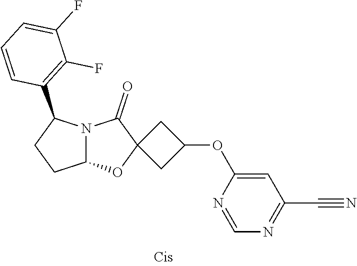 | 6-{[(5′S,7a′R)-5′-(2,3- difluorophenyl)-3′-oxotetrahydro- 3′H-spiro[cyclobutane-1,2′- pyrrolo[2,1-b][1,3]oxazol]-3- yl]oxy}pyrimidine-4-carbonitrile | Calc'd 399, found 399 |
| 9.41 | 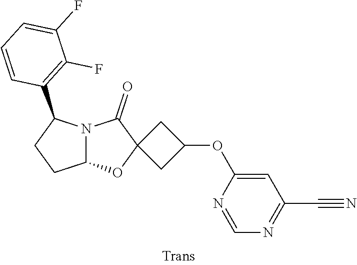 | 6-{[(5′S,7a′R)-5′-(2,3- difluorophenyl)-3′-oxotetrahydro- 3′H-spiro[cyclobutane-1,2′- pyrrolo[2,1-b][1,3]oxazol]-3- yl]oxy}pyrimidine-4-carbonitrile | Calc'd 399, found 399 |
| 9.42 | 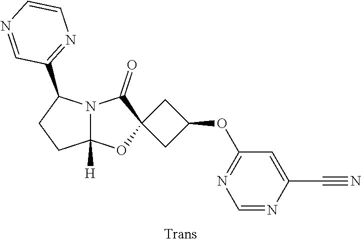 [SMILES: N#Cc1cc(O[C@H]2C[C@@]3(C2)O[C@@H]2CC[C@@H](c4cnccn4)N2C3=O)ncn1] | 6-(((5′S,7a′R)-3′-oxo-5′-(pyrazin-2- yl)tetrahydro-3′H-spiro[cyclobutane- 1,2′-pyrrolo[2,1-b]oxazol]-3- yl)oxy)pyrimidine-4-carbonitrile | Calc'd 365, found 365 |
| 9.43 | 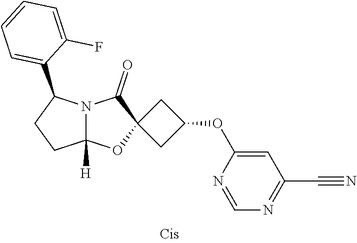 [SMILES: N#Cc1cc(O[C@H]2C[C@]3(C2)O[C@@H]2CC[C@@H](c4ccccc4F)N2C3=O)ncn1] | 6-{[(5′S,7a′R)-5′-(2-fluorophenyl)- 3′-oxotetrahydro-3′H- spiro[cyclobutane-1,2′-pyrrolo[2,1- b][1,3]oxazol]-3-yl]oxy}pyrimidine- 4-carbonitrile | Calc'd 381, found 381 |
| 9.44 | 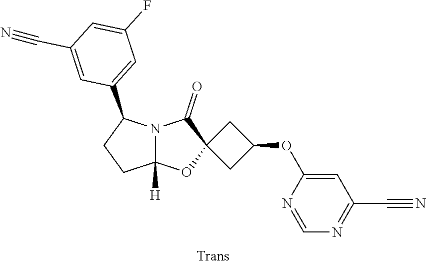 [SMILES: N#Cc1cc(F)cc([C@@H]2CC[C@H]3O[C@]4(C[C@H](Oc5cc(C#N)ncn5)C4)C(=O)N32)c1] | 6-(((5′S,7a′R)-5′-(3-cyano-5- fluorophenyl)-3′-oxotetrahydro-3′H- spiro[cyclobutane-1,2′-pyrrolo[2,1- b]oxazol]-3-yl)oxy)pyrimidine-4- carbonitrile | Calc'd 406, found 406 |
| 9.45 | 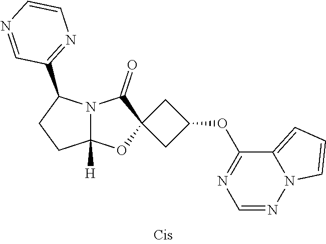 [SMILES: O=C1N2[C@@H](CC[C@H]2c2cnccn2)O[C@]12C[C@@H](Oc1ncnn3cccc13)C2] | (1s,3S,5′S,7a′R)-5′-(pyrazin-2-yl)-3- (pyrrolo[2,1-f][1,2,4]triazin-4- yloxy)tetrahydro-3′H- spiro[cyclobutane-1,2′-pyrrolo[2,1- b]oxazol]-3′-one | Calc'd 379, found 379 |
| 9.46 | 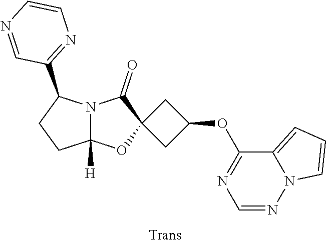 [SMILES: O=C1N2[C@@H](CC[C@H]2c2cnccn2)O[C@]12C[C@H](Oc1ncnn3cccc13)C2] | (1r,3R,5′S,7a′R)-5′-(pyrazin-2-yl)-3- (pyrrolo[2,1-f][1,2,4]triazin-4- yloxy)tetrahydro-3′H- spiro[cyclobutane-1,2′-pyrrolo[2,1- b]oxazol]-3′-one | Calc'd 379, found 379 |
| 9.47 | 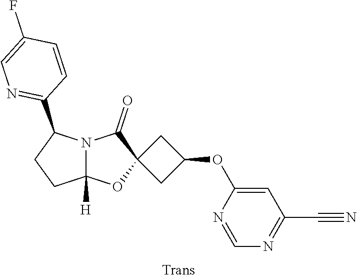 [SMILES: N#Cc1cc(O[C@H]2C[C@@]3(C2)O[C@@H]2CC[C@@H](c4ccc(F)cn4)N2C3=O)ncn1] | 6-{[(5′S,7a′R)-5′-(5-fluoropyridin-2- yl)-3′-oxotetrahydro-3′H- spiro[cyclobutane-1,2′-pyrrolo[2,1- b][1,3]oxazol]-3-yl]oxy}pyrimidine- 4-carbonitrile | Calc'd 382, found 382 |
| 9.48 | 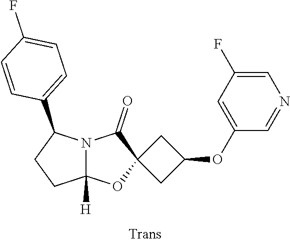 [SMILES: O=C1N2[C@@H](CC[C@H]2c2ccc(F)cc2)O[C@]12C[C@H](Oc1cncc(F)c1)C2] | (1r,3R,5′S,7a′R)-5′-(4-fluorophenyl)- 3-[(5-fluoropyridin-3- yl)oxy]tetrahydro-3′H- spiro[cyclobutane-1,2′-pyrrolo[2,1- b][1,3]oxazol]-3′-one | Calc'd 373, found 373 |
| 9.49 | 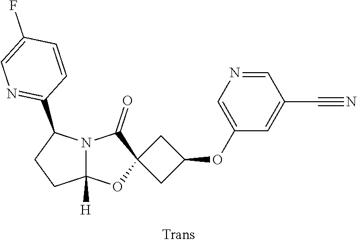 [SMILES: N#Cc1cncc(O[C@H]2C[C@@]3(C2)O[C@@H]2CC[C@@H](c4ccc(F)cn4)N2C3=O)c1] | 5-{[(1r,3R,5′S,7a′R)-5′-(4- fluorophenyl)-3′-oxotetrahydro-3′H- spiro[cyclobutane-1,2′-pyrrolo[2,1- b][1,3]oxazol]-3-yl]oxy}pyridine-3- carbonitrile | Calc'd 380, found 380 |
| 9.50 | 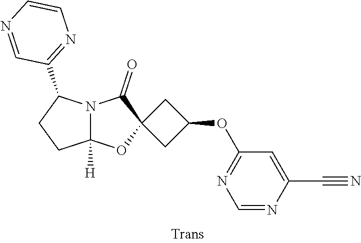 [SMILES: N#Cc1cc(O[C@H]2C[C@@]3(C2)O[C@H]2CC[C@H](c4cnccn4)N2C3=O)ncn1] | 6-(((5′R,7a′S)-3′-oxo-5′-(pyrazin-2- yl)tetrahydro-3′H-spiro[cyclobutane- 1,2′-pyrrolo[2,1-b]oxazol]-3- yl)oxy)pyrimidine-4-carbonitrile | Calc'd 365, found 365 |
| 9.51 | 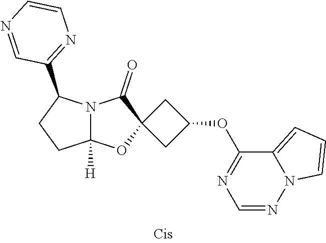 [SMILES: O=C1N2[C@H](CC[C@H]2c2cnccn2)O[C@]12C[C@@H](Oc1ncnn3cccc13)C2] | (1s,3R,5′R,7a′S)-5′-(pyrazin-2-yl)-3- (pyrrolo[2,1-f][1,2,4]triazin-4- yloxy)tetrahydro-3′H- spiro[cyclobutane-1,2′-pyrrolo[2,1- b]oxazol]-3′-one | Calc'd 379, found 379 |
| 9.52 | 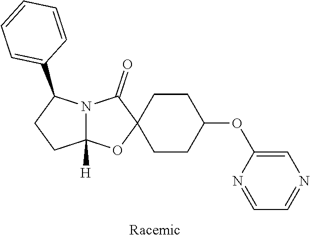 [SMILES: O=C1N2[C@@H](CC[C@H]2c2ccccc2)OC12CCC(Oc1cnccn1)CC2] | (5′S,7a′R)-5′-phenyl-4-[(pyrazin-2- yl)oxy]tetrahydro-3′H- spiro[cyclohexane-1,2′-pyrrolo[2,1- b][1,3]oxazol]-3′-one | Calc'd 366, found 366 |
| 9.53 | 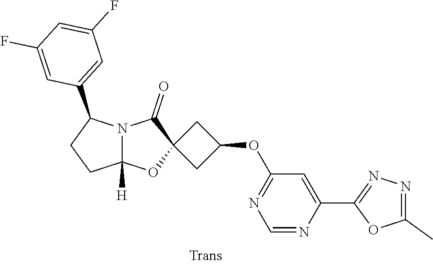 [SMILES: Cc1nnc(-c2cc(O[C@H]3C[C@@]4(C3)O[C@@H]3CC[C@@H](c5cc(F)cc(F)c5)N3C4=O)ncn2)o1] | (5′S,7a′R)-5′-(3,5-difluorophenyl)-3- ((6-(5-methyl-1,3,4-oxadiazol-2- yl)pyrimidin-4-yl)oxy)tetrahydro- 3′H-spiro[cyclobutane-1,2′- pyrrolo[2,1-b]oxazol]-3′-one | Calc'd 456, found 456 |
| 9.54 | 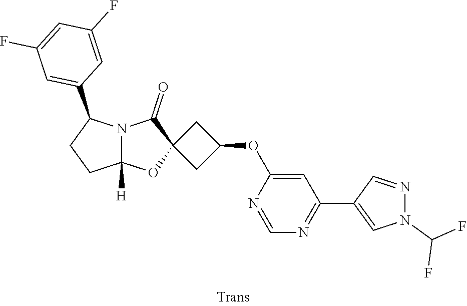 [SMILES: O=C1N2[C@@H](CC[C@H]2c2cc(F)cc(F)c2)O[C@]12C[C@H](Oc1cc(-c3cnn(C(F)F)c3)ncn1)C2] | (5′S,7a′R)-3-((6-(1-(difluoromethyl)- 1H-pyrazol-4-yl)pyrimidin-4- yl)oxy)-5′-(3,5- difluorophenyl)tetrahydro-3′H- spiro[cyclobutane-1,2′-pyrrolo[2,1- b]oxazol]-3′-one | Calc'd 490, found 490 |
| 9.55 | 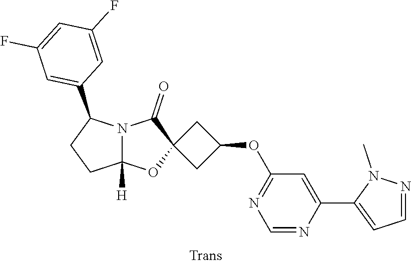 [SMILES: Cn1nccc1-c1cc(O[C@H]2C[C@@]3(C2)O[C@@H]2CC[C@@H](c4cc(F)cc(F)c4)N2C3=O)ncn1] | (5′S,7a′R)-5′-(3,5-difluorophenyl)-3- ((6-(1-methyl-1H-pyrazol-5- yl)pyrimidin-4-yl)oxy)tetrahydro- 3′H-spiro[cyclobutane-1,2′- pyrrolo[2,1-b]oxazol]-3′-one | Calc'd 454, found 454 |
| 9.56 | 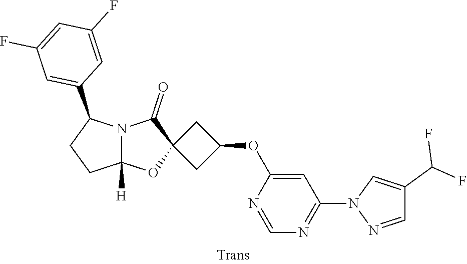 [SMILES: O=C1N2[C@@H](CC[C@H]2c2cc(F)cc(F)c2)O[C@]12C[C@H](Oc1cc(-n3cc(C(F)F)cn3)ncn1)C2] | (5′S,7a′R)-3-((6-(4-(difluoromethyl)- 1H-pyrazol-1-yl)pyrimidin-4- yl)oxy)-5′-(3,5- difluorophenyl)tetrahydro-3′H- spiro[cyclobutane-1,2′-pyrrolo[2,1- b]oxazol]-3′-one | Calc'd 490, found 490 |
| 9.57 | 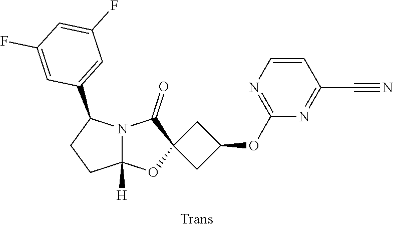 [SMILES: N#Cc1ccnc(O[C@H]2C[C@@]3(C2)O[C@@H]2CC[C@@H](c4cc(F)cc(F)c4)N2C3=O)n1] | 2-(((1r,3R,5′S,7a′R)-5′-(3,5- difluorophenyl)-3′-oxotetrahydro- 3′H-spiro[cyclobutane-1,2′- pyrrolo[2,1-b]oxazol]-3- yl)oxy)pyrimidine-4-carbonitrile | Calc'd 398, found 398 |
| 9.58 | 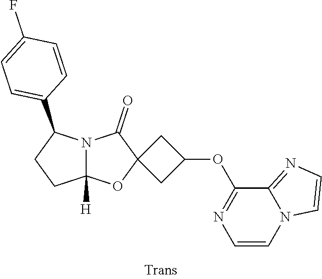 [SMILES: O=C1N2[C@@H](CC[C@H]2c2ccc(F)cc2)OC12CC(Oc1nccn3ccnc13)C2] | (5′S,7a′R)-5′-(4-fluorophenyl)-3- [(imidazo[1,2-a]pyrazin-8- yl)oxy]tetrahydro-3′H- spiro[cyclobutane-1,2′-pyrrolo[2,1- b][1,3]oxazol]-3′-one | Calc'd 395, found 395 |
| 9.59 | 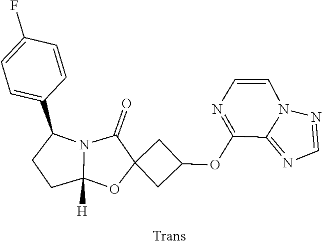 [SMILES: O=C1N2[C@@H](CC[C@H]2c2ccc(F)cc2)OC12CC(Oc1nccn3ncnc13)C2] | (5′S,7a′R)-5′-(4-fluorophenyl)-3- [([1,2,4]triazolo[1,5-a]pyrazin-8- yl)oxy]tetrahydro-3′H- spiro[cyclobutane-1,2′-pyrrolo[2,1- b][1,3]oxazol]-3′-one | Calc'd 396, found 396 |
| 9.60 | 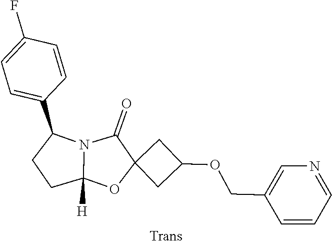 [SMILES: O=C1N2[C@@H](CC[C@H]2c2ccc(F)cc2)OC12CC(OCc1cccnc1)C2] | (5′S,7a′R)-5′-(4-fluorophenyl)-3- [(pyridin-3-yl)methoxy]tetrahydro- 3′H-spiro[cyclobutane-1,2′- pyrrolo[2,1-b][1,3]oxazol]-3′-one | Calc'd 369, found 369 |
| 9.61 | 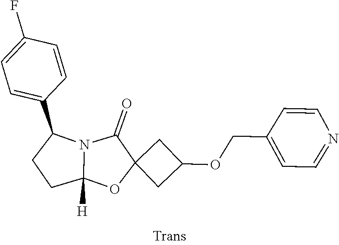 [SMILES: O=C1N2[C@@H](CC[C@H]2c2ccc(F)cc2)OC12CC(OCc1ccncc1)C2] | (5′S,7a′R)-5′-(4-fluorophenyl)-3- [(pyridin-4-yl)methoxy]tetrahydro- 3′H-spiro[cyclobutane-1,2′- pyrrolo[2,1-b][1,3]oxazol]-3′-one | Calc'd 369, found 369 |
| 9.62 | 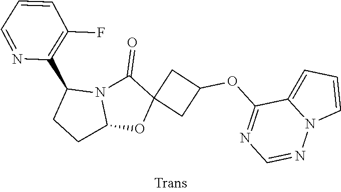 | (5′S,7a′R)-5′-(3-fluoropyridin-2-yl)- 3-[(pyrrolo[2,1-f][1,2,4]triazin-4- yl)oxy]tetrahydro-3′H- spiro[cyclobutane-1,2′-pyrrolo[2,1- b][1,3]oxazol]-3′-one | Calc'd 369, found 369 |
| 9.63 | 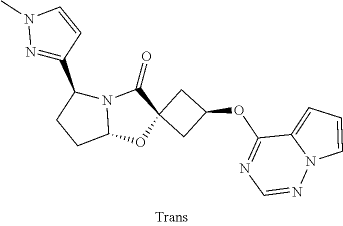 [SMILES: Cn1ccc([C@@H]2CC[C@H]3O[C@]4(C[C@H](Oc5ncnn6cccc56)C4)C(=O)N32)n1] | (1r,3R,5′S,7a′R)-5′-(1-methyl-1H- pyrazol-3-yl)-3-[(pyrrolo[2,1- f][1,2,4]triazin-4-yl)oxy]tetrahydro- 3′H-spiro[cyclobutane-1,2′- pyrrolo[2,1-b][1,3]oxazol]-3′-one | Calc'd 381, found 381 |
| 9.64 | 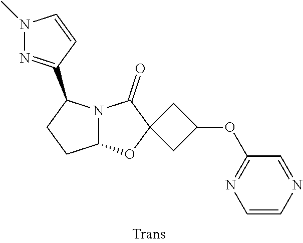 | (5′S,7a′R)-5′-(1-methyl-1H-pyrazol- 3-yl)-3-[(pyrazin-2- yl)oxy]tetrahydro-3′H- spiro[cyclobutane-1,2′-pyrrolo[2,1- b][1,3]oxazol]-3′-one | Calc'd 342, found 342 |
| 9.65 | 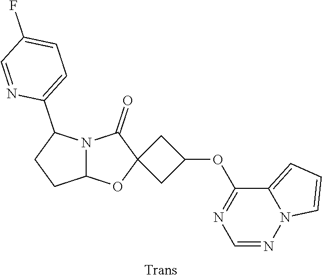 [SMILES: O=C1N2C(CCC2c2ccc(F)cn2)OC12CC(Oc1ncnn3cccc13)C2] | 5′-(5-fluoropyridin-2-yl)-3- [(pyrrolo[2,1-f][1,2,4]triazin-4- yl)oxy]tetrahydro-3′H- spiro[cyclobutane-1,2′-pyrrolo[2,1- b][1,3]oxazol]-3′-one | Calc'd 396, found 396 |
| 9.66 | 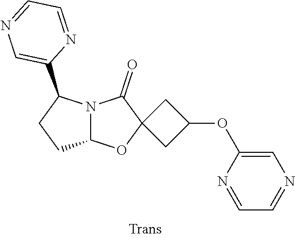 [SMILES: O=C1N2[C@@H](CC[C@H]2c2cnccn2)OC12CC(Oc1cnccn1)C2] | (5′S,7a′R)-5′-(pyrazin-2-yl)-3- [(pyrazin-2-yl)oxy]tetrahydro-3′H- spiro[cyclobutane-1,2′-pyrrolo[2,1- b][1,3]oxazol]-3′-one | Calc'd 340, found 340 |

### Example 9.46

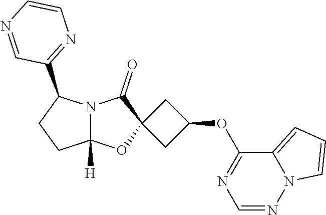 [SMILES: O=C1N2[C@@H](CC[C@H]2c2cnccn2)O[C@]12C[C@H](Oc1ncnn3cccc13)C2]

### (1r,3R,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one

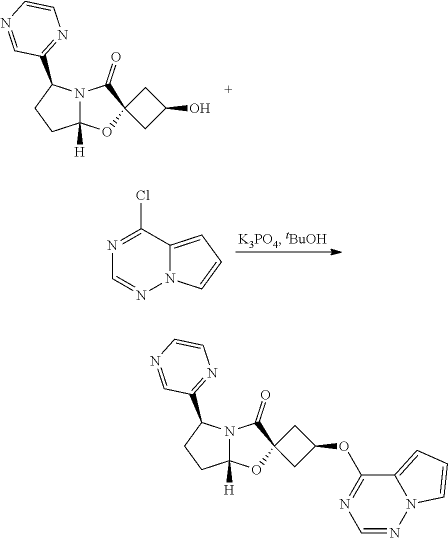 [SMILES: O=C1N2[C@@H](CC[C@H]2c2cnccn2)O[C@]12C[C@H](O)C2 ; Clc1ncnn2cccc12 ; O=C1N2[C@@H](CC[C@H]2c2cnccn2)O[C@]12C[C@H](Oc1ncnn3cccc13)C2]

An oven-dried 250-mL RBF was charged with K₃PO₄ (3.8 g, 17.91 mmol) inside of glovebox. The flask was sealed. Solution of (1r,3R,5′S,7a′R)-3-hydroxy-5′-(pyrazin-2-yl)tetrahydro-3′H-spiro[cyclobutane-1,2′-pyrrolo[2,1-b]oxazol]-3′-one I-30 (3.6 g, 13.78 mmol) and 4-chloropyrrolo[2,1-f][1,2,4]triazine (3174 mg, 20.67 mmol) in tert-butanol (68.9 mL, 13.78 mmol) was added under inert atmosphere. The resulting solution was heated to 70° C. for 7 h. Upon completion as was judged by LCMS the reaction mixture was cooled to 20° C. and quenched with NH₄Cl/EtOAc (75 mL). The content of the flask was vigorously stirred until all residue was dissolved and transferred into separatory funnel and partitioned between EtOAc (300 mL) and aq. sat. NH₄Cl (300 mL). Phases were separated, organic layer was washed with brine (200 mL). Combined aqueous layer was backwashed with EtOAc (100 mL) twice. The combined organic layer was dried over MgSO₄, filtered, and concentrated in vacuo. Crude material was dissolved in 5V of IPAc at 50° C., then 5V of Heptane was added. Solution was cooled to 35° C. and stirred for 3 hours. The solid was filtered off, washed with cold IPAc followed by Heptane. Yielding (1r,3R,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one. MS (ESI) m/z [M+H]⁺ C₁₉H₁₈N₆O₃: 379. ¹H NMR (499 MHz, DMSO) δ 8.73 (s, 1H), 8.64 (s, 1H), 8.61 (d, J=2.4 Hz, 1H), 8.18 (s, 1H), 7.96 (s, 1H), 6.88 (dd, J=4.4, 1.4 Hz, 1H), 6.85 (dd, J=4.3, 2.7 Hz, 1H), 5.83-5.72 (m, 1H), 5.67-5.58 (m, 1H), 5.07 (t, J=7.6 Hz, 1H), 2.98 (dt, J=13.2, 6.6 Hz, 1H), 2.86 (dd, J=13.3, 7.0 Hz, 1H), 2.76-2.69 (m, 1H), 2.64-2.54 (m, 2H), 2.26 (s, 1H), 2.18-2.07 (m, 1H), 1.77-1.68 (m, 1H).

### Example 10.1

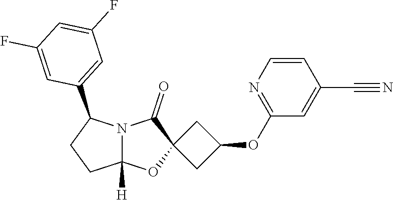 [SMILES: N#Cc1ccnc(O[C@H]2C[C@@]3(C2)O[C@@H]2CC[C@@H](c4cc(F)cc(F)c4)N2C3=O)c1]

### 2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile

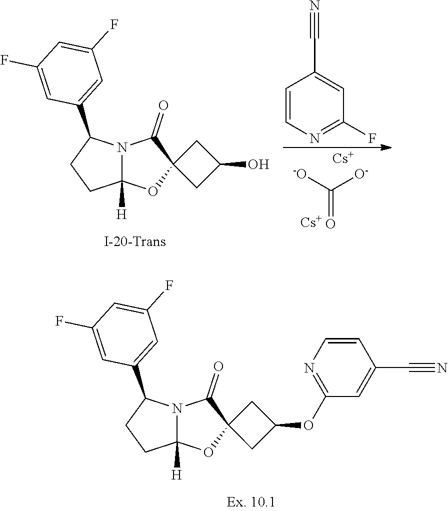

(1r,3R,5′S,7a′R)-5′-(3,5-difluorophenyl)-3-hydroxytetrahydro-3′H-spiro[cyclobutane-1,2′-pyrrolo[2,1-b]oxazol]-3′-one (23 mg, 0.078 mmol, I-20-Trans), DMF (3 mL), 4-cyano-2-fluoropyridine (11.9 mg, 0.097 mmol) and Cs₂CO₃ (38.1 mg, 0.117 mmol) were sealed in a microwave vessel and stirred at room temp overnight. The reaction was partitioned between pH7 buffer and ethyl acetate. The organic layer was dried over Na₂SO₄, filtered and evaporated. The resulting crude material was purified by flash chromatography [SiO₂, 80 g cartridge [3:1(v/v)/EtOAc:EtOH]/hexanes mixture (0% to 70%)] to afford 2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile. MS (ESI) m/z [M+H]⁺: 398. ¹H NMR (500 MHz, CDCl₃-d) δ 8.31-8.27 (m, 1H), 7.09 (dd, J=5.2, 1.3 Hz, 1H), 7.04-7.00 (m, 1H), 6.84-6.77 (m, 2H), 6.73 (m, 1H), 5.63 (m, 1H), 5.47 (m, 1H), 5.00 (t, J=7.4 Hz, 1H), 3.03-2.92 (m, 2H), 2.77-2.57 (m, 3H), 2.25 (ddd, J=16.0, 7.9, 4.7 Hz, 1H), 2.01 (m, 1H), 1.82-1.71 (m, 1H).

### Example 10.2

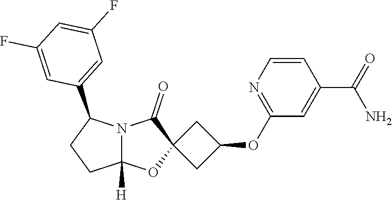 [SMILES: NC(=O)c1ccnc(O[C@H]2C[C@@]3(C2)O[C@@H]2CC[C@@H](c4cc(F)cc(F)c4)N2C3=O)c1]

### 2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinamide

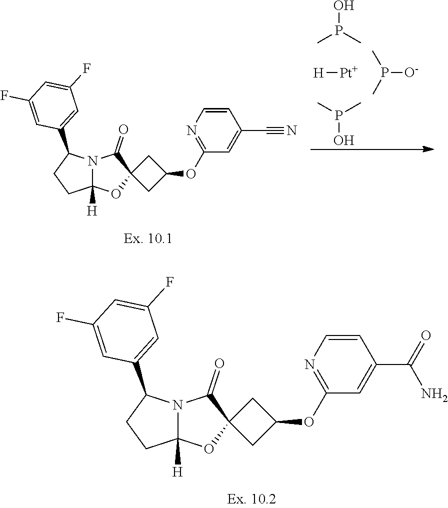

2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile (12 mg, 0.030 mmol, Ex. 10.1), ethanol (0.5 ml) and Ghaffar-Parkins catalyst (2.59 mg, 6.04 μmol) were sealed in a microwave vessel and stirred at 100° C. overnight. The reaction was partitioned between pH7 buffer and ethyl acetate. The organic layer was dried over Na₂SO₄, filtered and evaporated. The resulting crude material was purified by preparative TLC (silica gel) developed by a [3:1(v/v)/EtOAc:EtOH]/hexanes mixture (1:1, v/v) to give 2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinamide MS (ESI) m/z [M+H]⁺ calc'd for C₂₁H₂₀F₂N₃O₄: 417, found: 417. ¹H NMR (500 MHz, Methanol-d₄) δ 8.24 (d, J=5.3 Hz, 1H), 7.34 (dd, J=5.3, 1.4 Hz, 1H), 7.20 (s, 2H), 6.98-6.91 (m, 3H), 6.85 (m, 1H), 5.77 (dd, J=7.2, 5.0 Hz, 1H), 5.52-5.40 (m, 1H), 4.97 (t, J=7.8 Hz, 1H), 3.07-2.98 (m, 1H), 2.89-2.81 (m, 1H), 2.71 (m, 2H), 2.58 (dd, J=13.1, 6.9 Hz, 1H), 2.27 (ddd, J=11.8, 7.1, 2.8 Hz, 1H), 2.06-1.95 (m, 1H), 1.75 (m, 1H), 1.31 (s, 1H).

### Example 10.3

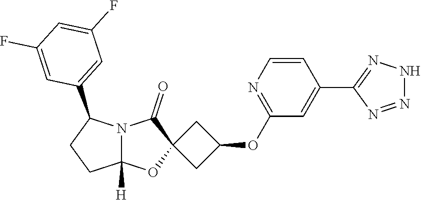 [SMILES: O=C1N2[C@@H](CC[C@H]2c2cc(F)cc(F)c2)O[C@]12C[C@H](Oc1cc(-c3nn[nH]n3)ccn1)C2]

### (1r,3R,5′S,7a′R)-3-((4-(2H-tetrazol-5-yl)pyridin-2-yl)oxy)-5′-(3,5-difluorophenyl)tetrahydro-3′H-spiro[cyclobutane-1,2′-pyrrolo[2,1-b]oxazol]-3′-one

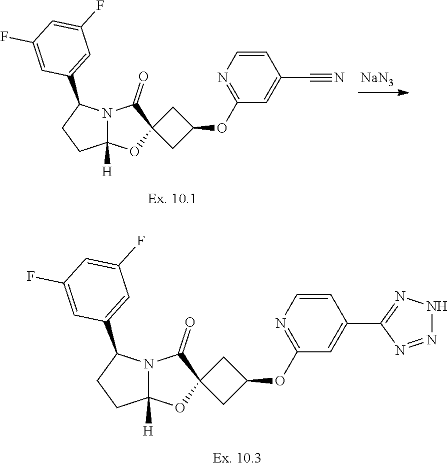 [SMILES: N#Cc1ccnc(O[C@H]2C[C@@]3(C2)O[C@@H]2CC[C@@H](c4cc(F)cc(F)c4)N2C3=O)c1 ; O=C1N2[C@@H](CC[C@H]2c2cc(F)cc(F)c2)O[C@]12C[C@H](Oc1cc(-c3nn[nH]n3)ccn1)C2 ; "NaN3"]

2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile (10 mg, 0.025 mmol, Ex. 10.1), DMSO (0.5 mL), sodium azide (4.91 mg, 0.075 mmol) and aluminum sulfate (3.18 μl, 0.025 mmol) were sealed in a microwave vessel and stirred at 140° C. for 2 d. The reaction was quenched by 0.5 mL of pH 7 buffer. The crude mixture was partitioned between water/ethyl acetate and the combined organic extracts were dried over Na₂SO₄, filtered and evaporated. The residue was submitted for reversed-phase purification to afford (2r,3R,5-S,7a(R)-3-((4-(2H-tetrazol-5-yl)pyridin-2-yl)oxy)-5′-(3,5-difluorophenyl)tetrahydro-3H-spiro[cyclobutane-1,2′-pyrrolo[2,1-b]oxazol]-3′-one. MS (ESI) m/z [M+H]⁺ calc'd for C₂₁H₁₉F₂N₆O₃: 441, found: 441. ¹H NMR (500 MHz, CD₃OD) δ 8.32 (d, J=5.3 Hz, 1H), 7.37 (dd, J=5.3, 1.4 Hz, 1H), 7.22 (s, 1H), 6.98-6.91 (m, 3H), 6.85 (tt, J=9.1, 2.3 Hz, 1H), 5.77 (dd, J=7.2, 5.0 Hz, 1H), 5.46 (m, 1H), 4.97 (t, J=7.9 Hz, 1H), 3.08-3.00 (m, 1H), 2.89-2.82 (m, 1H), 2.77-2.69 (m, 1H), 2.67 (s, 1H), 2.59 (dd, J=12.8, 6.5 Hz, 1H), 2.27 (ddd, J=11.7, 7.1, 2.7 Hz, 1H), 2.06-1.96 (m, 1H), 1.80-1.70 (m, 1H).

The compounds presented in Table 10 below were prepared using procedure analogous to one described for Example 10.1, 10.2 and 10.3 with the appropriately chosen intermediate from Table 4 or I-18. All products are in the trans configuration unless otherwise note.

TABLE 10

| | | | Exact Mass |
|---|---|---|---|
| Example | Structure | IUPAC Name | [M + H]+ |
| 10.4 | 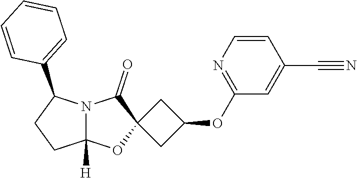 [SMILES: N#Cc1ccnc(O[C@H]2C[C@@]3(C2)O[C@@H]2CC[C@@H](c4ccccc4)N2C3=O)c1] | 2-(((1r,3R,5′S,7a′R)-3′-oxo-5′- phenyltetrahydro-3′H- spiro[cyclobutane-1,2′-pyrrolo[2,1- b]oxazol]-3-yl)oxy)isonicotinonitrile | Calc'd 361, found 361 |
| 10.5 | 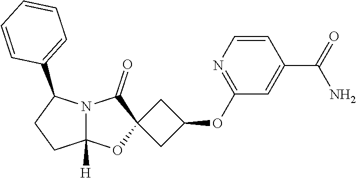 [SMILES: NC(=O)c1ccnc(O[C@H]2C[C@@]3(C2)O[C@@H]2CC[C@@H](c4ccccc4)N2C3=O)c1] | 2-(((1r,3R,5′S,7a′R)-3′-oxo-5′- phenyltetrahydro-3′H- spiro[cyclobutane-1,2′-pyrrolo[2,1- b]oxazol]-3-yl)oxy)isonicotinamide | Calc'd 379, found 379 |
| 10.6 | 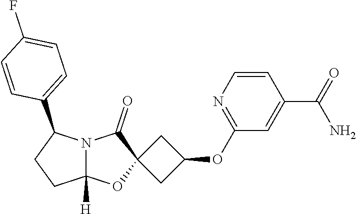 [SMILES: NC(=O)c1ccnc(O[C@H]2C[C@@]3(C2)O[C@@H]2CC[C@@H](c4ccc(F)cc4)N2C3=O)c1] | 2-(((1r,3R,5′S,7a′R)-5′-(4- fluorophenyl)-3′-oxotetrahydro-3′H- spiro[cyclobutane-1,2′-pyrrolo[2,1- b]oxazol]-3-yl)oxy)isonicotinamide | Calc'd 397, found 397 |
| 10.7 | 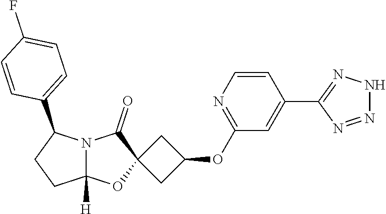 [SMILES: O=C1N2[C@@H](CC[C@H]2c2ccc(F)cc2)O[C@]12C[C@H](Oc1cc(-c3nn[nH]n3)ccn1)C2] | (1r,3R,5′S,7a′R)-3-((4-(2H-tetrazol-5- yl)pyridin-2-yl)oxy)-5′-(4- fluorophenyl)tetrahydro-3′H- spiro[cyclobutane-1,2′-pyrrolo[2,1- b]oxazol]-3′-one | Calc'd 422, found 422 |

### Example 11.1

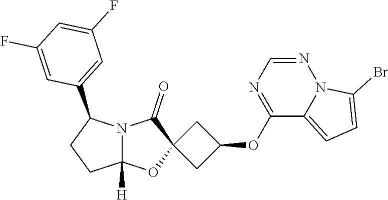 [SMILES: O=C1N2[C@@H](CC[C@H]2c2cc(F)cc(F)c2)O[C@]12C[C@H](Oc1ncnn3c(Br)ccc13)C2]

### (1r,3R,5′S,7a′R)-3-((7-bromopyrrolo[2,1-f][1,2,4]triazin-4-yl)oxy)-5′-(3,5-difluorophenyl)tetrahydro-3′H-spiro[cyclobutane-1,2′-pyrrolo[2,1-b]oxazol]-3′-one

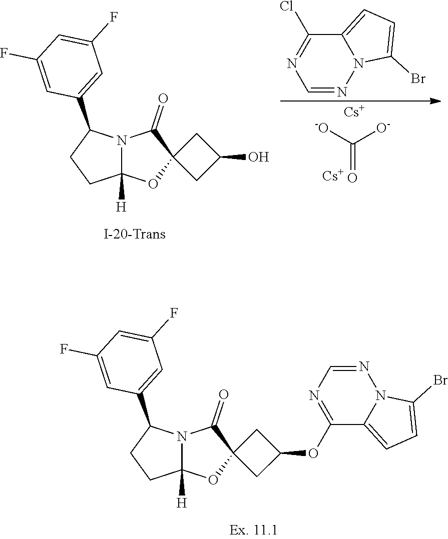

A microwave vial was charged with (1r,3R,5′S,7a′R)-5′-(3,5-difluorophenyl)-3-hydroxytetrahydro-3′H-spiro[cyclobutane-1,2′-pyrrolo[2,1-b]oxazol]-3′-one (I-20-Trans) (30 mg, 0.10 mmol), DMF (1 mL), 7-bromo-4-chloropyrrolo[2,1-F][1,2,4]triazine (28.3 mg, 0.12 mmol) and Cs₂CO₃ (49.7 mg, 0.15 mmol). The vial was capped and heated at 60° C. for 3 h. The reaction was quenched with pH 7 buffer and extracted with ethyl acetate, the combined organic extracts were dried over Na₂SO₄, filtered, and evaporated to afford crude material which was purified by revered-phase HPLC (TFA modified) to afford (1r,3R,5′S,7a′R)-3-((7-bromopyrrolo[2,1-f][1,2,4]triazin-4-yl)oxy)-5′-(3,5-difluorophenyl)tetrahydro-3′H-spiro[cyclobutane-1,2′-pyrrolo[2,1-b]oxazol]-3′-one. MS (ESI) m/z [M+H]⁺ calc'd for C₂₁H₁₈BrF₂N₄O₃: 491, found 491. ¹H NMR (500 MHz, CDCl₃-d) δ 8.15 (s, 1H), 6.93 (d, J=4.6 Hz, 1H), 6.85-6.78 (m, 2H), 6.77-6.70 (m, 2H), 5.72 (m, 1H), 5.67-5.63 (m, 1H), 5.01 (t, J=7.4 Hz, 1H), 3.14-3.00 (m, 2H), 2.79 (m, 2H), 2.64 (m, 1H), 2.27 (m, 1H), 2.02 (ddt, J=13.8, 10.1, 7.0 Hz, 1H), 1.78 (m, 1H).

### Example 11.2

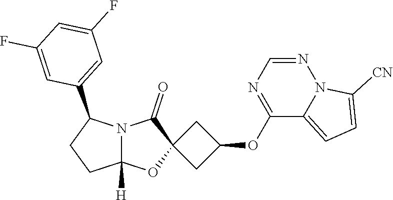 [SMILES: N#Cc1ccc2c(O[C@H]3C[C@@]4(C3)O[C@@H]3CC[C@@H](c5cc(F)cc(F)c5)N3C4=O)ncnn12]

### 4-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile

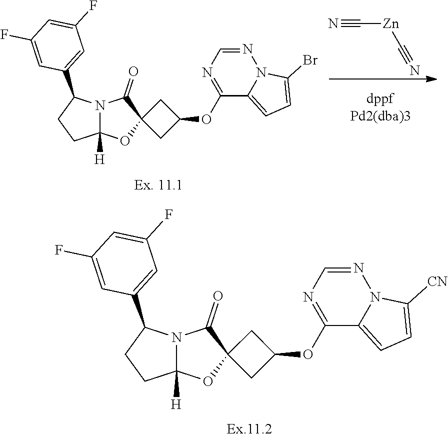

A reaction vial was charged with (1r,3R,5′S,7a′R)-3-((7-bromopyrrolo[2,1-f][1,2,4]triazin-4-yl)oxy)-5′-(3,5-difluorophenyl)tetrahydro-3′H-spiro[cyclobutane-1,2′-pyrrolo[2,1-b]oxazol]-3′-one (4 mg, 8 μmol) Pd₂(dba)₃ (0.74 mg, 0.81 μmol), 1,1′-bis(diphenylphosphino)ferrocene (0.45 mg, 0.81 μmol), zinc cyanide (1.9 mg, 0.016 mmol) and DMF/H₂O (99:1, v/v) (3 mL). The sealed vial was then bubbled w/N₂ for 30 min and the reaction mixture was placed in a 140° C. oil bath for 3 h and allowed to slowly cool to ambient. The crude was quenched with water and extracted with EtOAc, the combined extracts were dried over Na₂SO₄, filtered, and evaporated. The residue was purified by flash chromatography (SiO₂, 80 g) with [3:1(v/v)/EtOAc:EtOH]/hexanes mixture (0 to 70%) to afford 4-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile. MS (ESI) m/z [M+H]⁺ calc'd for C₂₂H₁₈F₂N₅O₃: 438, found: 438. ¹H NMR (500 MHz, CDCl₃-d) δ 8.25 (s, 1H), 7.24 (d, J=4.2 Hz, 1H), 6.87 (d, J=4.3 Hz, 1H), 6.80 (d, J=6.0 Hz, 2H), 6.74 (m, 1H), 5.78-5.69 (m, 1H), 5.66 (s, 1H), 5.01 (s, 1H), 3.08 (m, 2H), 2.81 (dd, J=12.9, 6.1 Hz, 2H), 2.64 (s, 1H), 2.33-2.22 (m, 1H), 2.05 (m, 2H).

The compounds presented in Table 11 below were prepared using procedure analogous that described for Example 11.1 and 11.2 with the appropriately chosen intermediate from Table 4. All products are in the trans configuration unless otherwise note.

TABLE 11

| | | | Exact Mass |
|---|---|---|---|
| Example | Structure | IUPAC Name | [M + H]+ |
| 11.3 | 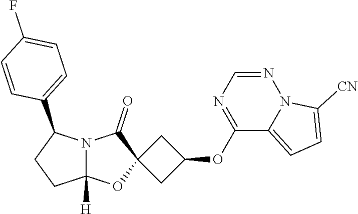 [SMILES: N#Cc1ccc2c(O[C@H]3C[C@@]4(C3)O[C@@H]3CC[C@@H](c5ccc(F)cc5)N3C4=O)ncnn12] | 4-(((1r,3R,5′S,7a′R)-5′-(4- fluorophenyl)-3′-oxotetrahydro-3′H- spiro[cyclobutane-1,2′-pyrrolo[2,1- b]oxazol]-3-yl)oxy)pyrrolo[2,1- f][1,2,4]triazine-7-carbonitrile | Calc'd 420, found 420 |

### Example 12.1 and 12.2

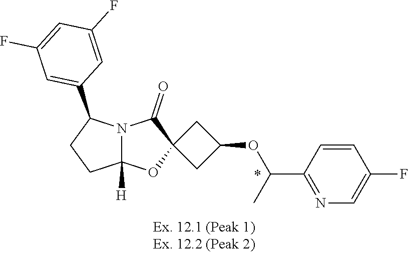 [SMILES: CC(O[C@H]1C[C@@]2(C1)O[C@@H]1CC[C@@H](c3cc(F)cc(F)c3)N1C2=O)c1ccc(F)cn1]

### (1R,3R,5′S,7a′R)-5′-(3,5-difluorophenyl)-3-((R or S)-1-(5-fluoropyridin-2-yl)ethoxy)tetrahydro-3′H-spiro[cyclobutane-1,2′-pyrrolo[2,1-b]oxazol]-3′-one and (1R,3R,5′S,7a′R)-5′-(3,5-difluorophenyl)-3-((S or R)-1-(5-fluoropyridin-2-yl)ethoxy)tetrahydro-3′H-spiro[cyclobutene-1,2′-pyrrolo[2,1-b]oxazol]-3′-one

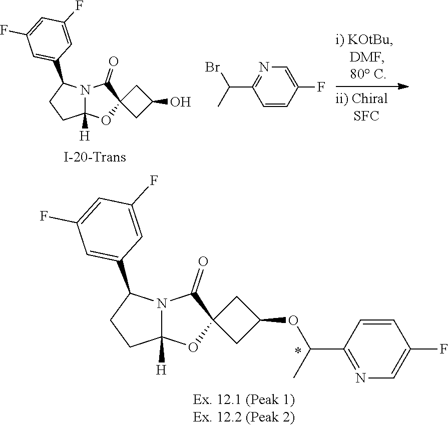

To a solution of (1r,3R,5′S,7a′R)-5′-(3,5-difluorophenyl)-3-hydroxytetrahydro-3′H-spiro[cyclobutane-1,2′-pyrrolo[2,1-b]oxazol]-3′-one (I-20-Trans) (30.0 mg, 0.10 mmol) in DMF (1 ml) was added 2-(1-bromoethyl)-5-fluoropyridine (24.9 mg, 0.12 mmol), potassium 2-methylpropan-2-olate (34.2 mg, 0.305 mmol). The resulting mixture was capped and stirred at 80° C. for 2 h. The reaction was cooled, filtered, and the filtrate was concentrated and purified by reverse phase Prep-HPLC (Column Boston Green ODS 150 mm×30 mm×5 m; H₂O (0.01% TFA)-CAN) to afford crude (5′S,7a′R)-5′-(3,5-difluorophenyl)-3-(1-(5-fluoropyridin-2-yl)ethoxy)tetrahydro-3′H-spiro[cyclobutane-1,2′-pyrrolo[2,1-b]oxazol]-3′-one. This material was separated via chiral SFC (ChiralPak AD (250 mm×30 mm×10 m) Condition 0.1% NH₃H₂O in MeOH) to afford two diastereomers, Peak 1 and Peak 2 as white solids. MS (ESI) m/z [M+H]⁺ calc'd for C₂₂H₂₂F₃N₂O₃: 419, found: 419.

Ex. 12.1 (SFC Peak 1): ¹H NMR (400 MHz, CD₃OD): δ 8.39 (d, J=2.86 Hz, 1H), 7.54-7.67 (m, 2H), 6.75-6.97 (m, 3H), 5.63 (dd, J=5.07, 7.21 Hz, 1H), 4.89-4.94 (m, 1H), 4.56 (q, J=6.56 Hz, 1H), 4.21 (quin, J=7.36 Hz, 1H), 2.61-2.71 (m, 2H), 2.38-2.54 (m, 2H), 2.14-2.27 (m, 2H), 1.94 (dddd, J=6.62, 7.99, 11.37, 13.13 Hz, 1H), 1.66 (tt, J=7.58, 11.67 Hz, 1H), 1.43 (d, J=6.56 Hz, 3H)

Ex. 12.2 (SFC Peak 2): ¹H NMR (400 MHz, CD₃OD): δ 8.39 (d, J=2.74 Hz, 1H), 7.54-7.68 (m, 2H), 6.78-6.98 (m, 3H), 5.66 (dd, J=5.01, 7.27 Hz, 1H), 4.89-4.94 (m, 1H), 4.56 (q, J=6.56 Hz, 1H), 4.21 (quin, J=7.36 Hz, 1H), 2.80-2.80 (m, 1H), 2.75 (td, J=6.53, 12.70 Hz, 1H), 2.65 (dtd, J=2.62, 7.78, 13.17 Hz, 1H), 2.54 (dd, J=7.81, 12.82 Hz, 1H), 2.39 (dd, J=7.27, 12.52 Hz, 1H), 2.14-2.21 (m, 2H), 1.87-1.99 (m, 1H), 1.59-1.69 (m, 1H), 1.43 (d, J=6.56 Hz, 3H)

The compounds presented in Table 12 below were prepared using procedure analogous that described for Ex. 12.1/12.2 using the appropriately chosen intermediates and reagents. SFC Peak 4 of the below example was inactive.

TABLE 12

| Ex. | | | Exact Mass |
|---|---|---|---|
| No. | Structure | IUPAC Name | [M + H]+ |
| 12.3 | 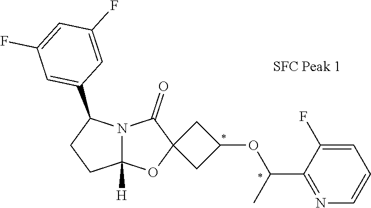 [SMILES: CC(OC1CC2(C1)O[C@@H]1CC[C@@H](c3cc(F)cc(F)c3)N1C2=O)c1ncccc1F] | (1R or S,3R or S,5′S,7a′R)-5′-(3,5- difluorophenyl)-3-((R or S)-1-(3- fluoropyridin-2- yl)ethoxy)tetrahydro-3′H- spiro[cyclobutane-1,2′- pyrrolo[2,1-b]oxazol]-3′-one | Calc'd 419, found 419 |
| 12.4 | 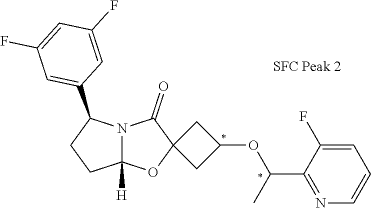 [SMILES: CC(OC1CC2(C1)O[C@@H]1CC[C@@H](c3cc(F)cc(F)c3)N1C2=O)c1ncccc1F] | (1S or R,3S or R,5′S,7a′R)-5′-(3,5- difluorophenyl)-3-((S or R)-1-(3- fluoropyridin-2- yl)ethoxy)tetrahydro-3′H- spiro[cyclobutane-1,2′- pyrrolo[2,1-b]oxazol]-3′-one | Calc'd 419, found 419 |
| 12.5 | 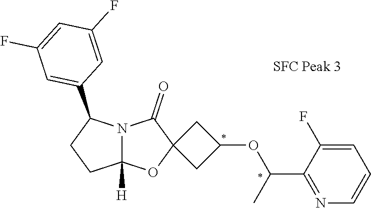 [SMILES: CC(OC1CC2(C1)O[C@@H]1CC[C@@H](c3cc(F)cc(F)c3)N1C2=O)c1ncccc1F] | (1R or S,3S or r,5′S,7a′R)-5′-(3,5- difluorophenyl)-3-((S or R)-1-(3- fluoropyridin-2- yl)ethoxy)tetrahydro-3′H- spiro[cyclobutane-1,2′- pyrrolo[2,1-b]oxazol]-3′-one | Calc'd 419, found 419 |
| 12.6 | 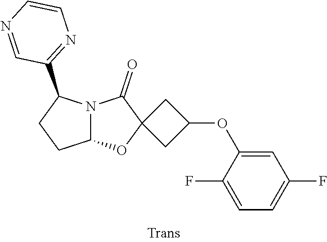 | (5′S,7a′R)-3-(2,5- difluorophenoxy)-5′-(pyrazin-2- yl)tetrahydro-3′H- spiro[cyclobutane-1,2′- pyrrolo[2,1-b][1,3]oxazol]-3′-one | Calc'd 374, found 374 |

### Example 13

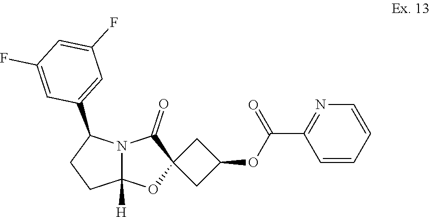 [SMILES: O=C(O[C@H]1C[C@@]2(C1)O[C@@H]1CC[C@@H](c3cc(F)cc(F)c3)N1C2=O)c1ccccn1]

### (1r,3R,5′S,7a′R)-5′-(3,5-difluorophenyl)-3′-oxotetrahydro-3′H-spiro[cyclobutane-1,2′-pyrrolo[2,1-b]oxazol]-3-yl picolinate

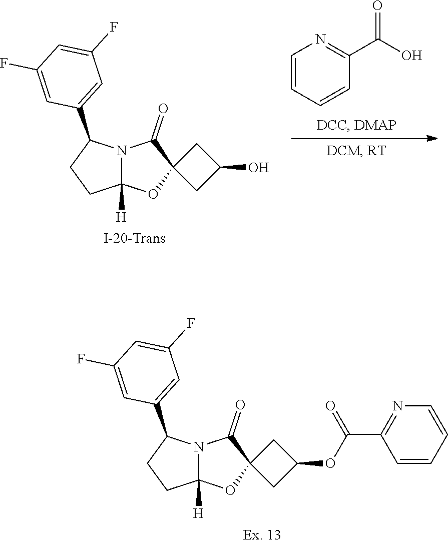

To a stirred solution of (5′S,7a′R)-5′-(3,5-difluorophenyl)-3-hydroxytetrahydro-3′H-spiro[cyclobutane-1,2′-pyrrolo[2,1-b]oxazol]-3′-one (I-20-Trans) (30 mg, 0.10 mmol) in DCM (1 ml) were added picolinic acid (17.5 mg, 0.142 mmol), DCC (31.4 mg, 0.152 mmol) and DMAP (2.48 mg, 0.020 mmol). After stirring at room temperature for 2.5 h, the reaction mixture was diluted with CH₂Cl₂ (25 mL) and washed successively with cold water (10 mL) and aq. sat. NaHCO₃ (10 mL). The organic layer was collected, dried over Na₂SO₄, filtered and concentrated. The resulting oil was purified by reverse phase Prep-HPLC (water (10 mM-NH₄HCO₃)-ACN Begin B 35 End B 65 Gradient) to afford (5′S,7a′R)-5′-(3,5-difluorophenyl)-3′-oxotetrahydro-3′H-spiro[cyclobutane-1,2′-pyrrolo[2,1-b]oxazol]-3-yl picolinate. MS (ESI) m/z [M+H]⁺ calc'd for C₂₁H₁₉F₂N₂O₄: 401, found: 401. ¹H NMR (400 MHz, CD₃OD-d₄) δ 8.69 (dd, J=0.78, 4.8 Hz, 1H), 8.21 (d, J=7.8 Hz, 1H), 8.02 (dt, J=1.7, 7.7 Hz, 1H), 7.65 (ddd, J=1.2, 4.8, 7.7 Hz, 1H), 7.01-6.80 (m, 3H), 5.76 (dd, J=5.0, 7.3 Hz, 1H), 5.47 (quin, J=7.3 Hz, 1H), 4.96 (t, J=7.8 Hz, 1H), 3.07-2.95 (m, 2H), 2.77-2.68 (m, 3H), 2.29-2.22 (m, 1H), 2.04-1.94 (m, 1H), 1.77-1.7 (m, 1H).

### Example 14

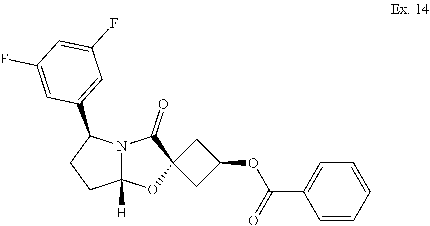 [SMILES: O=C(O[C@H]1C[C@@]2(C1)O[C@@H]1CC[C@@H](c3cc(F)cc(F)c3)N1C2=O)c1ccccc1]

### (1r,3R,5′S,7a′R)-5′-(3,5-difluorophenyl)-3′-oxotetrahydro-3′H-spiro[cyclobutane-1,2′-pyrrolo[2,1-b]oxazol]-3-yl benzoate

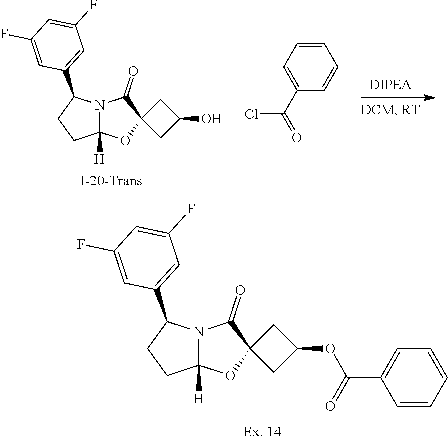

To a solution of (5′S,7a′R)-5′-(3,5-difluorophenyl)-3-hydroxytetrahydro-3′H-spiro[cyclobutane-1,2′-pyrrolo[2,1-b]oxazol]-3′-one (I-20-Trans) (50 mg, 0.17 mmol), DIEA (89 μl, 0.51 mmol) in DCM (1 ml) was added benzoyl chloride (36 mg, 0.25 mmol) and the resulting mixture was stirred at 20° C. for 12 h. The reaction was concentrated directly and the residue was purified by reverse phase Prep-HPLC (TFA) to give (5′S,7a′R)-5′-(3,5-difluorophenyl)-3′-oxotetrahydro-3′H-spiro[cyclobutane-1,2′-pyrrolo[2,1-b]oxazol]-3-yl benzoate as white solid. MS (ESI) m/z [M+H]⁺ calc'd for C₂₂H₂₀F₂NO₄: 400, found: 400. ¹H NMR (400 MHz, CD₃OD-d₄) δ 8.07-8.00 (m, 2H), 7.58-7.65 (m, 1H), 7.52-7.44 (m, 2H), 6.99-6.90 (m, 2H), 6.85 (tt, J=2.3, 9.1 Hz, 1H), 5.76 (dd, J=5.0, 7.3 Hz, 1H), 5.42 (quin, J=7.3 Hz, 1H), 4.96 (t, J=7.8 Hz, 1H), 3.04-2.96 (m, 1H), 2.91 (ddd, J=1.6, 7.0, 13.6 Hz, 1H), 2.73-2.63 (m, 3H), 2.29-2.21 (m, 1H), 2.03-1.93 (m, 1H), 1.73 (tt, J=7.6, 11.7 Hz, 1H).

### Example 15.1, 15.2, 15.3 and 15.4

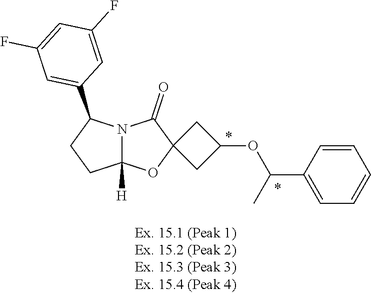 [SMILES: CC(OC1CC2(C1)O[C@@H]1CC[C@@H](c3cc(F)cc(F)c3)N1C2=O)c1ccccc1]

### ((1S or R),3R,5′S,7a′R)-5′-(3,5-difluorophenyl)-3-((S or R)-1-phenylethoxy)tetrahydro-3′H-spiro[cyclobutane-1,2′-pyrrolo[2,1-b]oxazol]-3′-one and ((1R or S),3R,5′S,7a′R)-5′-(3,5-difluorophenyl)-3-((S or R)-1-phenylethoxy)tetrahydro-3′H-spiro[cyclobutane-1,2′-pyrrolo[2,1-b]oxazol]-3′-one and ((1S or R),3R,5′S,7a′R)-5′-(3,5-difluorophenyl)-3-((R or S)-1-phenylethoxy)tetrahydro-3′H-spiro[cyclobutane-1,2′-pyrrolo[2,1-b]oxazol]-3′-one and ((1R or S),3R,5′S,7a′R)-5′-(3,5-difluorophenyl)-3-((R or S)-1-phenylethoxy)tetrahydro-3′H-spiro[cyclobutane-1,2′-pyrrolo[2,1-b]oxazol]-3′-one

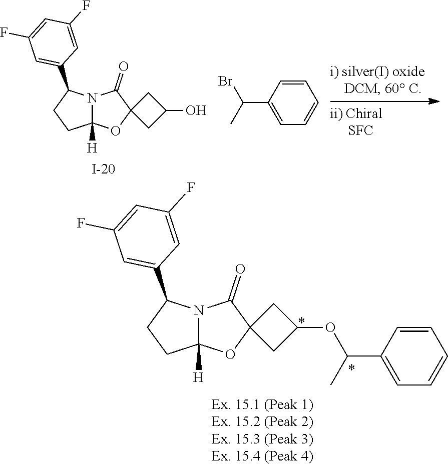

To a solution of (5′S,7a′R)-5′-(3,5-difluorophenyl)-3-hydroxytetrahydro-3′H-spiro[cyclobutane-1,2′-pyrrolo[2,1-b]oxazol]-3′-one (I-20) (10 mg, 0.034 mmol) in DCM (0.2 ml), n-hexane (0.8 ml) was added along with (1-bromoethyl)benzene (12.5 mg, 0.068 mmol), monosilver(I) monosilver(III) monoxide (23.5 mg, 0.102 mmol) and 4 A molecular sieves. The reaction mixture was flushed with N₂, sealed, and stirred at 60° C. in the dark under N₂ for 2 h. The mixture was cooled and filtered through Celite and concentrated. The residue was purified by reverse phase Prep-HPLC (TFA) to the crude product. The product mixture was resolved by Chiral-SFC (Column: DAICEL CHIRALPAK IG (250 mm×10 mm, 10 m)), Mobile phase: A: CO₂ B: 0.1% NH₃H₂O in MeOH) to give 4 diastereomers of (5′S,7a′R)-5′-(3,5-difluorophenyl)-3-(1-phenylethoxy)tetrahydro-3′H-spiro[cyclobutane-1,2′-pyrrolo[2,1-b]oxazol]-3′-one as white solid.

Ex. 15.1 (Peak 1): MS (ESI) m/z [M+Na]⁺ calc'd for C₂₃H₂₃F₂NNaO₃: 422, found: 422. ¹H NMR (400 MHz, CD₃OD-d₄) δ 7.38-7.23 (m, 5H), 6.94-6.78 (m, 3H), 5.61 (dd, J=5.0, 7.2 Hz, 1H), 4.92 (br s, 1H), 4.46 (q, J=6.5 Hz, 1H), 4.18-4.09 (m, 1H), 2.70-2.60 (m, 2H), 2.48-2.33 (m, 2H), 2.26-2.13 (m, 2H), 1.93 (dddd, J=6.6, 7.9, 11.3, 13.1 Hz, 1H), 1.65 (tt, J=7.6, 11.7 Hz, 1H), 1.41 (d, J=6.6 Hz, 3H).

Ex. 15.2 (Peak 2): MS (ESI) m/z [M+Na]⁺ calc'd for C₂₃H₂₃F₂NNaO₃: 422, found: 422. ¹H NMR (400 MHz, CD₃OD-d₄) δ 7.42-7.19 (m, 5H), 6.99-6.74 (m, 3H), 5.64 (dd, J=5.1, 7.1 Hz, 1H), 4.87 (br s, 1H), 4.45 (q, J=6.4 Hz, 1H), 4.13 (quin, J=7.5 Hz, 1H), 2.76-2.58 (m, 2H), 2.51 (dd, J=7.9, 12.9 Hz, 1H), 2.34 (dd, J=7.6, 12.4 Hz, 1H), 2.22-2.12 (m, 1H), 2.08 (td, J=6.7, 13.0 Hz, 1H), 1.98-1.86 (m, 1H), 1.62 (tt, J=7.5, 11.7 Hz, 1H), 1.41 (d, J=6.6 Hz, 3H).

Ex. 15.3 (Peak 3): MS (ESI) m/z [M+H]⁺ calc'd for C₂₃H₂₄F₂NO₃: 400, found: 400. ¹H NMR (400 MHz, CD₃OD-d₄) δ 7.19-7.39 (m, 4H), 6.76-6.95 (m, 3H), 5.68 (dd, J=4.89, 7.63 Hz, 1H), 4.83-4.87 (m, 1H), 4.45 (q, J=6.44 Hz, 1H), 3.82 (quin, J=7.06 Hz, 1H), 2.84 (td, J=6.11, 12.10 Hz, 1H), 2.65 (dtd, J=2.21, 7.76, 13.28 Hz, 1H), 2.50 (td, J=6.24, 12.19 Hz, 1H), 2.40 (dd, J=7.33, 11.98 Hz, 1H), 2.11-2.21 (m, 2H), 1.91 (dddd, J=6.56, 8.23, 11.74, 13.29 Hz, 1H), 1.58 (tt, J=7.75, 11.92 Hz, 1H), 1.41 (d, J=6.44 Hz, 3H).

Ex. 15.4 (Peak 4): MS (ESI) m/z [M+H]⁺ calc'd for C₂₃H₂₄F₂NO₃: 400, found: 400. ¹H NMR (400 MHz, CD₃OD-d₄δ 7.22-7.36 (m, 5H), 6.77-6.92 (m, 3H), 5.65 (dd, J=4.89, 7.63 Hz, 1H), 4.85-4.88 (m, 1H), 4.46 (q, J=6.56 Hz, 1H), 3.83 (quin, J=7.06 Hz, 1H), 2.81 (td, J=6.24, 12.19 Hz, 1H), 2.66 (dtd, J=2.21, 7.77, 13.25 Hz, 1H), 2.48-2.58 (m, 1H), 2.27 (dd, J=7.33, 12.58 Hz, 2H), 2.14-2.22 (m, 1H), 1.91 (dddd, J=6.62, 8.23, 11.73, 13.25 Hz, 1H), 1.61 (tt, J=7.73, 11.88 Hz, 1H), 1.41 (d, J=6.44 Hz, 3H).

### Example 16.1 and 16.2

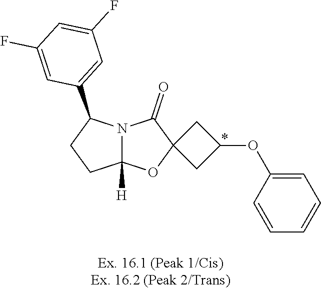 [SMILES: O=C1N2[C@@H](CC[C@H]2c2cc(F)cc(F)c2)OC12CC(Oc1ccccc1)C2]

### ((1s or r),3R,5′S,7a′R)-5′-(3,5-difluorophenyl)-3-phenoxytetrahydro-3′H-spiro[cyclobutane-1,2′-pyrrolo[2,1-b]oxazol]-3′-one and ((1r or s),3R,5′S,7a′R)-5′-(3,5-difluorophenyl)-3-phenoxytetrahydro-3′H-spiro[cyclobutane-1,2′-pyrrolo[2,1-b]oxazol]-3′-one

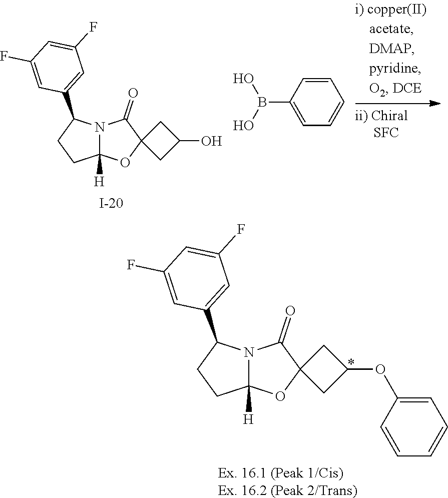

To a solution of (5′S,7a′R)-5′-(3,5-difluorophenyl)-3-hydroxytetrahydro-3′H-spiro[cyclobutane-1,2′-pyrrolo[2,1-b]oxazol]-3′-one (I-20) (80 mg, 0.27 mmol) in DCE (4 ml) was added phenylboronic acid (165 mg, 1.35 mmol), N,N-dimethylpyridin-4-amine (6.6 mg, 0.05 mmol), pyridine (0.065 ml, 0.81 mmol) and molecular sieves (4 A) in a 20 mL, pressure relief vial. The reaction was stirred at 80° C. for 10 min, then diacetoxycopper (49 mg, 0.27 mmol) was added. The resulting mixture was stirred at 80° C. for 12 h under a balloon of O₂ (1 atm). The reaction was cooled and the solvent removed under reduced pressure. The residue was purified by reverse phase Prep-HPLC to give the two diastereomers. MS (ESI) m/z [M+H]⁺ calc'd for C₂₁H₂₀F₂NO₃: 371, found: 371.

Ex. 16.1 (Peak 1/Cis): ¹H NMR (400 MHz, CDCl₃-d) 6=7.25-7.21 (m, 2H), 6.93 (br t, J=7.3 Hz, 1H), 6.84-6.55 (m, 5H), 5.60 (br t, J=5.7 Hz, 1H), 5.00-4.85 (m, 2H), 2.91 (ddd, J=6.9, 12.9, 19.4 Hz, 2H), 2.69-2.54 (m, 3H), 2.26-2.16 (m, 1H), 2.00-1.91 (m, 1H), 1.23 (s, 1H).

Ex. 16.2 (Peak 2/Trans): ¹H NMR (400 MHz, CDCl₃-d) 6=7.27 (br s, 2H), 7.02-6.90 (m, 1H), 6.89-6.65 (m, 5H), 5.69-5.58 (m, 1H), 5.02-4.92 (m, 1H), 4.71-4.58 (m, 1H), 3.22-3.03 (m, 2H), 2.70-2.56 (m, 2H), 2.50 (br d, J=6.6 Hz, 1H), 2.21 (br d, J=3.2 Hz, 1H), 2.08-1.88 (m, 1H), 1.23 (br s, 1H).

The compounds presented in Table 13 below were prepared using procedure analogous that described for Ex. 16.1/16.2 using the intermediate I-26 and reagents.

TABLE 13

| Ex. | | | Exact Mass |
|---|---|---|---|
| No. | Structure | IUPAC Name | [M + H]+ |
| 16.3 | 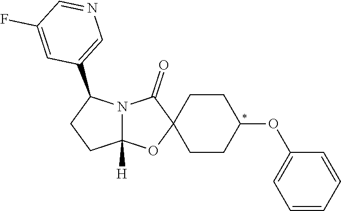 [SMILES: O=C1N2[C@@H](CC[C@H]2c2cncc(F)c2)OC12CCC(Oc1ccccc1)CC2] | (1r or s),(5′S,7a′R)-5′-(5-fluoropyridin-3- yl)-4-phenoxytetrahydro-3′H- spiro[cyclohexane-1,2′-pyrrolo[2,1- b]oxazol]-3′-one | Calc'd 383, found 383 |
| 16.4 | 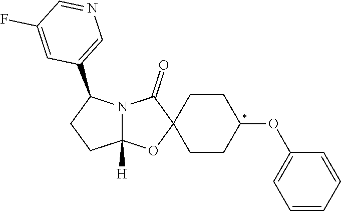 [SMILES: O=C1N2[C@@H](CC[C@H]2c2cncc(F)c2)OC12CCC(Oc1ccccc1)CC2] | (1s or r), (5′S,7a′R)-5′-(5-fluoropyridin-3- yl)-4-phenoxytetrahydro-3′H- spiro[cyclohexane-1,2′-pyrrolo[2,1- b]oxazol]-3′-one | Calc'd 383, found 383 |

### Example 17.1 and 17.2

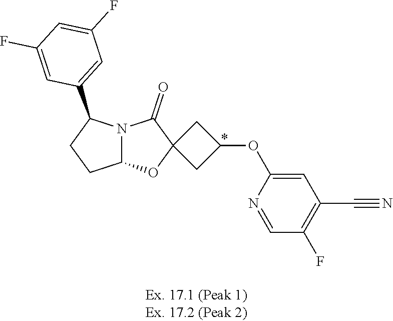

### 2-((((Is or r),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile and 2-((((1r or s),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile

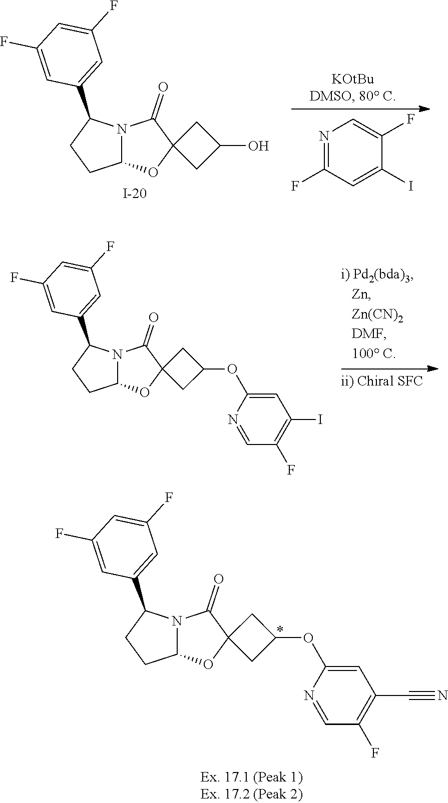

### Step 1. (5′S,7a′R)-5′-(3,5-difluorophenyl)-3-((5-fluoro-4-iodopyridin-2-yl)oxy)tetrahydro-3′H-spiro[cyclobutane-1,2′-pyrrolo[2,1-b]oxazol]-3′-one

To a solution of (5′S,7a′R)-5′-(3,5-difluorophenyl)-3-hydroxytetrahydro-3′H-spiro[cyclobutane-1,2′-pyrrolo[2,1-b]oxazol]-3′-one (I-20) (50 mg, 0.17 mmol) in DMSO (2 ml) was added potassium tert-butoxide (38 mg, 0.034 mmol) and 2,5-difluoro-4-iodopyridine (61.2 mg, 0.254 mmol). The reaction was stirred at 80° C. for 12 h. The reaction solution was filtered and purified by prep HPLC (Instrument EJ; water (TFA)-MeCN) to give (5′S,7a′R)-5′-(3,5-difluorophenyl)-3-((5-fluoro-4-iodopyridin-2-yl)oxy)tetrahydro-3′H-spiro[cyclobutane-1,2′-pyrrolo[2,1-b]oxazol]-3′-one (15 mg) as yellow oil. MS (ESI) m/z [M+H]⁺ calc'd for C₂₀H₁₇F₃IN₂O₃: 517, found: 517.

### Step 2. 2-(((5'S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile

To a solution of (5′S,7a′R)-5′-(3,5-difluorophenyl)-3-((5-fluoro-4-iodopyridin-2-yl)oxy)tetrahydro-3′H-spiro[cyclobutane-1,2′-pyrrolo[2,1-b]oxazol]-3′-one (10 mg, 0.019 mmol) in DMF (1 mL) was added dicyanozinc (6.8 mg, 0.058 mmol), zinc (0.51 mg, 7.7 μmol), 1,1′-bis(diphenylphosphino)ferrocene (4.3 mg, 7.7 μmol) and Pd₂(dba)₃ (3.6 mg, 3.9 μmol) under N₂ atmosphere. The mixture was stirred at 100° C. for 12 h, and darkened to dark brown over this time. The mixture was cooled, filtered, and purified by Prep HPLC (Instrument EJ; water (TFA)-MeCN to give 2-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile as colorless oil. The purified diastereomeric mixture of 2-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile (10 mg, 0.024 mmol) was separated by SFC (Instrument SFC-25 Method Column DAICEL CHIRALPAK AD (250 mm×30 mm, 10 m) Condition Neutral-IPA) to give two diastereomers of 2-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile SFC-Peak 1, (100 ee %, RT=3.81 min) and SFC-Peak 2, (99 ee %, RT=4.55 min) both as a white solids. MS (ESI) m/z [M+H]⁺ calc'd for C₂₁H₁₇F₃N₃O₃: 416, found: 416.

Ex. 17.1 (SFC-Peak 1/Cis): ¹H NMR (400 MHz, CDCl₃) δ 8.17 (s, 1H), 6.96 (d, J=3.81 Hz, 1H), 6.80 (br d, J=6.44 Hz, 2H), 6.69-6.76 (m, 1H), 5.64 (dd, J=5.13, 6.91 Hz, 1H), 5.06-5.15 (m, 1H), 4.99 (t, J=7.7 Hz, 1H), 3.10-3.17 (m, 2H), 2.55-2.69 (m, 2H), 2.44-2.51 (m, 1H), 2.20-2.28 (m, 1H), 1.98 (tdd, J=6.94, 11.0, 13.5 Hz, 1H), 1.64-1.74 (m, 1H) Ex. 17.2 (SFC-Peak 2/Trans): ¹H NMR (400 MHz, CDCl₃) δ 8.20 (d, J=3.1 Hz, 1H), 7.65 (dd, J=3.0, 6.9 Hz, 1H), 6.77-6.83 (m, 2H), 6.72 (tt, J=2.4, 8.7 Hz, 1H), 5.65 (dd, J=5.0, 7.0 Hz, 1H), 5.20 (t, J=7.0 Hz, 1H), 4.99 (t, J=7.6 Hz, 1H), 3.12-3.19 (m, 2H), 2.55-2.71 (m, 3H), 2.20-2.29 (m, 1H), 1.93-2.04 (m, 1H), 1.65-1.76 (m, 1H)

### Example 18.1

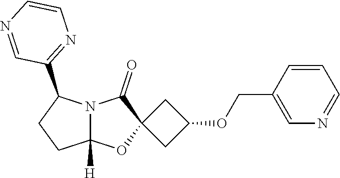 [SMILES: O=C1N2[C@@H](CC[C@H]2c2cnccn2)O[C@]12C[C@@H](OCc1cccnc1)C2]

### (1S,3S,5′S,7a′R)-5′-(pyrazin-2-yl)-3-(pyridin-3-ylmethoxy)tetrahydro-3′H-spiro[cyclobutane-1,2′-pyrrolo[2,1-b]oxazol]-3′-one

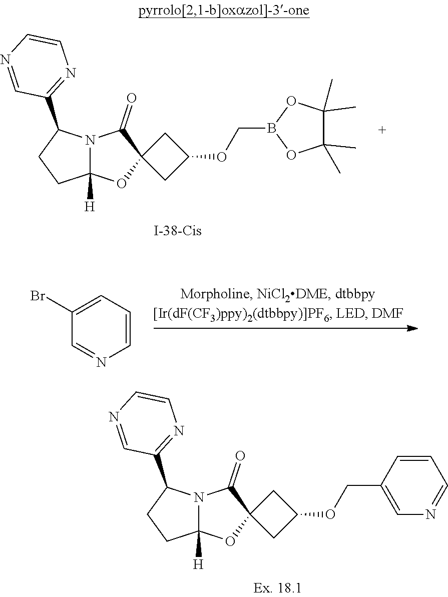

(1S,3S,5′S,7a′R)-5′-(pyrazin-2-yl)-3-((4,4,5,5-tetramethyl-1,3,2-dioxaborolan-2-yl)methoxy)tetrahydro-3′H-spiro[cyclobutane-1,2′-pyrrolo[2,1-b]oxazol]-3′-one (61.0 mg, 0.152 mmol) (I-38-Cis), [Ir(dF(CF₃)ppy)₂(dtbbpy)]PF₆ (1 mol %), 3-bromopyridine (16 mg, 0.101 mmol) and were added to a 4 mL glass vial equipped with a magnetic stir bar and dissolved in DMF. Morpholine (13.23 μl, 0.152 mmol) was added. In a second vial, Nickel(II) chloride ethylene glycol dimethyl ether complex (2.225 mg, 10.13 μmol) and 4,4′-di-tert-butyl-2,2′-bipyridine (2.72 mg, 10.13 μmol) were added and dissolved in DMF. The mixture was sonicated for 30 seconds and heated with a heat gun until a clear green solution was obtained. Both mixtures were combined, and the resulting reaction mixture with a total molarity of 0.1 M was irradiated with blue LEDs (445 nm at 220 mW) for 2 h. The mixture was assayed, and the product was found. The crude mixture was directly purified. The reaction crude was purified by reverse phase chromatography eluting with acetonitrile/water+0.05% TFA affording (1S,3S,5′S,7a′R)-5′-(pyrazin-2-yl)-3-(pyridin-3-ylmethoxy)tetrahydro-3′H-spiro[cyclobutane-1,2′-pyrrolo[2,1-b]oxazol]-3′-one (Ex. 18.1). MS (ESI) m/z [M+H]⁺: 353; ¹H NMR (499 MHz, CDCl3) δ 8.65 (d, J=1.3 Hz, 1H), 8.59 (s, 1H), 8.55 (d, J=3.8 Hz, 1H), 8.53-8.52 (m, 1H), 8.51 (d, J=2.5 Hz, 1H), 7.71 (d, J=7.8 Hz, 1H), 7.31 (dd, J=7.8, 4.9 Hz, 1H), 5.67 (dd, J=7.0, 5.0 Hz, 1H), 5.12-5.07 (m, 1H), 4.48 (s, 2H), 4.07 (p, J=6.9 Hz, 1H), 2.91 (dt, J=11.8, 6.1 Hz, 1H), 2.85 (dt, J=12.2, 6.1 Hz, 1H), 2.64-2.56 (m, 1H), 2.43 (dd, J=11.9, 7.2 Hz, 1H), 2.34 (dd, J=11.9, 7.1 Hz, 3H), 1.76-1.65 (m, 1H).

The compounds presented in Table 14 below were prepared using a procedure analogous to one described for Example 18.1 with the appropriately chosen intermediate from Table 6, commercially available or known aromatic electrophiles. All products are drawn in either cis or trans geometry unless unknown.

TABLE 14

| Ex. | | | Exact Mass |
|---|---|---|---|
| No. | Structure | IUPAC Name | [M + H]+ |
| 18.2 | 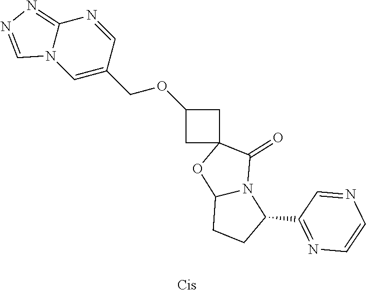 [SMILES: O=C1N2C(CC[C@H]2c2cnccn2)OC12CC(OCc1cnc3nncn3c1)C2] | (5′S)-5′-(pyrazin-2-yl)-3- [([1,2,4]triazolo[4,3-a]pyrimidin-6- yl)methoxy]tetrahydro-3′H- spiro[cyclobutane-1,2′-pyrrolo[2,1- b][1,3]oxazol]-3′-one | Calc'd 394, found 394 |
| 18.3 | 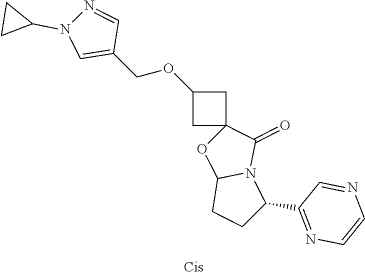 [SMILES: O=C1N2C(CC[C@H]2c2cnccn2)OC12CC(OCc1cnn(C3CC3)c1)C2] | (5′S)-3-[(1-cyclopropyl-1H-pyrazol- 4-yl)methoxy]-5′-(pyrazin-2- yl)tetrahydro-3′H-spiro[cyclobutane- 1,2′-pyrrolo [2,1-b][1,3]oxazol]-3′-one | Calc'd 382, found 382 |
| 18.4 | 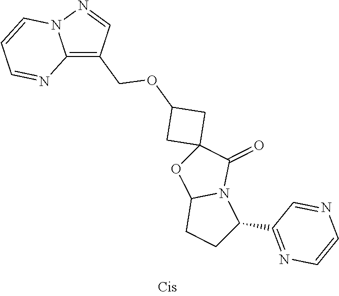 [SMILES: O=C1N2C(CC[C@H]2c2cnccn2)OC12CC(OCc1cnn3cccnc13)C2] | (5′S)-5′-(pyrazin-2-yl)-3- [(pyrazolo[1,5-a]pyrimidin-3- yl)methoxy]tetrahydro-3′H- spiro[cyclobutane-1,2′-pyrrolo[2,1- b][1,3]oxazol]-3′-one | Calc'd 393, found 393 |
| 18.5 | 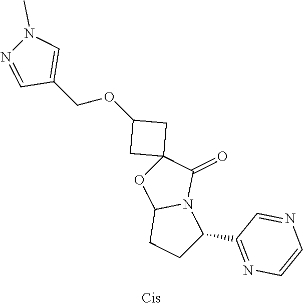 [SMILES: Cn1cc(COC2CC3(C2)OC2CC[C@@H](c4cnccn4)N2C3=O)cn1] | (5′S)-3-[(1-methyl-1H-pyrazol-4- yl)methoxy]-5′-(pyrazin-2- yl)tetrahydro-3′H-spiro[cyclobutane- 1,2′-pyrrolo[2,1-b][1,3]oxazol]-3′-one | Calc'd 356, found 356 |
| 18.6 | 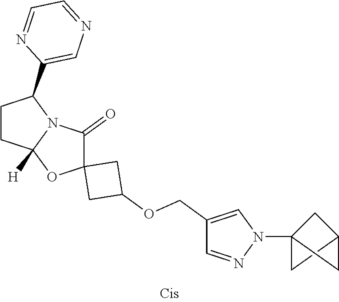 [SMILES: O=C1N2[C@@H](CC[C@H]2c2cnccn2)OC12CC(OCc1cnn(C34CC(C3)C4)c1)C2] | (5′S, 7a′R)-3-{[1- (bicyclo[1.1.1]pentan-1-yl)-1H- pyrazol-4-yl]methoxy}-5′-(pyrazin-2- yl)tetrahydro-3′H-spiro[cyclobutane- 1,2′-pyrrolo[2,1-b][1,3]oxazol]-3′-one | Calc'd 408, found 408 |
| 18.7 | 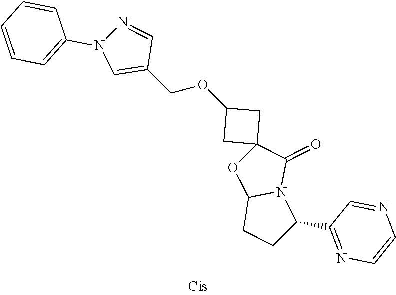 [SMILES: O=C1N2C(CC[C@H]2c2cnccn2)OC12CC(OCc1cnn(-c3ccccc3)c1)C2] | (5′S)-3-[(1-phenyl-1H-pyrazol-4- yl)methoxy]-5′-(pyrazin-2- yl)tetrahydro-3′H-spiro[cyclobutane- 1,2′-pyrrolo [2,1-b][1,3]oxazol]-3′-one | Calc'd 418, found 418 |
| 18.8 | 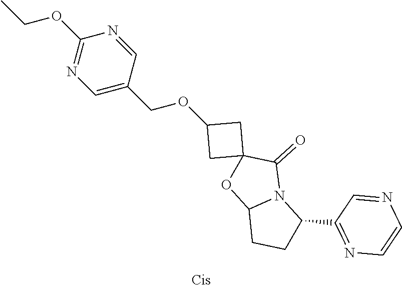 [SMILES: CCOc1ncc(COC2CC3(C2)OC2CC[C@@H](c4cnccn4)N2C3=O)cn1] | (5′S)-3-[(2-ethoxypyrimidin-5- yl)methoxy]-5′-(pyrazin-2- yl)tetrahydro-3′H-spiro[cyclobutane- 1,2′-pyrrolo[2,1-b][1,3]oxazol]-3′-one | Calc'd 398, found 398 |
| 18.9 | 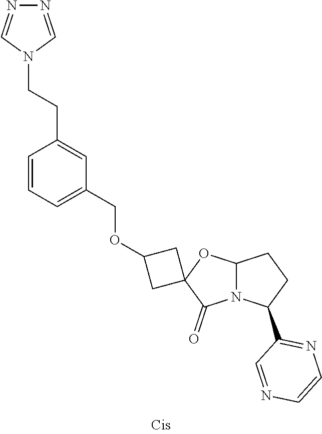 [SMILES: O=C1N2C(CC[C@H]2c2cnccn2)OC12CC(OCc1cccc(CCn3cnnc3)c1)C2] | (5′S)-5′-(pyrazin-2-yl)-3-({3-[2-(4H- 1,2,4-triazol-4- yl)ethyl]phenyl}methoxy)tetrahydro- 3′H-spiro[cyclobutane-1,2′- pyrrolo[2,1-b][1,3]oxazol]-3′-one | Calc'd 447, found 447 |
| 18.10 | 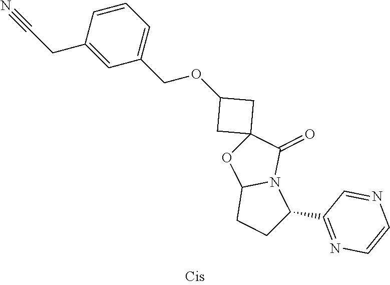 [SMILES: N#CCc1cccc(COC2CC3(C2)OC2CC[C@@H](c4cnccn4)N2C3=O)c1] | [3-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)phenyl]acetonitrile | Calc'd 391, found 391 |
| 18.11 | 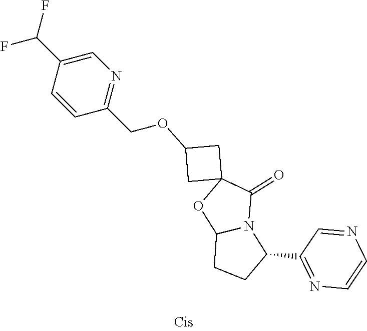 [SMILES: O=C1N2C(CC[C@H]2c2cnccn2)OC12CC(OCc1ccc(C(F)F)cn1)C2] | (5′S)-3-{[5-(difluoromethyl)pyridin- 2-yl]methoxy}-5′-(pyrazin-2- yl)tetrahydro-3′H-spiro[cyclobutane- 1,2′-pyrrolo[2,1-b][1,3]oxazol]-3′-one | Calc'd 403, found 403 |
| 18.12 | 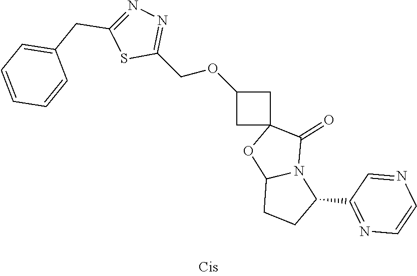 [SMILES: O=C1N2C(CC[C@H]2c2cnccn2)OC12CC(OCc1nnc(Cc3ccccc3)s1)C2] | (5′S)-3-[(5-benzyl-1,3,4-thiadiazol-2- yl)methoxy]-5′-(pyrazin-2- yl)tetrahydro-3′H-spiro[cyclobutane- 1,2′-pyrrolo[2,1-b][1,3]oxazol]-3′-one | Calc'd 450, found 450 |
| 18.13 | 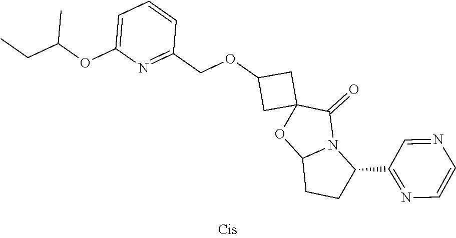 [SMILES: CCC(C)Oc1cccc(COC2CC3(C2)OC2CC[C@@H](c4cnccn4)N2C3=O)n1] | (5′S)-3-({6-[(butan-2-yl)oxy]pyridin- 2-yl}methoxy)-5′-(pyrazin-2- yl)tetrahydro-3′H-spiro[cyclobutane- 1,2′-pyrrolo[2,1-b][1,3]oxazol]-3′-one | Calc'd 425, found 425 |
| 18.14 | 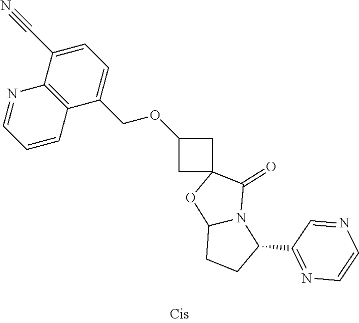 [SMILES: N#Cc1ccc(COC2CC3(C2)OC2CC[C@@H](c4cnccn4)N2C3=O)c2cccnc12] | 5-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)quinoline-8- carbonitrile | Calc'd 428, found 428 |
| 18.15 | 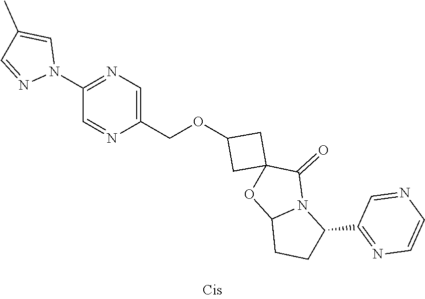 [SMILES: Cc1cnn(-c2cnc(COC3CC4(C3)OC3CC[C@@H](c5cnccn5)N3C4=O)cn2)c1] | (5′S)-3-{[5-(4-methyl-1H-pyrazol-1- yl)pyrazin-2-yl]methoxy}-5′-(pyrazin- 2-yl)tetrahydro-3′H- spiro[cyclobutane-1,2′-pyrrolo[2,1- b][1,3]oxazol]-3′-one | Calc'd 434, found 434 |
| 18.16 | 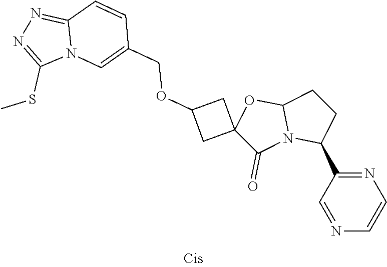 [SMILES: CSc1nnc2ccc(COC3CC4(C3)OC3CC[C@@H](c5cnccn5)N3C4=O)cn12] | (5′S)-3-{[3- (methylsulfanyl)[1,2,4]triazolo[4,3- a]pyridin-6-yl]methoxy}-5′-(pyrazin- 2-yl)tetrahydro-3′H- spiro[cyclobutane-1,2′-pyrrolo[2,1- b][1,3]oxazol]-3′-one | Calc'd 439, found 439 |
| 18.17 | 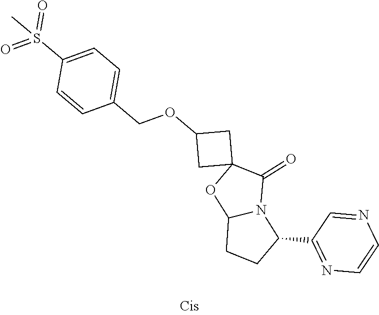 [SMILES: CS(=O)(=O)c1ccc(COC2CC3(C2)OC2CC[C@@H](c4cnccn4)N2C3=O)cc1] | (5′S)-3-{[4- (methylsulfonyl)phenyl]methoxy}-5′- (pyrazin-2-yl)tetrahydro-3′H- spiro[cyclobutane-1,2′-pyrrolo[2,1- b][1,3]oxazol]-3′-one | Calc'd 430, found 430 |
| 18.18 | 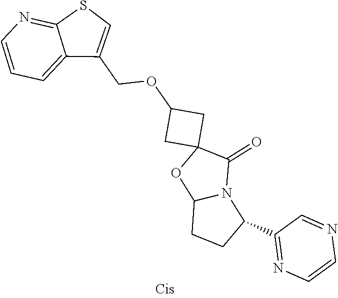 [SMILES: O=C1N2C(CC[C@H]2c2cnccn2)OC12CC(OCc1csc3ncccc13)C2] | (5′S)-5′-(pyrazin-2-yl)-3-[(thieno[2,3- b]pyridin-3-yl)methoxy]tetrahydro- 3′H-spiro[cyclobutane-1,2′- pyrrolo[2,1-b][1,3]oxazol]-3′-one | Calc'd 409, found 409 |
| 18.19 | 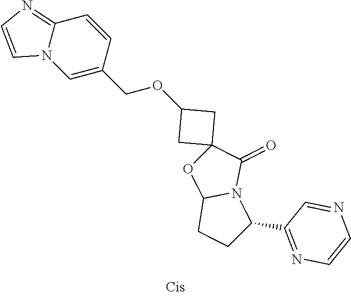 [SMILES: O=C1N2C(CC[C@H]2c2cnccn2)OC12CC(OCc1ccc3nccn3c1)C2] | (5′S)-3-[(imidazo[1,2-a]pyridin-6- yl)methoxy]-5′-(pyrazin-2- yl)tetrahydro-3′H-spiro[cyclobutane- 1,2′-pyrrolo[2,1-b][1,3]oxazol]-3′-one | Calc'd 392, found 392 |
| 18.20 | 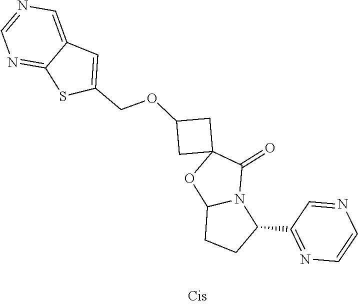 [SMILES: O=C1N2C(CC[C@H]2c2cnccn2)OC12CC(OCc1cc3cncnc3s1)C2] | (5′S)-5′-(pyrazin-2-yl)-3-[(thieno[2,3- d]pyrimidin-6-yl)methoxy]tetrahydro- 3′H-spiro[cyclobutane-1,2′- pyrrolo[2,1-b][1,3]oxazol]-3′-one | Calc'd 410, found 410 |
| 18.21 | 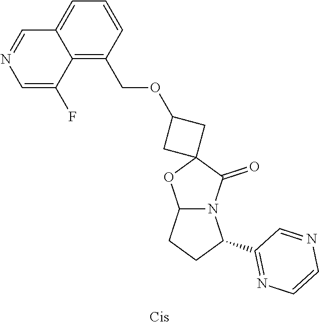 [SMILES: O=C1N2C(CC[C@H]2c2cnccn2)OC12CC(OCc1cccc3cncc(F)c13)C2] | (5′S)-3-[(4-fluoroisoquinolin-5- yl)methoxy]-5′-(pyrazin-2- yl)tetrahydro-3′H-spiro[cyclobutane- 1,2′-pyrrolo[2,1-b][1,3]oxazol]-3′-one | Calc'd 421, found 421 |
| 18.22 | 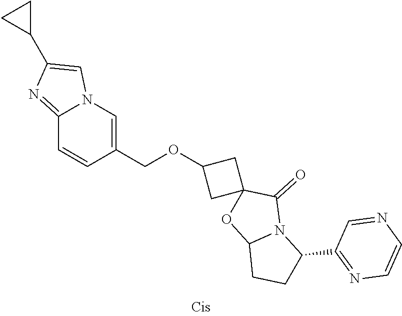 [SMILES: O=C1N2C(CC[C@H]2c2cnccn2)OC12CC(OCc1ccc3nc(C4CC4)cn3c1)C2] | (5′S)-3-[(2-cyclopropylimidazo[1,2- a]pyridin-6-yl)methoxy]-5′-(pyrazin- 2-yl)tetrahydro-3′H- spiro[cyclobutane-1,2′-pyrrolo[2,1- b][1,3]oxazol]-3′-one | Calc'd 432, found 432 |
| 18.23 | 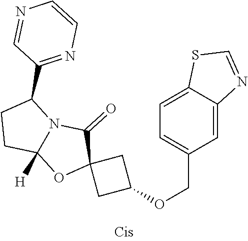 [SMILES: O=C1N2[C@@H](CC[C@H]2c2cnccn2)O[C@]12C[C@@H](OCc1ccc3scnc3c1)C2] | (1s,3S,5′S,7a′R)-3-[(1,3-benzothiazol- 5-yl)methoxy]-5′-(pyrazin-2- yl)tetrahydro-3′H-spiro[cyclobutane- 1,2′-pyrrolo [2,1-b][1,3]oxazol]-3′-one | Calc'd 409, found 409 |
| 18.24 | 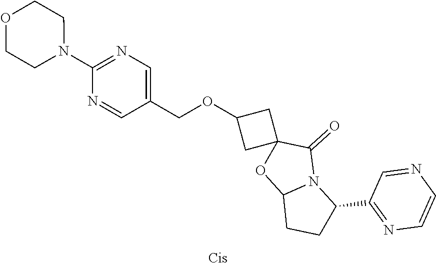 [SMILES: O=C1N2C(CC[C@H]2c2cnccn2)OC12CC(OCc1cnc(N3CCOCC3)nc1)C2] | (5′S)-3-{[2-(morpholin-4- yl)pyrimidin-5-yl]methoxy}-5′- (pyrazin-2-yl)tetrahydro-3′H- spiro[cyclobutane-1,2′-pyrrolo[2,1- b][1,3]oxazol]-3′-one | Calc'd 439, found 439 |
| 18.25 | 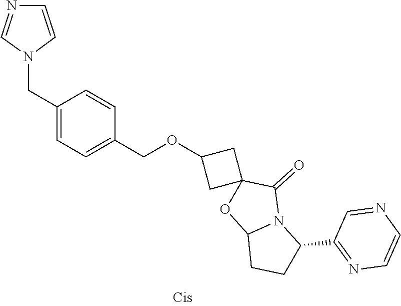 [SMILES: O=C1N2C(CC[C@H]2c2cnccn2)OC12CC(OCc1ccc(Cn3ccnc3)cc1)C2] | (5′S)-3-({4-[(1H-imidazol-1- yl)methyl]phenyl}methoxy)-5′- (pyrazin-2-yl)tetrahydro-3′H- spiro[cyclobutane-1,2′-pyrrolo[2,1- b][1,3]oxazol]-3′-one | Calc'd 432, found 432 |
| 18.26 | 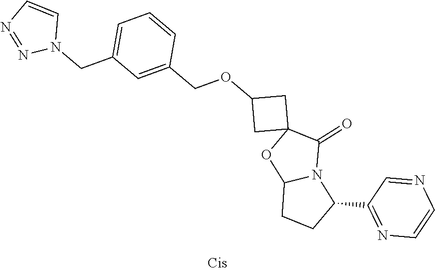 [SMILES: O=C1N2C(CC[C@H]2c2cnccn2)OC12CC(OCc1cccc(Cn3ccnn3)c1)C2] | (5′S)-5′-(pyrazin-2-yl)-3-({3-[(1H- 1,2,3-triazol-1- yl)methyl]phenyl}methoxy) tetrahydro-3′H-spiro[cyclobutane- 1,2′-pyrrolo[2,1-b][1,3]oxazol]-3′- one | Calc'd 433, found 433 |
| 18.27 | 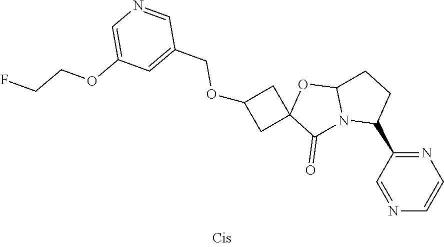 | (5′S)-3-{[5-(2-fluoroethoxy)pyridin- 3-yl]methoxy}-5′-(pyrazin-2- yl)tetrahydro-3′H-spiro[cyclobutane- 1,2′-pyrrolo[2,1-b][1,3]oxazol]-3′-one | Calc'd 415, found 415 |
| 18.28 | 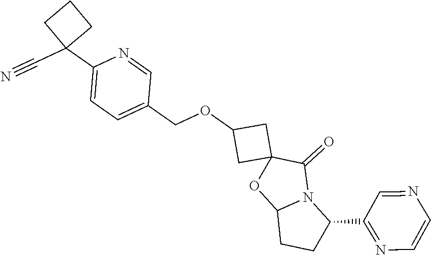 [SMILES: N#CC1(c2ccc(COC3CC4(C3)OC3CC[C@@H](c5cnccn5)N3C4=O)cn2)CCC1] | 1-[5-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)pyridin-2- yl]cyclobutane-1-carbonitrile | Calc'd 432, found 432 |
| | Cis | | |
| 18.29 | 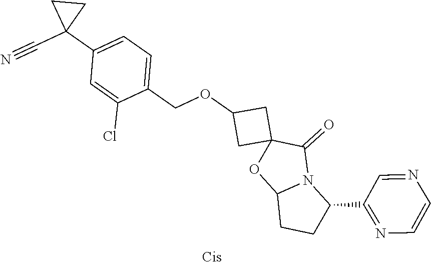 [SMILES: N#CC1(c2ccc(COC3CC4(C3)OC3CC[C@@H](c5cnccn5)N3C4=O)c(Cl)c2)CC1] | 1-[3-chloro-4-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)phenyl]cyclopropane- 1-carbonitrile | Calc'd 451, found 451 |
| 18.30 | 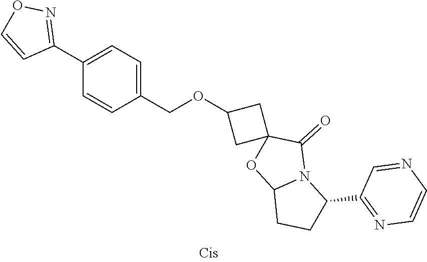 [SMILES: O=C1N2C(CC[C@H]2c2cnccn2)OC12CC(OCc1ccc(-c3ccon3)cc1)C2] | (5′S)-3-{[4-(1,2-oxazol-3- yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 419, found 419 |
| 18.31 | 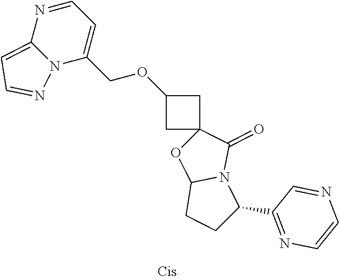 [SMILES: O=C1N2C(CC[C@H]2c2cnccn2)OC12CC(OCc1ccnc3ccnn13)C2] | (5′S)-5′-(pyrazin-2-yl)-3- [(pyrazolo[1,5-a]pyrimidin-7- yl)methoxy]tetrahydro-3′H- spiro[cyclobutane-1,2′-pyrrolo[2,1- b][1,3]oxazol]-3′-one | Calc'd 393, found 393 |
| 18.32 | 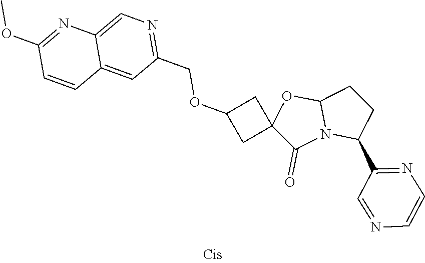 [SMILES: COc1ccc2cc(COC3CC4(C3)OC3CC[C@@H](c5cnccn5)N3C4=O)ncc2n1] | (5′S)-3-[(2-methoxy-1,7- naphthyridin-6-yl)methoxy]-5′- (pyrazin-2-yl)tetrahydro-3′H- spiro[cyclobutane-1,2′-pyrrolo[2,1- b][1,3]oxazol]-3′-one | Calc'd 434, found 434 |
| 18.33 | 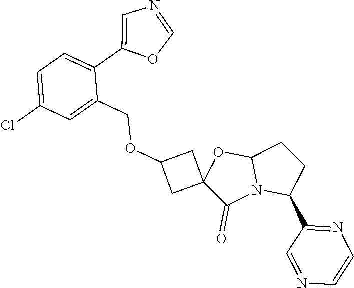 [SMILES: O=C1N2C(CC[C@H]2c2cnccn2)OC12CC(OCc1cc(Cl)ccc1-c1cnco1)C2] | (5′S)-3-{[5-chloro-2-(1,3-oxazol-5- yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 453, found 453 |
| | Cis | | |
| 18.34 | 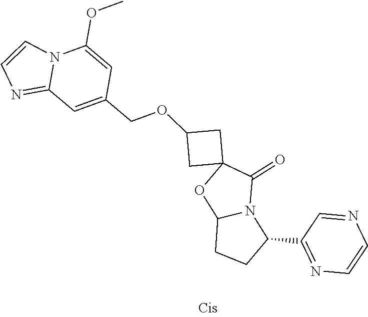 [SMILES: COc1cc(COC2CC3(C2)OC2CC[C@@H](c4cnccn4)N2C3=O)cc2nccn12] | (5′S)-3-[(5-methoxyimidazo[1,2- a]pyridin-7-yl)methoxy]-5′-(pyrazin- 2-yl)tetrahydro-3′H- spiro[cyclobutane-1,2′-pyrrolo[2,1- b][1,3]oxazol]-3′-one | Calc'd 422, found 422 |
| 18.35 | 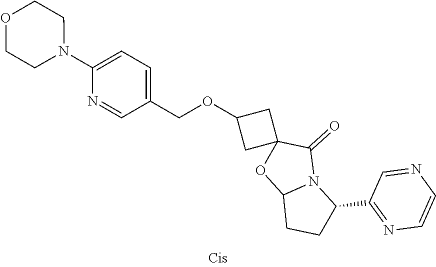 [SMILES: O=C1N2C(CC[C@H]2c2cnccn2)OC12CC(OCc1ccc(N3CCOCC3)nc1)C2] | (5′S)-3-{[6-(morpholin-4-yl)pyridin- 3-yl]methoxy}-5′-(pyrazin-2- yl)tetrahydro-3′H-spiro[cyclobutane- 1,2′-pyrrolo[2,1-b][1,3]oxazol]-3′-one | Calc'd 438, found 438 |
| 18.36 | 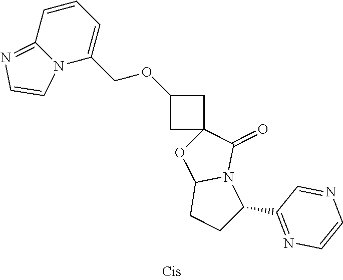 [SMILES: O=C1N2C(CC[C@H]2c2cnccn2)OC12CC(OCc1cccc3nccn13)C2] | (5′S)-3-[(imidazo[1,2-a]pyridin-5- yl)methoxy]-5′-(pyrazin-2- yl)tetrahydro-3′H-spiro[cyclobutane- 1,2′-pyrrolo[2,1-b][1,3]oxazol]-3′-one | Calc'd 392, found 392 |
| 18.37 | 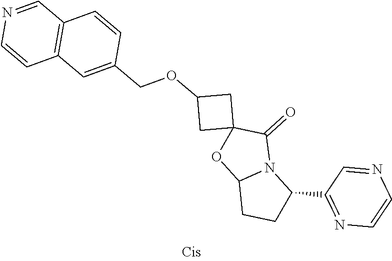 [SMILES: O=C1N2C(CC[C@H]2c2cnccn2)OC12CC(OCc1ccc3cnccc3c1)C2] | (5′S)-3-[(isoquinolin-6-yl)methoxy]- 5′-(pyrazin-2-yl)tetrahydro-3′H- spiro[cyclobutane-1,2′-pyrrolo[2,1- b][1,3]oxazol]-3′-one | Calc'd 403, found 403 |
| 18.38 | 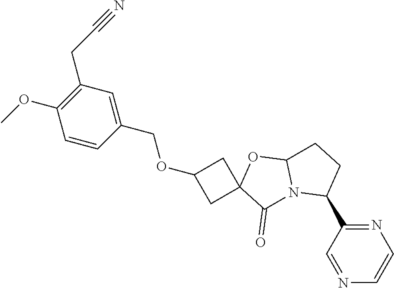 [SMILES: COc1ccc(COC2CC3(C2)OC2CC[C@@H](c4cnccn4)N2C3=O)cc1CC#N] | [2-methoxy-5-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)phenyl]acetonitrile | Calc'd 421, found 421 |
| | Cis | | |
| 18.39 | 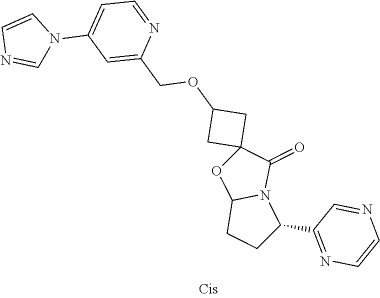 [SMILES: O=C1N2C(CC[C@H]2c2cnccn2)OC12CC(OCc1cc(-n3ccnc3)ccn1)C2] | (5′S)-3-{[4-(1H-imidazol-1- yl)pyridin-2-yl\|methoxy}-5′-(pyrazin- 2-yl)tetrahydro-3′H- spiro[cyclobutane-1,2′-pyrrolo[2,1- b][1,3]oxazol]-3′-one | Calc'd 419, found 419 |
| 18.40 | 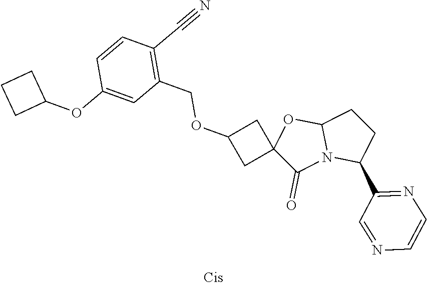 [SMILES: N#Cc1ccc(OC2CCC2)cc1COC1CC2(C1)OC1CC[C@@H](c3cnccn3)N1C2=O] | 4-(cyclobutyloxy)-2-({[(5′S)-3′-oxo- 5′-(pyrazin-2-yl)tetrahydro-3′H- spiro[cyclobutane-1,2′-pyrrolo[2,1- b][1,3]oxazol]-3- yl]oxy}methyl)benzonitrile | Calc'd 447, found 447 |
| 18.41 | 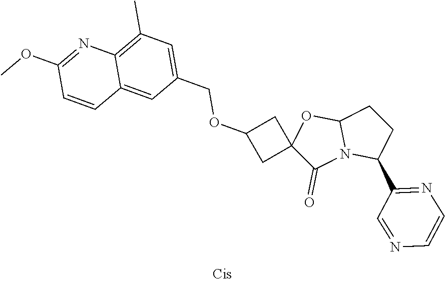 [SMILES: COc1ccc2cc(COC3CC4(C3)OC3CC[C@@H](c5cnccn5)N3C4=O)cc(C)c2n1] | (5′S)-3-[(2-methoxy-8- methylquinolin-6-yl)methoxy]-5′- (pyrazin-2-yl)tetrahydro-3′H- spiro[cyclobutane-1,2′-pyrrolo[2,1- b][1,3]oxazol]-3′-one | Calc'd 447, found 447 |
| 18.42 | 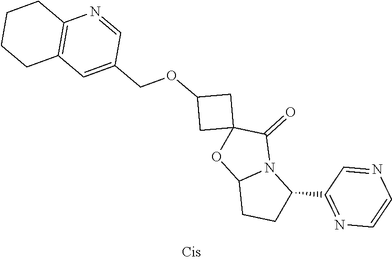 [SMILES: O=C1N2C(CC[C@H]2c2cnccn2)OC12CC(OCc1cnc3c(c1)CCCC3)C2] | (5′S)-5′-(pyrazin-2-yl)-3-[(5,6,7,8- tetrahydroquinolin-3- yl)methoxy]tetrahydro-3′H- spiro[cyclobutane-1,2′-pyrrolo[2,1- b][1,3]oxazol]-3′-one | Calc'd 407, found 407 |
| 18.43 | 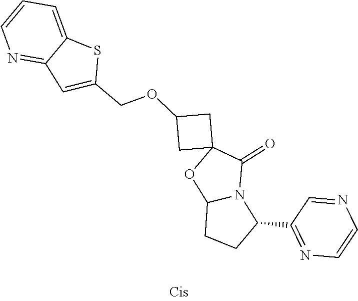 [SMILES: O=C1N2C(CC[C@H]2c2cnccn2)OC12CC(OCc1cc3ncccc3s1)C2] | (5′S)-5′-(pyrazin-2-yl)-3-[(thieno[3,2- b]pyridin-2-yl)methoxy]tetrahydro- 3′H-spiro[cyclobutane-1,2′- pyrrolo[2,1-b][1,3]oxazol]-3′-one | Calc'd 409, found 409 |
| 18.44 | 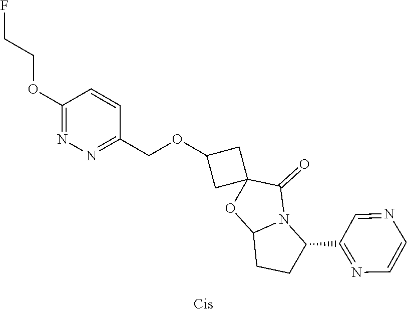 [SMILES: O=C1N2C(CC[C@H]2c2cnccn2)OC12CC(OCc1ccc(OCCF)nn1)C2] | (5′S)-3-{[6-(2- fluoroethoxy)pyridazin-3- yl]methoxy}-5′-(pyrazin-2- yl)tetrahydro-3′H-spiro[cyclobutane- 1,2′-pyrrolo[2,1-b][1,3]oxazol]-3′-one | Calc'd 416, found 416 |
| 18.45 | 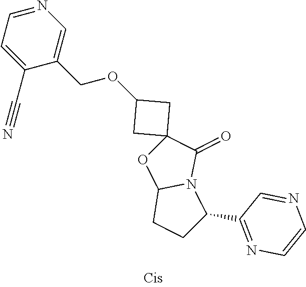 [SMILES: N#Cc1ccncc1COC1CC2(C1)OC1CC[C@@H](c3cnccn3)N1C2=O] | 3-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)pyridine-4-carbonitrile | Calc'd 378, found 378 |
| 18.46 | 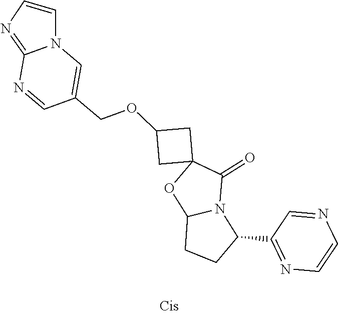 [SMILES: O=C1N2C(CC[C@H]2c2cnccn2)OC12CC(OCc1cnc3nccn3c1)C2] | (5′S)-3-[(imidazo[1,2-a]pyrimidin-6- yl)methoxy]-5′-(pyrazin-2- yl)tetrahydro-3′H-spiro[cyclobutane- 1,2′-pyrrolo[2,1-b][1,3]oxazol]-3′-one | Calc'd 393, found 393 |
| 18.47 | 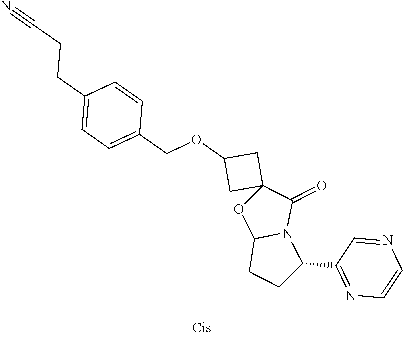 [SMILES: N#CCCc1ccc(COC2CC3(C2)OC2CC[C@@H](c4cnccn4)N2C3=O)cc1] | 3-[4-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)phenyl]propanenitrile | Calc'd 405, found 405 |
| 18.48 | 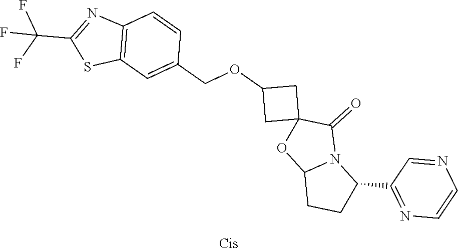 [SMILES: O=C1N2C(CC[C@H]2c2cnccn2)OC12CC(OCc1ccc3nc(C(F)(F)F)sc3c1)C2] | (5′S)-5′-(pyrazin-2-yl)-3-{[2- (trifluoromethyl)-1,3-benzothiazol-6- yl\|methoxy}tetrahydro-3′H- spiro[cyclobutane-1,2′-pyrrolo[2,1- b][1,3]oxazol]-3′-one | Calc'd 477, found 477 |
| 18.49 | 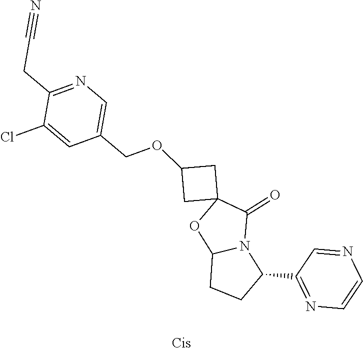 [SMILES: N#CCc1ncc(COC2CC3(C2)OC2CC[C@@H](c4cnccn4)N2C3=O)cc1Cl] | [3-chloro-5-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)pyridin-2- yl]acetonitrile | Calc'd 426, found 426 |
| 18.50 | 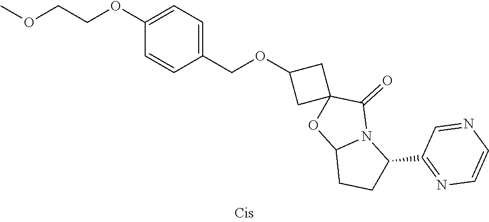 [SMILES: COCCOc1ccc(COC2CC3(C2)OC2CC[C@@H](c4cnccn4)N2C3=O)cc1] | (5′S)-3-{[4-(2- methoxyethoxy)phenyl]methoxy}-5′- (pyrazin-2-yl)tetrahydro-3′H- spiro[cyclobutane-1,2′-pyrrolo[2,1- b][1,3]oxazol]-3′-one | Calc'd 426, found 426 |
| 18.51 | 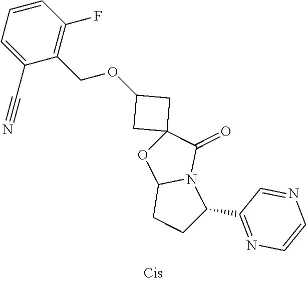 [SMILES: N#Cc1cccc(F)c1COC1CC2(C1)OC1CC[C@@H](c3cnccn3)N1C2=O] | 3-fluoro-2-({[(5′S)-3′-oxo-5′-(pyrazin- 2-yl)tetrahydro-3′H- spiro[cyclobutane-1,2′-pyrrolo[2,1- b][1,3]oxazol]-3- yl]oxy}methyl)benzonitrile | Calc'd 395, found 395 |
| 18.52 | 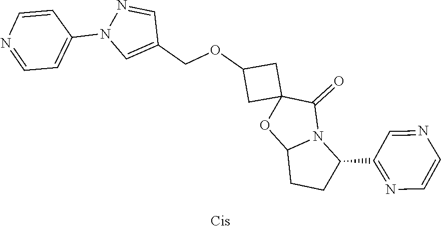 [SMILES: O=C1N2C(CC[C@H]2c2cnccn2)OC12CC(OCc1cnn(-c3ccncc3)c1)C2] | (5′S)-5′-(pyrazin-2-yl)-3-{[1-(pyridin- 4-yl)-1H-pyrazol-4- yl]methoxy}tetrahydro-3′H- spiro[cyclobutane-1,2′-pyrrolo[2,1- b][1,3]oxazol]-3′-one | Calc'd 419, found 419 |
| 18.53 | 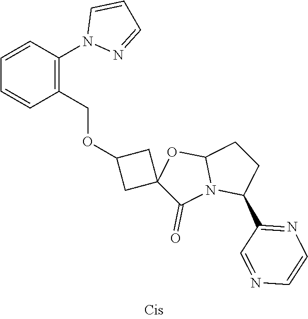 [SMILES: O=C1N2C(CC[C@H]2c2cnccn2)OC12CC(OCc1ccccc1-n1cccn1)C2] | (5′S)-5′-(pyrazin-2-yl)-3-{[2-(1H- pyrazol-1- yl)phenyl]methoxy}tetrahydro-3′H- spiro[cyclobutane-1,2′-pyrrolo[2,1- b][1,3]oxazol]-3′-one | Calc'd 418, found 418 |
| 18.54 | 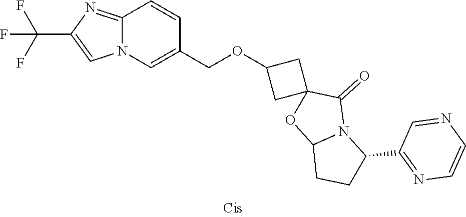 [SMILES: O=C1N2C(CC[C@H]2c2cnccn2)OC12CC(OCc1ccc3nc(C(F)(F)F)cn3c1)C2] | (5′S)-5′-(pyrazin-2-yl)-3-{[2- (trifluoromethyl)imidazo[1,2- a]pyridin-6-yl]methoxy}tetrahydro- 3′H-spiro[cyclobutane-1,2′- pyrrolo[2,1-b][1,3]oxazol]-3′-one | Calc'd 460, found 460 |
| 18.55 | 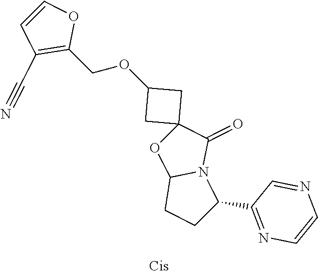 [SMILES: N#Cc1ccoc1COC1CC2(C1)OC1CC[C@@H](c3cnccn3)N1C2=O] | 2-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)furan-3-carbonitrile | Calc'd 367, found 367 |
| 18.56 | 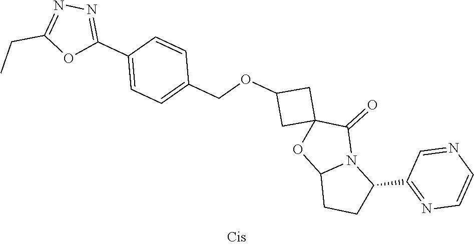 [SMILES: CCc1nnc(-c2ccc(COC3CC4(C3)OC3CC[C@@H](c5cnccn5)N3C4=O)cc2)o1] | (5′S)-3-{[4-(5-ethyl-1,3,4-oxadiazol- 2-yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 448, found 448 |
| 18.57 | 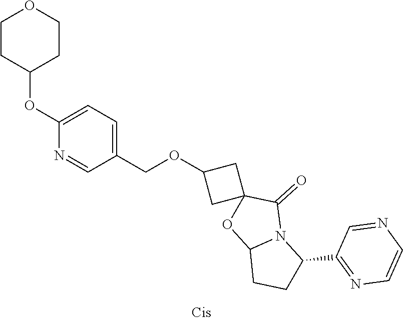 [SMILES: O=C1N2C(CC[C@H]2c2cnccn2)OC12CC(OCc1ccc(OC3CCOCC3)nc1)C2] | (5′S)-3-({6-[(oxan-4-yl)oxy]pyridin- 3-yl}methoxy)-5′-(pyrazin-2- yl)tetrahydro-3′H-spiro[cyclobutane- 1,2′-pyrrolo[2,1-b][1,3]oxazol]-3′-one | Calc'd 453, found 453 |
| 18.58 | 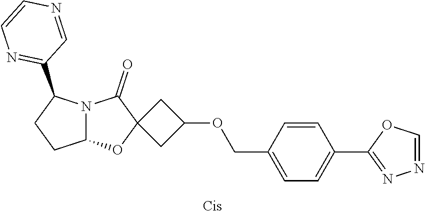 | (5′S, 7a′R)-3-{[4-(1,3,4-oxadiazol-2- yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 420, found 420 |
| 18.59 | 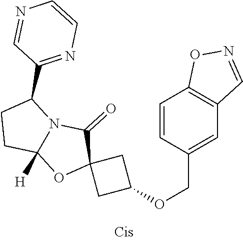 [SMILES: O=C1N2[C@@H](CC[C@H]2c2cnccn2)O[C@]12C[C@@H](OCc1ccc3oncc3c1)C2] | (5′S)-3-[(1,2-benzoxazol-5- yl)methoxy]-5′-(pyrazin-2- yl)tetrahydro-3′H-spiro[cyclobutane- 1,2′-pyrrolo[2,1-b][1,3]oxazol]-3′-one | Calc'd 393, found 393 |
| 18.60 | 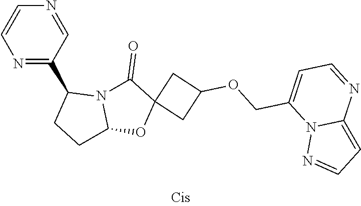 | (5′S)-5′-(pyrazin-2-yl)-3- [(pyrazolo[1,5-a]pyrimidin-7- yl)methoxy]tetrahydro-3′H- spiro[cyclobutane-1,2′-pyrrolo[2,1- b][1,3]oxazol]-3′-one | Calc'd 393, found 393 |
| 18.61 | 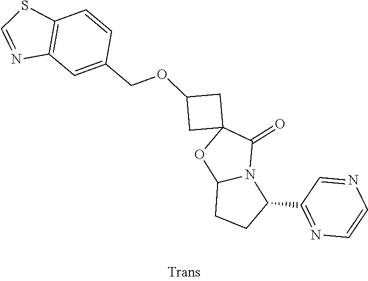 [SMILES: O=C1N2C(CC[C@H]2c2cnccn2)OC12CC(OCc1ccc3scnc3c1)C2] | (5′S)-3-[(1,3-benzothiazol-5- yl)methoxy]-5′-(pyrazin-2- yl)tetrahydro-3′H-spiro[cyclobutane- 1,2′-pyrrolo[2,1-b][1,3]oxazol]-3′-one | Calc'd 409, found 409 |
| 18.62 | 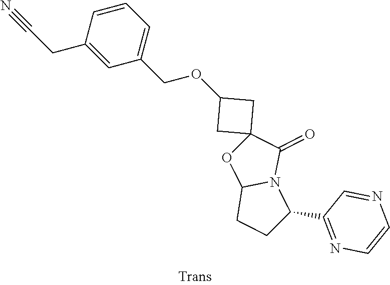 [SMILES: N#CCc1cccc(COC2CC3(C2)OC2CC[C@@H](c4cnccn4)N2C3=O)c1] | [3-({[(5′S)-3′-oxo-5′-(pyrazin-2 yl)tetrahydro-3′H-spiro[cyclobutane- 1,2′-pyrrolo[2,1-b][1,3]oxazol]-3- yl]oxy}methyl)phenyl]acetonitrile | Calc'd 391, found 391 |
| 18.63 | 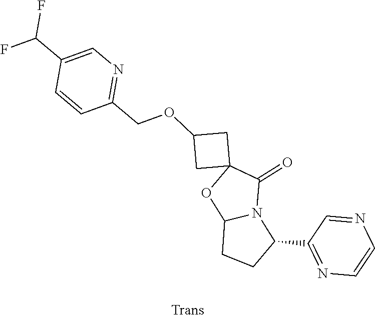 [SMILES: O=C1N2C(CC[C@H]2c2cnccn2)OC12CC(OCc1ccc(C(F)F)cn1)C2] | (5′S)-3-{[5-(difluoromethyl)pyridin- 2-yl]methoxy}-5′-(pyrazin-2- yl)tetrahydro-3′H-spiro[cyclobutane- 1,2′-pyrrolo[2,1-b][1,3]oxazol]-3′-one | Calc'd 403, found 403 |
| 18.64 | 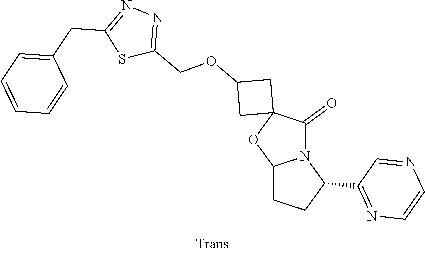 [SMILES: O=C1N2C(CC[C@H]2c2cnccn2)OC12CC(OCc1nnc(Cc3ccccc3)s1)C2] | (5′S)-3-[(5-benzyl-1,3,4-thiadiazol-2- yl)methoxy]-5′-(pyrazin-2- yl)tetrahydro-3′H-spiro[cyclobutane- 1,2′-pyrrolo[2,1-b][1,3]oxazol]-3′-one | Calc'd 450, found 450 |
| 18.65 | 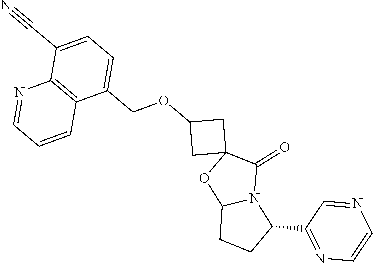 [SMILES: N#Cc1ccc(COC2CC3(C2)OC2CC[C@@H](c4cnccn4)N2C3=O)c2cccnc12] | 5-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)quinoline-8- carbonitrile | Calc'd 428, found 428 |
| | Trans | | |
| 18.66 | 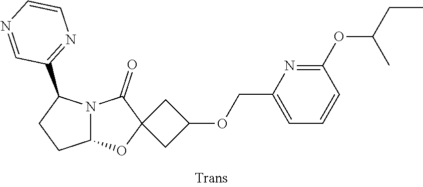 | (5′S,7a′R)-3-({6-[(butan-2- yl)oxy]pyridin-2-yl}methoxy)-5′- (pyrazin-2-yl)tetrahydro-3′H- spiro[cyclobutane-1,2′-pyrrolo[2,1- b][1,3]oxazol]-3′-one | Calc'd 425, found 425 |
| 18.67 | 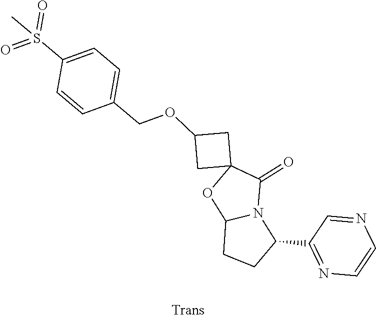 [SMILES: CS(=O)(=O)c1ccc(COC2CC3(C2)OC2CC[C@@H](c4cnccn4)N2C3=O)cc1] | (5′S)-3-{[4- (methylsulfonyl)phenyl]methoxy}-5′- (pyrazin-2-yl)tetrahydro-3′H- spiro[cyclobutane-1,2′-pyrrolo[2,1- b][1,3]oxazol]-3′-one | Calc'd 430, found 430 |
| 18.68 | 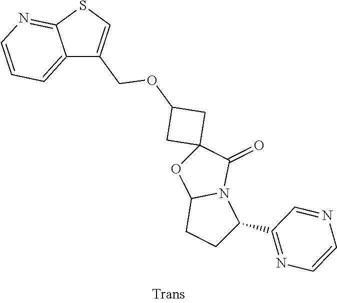 [SMILES: O=C1N2C(CC[C@H]2c2cnccn2)OC12CC(OCc1csc3ncccc13)C2] | (5′S)-5′-(pyrazin-2-yl)-3-[(thieno[2,3- b]pyridin-3-yl)methoxy]tetrahydro- 3′H-spiro[cyclobutane-1,2′- pyrrolo[2,1-b][1,3]oxazol]-3′-one | Calc'd 409, found 409 |
| 18.69 | 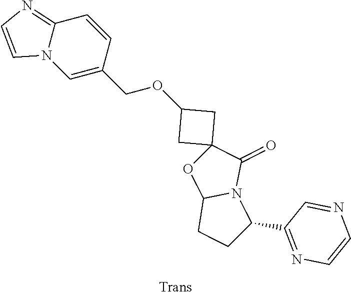 [SMILES: O=C1N2C(CC[C@H]2c2cnccn2)OC12CC(OCc1ccc3nccn3c1)C2] | (5′S)-3-[(imidazo[1,2-a]pyridin-6- yl)methoxy]-5′-(pyrazin-2- yl)tetrahydro-3′H-spiro[cyclobutane- 1,2′-pyrrolo[2,1-b][1,3]oxazol]-3′-one | Calc'd 392, found 392 |
| 18.70 | 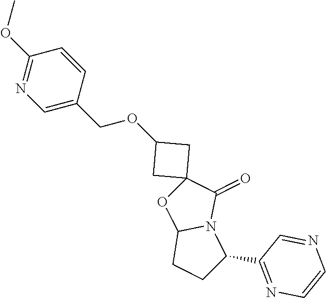 [SMILES: COc1ccc(COC2CC3(C2)OC2CC[C@@H](c4cnccn4)N2C3=O)cn1] | (5′S)-3-[(6-methoxypyridin-3- yl)methoxy]-5′-(pyrazin-2- yl)tetrahydro-3′H-spiro[cyclobutane- 1,2′-pyrrolo[2,1-b][1,3]oxazol]-3′-one | Calc'd 383, found 383 |
| | Trans | | |
| 18.71 | 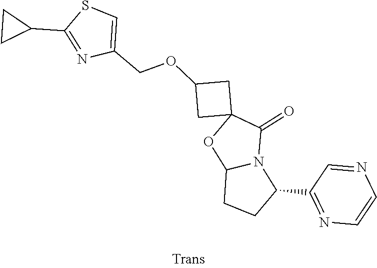 [SMILES: O=C1N2C(CC[C@H]2c2cnccn2)OC12CC(OCc1csc(C3CC3)n1)C2] | (5′S)-3-[(2-cyclopropyl-1,3-thiazol-4- yl)methoxy]-5′-(pyrazin-2- yl)tetrahydro-3′H-spiro[cyclobutane- 1,2′-pyrrolo[2,1-b][1,3]oxazol]-3′-one | Calc'd 399, found 399 |
| 18.72 | 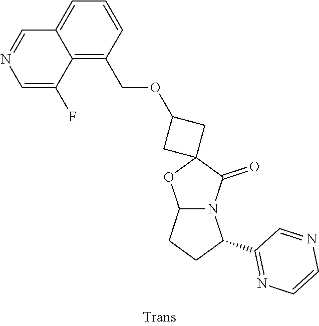 [SMILES: O=C1N2C(CC[C@H]2c2cnccn2)OC12CC(OCc1cccc3cncc(F)c13)C2] | (5′S)-3-[(4-fluoroisoquinolin-5- yl)methoxy]-5′-(pyrazin-2- yl)tetrahydro-3′H-spiro[cyclobutane- 1,2′-pyrrolo[2,1-b][1,3]oxazol]-3′-one | Calc'd 421, found 421 |
| 18.73 | 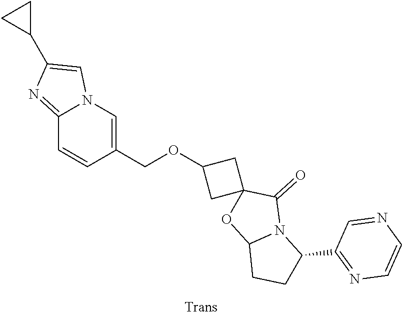 [SMILES: O=C1N2C(CC[C@H]2c2cnccn2)OC12CC(OCc1ccc3nc(C4CC4)cn3c1)C2] | (5′S)-3-[(2-cyclopropylimidazo[1,2- a]pyridin-6-yl)methoxy]-5′-(pyrazin- 2-yl)tetrahydro-3′H- spiro[cyclobutane-1,2′-pyrrolo[2,1- b][1,3]oxazol]-3′-one | Calc'd 432, found 432 |
| 18.74 | 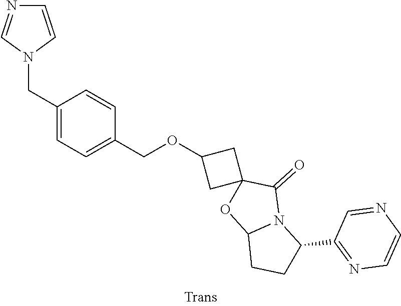 [SMILES: O=C1N2C(CC[C@H]2c2cnccn2)OC12CC(OCc1ccc(Cn3ccnc3)cc1)C2] | (5′S)-3-({4-[(1H-imidazol-1- yl)methyl]phenyl}methoxy)-5′- (pyrazin-2-yl)tetrahydro-3′H- spiro[cyclobutane-1,2′-pyrrolo[2,1- b][1,3]oxazol]-3′-one | Calc'd 432, found 432 |
| 18.75 | 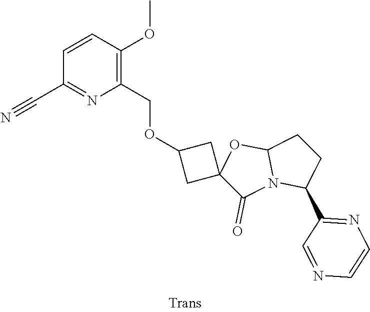 [SMILES: COc1ccc(C#N)nc1COC1CC2(C1)OC1CC[C@@H](c3cnccn3)N1C2=O] | 5-methoxy-6-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)pyridine-2-carbonitrile | Calc'd 408, found 408 |
| 18.76 | 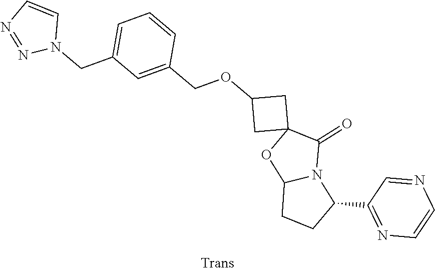 [SMILES: O=C1N2C(CC[C@H]2c2cnccn2)OC12CC(OCc1cccc(Cn3ccnn3)c1)C2] | (5′S)-5′-(pyrazin-2-yl)-3-({3-[(1H- 1,2,3-triazol-1- yl)methyl]phenyl}methoxy)tetrahydro- 3′H-spiro[cyclobutane-1,2′- pyrrolo[2,1-b][1,3]oxazol]-3′-one | Calc'd 433, found 433 |
| 18.77 | 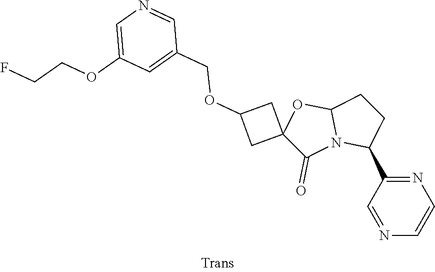 [SMILES: O=C1N2C(CC[C@H]2c2cnccn2)OC12CC(OCc1cncc(OCCF)c1)C2] | (5′S)-3-{[5-(2-fluoroethoxy)pyridin- 3-yl]methoxy}-5′-(pyrazin-2- yl)tetrahydro-3′H-spiro[cyclobutane- 1,2′-pyrrolo[2,1-b][1,3]oxazol]-3′-one | Calc'd 415, found 415 |
| 18.78 | 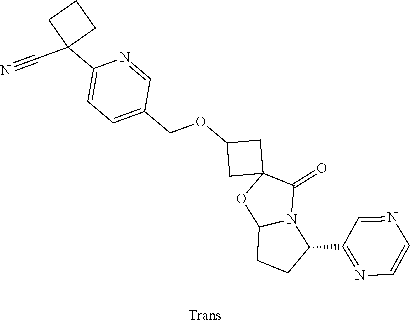 [SMILES: N#CC1(c2ccc(COC3CC4(C3)OC3CC[C@@H](c5cnccn5)N3C4=O)cn2)CCC1] | 1-[5-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)pyridin-2- yl]cyclobutane-1-carbonitrile | Calc'd 432, found 432 |
| 18.79 | 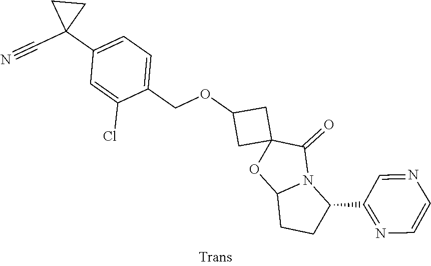 [SMILES: N#CC1(c2ccc(COC3CC4(C3)OC3CC[C@@H](c5cnccn5)N3C4=O)c(Cl)c2)CC1] | 1-[3-chloro-4-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)phenyl]cyclopropane- 1-carbonitrile | Calc'd 451, found 451 |
| 18.80 | 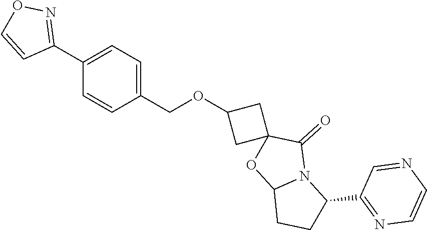 [SMILES: O=C1N2C(CC[C@H]2c2cnccn2)OC12CC(OCc1ccc(-c3ccon3)cc1)C2] | (5′S)-3-{[4-(1,2-oxazol-3- yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 419, found 419 |
| | Trans | | |
| 18.81 | 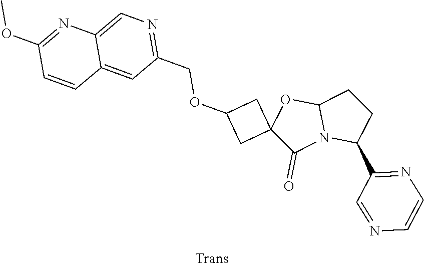 [SMILES: COc1ccc2cc(COC3CC4(C3)OC3CC[C@@H](c5cnccn5)N3C4=O)ncc2n1] | (5′S)-3-[(2-methoxy-1,7- naphthyridin-6-yl)methoxy]-5′- (pyrazin-2-yl)tetrahydro-3′H- spiro[cyclobutane-1,2′-pyrrolo[2,1- b][1,3]oxazol]-3′-one | Calc'd 434, found 434 |
| 18.82 | 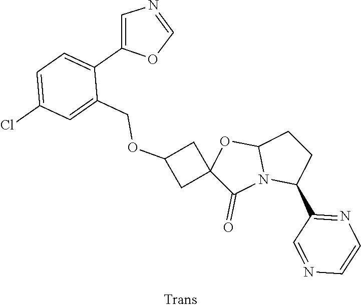 [SMILES: O=C1N2C(CC[C@H]2c2cnccn2)OC12CC(OCc1cc(Cl)ccc1-c1cnco1)C2] | (5′S)-3-{[5-chloro-2-(1,3-oxazol-5- yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 453, found 453 |
| 18.83 | 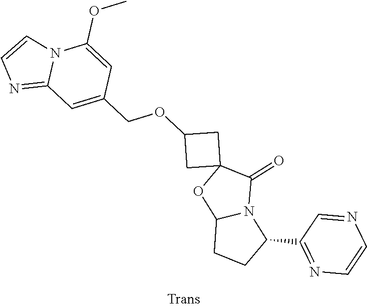 [SMILES: COc1cc(COC2CC3(C2)OC2CC[C@@H](c4cnccn4)N2C3=O)cc2nccn12] | (5′S)-3-[(5-methoxyimidazo[1,2- a]pyridin-7-yl)methoxy]-5′-(pyrazin- 2-yl)tetrahydro-3′H- spiro[cyclobutane-1,2′-pyrrolo[2,1- b][1,3]oxazol]-3′-one | Calc'd 422, found 422 |
| 18.84 | 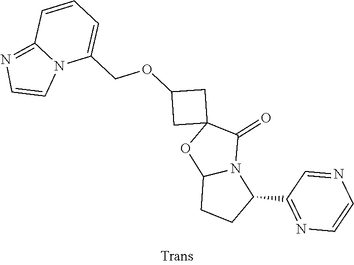 [SMILES: O=C1N2C(CC[C@H]2c2cnccn2)OC12CC(OCc1cccc3nccn13)C2] | (5′S)-3-[(imidazo[1,2-a]pyridin-5- yl)methoxy]-5′-(pyrazin-2- yl)tetrahydro-3′H-spiro[cyclobutane- 1,2′-pyrrolo[2,1-b][1,3]oxazol]-3′-one | Calc'd 392, found 392 |
| 18.85 | 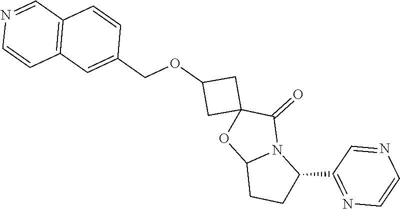 [SMILES: O=C1N2C(CC[C@H]2c2cnccn2)OC12CC(OCc1ccc3cnccc3c1)C2] | (5′S)-3-[(isoquinolin-6-yl)methoxy]- 5′-(pyrazin-2-yl)tetrahydro-3′H- spiro[cyclobutane-1,2′-pyrrolo[2,1- b][1,3]oxazol]-3′-one | Calc'd 403, found 403 |
| | Trans | | |
| 18.86 | 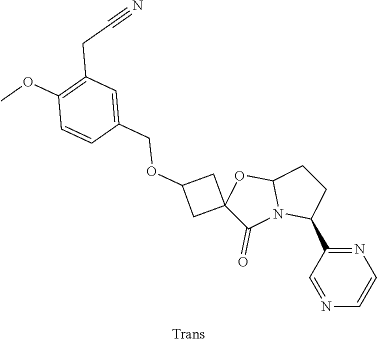 [SMILES: COc1ccc(COC2CC3(C2)OC2CC[C@@H](c4cnccn4)N2C3=O)cc1CC#N] | [2-methoxy-5-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)phenyl]acetonitrile | Calc'd 421, found 421 |
| 18.87 | 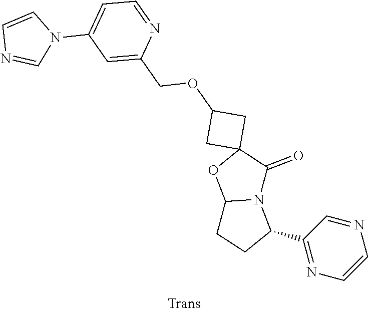 [SMILES: O=C1N2C(CC[C@H]2c2cnccn2)OC12CC(OCc1cc(-n3ccnc3)ccn1)C2] | (5′S)-3-{[4-(1H-imidazol-1- yl)pyridin-2-yl\|methoxy}-5′-(pyrazin- 2-yl)tetrahydro-3′H- spiro[cyclobutane-1,2′-pyrrolo[2,1- b][1,3]oxazol]-3′-one | Calc'd 419, found 419 |
| 18.88 | 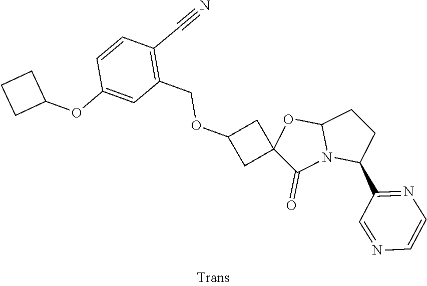 [SMILES: N#Cc1ccc(OC2CCC2)cc1COC1CC2(C1)OC1CC[C@@H](c3cnccn3)N1C2=O] | 4-(cyclobutyloxy)-2-({[(5′S)-3′-oxo- 5′-(pyrazin-2-yl)tetrahydro-3′H- spiro[cyclobutane-1,2′-pyrrolo[2,1- b][1,3]oxazol]-3- yl]oxy}methyl)benzonitrile | Calc'd 447, found 447 |
| 18.89 | 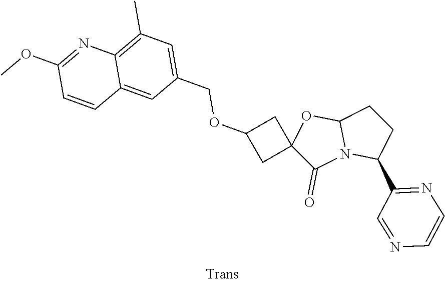 [SMILES: COc1ccc2cc(COC3CC4(C3)OC3CC[C@@H](c5cnccn5)N3C4=O)cc(C)c2n1] | (5′S)-3-[(2-methoxy-8- methylquinolin-6-yl)methoxy]-5′- (pyrazin-2-yl)tetrahydro-3′H- spiro[cyclobutane-1,2′-pyrrolo[2,1- b][1,3]oxazol]-3′-one | Calc'd 447, found 447 |
| 18.90 | 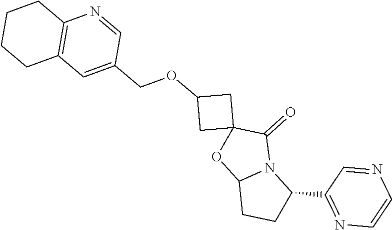 [SMILES: O=C1N2C(CC[C@H]2c2cnccn2)OC12CC(OCc1cnc3c(c1)CCCC3)C2] | (5′S)-5′-(pyrazin-2-yl)-3-[(5,6,7,8- tetrahydroquinolin-3- yl)methoxy]tetrahydro-3′H- spiro[cyclobutane-1,2′-pyrrolo[2,1- b][1,3]oxazol]-3′-one | Calc'd 407, found 407 |
| | Trans | | |
| 18.91 | 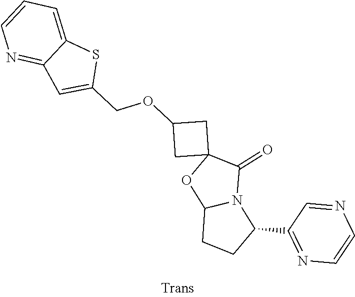 [SMILES: O=C1N2C(CC[C@H]2c2cnccn2)OC12CC(OCc1cc3ncccc3s1)C2] | (5′S)-5′-(pyrazin-2-yl)-3-[(thieno[3,2- b]pyridin-2-yl)methoxy]tetrahydro- 3′H-spiro[cyclobutane-1,2′- pyrrolo[2,1-b][1,3]oxazol]-3′-one | Calc'd 409, found 409 |
| 18.92 | 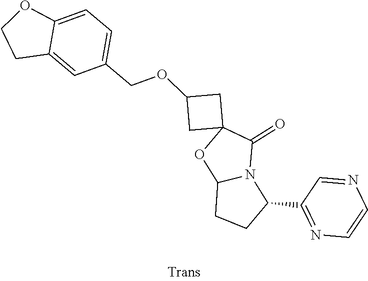 [SMILES: O=C1N2C(CC[C@H]2c2cnccn2)OC12CC(OCc1ccc3c(c1)CCO3)C2] | (5′S)-3-[(2,3-dihydro-1-benzofuran-5- yl)methoxy]-5′-(pyrazin-2- yl)tetrahydro-3′H-spiro[cyclobutane- 1,2′-pyrrolo[2,1-b][1,3]oxazol]-3′-one | Calc'd 394, found 394 |
| 18.93 | 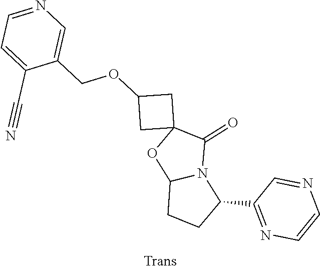 [SMILES: N#Cc1ccncc1COC1CC2(C1)OC1CC[C@@H](c3cnccn3)N1C2=O] | 3-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)pyridine-4-carbonitrile | Calc'd 378, found 378 |
| 18.94 | 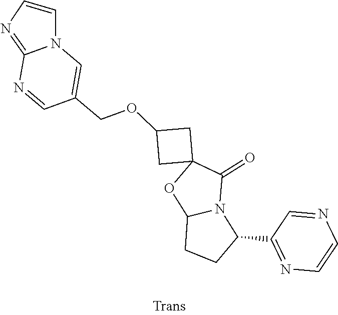 [SMILES: O=C1N2C(CC[C@H]2c2cnccn2)OC12CC(OCc1cnc3nccn3c1)C2] | (5′S)-3-[(imidazo[1,2-a]pyrimidin-6- yl)methoxy]-5′-(pyrazin-2- yl)tetrahydro-3′H-spiro[cyclobutane- 1,2′-pyrrolo[2,1-b][1,3]oxazol]-3′-one | Calc'd 393, found 393 |
| 18.95 | 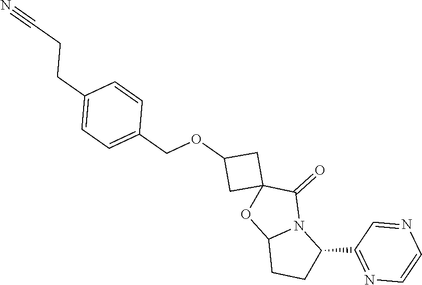 [SMILES: N#CCCc1ccc(COC2CC3(C2)OC2CC[C@@H](c4cnccn4)N2C3=O)cc1] | 3-[4-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)phenyl]propanenitrile | Calc'd 405, found 405 |
| | Trans | | |
| 18.96 | 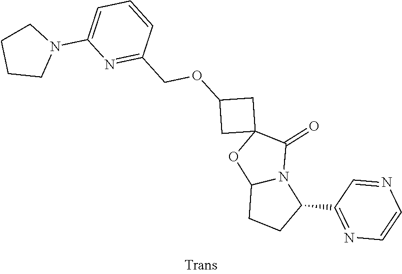 [SMILES: O=C1N2C(CC[C@H]2c2cnccn2)OC12CC(OCc1cccc(N3CCCC3)n1)C2] | (5′S)-5′-(pyrazin-2-yl)-3-{[6- (pyrrolidin-1-yl)pyridin-2- yl]methoxy}tetrahydro-3′H- spiro[cyclobutane-1,2′-pyrrolo[2,1- b][1,3]oxazol]-3′-one | Calc'd 422, found 422 |
| 18.97 | 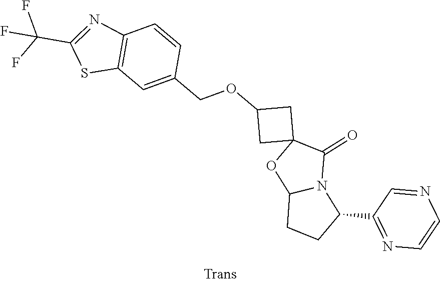 [SMILES: O=C1N2C(CC[C@H]2c2cnccn2)OC12CC(OCc1ccc3nc(C(F)(F)F)sc3c1)C2] | (5′S)-5′-(pyrazin-2-yl)-3-{[2- (trifluoromethyl)-1,3-benzothiazol-6- yl]methoxy}tetrahydro-3′H- spiro[cyclobutane-1,2′-pyrrolo[2,1- b][1,3]oxazol]-3′-one | Calc'd 477, found 477 |
| 18.98 | 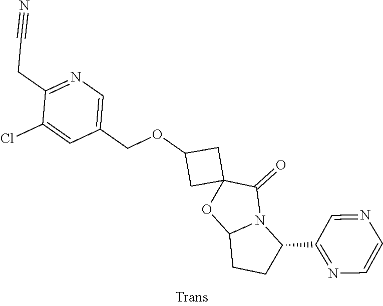 [SMILES: N#CCc1ncc(COC2CC3(C2)OC2CC[C@@H](c4cnccn4)N2C3=O)cc1Cl] | [3-chloro-5-({[(5′S)-3′-oxo-5′- (pyrazin-2-yl)tetrahydro-3′H- spiro[cyclobutane-1,2′-pyrrolo[2,1- b][1,3]oxazol]-3- yl]oxy}methyl)pyridin-2- yl]acetonitrile | Calc'd 426, found 426 |
| 18.99 | 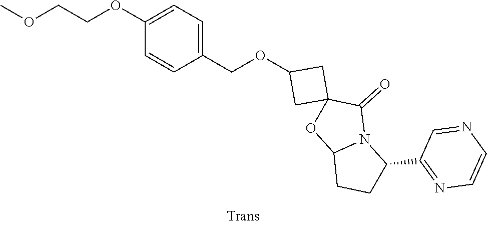 [SMILES: COCCOc1ccc(COC2CC3(C2)OC2CC[C@@H](c4cnccn4)N2C3=O)cc1] | (5′S)-3-{[4-(2- methoxyethoxy)phenyl]methoxy}-5′- (pyrazin-2-yl)tetrahydro-3′H- spiro[cyclobutane-1,2′-pyrrolo[2,1- b][1,3]oxazol]-3′-one | Calc'd 426, found 426 |
| 18.100 | 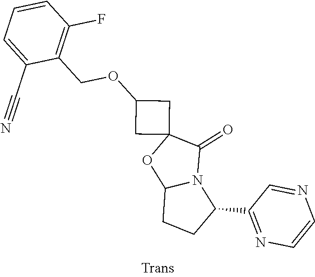 [SMILES: N#Cc1cccc(F)c1COC1CC2(C1)OC1CC[C@@H](c3cnccn3)N1C2=O] | 3-fluoro-2-({[(5′S)-3′-oxo-5′-(pyrazin- 2-yl)tetrahydro-3′H- spiro[cyclobutane-1,2′-pyrrolo[2,1- b][1,3]oxazol]-3- yl]oxy}methyl)benzonitrile | Calc'd 395, found 395 |
| 18.101 | 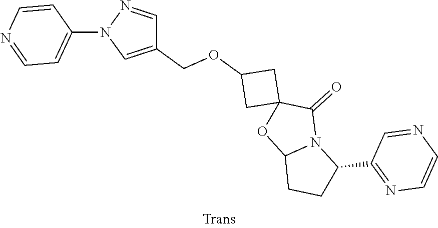 [SMILES: O=C1N2C(CC[C@H]2c2cnccn2)OC12CC(OCc1cnn(-c3ccncc3)c1)C2] | (5′S)-5′-(pyrazin-2-yl)-3-{[1-(pyridin- 4-yl)-1H-pyrazol-4- yl\|methoxy}tetrahydro-3′H- spiro[cyclobutane-1,2′-pyrrolo[2,1- b][1,3]oxazol]-3′-one | Calc'd 419, found 419 |
| 18.102 | 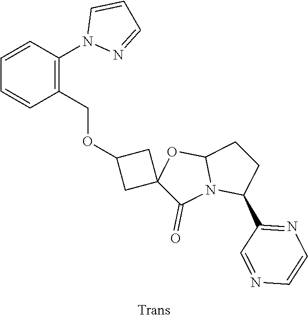 [SMILES: O=C1N2C(CC[C@H]2c2cnccn2)OC12CC(OCc1ccccc1-n1cccn1)C2] | (5′S)-5′-(pyrazin-2-yl)-3-{[2-(1H- pyrazol-1- yl)phenyl]methoxy}tetrahydro-3′H- spiro[cyclobutane-1,2′-pyrrolo[2,1- b][1,3]oxazol]-3′-one | Calc'd 418, found 418 |
| 18.103 | 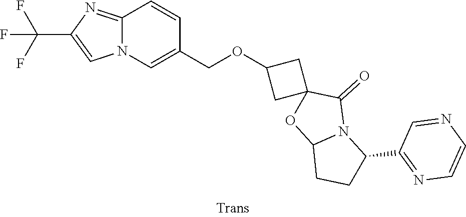 [SMILES: O=C1N2C(CC[C@H]2c2cnccn2)OC12CC(OCc1ccc3nc(C(F)(F)F)cn3c1)C2] | (5′S)-5′-(pyrazin-2-yl)-3-{[2- (trifluoromethyl)imidazo[1,2- a]pyridin-6-yl\|methoxy}tetrahydro- 3′H-spiro[cyclobutane-1,2′- pyrrolo[2,1-b][1,3 ]oxazol]-3′-one | Calc'd 460, found 460 |
| 18.104 | 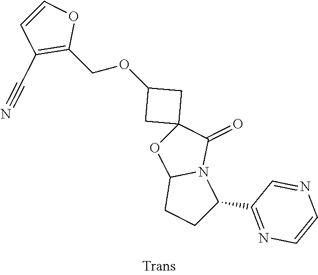 [SMILES: N#Cc1ccoc1COC1CC2(C1)OC1CC[C@@H](c3cnccn3)N1C2=O] | 2-({[(5′S)-3′-oxo-5′-(pyrazin-2- yl)tetrahydro-3′H-spiro[cyclobutane- 1,2′-pyrrolo[2,1-b][1,3]oxazol]-3- yl]oxy}methyl)furan-3-carbonitrile | Calc'd 367, found 367 |
| 18.105 | 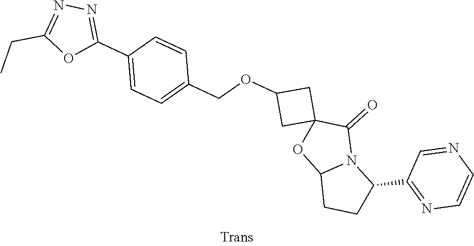 [SMILES: CCc1nnc(-c2ccc(COC3CC4(C3)OC3CC[C@@H](c5cnccn5)N3C4=O)cc2)o1] | (5′S)-3-{[4-(5-ethyl-1,3,4-oxadiazol- 2-yl)phenyl]methoxy}-5′-(pyrazin-2- yl)tetrahydro-3′H-spiro[cyclobutane- 1,2′-pyrrolo[2,1-b][1,3]oxazol]-3′-one | Calc'd 448, found 448 |
| 18.106 | 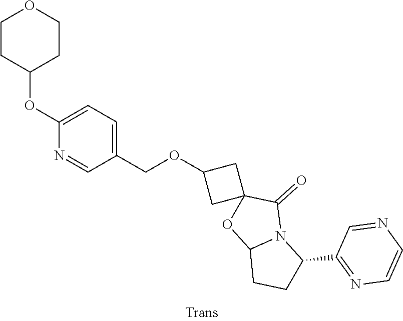 [SMILES: O=C1N2C(CC[C@H]2c2cnccn2)OC12CC(OCc1ccc(OC3CCOCC3)nc1)C2] | (5′S)-3-({6-[(oxan-4-yl)oxy]pyridin- 3-yl}methoxy)-5′-(pyrazin-2- yl)tetrahydro-3′H-spiro[cyclobutane- 1,2′-pyrrolo[2,1-b][1,3]oxazol]-3′-one | Calc'd 453, found 453 |
| 18.107 | 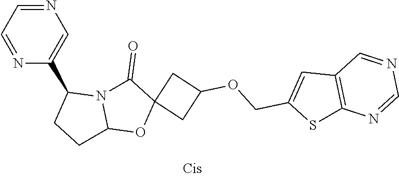 [SMILES: O=C1N2C(CC[C@H]2c2cnccn2)OC12CC(OCc1cc3cncnc3s1)C2] | (5′S)-5′-(pyrazin-2-yl)-3-(thieno[2,3- d]pyrimidin-6-ylmethoxy)tetrahydro- 3′H-spiro[cyclobutane-1,2′- pyrrolo[2,1-b]oxazol]-3′-one | Calc'd 410, found 410 |
| 18.108 | 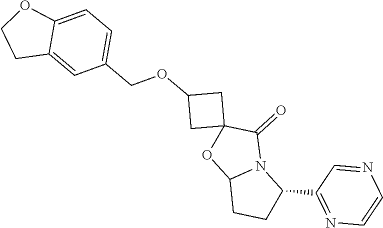 [SMILES: O=C1N2C(CC[C@H]2c2cnccn2)OC12CC(OCc1ccc3c(c1)CCO3)C2] | (5′S)-3-((2,3-dihydrobenzofuran-5- yl)methoxy)-5′-(pyrazin-2- yl)tetrahydro-3′H-spiro[cyclobutane- 1,2′-pyrrolo[2,1-b]oxazol]-3′-one | Calc'd 394, found 394 |
| 18.109 | 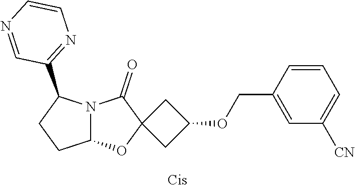 | 3-((((5′S,7a′R)-3′-oxo-5′-(pyrazin-2- yl)tetrahydro-3′H-spiro[cyclobutane- 1,2′-pyrrolo[2,1-b]oxazol]-3- yl)oxy)methyl)benzonitrile | Calc'd 377, found 377 |
| 18.110 | 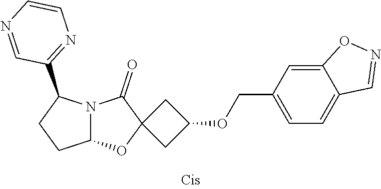 | (5′S,7a′R)-3-(benzo[d]isoxazol-6- ylmethoxy)-5′-(pyrazin-2- yl)tetrahydro-3′H-spiro[cyclobutane- 1,2′-pyrrolo [2,1-b]oxazol]-3′-one | Calc'd 393, found 393 |

### Example 19.1

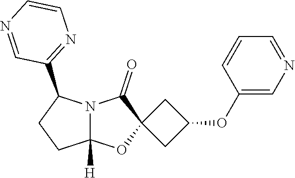 [SMILES: O=C1N2[C@@H](CC[C@H]2c2cnccn2)O[C@]12C[C@@H](Oc1cccnc1)C2]

### (5′S,7a′R)-5′-(pyrazin-2-yl)-3-(pyridin-3-yloxy)tetrahydro-3′H-spiro[cyclobutane-1,2′-pyrrolo[2,1-b]oxazol]-3′-one

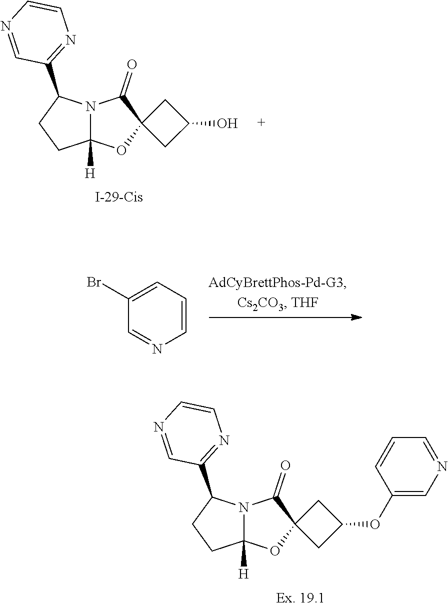 [SMILES: O=C1N2[C@@H](CC[C@H]2c2cnccn2)O[C@]12C[C@@H](O)C2 ; Brc1cccnc1 ; O=C1N2[C@@H](CC[C@H]2c2cnccn2)O[C@]12C[C@@H](Oc1cccnc1)C2]

To a mixture of 3-bromopyridine (27.2 mg, 0.172 mmol),3-hydroxy-5′-(pyrazin-2-yl)tetrahydro-3′H-spiro[cyclobutane-1,2′-pyrrolo[2,1-b]oxazol]-3′-one (I-29-Cis) (30 mg, 0.115 mmol), cesium carbonate (112 mg, 0.344 mmol) in THF (40 ml) was added AdCyBrettPhos-Pd-G3 (40 mg, 0.042 mmol) under N₂ which was stirred at 100° C. for 16 h. LCMS showed that the desired target was formed. The reaction crude was purified by reverse phase chromatography eluting with water (10 mM-NH₄—HCO₃)-MeCN) to give (5′S,7a′R)-5′-(pyrazin-2-yl)-3-(pyridin-3-yloxy)tetrahydro-3′H-spiro[cyclobutane-1,2′-pyrrolo[2,1-b]oxazol]-3′-one (Ex. 19.1) MS (ESI) m/z [M+H]⁺: 339; ¹H NMR (400 MHz, METHANOL-d4) δ 8.70 (d, J=1.19 Hz, 1H), 8.59-8.63 (m, 1H), 8.55 (d, J=2.50 Hz, 1H), 8.17-8.20 (m, 1H), 8.11-8.15 (m, 1H), 7.36 (t, J=2.09 Hz, 2H), 5.79 (dd, J=4.95, 7.33 Hz, 1H), 5.13 (t, J=7.69 Hz, 1H), 4.67 (quin, J=6.77 Hz, 1H), 3.06-3.20 (m, 2H), 2.65-2.76 (m, 1H), 2.57-2.64 (m, 1H), 2.43-2.53 (m, 1H), 2.19-2.38 (m, 2H), 1.65-1.84 (m, 1H)

### Example 19.2

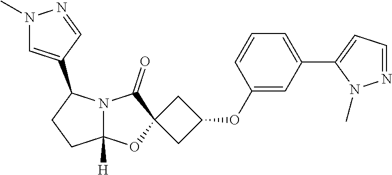 [SMILES: Cn1cc([C@@H]2CC[C@H]3O[C@]4(C[C@@H](Oc5cccc(-c6ccnn6C)c5)C4)C(=O)N32)cn1]

### (5′S,7a′R)-5′-(1-methyl-1H-pyrazol-3-yl)-3-(3-(1-methyl-1H-pyrazol-5-yl)phenoxy)tetrahydro-3′H-spiro[cyclobutane-1,2′-pyrrolo[2,1-b]oxazol]-3′-one

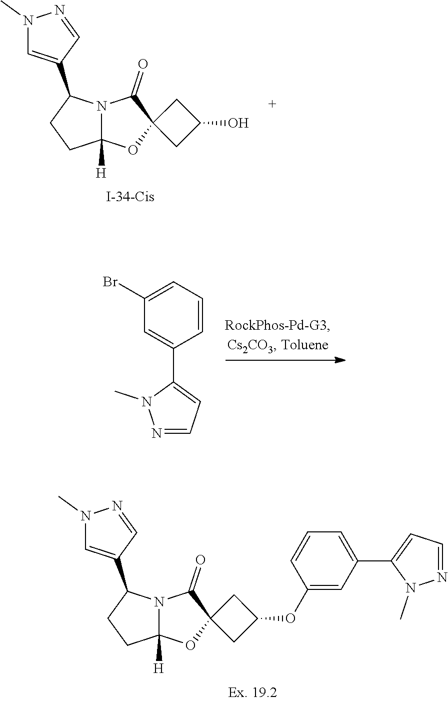 [SMILES: Cn1cc([C@@H]2CC[C@H]3O[C@]4(C[C@@H](O)C4)C(=O)N32)cn1 ; Cn1nccc1-c1cccc(Br)c1 ; Cn1cc([C@@H]2CC[C@H]3O[C@]4(C[C@@H](Oc5cccc(-c6ccnn6C)c5)C4)C(=O)N32)cn1]

To a solution of (1R,3R,5′S,7a′R)-3-hydroxy-5′-(1-methyl-1H-pyrazol-3-yl)tetrahydro-3′H-spiro[cyclobutane-1,2′-pyrrolo[2,1-b]oxazol]-3′-one (I-34-Cis) (30 mg, 0.114 mmol) in Toluene (2 ml) was added 5-(3-bromophenyl)-1-methyl-1H-pyrazole (32.4 mg, 0.137 mmol), Cs₂CO₃ (111 mg, 0.342 mmol) and RockPhos-Pd-G3 (9.55 mg, 0.011 mmol) under N₂. The resulting mixture was stirred at 100° C. for 12 h under N₂. LCMS showed SM was consumed, desired MS peak was observed. Filtered the solid and the filtrate was concentrated to give a residue. The reaction crude was purified by reverse phase chromatography eluting with water (10 mM-NH₄—HCO₃)-MeCN) to give (5′S,7a′R)-5′-(1-methyl-1H-pyrazol-3-yl)-3-(3-(1-methyl-1H-pyrazol-5-yl)phenoxy)tetrahydro-3′H-spiro[cyclobutane-1,2′-pyrrolo[2,1-b]oxazol]-3′-one (Ex. 19.2). MS (ESI) m/z [M+H]⁺: 420.2; ¹H NMR (400 MHz, METHANOL-d4) δ 7.50 (dd, J=2.09, 10.43 Hz, 2H), 7.37-7.42 (m, 1H), 7.04-7.08 (m, 1H), 6.93-6.96 (m, 2H), 6.36 (d, J=2.03 Hz, 1H), 6.23 (d, J=2.27 Hz, 1H), 5.69 (dd, J=5.07, 6.85 Hz, 1H), 5.00 (t, J=7.33 Hz, 1H), 4.62-4.66 (m, 1H), 3.86 (d, J=5.36 Hz, 6H), 3.04-3.12 (m, 2H), 2.51-2.59 (m, 2H), 2.39-2.46 (m, 1H), 2.15-2.26 (m, 2H), 1.61-1.73 (m, 1H)

The compounds presented in Table 15 below were prepared using a procedure analogous to one described for Example 19.1, 19.2 with the appropriately chosen intermediate from Table 5, along with the appropriate solvent, base, and commercially available or known aromatic electrophiles. All products are drawn in either cis or trans geometry unless unknown.

TABLE 15

| Ex. | | | Exact Mass |
|---|---|---|---|
| No. | Structure | IUPAC Name | [M + H]⁺ |
| 19.3 | 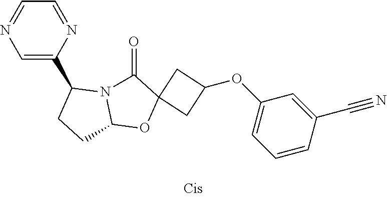 [SMILES: N#Cc1cccc(OC2CC3(C2)O[C@@H]2CC[C@@H](c4cnccn4)N2C3=O)c1] | 3-{[(5′S,7a′R)-3′-oxo-5′-(pyrazin-2- yl)tetrahydro-3′H-spiro[cyclobutane- 1,2′-pyrrolo[2,1-b][1,3]oxazol]-3- yl]oxy}benzonitrile | Calc'd 363, found 363 |
| 19.4 | 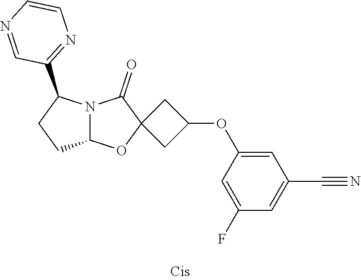 [SMILES: N#Cc1cc(F)cc(OC2CC3(C2)O[C@@H]2CC[C@@H](c4cnccn4)N2C3=O)c1] | 3-fluoro-5-{[(5′S,7a′R)-3′-oxo-5′- (pyrazin-2-yl)tetrahydro-3′H- spiro[cyclobutane-1,2′-pyrrolo[2,1- b][1,3]oxazol]-3-yl]oxy}benzonitrile | Calc'd 381, found 381 |
| 19.5 | 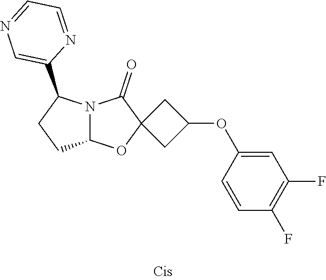 [SMILES: O=C1N2[C@@H](CC[C@H]2c2cnccn2)OC12CC(Oc1ccc(F)c(F)c1)C2] | (5′S,7a′R)-3-(3,4-difluorophenoxy)-5′- (pyrazin-2-yl)tetrahydro-3′H- spiro[cyclobutane-1,2′-pyrrolo[2,1- b][1,3]oxazol]-3′-one | Calc'd 374, found 374 |
| 19.6 | 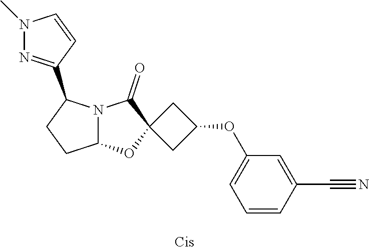 [SMILES: Cn1ccc([C@@H]2CC[C@H]3O[C@]4(C[C@@H](Oc5cccc(C#N)c5)C4)C(=O)N32)n1] | 3-{[(1s,3S,5′S,7a′R)-5′-(1-methyl-1H- pyrazol-3-yl)-3′-oxotetrahydro-3′H- spiro[cyclobutane-1,2′-pyrrolo[2,1- b][1,3]oxazol]-3-yl]oxy}benzonitrile | Calc'd 365, found 365 |
| 19.7 | 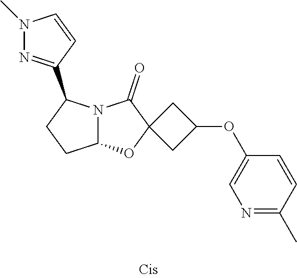 | (5′S,7a′R)-5′-(1-methyl-1H-pyrazol-3- yl)-3-[(6-methylpyridin-3- yl)oxy]tetrahydro-3′H- spiro[cyclobutane-1,2′-pyrrolo[2,1- b][1,3]oxazol]-3′-one | Calc'd 355, found 355 |
| 19.8 | 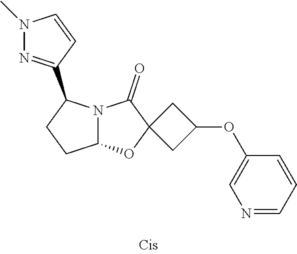 | (5′S,7a′R)-5′-(1-methyl-1H-pyrazol-3- yl)-3-[(pyridin-3-yl)oxy]tetrahydro- 3′H-spiro[cyclobutane-1,2′- pyrrolo[2,1-b][1,3]oxazol]-3′-one | Calc'd 341, found 341 |
| 19.9 | 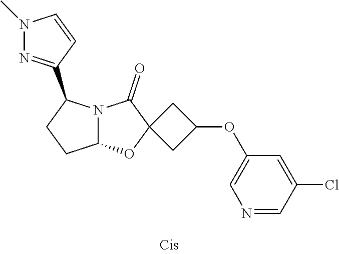 | (5′S,7a′R)-3-[(5-chloropyridin-3- yl)oxy]-5′-(1-methyl-1H-pyrazol-3- yl)tetrahydro-3′H-spiro[cyclobutane- 1,2′-pyrrolo[2,1-b][1,3]oxazol]-3′-one | Calc'd 375, found 375 |
| 19.10 | 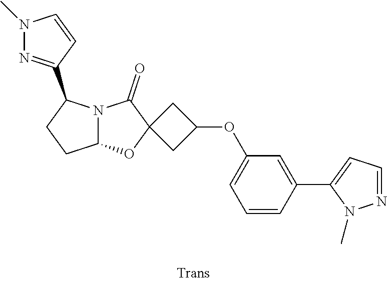 | (5′S,7a′R)-5′-(1-methyl-1H-pyrazol-3- yl)-3-[3-(1-methyl-1H-pyrazol-5- yl)phenoxy]tetrahydro-3′H- spiro[cyclobutane-1,2′-pyrrolo[2,1- b][1,3]oxazol]-3′-one | Calc'd 420, found 420 |
| 19.11 | 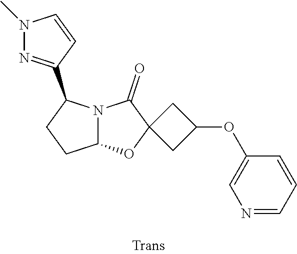 | (5′S,7a′R)-5′-(1-methyl-1H-pyrazol-3- yl)-3-[(pyridin-3-yl)oxy]tetrahydro- 3′H-spiro[cyclobutane-1,2′- pyrrolo[2,1-b][1,3]oxazol]-3′-one | Calc'd 341, found 341 |
| 19.12 | 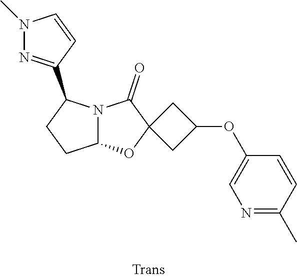 | (5′S,7a′R)-5′-(1-methyl-1H-pyrazol-3- yl)-3-[(6-methylpyridin-3- yl)oxy]tetrahydro-3′H- spiro[cyclobutane-1,2′-pyrrolo[2,1- b][1,3]oxazol]-3′-one | Calc'd 355, found 355 |
| 19.13 | 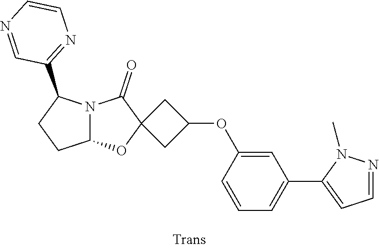 | (5′S,7a′R)-3-[3-(1-methyl-1H- pyrazol-5-yl)phenoxy]-5′-(pyrazin- 2-yl)tetrahydro-3′H- spiro[cyclobutane-1,2′- pyrrolo[2,1-b][1,3]oxazol]-3′-one | Calc'd 418, found 418 |
| 19.14 | 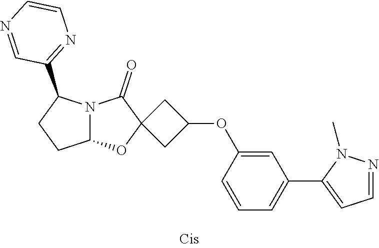 | (5′S,7a′R)-3-[3-(1-methyl-1H- pyrazol-5-yl)phenoxy]-5′-(pyrazin- 2-yl)tetrahydro-3′H- spiro[cyclobutane-1,2′- pyrrolo[2,1-b][1,3]oxazol]-3′-one | Calc'd 418, found 418 |
| 19.15 | 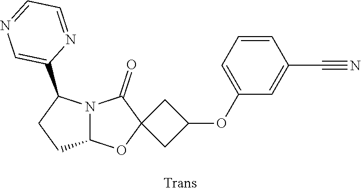 [SMILES: N#Cc1cccc(OC2CC3(C2)O[C@@H]2CC[C@@H](c4cnccn4)N2C3=O)c1] | 3-{[(5′S,7a′R)-3′-oxo-5′-(pyrazin- 2-yl)tetrahydro-3′H- spiro[cyclobutane-1,2′- pyrrolo[2,1-b][1,3]oxazol]-3- yl]oxy}benzonitrile | Calc'd 363 found 363 |
| 19.16 | 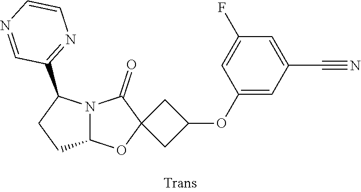 [SMILES: N#Cc1cc(F)cc(OC2CC3(C2)O[C@@H]2CC[C@@H](c4cnccn4)N2C3=O)c1] | 3-fluoro-5-{[(5′S,7a′R)-3′-oxo-5′- (pyrazin-2-yl)tetrahydro-3′H- spiro[cyclobutane-1,2′- pyrrolo[2,1- | Calc'd 381 found 381 |
| 19.17 | 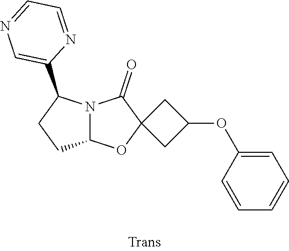 [SMILES: O=C1N2[C@@H](CC[C@H]2c2cnccn2)OC12CC(Oc1ccccc1)C2] | (5′S,7a′R)-3-phenoxy-5′-(pyrazin- 2-yl)tetrahydro-3′H- spiro[cyclobutane-1,2′- pyrrolo[2,1-b][1,3]oxazol]-3′-one | Calc'd 338 found 338 |
| 19.18 | 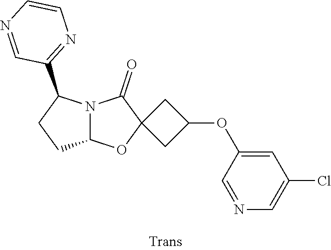 [SMILES: O=C1N2[C@@H](CC[C@H]2c2cnccn2)OC12CC(Oc1cncc(Cl)c1)C2] | (5′S,7a′R)-3-[(5-chloropyridin-3- yl)oxy]-5′-(pyrazin-2- yl)tetrahydro-3′H- spiro[cyclobutane-1,2′- pyrrolo[2,1-b][1,3]oxazol]-3′-one | Calc'd 373 found 373 |
| 19.19 | 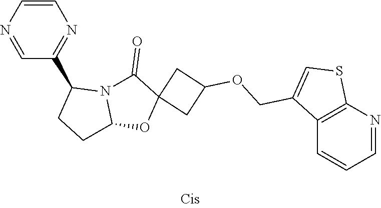 | (5′S,7a′R)-5′-(pyrazin-2-yl)-3- [(thieno[2,3-b]pyridin-3- yl)methoxy]tetrahydro-3′H- spiro[cyclobutane-1,2′- pyrrolo[2,1-b][1,3]oxazol]-3′-one | Calc'd 409 found 409 |
| 19.20 | 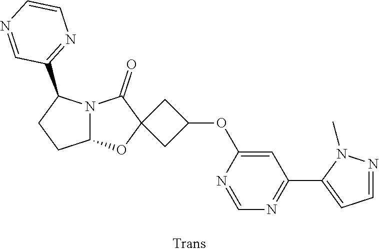 | (5′S,7a′R)-3-{[6-(1-methyl-1H- pyrazol-5-yl)pyrimidin-4-yl]oxy}- 5′-(pyrazin-2-yl)tetrahydro-3′H- spiro[cyclobutane-1,2′- pyrrolo[2,1-b][1,3]oxazol]-3′-one | Calc'd 420, found 420 |
| 19.21 | 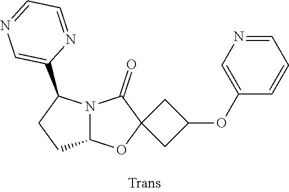 [SMILES: O=C1N2[C@@H](CC[C@H]2c2cnccn2)OC12CC(Oc1cccnc1)C2] | (5′S,7a′R)-5′-(pyrazin-2-yl)-3- [(pyridin-3-yl)oxy]tetrahydro-3′H- spiro[cyclobutane-1,2′- pyrrolo[2,1-b][1,3]oxazol]-3′-one | Calc'd 339, found 339 |
| 19.22 | 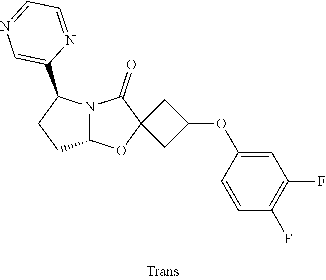 [SMILES: O=C1N2[C@@H](CC[C@H]2c2cnccn2)OC12CC(Oc1ccc(F)c(F)c1)C2] | (5′S,7a′R)-3-(3,4- difluorophenoxy)-5′-(pyrazin-2- yl)tetrahydro-3′H- spiro[cyclobutane-1,2′- pyrrolo[2,1-b][1,3]oxazol]-3′-one | Calc'd 355, found 355 |

### Example 20.1

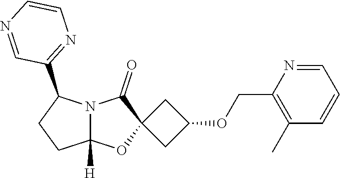 [SMILES: Cc1cccnc1CO[C@H]1C[C@]2(C1)O[C@@H]1CC[C@@H](c3cnccn3)N1C2=O]

### (5′S,7a′R)-5′-(1-methyl-1H-pyrazol-3-yl)-3-(3-(1-methyl-1H-pyrazol-5-yl)phenoxy)tetrahydro-3′H-spiro[cyclobutane-1,2′-pyrrolo[2,1-b]oxazol]-3′-one

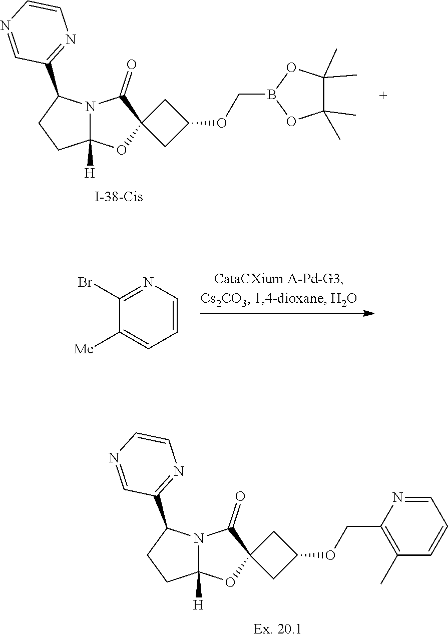 [SMILES: CC1(C)OB(CO[C@H]2C[C@]3(C2)O[C@@H]2CC[C@@H](c4cnccn4)N2C3=O)OC1(C)C ; Cc1cccnc1Br ; Cc1cccnc1CO[C@H]1C[C@]2(C1)O[C@@H]1CC[C@@H](c3cnccn3)N1C2=O]

To a solution of (5′S,7a′R)-5′-(pyrazin-2-yl)-3-((4,4,5,5-tetramethyl-1,3,2-dioxaborolan-2-yl)methoxy)tetrahydro-3′H-spiro[cyclobutane-1,2′-pyrrolo[2,1-b]oxazol]-3′-one (140 mg, 0.279 mmol) (I-38-Cis) in Dioxane (3 ml) and H₂O (0.600 ml) was added Cesium carbonate (455 mg, 1.396 mmol), 2-bromo-3-methylpyridine (144 mg, 0.837 mmol) and CataCXium A-Pd-G3 (68.1 mg, 0.084 mmol) in glove box. The mixture was stirred at 80° C. for 12 h under N₂. LCMS showed the desired product mass. The reaction crude was purified by reverse phase chromatography eluting with water (10 mM-NH₄—HCO₃)-MeCN) to give (5′S,7a′R)-3-((3-methylpyridin-2-yl)methoxy)-5′-(pyrazin-2-yl)tetrahydro-3′H-spiro[cyclobutane-1,2′-pyrrolo[2,1-b]oxazol]-3′-one (Ex. 20.1) MS (ESI) m/z [M+H]⁺: 367; ¹H NMR (400 MHz, METHANOL-d4) δ ppm 8.66 (d, J=1.31 Hz, 1H), 8.56-8.62 (m, 1H), 8.53 (d, J=2.50 Hz, 1H), 8.28-8.34 (m, 1H), 7.66 (d, J=7.63 Hz, 1H), 7.30 (dd, J=7.63, 4.89 Hz, 1H), 5.69-5.75 (m, 1H), 5.07 (t, J=7.63 Hz, 1H), 4.59 (s, 2H), 4.01 (quin, J=6.94 Hz, 1H), 2.75-2.86 (m, 2H), 2.61-2.72, (m, 1H), 2.43 (s, 3H), 2.33-2.40 (m, 1H), 2.16-2.30 (m, 3H), 1.62-1.76 (m, 1H).

The compounds presented in Table 16 below were prepared using a procedure analogous to one described for Example 20.1 with the appropriately chosen intermediate from Table 6, commercially available or known aromatic electrophiles. All products are drawn in either cis or trans geometry unless unknown.

TABLE 16

| Ex. | | | Exact Mass |
|---|---|---|---|
| No. | Structure | IUPAC Name | [M + H]⁺ |
| 20.2 | 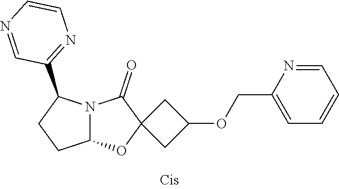 | (5′S,7a′R)-5′-(pyrazin-2-yl)-3- [(pyridin-2-yl)methoxy]tetrahydro- 3′H-spiro[cyclobutane-1,2′- pyrrolo[2,1-b][1,3]oxazol]-3′-one | Calc'd 353, found 353 |
| 20.3 | 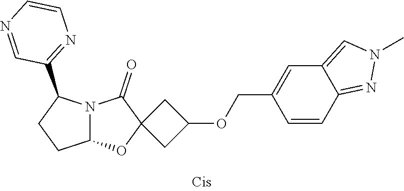 | (5′S,7a′R)-3-[(2-methyl-2H-indazol- 5-yl)methoxy]-5′-(pyrazin-2- yl)tetrahydro-3′H-spiro[cyclobutane- 1,2′-pyrrolo[2,1-b][1,3]oxazol]-3′-one | Calc'd 406, found 406 |
| 20.4 | 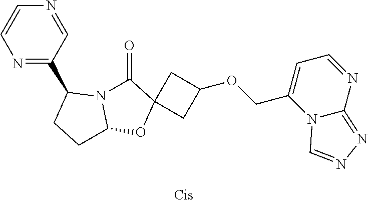 | (5′S,7a′R)-5′-(pyrazin-2-yl)-3- [([1,2,4]triazolo[4,3-a]pyrimidin-5- yl)methoxy]tetrahydro-3′H- spiro[cyclobutane-1,2′-pyrrolo[2,1- b][1,3]oxazol]-3′-one | Calc'd 394, found 394 |
| 20.5 | 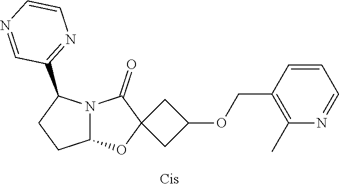 | (5′S,7a′R)-3-[(2-methylpyridin-3- yl)methoxy]-5′-(pyrazin-2- yl)tetrahydro-3′H-spiro[cyclobutane- 1,2′-pyrrolo[2,1-b][1,3]oxazol]-3′-one | Calc'd 367, found 367 |
| 20.6 | 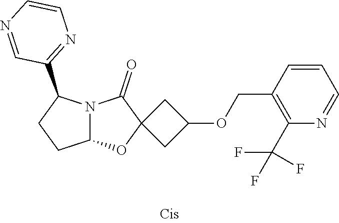 | (5′S,7a′R)-5′-(pyrazin-2-yl)-3-{[2- (trifluoromethyl)pyridin-3- yl]methoxy}tetrahydro-3′H- spiro[cyclobutane-1,2′-pyrrolo[2,1- b][1,3]oxazol]-3′-one | Calc'd 421, found 421 |
| 20.7 | 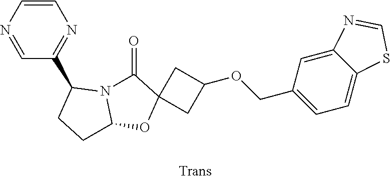 | (5′S,7a′R)-3-[(1,3-benzothiazol-5- yl)methoxy]-5′-(pyrazin-2- yl)tetrahydro-3′H-spiro[cyclobutane- 1,2′-pyrrolo[2,1-b][1,3]oxazol]-3′-one | Calc'd 409, found 409 |
| 20.8 | 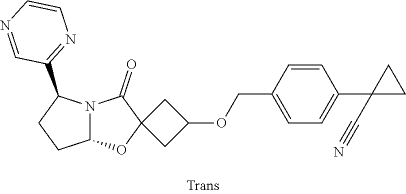 | 1-[4-({[(5′S,7a′R)-3′-oxo-5′-(pyrazin- 2-yl)tetrahydro-3′H- spiro[cyclobutane-1,2′-pyrrolo[2,1- b][1,3]oxazol]-3- yl]oxy}methyl)phenyl]cyclopropane- 1-carbonitrile | Calc'd 417, found 417 |
| 20.9 | 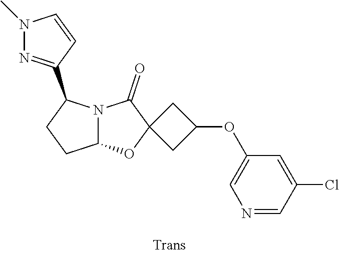 | (5′S,7a′R)-3-[(5-chloropyridin-3- yl)oxy]-5′-(1-methyl-1H-pyrazol-3- yl)tetrahydro-3′H-spiro[cyclobutane- 1,2′-pyrrolo[2,1-b][1,3]oxazol]-3′-one | Calc'd 375, found 375 |

### Examples 20.10

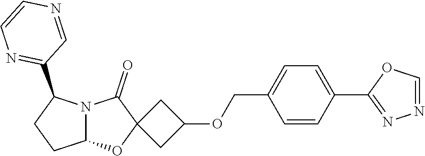

### Cis-(5′S,7a′R)-3-{[4-(1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one

To a solution of (5′S,7a′R)-5′-(pyrazin-2-yl)-3-((4,4,5,5-tetramethyl-1,3,2-dioxaborolan-2-yl)methoxy)tetrahydro-3′H-spiro[cyclobutane-1,2′-pyrrolo[2,1-b]oxazol]-3′-one ((85.0 g, 211 mmol) (I-38-Cis) in dioxane (1.1 L) and H₂O (75 ml) was added Cesium carbonate (345 g, 1.06 mmol), 2-(4-bromophenyl)-1,3,4-oxadiazole (52.4 g, 233 mmol) and CataCXium A-Pd-G3 (46.2 g, 63.5 mmol). The mixture was stirred at 80° C. for 12 h under N₂. LCMS showed the desired product mass. The mixture was added into EDTA 1000 mL, and stirred for 1 hr at 25° C. Then extracted with DCM 1500 mL (500 mL*3) and washed with brine 500 mL (500 mL*1). Dried over anhydrous Na₂SO₄, filtered and concentrated under reduced pressure to give a residue. The crude residue was purified by column chromatography (SiO₂, Ethyl acetate/Methanol=1/0 to 10/1) first and then by prep-HPLC (column: Welch Xtimate C18 250*100 mm #10 um; mobile phase: [H₂O (0.05% NH₃H₂O+10 mM NH₄HCO₃)−ACN]; gradient: 15%-45% B over 20.0 min) to provide Cis-(5′S,7a′R)-3-{[4-(1,3,4-oxadiazol-2-yl)phenyl]methoxy}-5′-(pyrazin-2-yl)tetrahydro-3′H-spiro[cyclobutane-1,2′-pyrrolo[2,1-b][1,3]oxazol]-3′-one (13.3 g, 31.7 mmol). MS (ESI) m/z [M+H]⁺: 420.2, ¹H NMR: (400 MHz, DMSO-d6) δ=9.34 (s, 1H), 8.71-8.67 (m, 1H), 8.64-8.61 (m, 1H), 8.60-8.57 (m, 1H), 8.01 (d, 1H, J=8.4 Hz), 7.56 (d, 1H, J=8.4 Hz), 5.74-5.64 (m, 1H), 5.07-4.97 (m, 1H), 4.51 (s, 2H), 3.98-3.85 (m, 1H), 2.85-2.73 (m, 2H), 2.63-2.53 (m, 1H), 2.34-2.26 (m, 1H), 2.25-2.16 (m, 2H), 2.13-2.01 (m, 1H), 1.66-1.54 (m, 1H).

### Examples 21.1 and 21.2

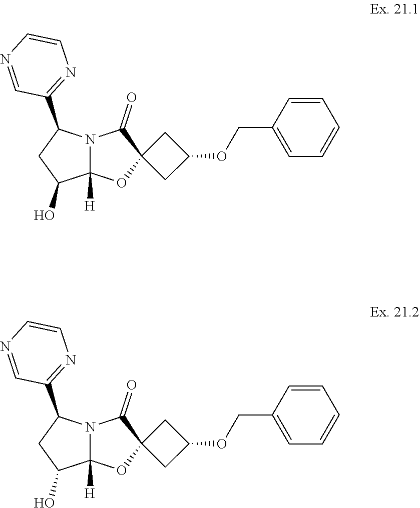 [SMILES: O=C1N2[C@H](O[C@]13C[C@@H](OCc1ccccc1)C3)[C@@H](O)C[C@H]2c1cnccn1 ; O=C1N2[C@H](O[C@]13C[C@@H](OCc1ccccc1)C3)[C@H](O)C[C@H]2c1cnccn1]

### (1s,3S,5′S,7′S,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one

### (1s,3S,5′S,7′R,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one

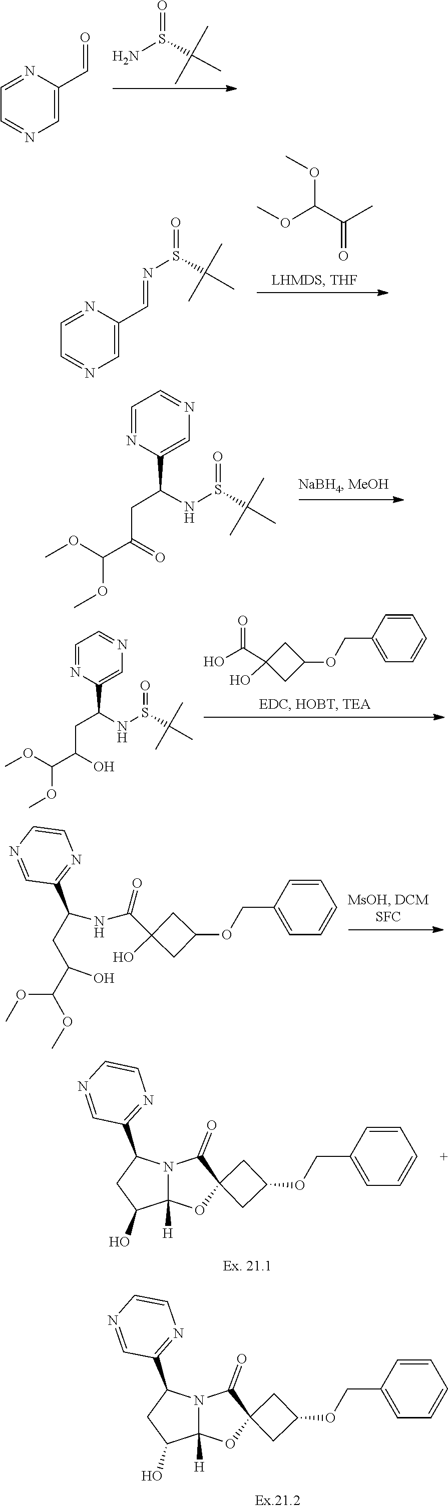

### Step 1. (R)-2-methyl-N-(pyrazin-2-ylmethylene)propane-2-sulfinamide

To a solution of pyrazine-2-carbaldehyde (10 g, 93 mmol) in DCM (300 ml) was added (S)-2-methylpropane-2-sulfinamide (13.4 g, 111 mmol) and Cs₂CO₃ (90 g, 278 mmol) at 25° C. and the mixture was stirred at 25° C. for 3 h. The reaction mixture was filtered and the filtrate was concentrated in vacuum to get the brown oil. The oil was purified by column chromatography (SiO₂, 330 g ISCO column eluting with 10-70% EtOAc/Hex) to obtain (R)-2-methyl-N-(pyrazin-2-ylmethylene)propane-2-sulfinamide. MS (ESI) m/z [M+H]⁺: 212

### Step 2. Preparation of (R)—N—((S)-4,4-dimethoxy-3-oxo-1-(pyrazin-2-yl)butyl)-2-methylpropane-2-sulfinamide

To a solution of pyruvic aldehyde dimethyl acetal (3.91 g, 33.1 mmol) in THF (50 mL) was added hexamethyldisilazane lithium salt (35.5 ml, 35.5 mmol) at −60° C. and stirred at −60° C. for 1 h, Added (R)-2-methyl-N-(pyrazin-2-ylmethylene)propane-2-sulfinamide (5 g, 23.66 mmol) in THF (50 mL), the mixture was stirred at −60° C. for 4 h. LCMS showed desired compound was found. The mixture was poured into aqueous NaHCO₃ (50 mL) and water (20 mL) and extracted with EA (100 mL*3). The mixture was concentrated under reduced pressure. The residue was purified by flash silica gel chromatography to give a crude product. The crude product was purified by reversed MPLC and the mixture was resolved by Chiral-SFC (Condition: DAICEL CHIRALCEL OD (250 mm*30 mm, 10 μm)), Mobile phase: A: CO2, B: CO2-ETOH (0.1% NH₃H₂O), Gradient: 0.15% B, FlowRate: 60 mL/min) to give (R)—N—((S)-4,4-dimethoxy-3-oxo-1-(pyrazin-2-yl)butyl)-2-methylpropane-2-sulfinamide (950 mg, 2.88 mmol, 67.9% yield) as a yellow oil. MS (ESI) m/z [M+H]⁺: 330

### Step 3. Preparation of (R)—N-(3-hydroxy-4,4-dimethoxy-1-(pyrazin-2-yl)butyl)-2-methylpropane-2-sulfinamide

To a solution of (R)—N-(4,4-dimethoxy-3-oxo-1-(pyrazin-2-yl)butyl)-2-methylpropane-2-sulfinamide (950 mg, 2.88 mmol) in MeOH (9 mL) was added sodium borohydride (218 mg, 5.77 mmol) at 0° C. The resulting mixture was stirred at 20° C. for 0.5 h. LCMS showed the desired compound was found. The mixture was added water (10 mL) and extracted with DCM:MeOH (10:1=10 mL*3). The mixture was concentrated to give (R)—N-(3-hydroxy-4,4-dimethoxy-1-(pyrazin-2-yl)butyl)-2-methylpropane-2-sulfinamide (950 mg, 2.87 mmol, 99% yield) as a yellow oil. MS (ESI) m/z [M+H]⁺: 332

### Step 4. Preparation of (4S)-4-amino-1,1-dimethoxy-4-(pyrazin-2-yl)butan-2-ol

To a solution of (R)—N-((1S)-3-hydroxy-4,4-dimethoxy-1-(pyrazin-2-yl)butyl)-2-methylpropane-2-sulfinamide (950 mg, 2.87 mmol) in MeOH (10 ml) was added acetyl chloride (0.245 ml, 3.44 mmol) at 0° C. under N₂ and the reaction was stirred at 20° C. for 12 h. LCMS showed the desired compound was found. The reaction was added TEA with PH (˜8). The mixture was directly concentrated to give (4S)-4-amino-1,1-dimethoxy-4-(pyrazin-2-yl)butan-2-ol (651 mg, 2.87 mmol, 100% yield) as yellow oil, which was used to next step without further purification. MS (ESI) m/z [M+H]⁺: 227

### Step 5. Preparation of 3-(benzyloxy)-1-hydroxy-N-((1S)-3-hydroxy-4,4-dimethoxy-1-(pyrazin-2-yl)butyl)cyclobutane-1-carboxamide

To a solution of 3-(benzyloxy)-1-hydroxycyclobutane-1-carboxylic acid (500 mg, 2.250 mmol) in DMF (3 mL) was added (4S)-4-amino-1,1-dimethoxy-4-(pyrazin-2-yl)butan-2-ol (614 mg, 2.70 mmol), TEA (0.627 mL, 4.50 mmol), HOBT (456 mg, 3.37 mmol), EDC (647 mg, 3.37 mmol) and the resulting mixture was stirred at 25° C. for 1 h. LCMS showed the desired compound was found. Filtered and concentrated. The mixture was extracted with water (30 mL) and EA (10 mL*3). The mixture was concentrated to give 3-(benzyloxy)-1-hydroxy-N-((1S)-3-hydroxy-4,4-dimethoxy-1-(pyrazin-2-yl)butyl)cyclobutane-1-carboxamide (971 mg, 2.250 mmol, 100% yield) as yellow oil. MS (ESI) m/z [M+H]⁺: 432

### Step 6. Preparation of (1s,3S,5′S,7′S,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.1) and (1s,3S,5′S,7′R,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.2)

To a solution of 3-(benzyloxy)-1-hydroxy-N-((1S)-3-hydroxy-4,4-dimethoxy-1-(pyrazin-2-yl)butyl)cyclobutane-1-carboxamide (500 mg, 1.159 mmol) in DCM (3 ml) was added MsOH (0.376 ml, 5.79 mmol) and the resulting mixture was stirred at 25° C. for 12 h. LCMS showed the reaction was completed, and the desired product was found. The mixture was adjusted to pH 9 with TEA (3 ml). The reaction mixture was concentrated to dryness. The residue was purified by Prep-HPLC (Prep HPLC condition: Preparative HPLC on eh instrument fitted with Phenomenex Gemini-NX 150*30 mm*5 um using the mobile phase A-B: water (10 mM HCOONH4)-ACN, Gradient: 20-50% B, 0-11 min; 100% B, 11-13.5 min; 10% B, 13.5-15.5 min. FlowRate: 25 mL/min) and the mixture was resolved by Chiral-SFC (Condition: sfc-21 (Column: REGIS (R,R)WHELK-O1 (250 mm*25 mm, 10 um)), Mobile phase: A: CO₂, B: CO₂-i-PrOH (0.1% NH3H2O), Gradient: 0.45% B, FlowRate: 80 mL/min) to give (1s,3S,5′S,7′S,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.1) and (1s,3S,5′S,7′R,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.2) (1s,3S,5′S,7′S,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.1) MS (ESI) m/z [M+H]⁺: 368; ¹H NMR (400 MHz, METHANOL-d₄) δ 8.68 (d, J=1.4 Hz, 1H), 8.62 (dd, J=1.8, 2.50 Hz, 1H), 8.54 (d, J=2.4 Hz, 1H), 7.34 (d, J=4.4 Hz, 4H), 7.25-7.31 (m, 1H), 5.40 (d, J=5.6 Hz, 1H), 5.08 (t, J=8.8 Hz, 1H), 4.45 (s, 2H), 3.91-4.07 (m, 2H), 2.74-2.88 (m, 3H), 2.34-2.47 (m, 1H), 2.18-2.34 (m, 2H) (1s,3S,5′S,7′R,7a′R)-3-(benzyloxy)-7′-hydroxy-5′-(pyrazin-2-yl)tetrahydro-3′H-spiro[cyclobutane-1,2′-pyrrolo[2,1-b]oxazol]-3′-one (Ex. 21.2) MS (ESI) m/z [M+H]⁺: 368; ¹H NMR (400 MHz, METHANOL-d₄) δ 8.68 (d, J=1.6 Hz, 1H), 8.60 (dd, J=1.2, 2.40 Hz, 1H), 8.53 (d, J=2.8 Hz, 1H), 7.34 (d, J=4.4 Hz, 4H), 7.22-7.31 (m, 1H), 5.62 (d, J=2.4 Hz, 1H), 5.04 (t, J=8.8 Hz, 1H), 4.46 (s, 2H), 4.11 (t, J=2.80 Hz, 1H), 3.99 (quin, J=6.4 Hz, 1H), 2.72-2.86 (m, 2H), 2.55 (dd, J=8.4, 13.6 Hz, 1H), 2.29-2.43 (m, 3H)

### Examples 22.1

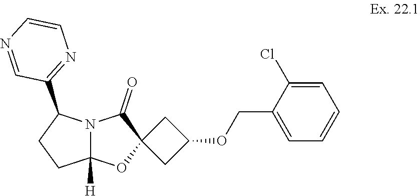 [SMILES: O=C1N2[C@@H](CC[C@H]2c2cnccn2)O[C@]12C[C@@H](OCc1ccccc1Cl)C2]

### (1S,3S,5′S,7a′R)-3-((2-chlorobenzyl)oxy)-5′-(pyrazin-2-yl)tetrahydro-3′H-spiro[cyclobutane-1,2′-pyrrolo[2,1-b]oxazol]-3′-one

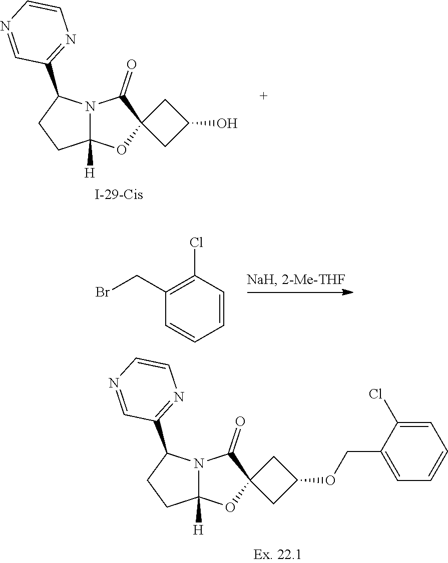

A oven-dried 4-mL dram vial was charged with NaH (6.12 mg, 0.230 mmol) inside of glovebox. Solution of (1S,3S,5′S,7a′R)-3-hydroxy-5′-(pyrazin-2-yl)tetrahydro-3′H-spiro[cyclobutane-1,2′-pyrrolo[2,1-b]oxazol]-3′-one (40 mg, 0.153 mmol) in 2-Me-THF was added and mixture was stirred for 10 min at ambient temperature. Then solution of 1-(bromomethyl)-2-chlorobenzene (62.9 mg, 0.306 mmol) in 2-Me-THF was added. The final concentration reached 0.1 M. The vial was sealed, taken out from the glovebox, and heated to 50° C. for 14 h. Nearly full conversion was attained, as judged by LCMS. Reaction was cooled down, slowly quenched with sat. aq. NH₄Cl (1 mL) was diluted with DCM (3 mL) and passed through a phase separator. Crude material was concentrated in vacuo and purified by reverse phase preparative HPLC to give (1s,3S,5′S,7a′R)-3-((2-chlorobenzyl)oxy)-5′-(pyrazin-2-yl)tetrahydro-3′H-spiro[cyclobutane-1,2′-pyrrolo[2,1-b]oxazol]-3′-one (Ex. 22.1)

MS (ESI) m/z [M+H]⁺: 386. 1H NMR (499 MHz, DMSO-d6) δ 8.71 (d, J=1.4 Hz, 1H), 8.64 (dd, J=2.4, 1.5 Hz, 1H), 8.61 (d, J=2.5 Hz, 1H), 7.53-7.49 (m, 1H), 7.48-7.45 (m, 1H), 7.38-7.34 (m, 2H), 5.70 (dd, J=7.3, 5.0 Hz, 1H), 5.03 (t, J=7.8 Hz, 1H), 4.50 (s, 2H), 3.94 (p, J=6.9 Hz, 1H), 2.80 (ddd, J=12.4, 6.1, 1.3 Hz, 2H), 2.59 (dtd, J=13.1, 8.1, 2.5 Hz, 1H), 2.33-2.28 (m, 1H), 2.21 (ddt, J=11.5, 7.0, 3.4 Hz, 2H), 2.08 (ddt, J=13.1, 11.1, 7.1 Hz, 1H), 1.61 (tt, J=11.3, 8.1 Hz, 1H).

The compounds presented in Table 17 below were prepared using a procedure analogous to one described for Example 22.1 with the appropriately chosen intermediate from Table 5, commercially available or known benzylic electrophiles. All products are drawn in either cis or trans geometry unless unknown.

TABLE 17

| Ex. | | | Exact Mass |
|---|---|---|---|
| No. | Structure | IUPAC Name | [M + H]⁺ |
| 22.2 | 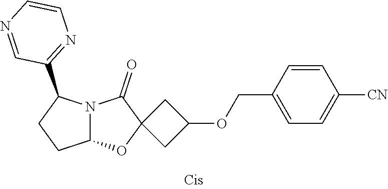 | 4-((((5′S,7a′R)-3′-oxo-5′-(pyrazin-2- yl)tetrahydro-3′H-spiro[cyclobutane- 1,2′-pyrrolo[2,1-b]oxazol]-3- yl)oxy)methyl)benzonitrile | Calc'd 377, found 377 |
| 22.3 | 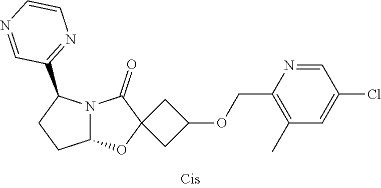 | (5′S,7a′R)-3-((5-chloro-3- methylpyridin-2-yl)methoxy)-5′- (pyrazin-2-yl)tetrahydro-3′H- spiro[cyclobutane-1,2′-pyrrolo[2,1- b]oxazol]-3′-one | Calc'd 400, found 400 |
| 22.4 | 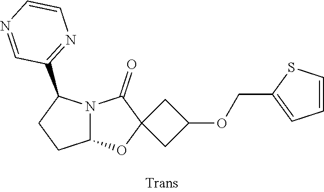 | (5′S,7a′R)-5′-(pyrazin-2-yl)-3- (thiophen-2-ylmethoxy)tetrahydro- 3′H-spiro[cyclobutane-1,2′- pyrrolo[2,1-b]oxazol]-3′-one | Calc'd 358, found 358 |
| 22.5 | 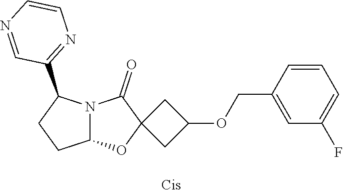 | (5′S,7a′R)-3-[(3- chlorophenyl)methoxy]-5′- (pyrazin-2-yl)tetrahydro-3′H- spiro[cyclobutane-1,2′- pyrrolo[2,1-b][1,3]oxazol]-3′-one | Calc'd 370, found 370 |
| 22.6 | 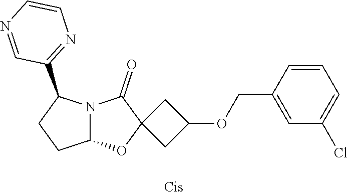 | (5′S,7a′R)-3-[(3- chlorophenyl)methoxy]-5′- (pyrazin-2-yl)tetrahydro-3′H- spiro[cyclobutane-1,2′- pyrrolo[2,1-b][1,3]oxazol]-3′-one | Calc'd 386, found 386 |
| 22.7 | 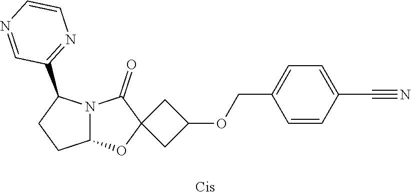 | 4-({[(5′S,7a′R)-3′-oxo-5′-(pyrazin- 2-yl)tetrahydro-3′H- spiro[cyclobutane-1,2′- pyrrolo[2,1-b][1,3]oxazol]-3- yl]oxy}methyl)benzonitrile | Calc'd 377, found 377 |
| 22.8 | 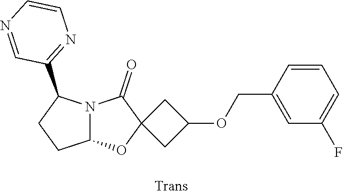 | (5′S,7a′R)-3-[(3- fluorophenyl)methoxy]-5′- (pyrazin-2-yl)tetrahydro-3′H- spiro[cyclobutane-1,2′- pyrrolo[2,1-b][1,3]oxazol]-3′-one | Calc'd 370, found 370 |
| 22.9 | 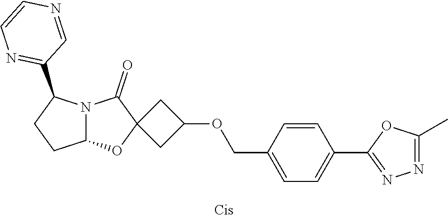 | (5′S,7a′R)-3-{[4-(5-methyl-1,3,4- oxadiazol-2-yl)phenyl]methoxy}- 5′-(pyrazin-2-yl)tetrahydro-3′H- spiro[cyclobutane-1,2′- pyrrolo[2,1-b][1,3]oxazol]-3′-one | Calc'd 434, found 434 |

### Examples 23.1

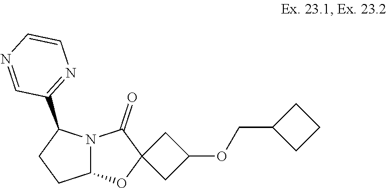

### (5′S,7a′R)-3-(cyclobutylmethoxy)-5′-(pyrazin-2-yl)tetrahydro-3′H-spiro[cyclobutane-1,2′-pyrrolo[2,1-b]oxazol]-3′-one

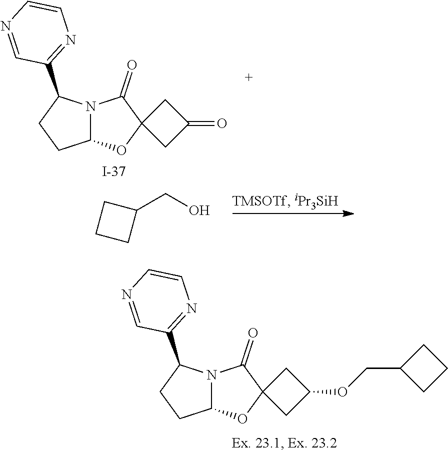

A oven-dried 4-mL dram vial was charged with cyclobutylmethanol (16.61 mg, 0.193 mmol) and triisopropylsilane (79 μl, 0.386 mmol). The solution of (5′S,7a′R)-5′-(pyrazin-2-yl)tetrahydro-3′H-spiro[cyclobutane-1,2′-pyrrolo[2,1-b]oxazole]-3,3′-dione (50 mg, 0.193 mmol) in dry acetonitrile (964 μl, 0.193 mmol) was added under inert atmosphere. The resulting solution was cooled to −10° C. and treated with trimethylsilyl trifluoromethanesulfonate (69.8 μl, 0.386 mmol). The final concentration reached 0.2 M. The reaction mixture was aged for 18 h. at the above temperature. Full conversion was achieved as was judged by LCMS; desired product was formed. The reaction mixture was quenched with aqueous sodium hydrogen carbonate (1 mL). The resulting suspension was transferred into phase separator with dichloromethane (4 mL). Organic phase was concentrated and purified on ISCO®; 4 g SepaFlash® Silica Flash Column, eluent isocratic 20%-30% of EtOAc:EtOH=3:1/Hexanes; 20CV. The isomers were further separated by chiral SFC (Column Lux-2 [250 mm×21 mm×5 μm]; 30% MeOH [0.1% NH4OH]/CO2; Flow Rate: 70 mL/min) delivering (5′S,7a′R)-3-(cyclobutylmethoxy)-5′-(pyrazin-2-yl)tetrahydro-3′H-spiro[cyclobutane-1,2′-pyrrolo[2,1-b]oxazol]-3′-one peak 1 (Ex. 23.1) and peak 2 (Ex. 23.2)

Peak 1 (Ex. 23.1): MS (ESI) m/z [M+H]⁺ calc'd for C₁₈H₂₃N₃O₃: 330, found: 330; 1H NMR (499 MHz, CDCl3) δ 8.67 (s, 1H), 8.53 (s, 2H), 5.66 (dd, J=6.9, 5.0 Hz, 1H), 5.13-5.06 (m, 1H), 3.93 (p, J=6.9 Hz, 1H), 3.34 (d, J=6.9 Hz, 2H), 2.88 (dt, J=11.7, 6.0 Hz, 1H), 2.83 (dt, J=12.1, 6.2 Hz, 1H), 2.60 (dt, J=11.6, 8.2 Hz, 1H), 2.53 (dt, J=15.0, 7.6 Hz, 1H), 2.36 (dd, J=11.8, 7.3 Hz, 1H), 2.28 (ddd, J=10.9, 8.5, 5.6 Hz, 3H), 2.09-2.01 (m, 2H), 1.94-1.82 (m, 2H), 1.75-1.66 (m, 3H).

Peak 2 (Ex. 23.2): MS (ESI) m/z [M+H]⁺ calc'd for C18H23N3O3: 330, found: 330; 1H NMR (499 MHz, CDCl3) δ 8.66 (s, 1H), 8.52 (s, 2H), 5.65-5.61 (m, 1H), 5.14-5.08 (m, 1H), 4.22 (p, J=7.4 Hz, 1H), 3.36-3.30 (m, 2H), 2.72 (dd, J=12.7, 7.8 Hz, 1H), 2.65 (dt, J=12.8, 6.6 Hz, 1H), 2.61-2.50 (m, 2H), 2.44-2.34 (m, 2H), 2.32-2.23 (m, 2H), 2.09-2.01 (m, 2H), 1.95-1.82 (m, 2H), 1.78-1.68 (m, 3H).

The compounds presented in Table 18 below were prepared using a procedure analogous to one described for Example 23.1 with the appropriately chosen commercially available or known alcohols. All products are drawn in either cis or trans geometry unless unknown.

TABLE 18

| Ex. | | | Exact Mass |
|---|---|---|---|
| No. | Structure | IUPAC Name | [M + H]⁺ |
| 23.3 | 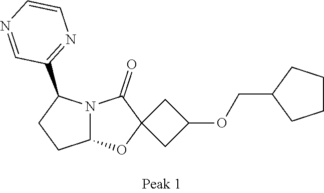 | (5′S,7a′R)-3-(cyclopentylmethoxy)-5′- (pyrazin-2-yl)tetrahydro-3′H- spiro[cyclobutane-1,2′-pyrrolo[2,1- b]oxazol]-3′-one | Calc'd 344, found 3444 |
| 23.4 | 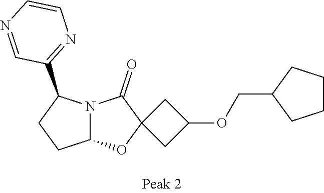 | (5′S,7a′R)-3-(cyclopentylmethoxy)-5′- (pyrazin-2-yl)tetrahydro-3′H- spiro[cyclobutane-1,2′-pyrrolo[2,1- b]oxazol]-3′-one | Calc'd 344, found 344 |

### Assay

### RIPK1-ADP-Glo Enzymatic Assay

The enzymatic activity of RIPK1 is measured using an assay derived from ADP-Glo kit (TMPromega), which provides a luminescent-based ADP detection system. Specifically, the ADP generated by RIPK1 kinase is proportionally detected as luminescent signals in a homogenous fashion. In this context, the assessment of the inhibitory effect of small molecules (EC50) is measured by the effectiveness of the compounds to inhibit the ATP to ADP conversion by RIPK1.

In this assay, the potency (EC50) of each compound was determined from a ten-point (1:3 serial dilution; top compound concentration of 100000 nM) titration curve using the following outlined procedure. To each well of a white ProxiPlus 384 well-plate, 30 nL of compound (1% DMSO in final assay volume of 3 μL) was dispensed, followed by the addition of 2 μL of 1× assay buffer (25 mM Hepes 7.3, 20 mM MgCl2, 50 mM NaCl, 1 mM DTT, 0.005% Tween20, and 0.02% BSA) containing 37.5 nM of GST-RIPK1 (recombinant GST-RIPK1 kinase domain (residues 1-327) enzyme produced from baculovirus-transfected Sf21 cells: MW=62 kDa). Plates were placed in an ambient temperature humidified chamber for a 30 minutes pre-incubation with compound. Subsequently, each reaction was initiated by the addition of 1 μL 1× assay buffer containing 900 μM ATP and 3 μM dephosphorylated-MBP substrate. The final reaction in each well of 3 μL consists of 25 nM of GST-RIPK1, 300 μM ATP, and 3 μM dephosphorylated-MBP. Kinase reactions were allowed to proceed for 150 minutes prior to adding ADP-Glo reagents per Promega's outlined kit protocol. Dose-response curves were generated by plotting percent effect (% product conversion; Y-axis) vs. Log 10 compound concentrations (X-axis). EC50 values were calculated using a non-linear regression, four-parameters sigmoidal dose-response model.

### Potency Table

| Example | RIPK1 EC₅₀ |
|---|---|
| 1.1 | 256 |
| 1.2 | 138 |
| 1.3 | 18 |
| 2.1 | 1132 |
| 2.2 | 595 |
| 3.1 | 108 |
| 4.1 | 49 |
| 4.2 | 24 |
| 5.1 | 57 |
| 5.2 | 121 |
| 6.1 | 93370 |
| 6.2 | 99010 |
| 6.3 | 129 |
| 6.4 | 2832 |
| 7.1 | 20 |
| 7.2 | 18 |
| 8.1 | 31 |
| 8.2 | 251 |
| 8.3 | 20 |
| 8.4 | 86 |
| 8.5 | 20 |
| 8.6 | 65 |
| 8.7 | 74 |
| 8.8 | 155 |
| 8.9 | 72 |
| 8.10 | 11 |
| 8.11 | 32 |
| 8.12 | 20 |
| 8.13 | 62 |
| 8.14 | 83 |
| 8.15 | 37 |
| 8.16 | 58 |
| 8.17 | 35 |
| 8.18 | 21 |
| 8.19 | 254 |
| 8.20 | 20 |
| 8.21 | 39 |
| 8.22 | 29 |
| 8.23 | 29 |
| 9.1 | 14 |
| 9.2 | 16 |
| 9.3 | 10 |
| 9.4 | 105 |
| 9.5 | 328 |
| 9.6 | 12 |
| 9.7 | 201 |
| 9.8 | 24 |
| 9.9 | 475 |
| 9.10 | 35 |
| 9.11 | 21 |
| 9.12 | 446 |
| 9.13 | 108 |
| 9.14 | 139 |
| 9.15 | 73 |
| 9.16 | 78 |
| 9.17 | 18 |
| 9.18 | 54 |
| 9.19 | 64 |
| 9.20 | 53 |
| 9.21 | 14 |
| 9.22 | 11 |
| 9.23 | 55 |
| 9.24 | 11 |
| 9.25 | 210 |
| 9.26 | 276 |
| 9.27 | 12 |
| 9.28 | 16 |
| 9.29 | 47 |
| 9.30 | 9 |
| 9.31 | 9 |
| 9.32 | 8 |
| 9.33 | 8 |
| 9.34 | 19 |
| 9.35 | 64 |
| 9.36 | 16 |
| 9.37 | 19 |
| 9.38 | 29 |
| 9.39 | 47 |
| 9.40 | 474 |
| 9.41 | 14 |
| 9.42 | 21 |
| 9.43 | 353 |
| 9.44 | 28 |
| 9.45 | 233 |
| 9.46 | 15 |
| 9.47 | 24 |
| 9.48 | 20 |
| 9.49 | 15 |
| 9.50 | 330 |
| 9.51 | 56 |
| 9.52 | 19 |
| 9.53 | 50 |
| 9.54 | 24 |
| 9.55 | 13 |
| 9.56 | 18 |
| 9.57 | 69 |
| 9.58 | 467.4 |
| 9.59 | 71.89 |
| 9.60 | 187.8 |
| 9.61 | 104 |
| 9.62 | 10.2 |
| 9.63 | 33.3 |
| 9.64 | 124 |
| 9.65 | 13 |
| 9.66 | 21 |
| 10.1 | 9 |
| 10.2 | 18 |
| 10.3 | 11 |
| 10.4 | 13 |
| 10.5 | 20 |
| 10.6 | 52 |
| 10.7 | 27 |
| 11.1 | 12 |
| 11.2 | 16 |
| 11.3 | 19 |
| 12.1 | 390 |
| 12.2 | 155 |
| 12.3 | 239 |
| 12.4 | 329 |
| 12.5 | 305 |
| 12.6 | 16 |
| 13 | 165 |
| 14 | 13 |
| 15.1 | 284 |
| 15.2 | 70 |
| 15.3 | 26 |
| 15.4 | 19 |
| 16.1 | 30 |
| 16.2 | 29 |
| 16.3 | 62 |
| 16.4 | 476 |
| 17.1 | 86 |
| 17.2 | 227 |
| 18.1 | 100 |
| 18.2 | 66 |
| 18.3 | 969 |
| 18.4 | 45 |
| 18.5 | 307 |
| 18.6 | 365 |
| 18.7 | 52 |
| 18.8 | 466 |
| 18.9 | 100 |
| 18.10 | 12 |
| 18.11 | 89 |
| 18.12 | 181 |
| 18.13 | 46 |
| 18.14 | 16 |
| 18.15 | 76 |
| 18.16 | 349 |
| 18.17 | 145 |
| 18.18 | 18 |
| 18.19 | 110 |
| 18.20 | 22 |
| 18.21 | 32 |
| 18.22 | 74 |
| 18.23 | 27 |
| 18.24 | 310 |
| 18.25 | 48 |
| 18.26 | 34 |
| 18.27 | 126 |
| 18.28 | 174 |
| 18.29 | 12 |
| 18.30 | 45 |
| 18.31 | 428 |
| 18.32 | 143 |
| 18.33 | 43 |
| 18.34 | 231 |
| 18.35 | 14 |
| 18.36 | 28 |
| 18.37 | 15 |
| 18.38 | 12 |
| 18.39 | 301 |
| 18.40 | 119 |
| 18.41 | 53 |
| 18.42 | 110 |
| 18.43 | 6 |
| 18.44 | 241 |
| 18.45 | 159 |
| 18.46 | 128 |
| 18.47 | 60 |
| 18.48 | 31 |
| 18.49 | 59 |
| 18.50 | 65 |
| 18.51 | 210 |
| 18.52 | 96 |
| 18.53 | 117 |
| 18.54 | 159 |
| 18.55 | 126 |
| 18.56 | 11 |
| 18.57 | 109 |
| 18.58 | 40 |
| 18.59 | 12 |
| 18.60 | 91 |
| 18.61 | 37 |
| 18.62 | 47 |
| 18.63 | 354 |
| 18.64 | 122 |
| 18.65 | 32 |
| 18.66 | 141 |
| 18.67 | 88 |
| 18.68 | 43 |
| 18.69 | 109 |
| 18.70 | 150 |
| 18.71 | 796 |
| 18.72 | 83 |
| 18.73 | 111 |
| 18.74 | 92 |
| 18.75 | 896 |
| 18.76 | 168 |
| 18.77 | 174 |
| 18.78 | 721 |
| 18.79 | 20 |
| 18.80 | 45 |
| 18.81 | 756 |
| 18.82 | 99 |
| 18.83 | 140 |
| 18.84 | 182 |
| 18.85 | 79 |
| 18.86 | 94 |
| 18.87 | 583 |
| 18.88 | 169 |
| 18.89 | 238 |
| 18.90 | 780 |
| 18.91 | 22 |
| 18.92 | 243 |
| 18.93 | 329 |
| 18.94 | 283 |
| 18.95 | 130 |
| 18.96 | 225 |
| 18.97 | 289 |
| 18.98 | 198 |
| 18.99 | 205 |
| 18.100 | 472 |
| 18.101 | 367 |
| 18.102 | 736 |
| 18.103 | 607 |
| 18.104 | 355 |
| 18.105 | 66 |
| 18.106 | 569 |
| 18.107 | 34 |
| 18.108 | 94 |
| 18.109 | 25 |
| 18.110 | 44 |
| 19.1 | 31 |
| 19.2 | 67 |
| 19.3 | 20 |
| 19.4 | 33 |
| 19.5 | 11 |
| 19.6 | 203 |
| 19.7 | 531 |
| 19.8 | 474 |
| 19.9 | 397 |
| 19.10 | 30 |
| 19.11 | 209 |
| 19.12 | 790 |
| 19.13 | 15 |
| 19.14 | 16 |
| 19.15 | 18 |
| 19.16 | 18 |
| 19.17 | 21 |
| 19.18 | 23 |
| 19.19 | 27 |
| 19.20 | 13 |
| 19.21 | 27 |
| 19.22 | 12 |
| 20.1 | 664 |
| 20.2 | 196 |
| 20.3 | 126 |
| 20.4 | 179 |
| 20.5 | 74 |
| 20.6 | 86 |
| 20.7 | 86 |
| 20.8 | 236 |
| 20.9 | 83 |
| 21.1 | 255 |
| 21.2 | 106 |
| 22.1 | 18 |
| 22.2 | 27 |
| 22.3 | 33 |
| 22.4 | 146 |
| 22.5 | 15 |
| 22.6 | 15 |
| 22.7 | 29 |
| 22.8 | 35 |
| 22.9 | 37 |
| 23.1 | 460 |
| 23.2 | 577 |
| 23.3 | 316 |
| 23.4 | 649 |

What is claimed is:

1. A compound of formula (1) 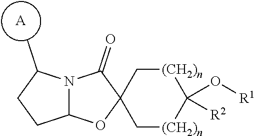
or a pharmaceutically acceptable salt thereof, wherein: 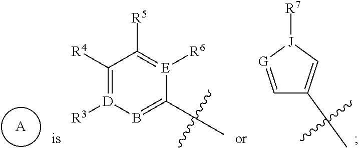
R¹ is —(R⁷)m-R⁸
R² is H or (C1-C6) alkyl;
R³ is H or halogen;
R⁴ is H or halogen;
R⁵ is H or halogen;
R⁶ is H or halogen;
R⁷ is (C₁-C₆)alkyl or (C₁-C₆)carbonyl;
R⁸ is an aryl optionally substituted by up to 3 halogen, —O(C₁-C₆)alkyl, —C(O)NH₂, (C₁-C₆) alkyl optionally substituted by up to 3 halogen, (C₁-C₆)nitrile, (C₅-C₆)heteroaryl having up to 3 heteroatoms selected from N, O, and S and further optionally substituted by (C₁-C₆)alkyl which is optionally substituted by up to 3 halogen; or a heteroaryl having up to 5 heteroatoms selected from N, O, and S and optionally substituted by up to 3 halogen, —O(C₁-C₆)alkyl, —C(O)NH₂, (C₁-C₆) alkyl optionally substituted by up to 3 halogen, (C₁-C₆)nitrile, (C₅-C₆)heteroaryl having up to 3 heteroatoms selected from N, O, and S and further optionally substituted by (C₁-C₆)alkyl which is optionally substituted by up to 3 halogen;
B is C or N;
D is C or N;
E is C or N;
G is C or N;
J is C or N;
n is 0 or 1; and
m is 0 or 1.

2. The compound of claim 1, or a pharmaceutically acceptable salt thereof, wherein A is

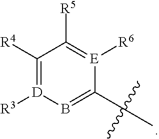

3. The compound of claim 2, or a pharmaceutically acceptable salt thereof, wherein B, D, and E are C.

4. The compound of claim 2, or a pharmaceutically acceptable salt thereof, wherein D and E are both N.

5. The compound of claim 2, or a pharmaceutically acceptable salt thereof, wherein E is N.

6. The compound of claim 2, or a pharmaceutically acceptable salt thereof, wherein R⁴ and R⁶ are both F.

7. The compound of claim 2, or a pharmaceutically acceptable salt thereof, wherein R⁵ is F.

8. The compound of claim 2, or a pharmaceutically acceptable salt thereof, wherein R² is H.

9. The compound of claim 1, or a pharmaceutically acceptable salt thereof, wherein R⁷ is (C₁-C₆) alkyl and m is 1.

10. The compound of claim 1, or a pharmaceutically acceptable salt thereof, wherein m is 0.

11. The compound of claim 1, or a pharmaceutically acceptable salt thereof, wherein R⁸ is an aryl substituted by up to 3 halogen.

12. The compound of claim 1, or a pharmaceutically acceptable salt thereof, wherein R⁸ is an aryl substituted by (C₁-C₆)nitrile.

13. The compound of claim 1, or a pharmaceutically acceptable salt thereof, wherein R⁸ is a heteroaryl having up to 5 heteroatoms selected from N, O and S.

14. The compound of claim 1, or a pharmaceutically acceptable salt thereof, wherein R8 is a heteroaryl having up to 5 heteroatoms selected from N, O and S and wherein the heteroaryl is substituted by up to 3 halogen.

15. The compound of claim 1, or a pharmaceutically acceptable salt thereof, wherein R8 is a heteroaryl having up to 5 heteroatoms selected from N, O and S and wherein the heteroaryl is substituted by (C₁-C₆)nitrile.

16. The compound of claim 1, or a pharmaceutically acceptable salt thereof, wherein R8 is a heteroaryl having up to 5 heteroatoms selected from N, O and S and wherein the heteroaryl is substituted by (C₁-C₆)alkyl.

17. The compound of claim 1, or a pharmaceutically acceptable salt thereof, wherein R⁸ is a heteroaryl having up to 5 heteroatoms selected from N, O, and S and wherein the heteroaryl is substituted by a (C₅-C₆) heteroaryl having up to 3 heteroatoms selected from N, O, and S.

18. The compound of claim 1, or a pharmaceutically acceptable salt thereof, wherein R⁸ is a heteroaryl having up to 5 heteroatoms selected from N, O, and S and wherein the heteroaryl is substituted by a (C₅-C₆) heteroaryl having up to 3 heteroatoms selected from N, O, and S which is further substituted by (C₁-C₆) alkyl.

19. The compound of claim 1, or a pharmaceutically acceptable salt thereof, wherein R⁸ is a heteroaryl having up to 5 heteroatoms selected from N, O, and S and wherein the heteroaryl is substituted by a (C₅-C₆) heteroaryl having up to 3 heteroatoms selected from N, O, and S which is further substituted by CF₂.

20. The compound of claim 1, or a pharmaceutically acceptable salt thereof, wherein n is 0.

21. The compound of claim 1, or a pharmaceutically acceptable salt thereof, wherein n is 1.

22. The compound of claim 1, or a pharmaceutically acceptable salt thereof, wherein the compound of formula 1 is selected from:
((1r or s),4R,5′S,7a′R)-4-(benzyloxy)-5′-phenyltetrahydro-3′H-spiro[cyclohexane-1,2′-pyrrolo[2,1-b]oxazol]-3′-one;
((1s or r),4R,5′S,7a′R)-4-(benzyloxy)-5′-phenyltetrahydro-3′H-spiro[cyclohexane-1,2′-pyrrolo[2,1-b]oxazol]-3′-one;
(5′S,7a′R)-3-(benzyloxy)-5′-phenyltetrahydro-3′H-spiro[cyclobutane-1,2′-pyrrolo[2,1-b]oxazol]-3′-one;
((1r or s),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one;
((1s or r),4R,5′S,7a′R)-4-(benzyloxy)-5′-(5-fluoropyridin-3-yl)tetrahydro-3′H-spiro[cyclohexane-1,2′-pyrrolo[2,1-b]oxazol]-3′-one;
(1s,3R,5′S,7a′R)-3-(benzyloxy)-5′-(5-fluoropyridin-2-yl)tetrahydro-3′H-spiro[cyclobutane-1,2′-pyrrolo[2,1-b]oxazol]-3′-one;
((1r),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one;
((1s),3R,5′S,7a′R)-3-(benzyloxy)-5′-(pyrazin-2-yl)tetrahydro-3′H-spiro[cyclobutane-1,2′-pyrrolo[2,1-b]oxazol]-3′-one;
6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carbonitrile;
6-(((5′S,7a′R)-5′-(3,5-difluorophenyl)-3-methyl-3′-oxotetrahydro-3′H-spiro[cyclobutane-1,2′-pyrrolo[2,1-b]oxazol]-3-yl)oxy)pyrimidine-4-carboxamide;
6-((((1r or s),3R,5′(S or R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
6-((((1s or r),3R,5′(S or R),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
6-((((1r or s),3R,5′(R or S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
6-((((1s or r),3R,5′(R or S),7a′R)-5′-(1-methyl-1H-pyrazol-4-yl)-3′-oxotetrahydro-3′H-spiro[cyclobutane-1,2′-pyrrolo[2,1-b]oxazol]-3-yl)oxy)pyrimidine-4-carbonitrile;
(1r,3R,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one;
3-(benzyloxy)-5′-(pyrazin-2-yl)tetrahydro-3′H-spiro[cyclobutane-1,2′-pyrrolo[2,1-b]oxazol]-3′-one;
(1s,3S,5′S,7a′R)-3-(2,4-difluorophenoxy)-5′-(pyrazin-2-yl)tetrahydro-3′H-spiro[cyclobutane-1,2′-pyrrolo[2,1-b]oxazol]-3′-one;
2-fluoro-4-(((1r,3R,5′S,7a′R)-3′-oxo-5′-phenyltetrahydro-3′H-spiro[cyclobutane-1,2′-pyrrolo[2,1-b]oxazol]-3-yl)oxy)benzonitrile;
5-{[(5′S,7a′R)-3′-oxo-5′-phenyltetrahydro-3′H-spiro[cyclobutane-1,2′-pyrrolo[2,1-b][1,3]oxazol]-3-yl]oxy}pyridine-2-carbonitrile;
4-fluoro-3-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-5′-phenyl-3-{[2-(trifluoromethyl)pyridin-4-yl]oxy}tetrahydro-3′H-spiro[cyclobutane-1,2′-pyrrolo[2,1-b][1,3]oxazol]-3′-one;
3-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-3-[(5-methoxypyridin-3-yl)oxy]-5′-phenyltetrahydro-3′H-spiro[cyclobutane-1,2′-pyrrolo[2,1-b][1,3]oxazol]-3′-one;
(5′S,7a′R)-3-[(3-fluoropyridin-4-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-{[5-(trifluoromethyl)pyridin-3-yl]oxy}tetrahydro-3′H-spiro[cyclobutane-1,2′-pyrrolo[2,1-b][1,3]oxazol]-3′-one;
4-{[(5′S,7a′R)-3′-oxo-5′-phenyltetrahydro-3′H-spiro[cyclobutane-1,2′-pyrrolo[2,1-b][1,3]oxazol]-3-yl]oxy}benzonitrile;
(5′S,7a′R)-3-(3-fluorophenoxy)-5′-phenyltetrahydro-3′H-spiro[cyclobutane-1,2′-pyrrolo[2,1-b][1,3]oxazol]-3′-one;
(5′S,7a′R)-3-[(2-chloropyridin-4-yl)oxy]-5′-phenyltetrahydro-3′H-spiro[cyclobutane-1,2′-pyrrolo[2,1-b][1,3]oxazol]-3′-one;
(5′S,7a′R)-3-[(1,2-benzothiazol-4-yl)oxy]-5′-phenyltetrahydro-3′H-spiro[cyclobutane-1,2′-pyrrolo[2,1-b][1,3]oxazol]-3′-one;
(5′S,7a′R)-3-(4-fluoro-3-methylphenoxy)-5′-phenyltetrahydro-3′H-spiro[cyclobutane-1,2′-pyrrolo[2,1-b][1,3]oxazol]-3′-one;
(5′S,7a′R)-3-[(1,2-benzothiazol-5-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-[3-(trifluoromethyl)phenoxy]tetrahydro-3′H-spiro[cyclobutane-1,2′-pyrrolo[2,1-b][1,3]oxazol]-3′-one;
(5′S,7a′R)-3-[3-(1,3,4-oxadiazol-2-yl)phenoxy]-5′-phenyltetrahydro-3′H-spiro[cyclobutane-1,2′-pyrrolo[2,1-b][1,3]oxazol]-3′-one;
(5′S,7a′R)-3-[(5-chloropyridin-3-yl)oxy]-5′-phenyltetrahydro-3′H-spiro[cyclobutane-1,2′-pyrrolo[2,1-b][1,3]oxazol]-3′-one;
(5′S,7a′R)-3-(4-fluoro-3-methoxyphenoxy)-5′-phenyltetrahydro-3′H-spiro[cyclobutane-1,2′-pyrrolo[2,1-b][1,3]oxazol]-3′-one;
(5′S,7a′R)-3-[(1,2-benzoxazol-7-yl)oxy]-5′-phenyltetrahydro-3′H-spiro[cyclobutane-1,2′-pyrrolo[2,1-b][1,3]oxazol]-3′-one;
(5′S,7a′R)-3-[(imidazo[1,2-c]pyrimidin-5-yl)oxy]-5′-phenyltetrahydro-3′H-spiro[cyclobutane-1,2′-pyrrolo[2,1-b][1,3]oxazol]-3′-one;
(5′S,7a′R)-5′-phenyl-3-[(pyrrolo[1,2-b]pyridazin-4-yl)oxy]tetrahydro-3′H-spiro[cyclobutane-1,2′-pyrrolo[2,1-b][1,3]oxazol]-3′-one;
(1r,3R,5′S,7a′R)-5′-(4-fluorophenyl)-3-(pyrrolo[1,2-b]pyridazin-4-yloxy)tetrahydro-3′H-spiro[cyclobutane-1,2′-pyrrolo[2,1-b]oxazol]-3′-one;
(5′S,7a′R)-5′-phenyl-3-[(pyrrolo[1,2-d][1,2,4]triazin-4-yl)oxy]tetrahydro-3′H-spiro[cyclobutane-1,2′-pyrrolo[2,1-b][1,3]oxazol]-3′-one;
6-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrimidine-4-carbonitrile;
6-(((5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
(5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one;
(1s,3S,5′S,7a′R)-5′-(3,5-difluorophenyl)-3-((3-fluoropyrazolo[1,5-a]pyrimidin-7-yl)oxy)tetrahydro-3′H-spiro[cyclobutane-1,2′-pyrrolo[2,1-b]oxazol]-3′-one;
(1r,3R,5′S,7a′R)-5′-(3,5-difluorophenyl)-3-((3-fluoropyrazolo[1,5-a]pyrimidin-7-yl)oxy)tetrahydro-3′H-spiro[cyclobutane-1,2′-pyrrolo[2,1-b]oxazol]-3′-one;
(5′S,7a′R)-5′-(3,5-difluorophenyl)-3-{[6-(1H-pyrazol-1-yl)pyrimidin-4-yl]oxy}tetrahydro-3′H-spiro[cyclobutane-1,2′-pyrrolo[2,1-b][1,3]oxazol]-3′-one;
(5′S,7a′R)-5′-(3,5-difluorophenyl)-3-[(2-methyl[1,3]thiazolo[5,4-d]pyrimidin-7-yl)oxy]tetrahydro-3′H-spiro[cyclobutane-1,2′-pyrrolo[2,1-b][1,3]oxazol]-3′-one;
(5′S,7a′R)-5′-(3,5-difluorophenyl)-3-{[6-(1H-pyrazol-1-yl)pyrimidin-4-yl]oxy}tetrahydro-3′H-spiro[cyclobutane-1,2′-pyrrolo[2,1-b][1,3]oxazol]-3′-one;
(1s,3S,5′S,7a′R)-5′-(3,5-difluorophenyl)-3-((6-(4-methyl-1H-imidazol-1-yl)pyrimidin-4-yl)oxy)tetrahydro-3′H-spiro[cyclobutane-1,2′-pyrrolo[2,1-b]oxazol]-3′-one;
(1s,3S,5′S,7a′R)-5′-(3,5-difluorophenyl)-3-((6-fluoropyrimidin-4-yl)oxy)tetrahydro-3′H-spiro[cyclobutane-1,2′-pyrrolo[2,1-b]oxazol]-3′-one;
(5′S,7a′R)-5′-(3,5-difluorophenyl)-3-{[6-(4-methyl-1H-imidazol-1-yl)pyrimidin-4-yl]oxy}tetrahydro-3′H-spiro[cyclobutane-1,2′-pyrrolo[2,1-b][1,3]oxazol]-3′-one;
6-(((1s,3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
(1s,3S,5′S,7a′R)-3-((6-chloropyrimidin-4-yl)oxy)-5′-(3,5-difluorophenyl)tetrahydro-3′H-spiro[cyclobutane-1,2′-pyrrolo[2,1-b]oxazol]-3′-one;
3-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyridine-2-carbonitrile;
5-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyridine-3-carbonitrile;
6-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrazine-2-carbonitrile;
6-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-4-carbonitrile;
4-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-2-carbonitrile;
2-{[(1r,3R,5′S,7a′R)-3′-oxo-5′-phenyltetrahydro-3′H-spiro[cyclobutane-1,2′-pyrrolo[2,1-b][1,3]oxazol]-3-yl]oxy}pyrimidine-4-carbonitrile;
2-{[(1r,3R,5′S,7a′R)-5′-(3,5-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
(1r,3R,5′S,7a′R)-5′-(3,5-difluorophenyl)-3-[(2-methylpyrrolo[2,1-f][1,2,4]triazin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(thieno[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(furo[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(6-methylfuro[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(3,5-difluorophenyl)-3-[(6-methylthieno[2,3-d]pyrimidin-4-yl)oxy]tetrahydro-3′H-spiro[cyclobutane-1,2′-pyrrolo[2,1-b][1,3]oxazol]-3′-one;
(1r,3R,5′S,7a′R)-5′-(4-fluorophenyl)-3-[(imidazo[1,2-c]pyrimidin-5-yl)oxy]tetrahydro-3′H-spiro[cyclobutane-1,2′-pyrrolo[2,1-b][1,3]oxazol]-3′-one;
(1s,3S,5′S,7a′R)-5′-(3,5-difluorophenyl)-3-((5-fluoropyridin-2-yl)oxy)tetrahydro-3′H-spiro[cyclobutane-1,2′-pyrrolo[2,1-b]oxazol]-3′-one;
(5′S,7a′R)-3-[(2-chloropyridin-3-yl)oxy]-5′-(3,5-difluorophenyl)tetrahydro-3′H-spiro[cyclobutane-1,2′-pyrrolo[2,1-b][1,3]oxazol]-3′-one;
(5′S,7a′R)-5′-(3,5-difluorophenyl)-3-[(5-fluoropyridin-2-yl)oxy]tetrahydro-3′H-spiro[cyclobutane-1,2′-pyrrolo[2,1-b][1,3]oxazol]-3′-one;
(5′S,7a′R)-5′-(3,5-difluorophenyl)-3-[(3-fluoropyridin-2-yl)oxy]tetrahydro-3′H-spiro[cyclobutane-1,2′-pyrrolo[2,1-b][1,3]oxazol]-3′-one;
(5′S,7a′R)-5′-(3,5-difluorophenyl)-3-[(3-fluoropyridin-2-yl)oxy]tetrahydro-3′H-spiro[cyclobutane-1,2′-pyrrolo[2,1-b][1,3]oxazol]-3′-one;
6-{[(5′S,7a′R)-5′-(2,3-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
6-{[(5′S,7a′R)-5′-(2,3-difluorophenyl)-3′-oxotetrahydro-3′H-spiro[cyclobutane-1,2′-pyrrolo[2,1-b][1,3]oxazol]-3-yl]oxy}pyrimidine-4-carbonitrile;
6-(((5′S,7a′R)-3′-oxo-5′-(pyrazin-2-yl)tetrahydro-3′H-spiro[cyclobutane-1,2′-pyrrolo[2,1-b]oxazol]-3-yl)oxy)pyrimidine-4-carbonitrile;
6-{[(5′S,7a′R)-5′-(2-fluorophenyl)-3′-oxotetrahydro-3′H-spiro[cyclobutane-1,2′-pyrrolo[2,1-b][1,3]oxazol]-3-yl]oxy}pyrimidine-4-carbonitrile;
6-(((5′S,7a′R)-5′-(3-cyano-5-fluorophenyl)-3′-oxotetrahydro-3′H-spiro[cyclobutane-1,2′-pyrrolo[2,1-b]oxazol]-3-yl)oxy)pyrimidine-4-carbonitrile;
(1s,3S,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
(1r,3R,5′S,7a′R)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
6-{[(5′S,7a′R)-5′-(5-fluoropyridin-2-yl)-3′-oxotetrahydro-3′H-spiro[cyclobutane-1,2′-pyrrolo[2,1-b][1,3]oxazol]-3-yl]oxy}pyrimidine-4-carbonitrile;
(1r,3R,5′S,7a′R)-5′-(4-fluorophenyl)-3-[(5-fluoropyridin-3-yl)oxy]tetrahydro-3′H-spiro[cyclobutane-1,2′-pyrrolo[2,1-b][1,3]oxazol]-3′-one;
5-{[(1r,3R,5′S,7a′R)-5′-(4-fluorophenyl)-3′-oxotetrahydro-3′H-spiro[cyclobutane-1,2′-pyrrolo[2,1-b][1,3]oxazol]-3-yl]oxy}pyridine-3-carbonitrile;
6-(((5′R,7a'S)-3′-oxo-5′-(pyrazin-2-yl)tetrahydro-3′H-spiro[cyclobutane-1,2′-pyrrolo[2,1-b]oxazol]-3-yl)oxy)pyrimidine-4-carbonitrile;
(1s,3R,5′R,7a'S)-5′-(pyrazin-2-yl)-3-(pyrrolo[2,1-f][1,2,4]triazin-4-yloxy)tetrahydro-3′H-spiro[cyclobutane-1,2′-pyrrolo[2,1-b]oxazol]-3′-one;
(5′S,7a′R)-5′-phenyl-4-[(pyrazin-2-yl)oxy]tetrahydro-3′H-spiro[cyclohexane-1,2′-pyrrolo[2,1-b][1,3]oxazol]-3′-one;
(5′S,7a′R)-5′-(3,5-difluorophenyl)-3-((6-(5-methyl-1,3,4-oxadiazol-2-yl)pyrimidin-4-yl)oxy)tetrahydro-3′H-spiro[cyclobutane-1,2′-pyrrolo[2,1-b]oxazol]-3′-one;
(5′S,7a′R)-3-((6-(1-(difluoromethyl)-1H-pyrazol-4-yl)pyrimidin-4-yl)oxy)-5′-(3,5-difluorophenyl)tetrahydro-3′H-spiro[cyclobutane-1,2′-pyrrolo[2,1-b]oxazol]-3′-one;
(5′S,7a′R)-5′-(3,5-difluorophenyl)-3-((6-(1-methyl-1H-pyrazol-5-yl)pyrimidin-4-yl)oxy)tetrahydro-3′H-spiro[cyclobutane-1,2′-pyrrolo[2,1-b]oxazol]-3′-one;
(5′S,7a′R)-3-((6-(4-(difluoromethyl)-1H-pyrazol-1-yl)pyrimidin-4-yl)oxy)-5′-(3,5-difluorophenyl)tetrahydro-3′H-spiro[cyclobutane-1,2′-pyrrolo[2,1-b]oxazol]-3′-one;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrimidine-4-carbonitrile;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinonitrile;
2-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)isonicotinamide;
(1r,3R,5′S,7a′R)-3-((4-(2H-tetrazol-5-yl)pyridin-2-yl)oxy)-5′-(3,5-difluorophenyl)tetrahydro-3′H-spiro[cyclobutane-1,2′-pyrrolo[2,1-b]oxazol]-3′-one;
2-(((1r,3R,5′S,7a′R)-3′-oxo-5′-phenyltetrahydro-3′H-spiro[cyclobutane-1,2′-pyrrolo[2,1-b]oxazol]-3-yl)oxy)isonicotinonitrile;
2-(((1r,3R,5′S,7a′R)-3′-oxo-5′-phenyltetrahydro-3′H-spiro[cyclobutane-1,2′-pyrrolo[2,1-b]oxazol]-3-yl)oxy)isonicotinamide;
2-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)isonicotinamide;
(1r,3R,5′S,7a′R)-3-((4-(2H-tetrazol-5-yl)pyridin-2-yl)oxy)-5′-(4-fluorophenyl)tetrahydro-3′H-spiro[cyclobutane-1,2′-pyrrolo[2,1-b]oxazol]-3′-one;
(1r,3R,5′S,7a′R)-3-((7-bromopyrrolo[2,1-f][1,2,4]triazin-4-yl)oxy)-5′-(3,5-difluorophenyl)tetrahydro-3′H-spiro[cyclobutane-1,2′-pyrrolo[2,1-b]oxazol]-3′-one;
4-(((1r,3R,5′S,7a′R)-5′-(3,5-difluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile;
4-(((1r,3R,5′S,7a′R)-5′-(4-fluorophenyl)-3′-oxotetrahydro-3′H-spiro[cyclobutane-1,2′-pyrrolo[2,1-b]oxazol]-3-yl)oxy)pyrrolo[2,1-f][1,2,4]triazine-7-carbonitrile;
(1R,3R,5′S,7a′R)-5′-(3,5-difluorophenyl)-3-((R or S)-1-(5-fluoropyridin-2-yl)ethoxy)tetrahydro-3′H-spiro[cyclobutane-1,2′-pyrrolo[2,1-b]oxazol]-3′-one;
(1R,3R,5′S,7a′R)-5′-(3,5-difluorophenyl)-3-((S or R)-1-(5-fluoropyridin-2-yl)ethoxy)tetrahydro-3′H-spiro[cyclobutane-1,2′-pyrrolo[2,1-b]oxazol]-3′-one;
(1R or S,3R or S,5′S,7a′R)-5′-(3,5-difluorophenyl)-3-((R or S)-1-(3-fluoropyridin-2-yl)ethoxy)tetrahydro-3′H-spiro[cyclobutane-1,2′-pyrrolo[2,1-b]oxazol]-3′-one;
1S or R,3S or R,5′S,7a′R)-5′-(3,5-difluorophenyl)-3-((S or R)-1-(3-fluoropyridin-2-yl)ethoxy)tetrahydro-3′H-spiro[cyclobutane-1,2′-pyrrolo[2,1-b]oxazol]-3′-one;
(1R or S,3S or r,5′S,7a′R)-5′-(3,5-difluorophenyl)-3-((s or r)-1-(3-fluoropyridin-2-yl)ethoxy)tetrahydro-3′H-spiro[cyclobutane-1,2′-pyrrolo[2,1-b]oxazol]-3′-one;
(1r,3R,5′S,7a′R)-5′-(3,5-difluorophenyl)-3′-oxotetrahydro-3′H-spiro[cyclobutane-1,2′-pyrrolo[2,1-b]oxazol]-3-yl picolinate;
(1r,3R,5′S,7a′R)-5′-(3,5-difluorophenyl)-3′-oxotetrahydro-3′H-spiro[cyclobutane-1,2′-pyrrolo[2,1-b]oxazol]-3-yl benzoate;
((1S or R),3R,5′S,7a′R)-5′-(3,5-difluorophenyl)-3-((S or R)-1-phenylethoxy)tetrahydro-3′H-spiro[cyclobutane-1,2′-pyrrolo[2,1-b]oxazol]-3′-one;
((1R or S),3R,5′S,7a′R)-5′-(3,5-difluorophenyl)-3-((S or R)-1-phenylethoxy)tetrahydro-3′H-spiro[cyclobutane-1,2′-pyrrolo[2,1-b]oxazol]-3′-one;
((1S or R),3R,5′S,7a′R)-5′-(3,5-difluorophenyl)-3-((R or S)-1-phenylethoxy)tetrahydro-3′H-spiro[cyclobutane-1,2′-pyrrolo[2,1-b]oxazol]-3′-one;
((1R or S),3R,5′S,7a′R)-5′-(3,5-difluorophenyl)-3-((R or S)-1-phenylethoxy)tetrahydro-3′H-spiro[cyclobutane-1,2′-pyrrolo[2,1-b]oxazol]-3′-one;
((1s or r),3R,5′S,7a′R)-5′-(3,5-difluorophenyl)-3-phenoxytetrahydro-3′H-spiro[cyclobutane-1,2′-pyrrolo[2,1-b]oxazol]-3′-one;
((1r or s),3R,5′S,7a′R)-5′-(3,5-difluorophenyl)-3-phenoxytetrahydro-3′H-spiro[cyclobutane-1,2′-pyrrolo[2,1-b]oxazol]-3′-one;
(1r or s),(5′S,7a′R)-5′-(5-fluoropyridin-3-yl)-4-phenoxytetrahydro-3′H-spiro[cyclohexane-1,2′-pyrrolo[2,1-b]oxazol]-3′-one;
(1s or r), (5′S,7a′R)-5′-(5-fluoropyridin-3-yl)-4-phenoxytetrahydro-3′H-spiro[cyclohexane-1,2′-pyrrolo[2,1-b]oxazol]-3′-one;
2-((((is or r),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile;
2-((((1r or s),3S,5′S,7a′R)-5′-(3,5-difluorophenyl)-3′-oxotetrahydro-3′H-spiro[cyclobutane-1,2′-pyrrolo[2,1-b]oxazol]-3-yl)oxy)-5-fluoroisonicotinonitrile.

23. The compound of claim 1, or a pharmaceutically acceptable salt thereof, wherein the compound of formula 1 is selected from:

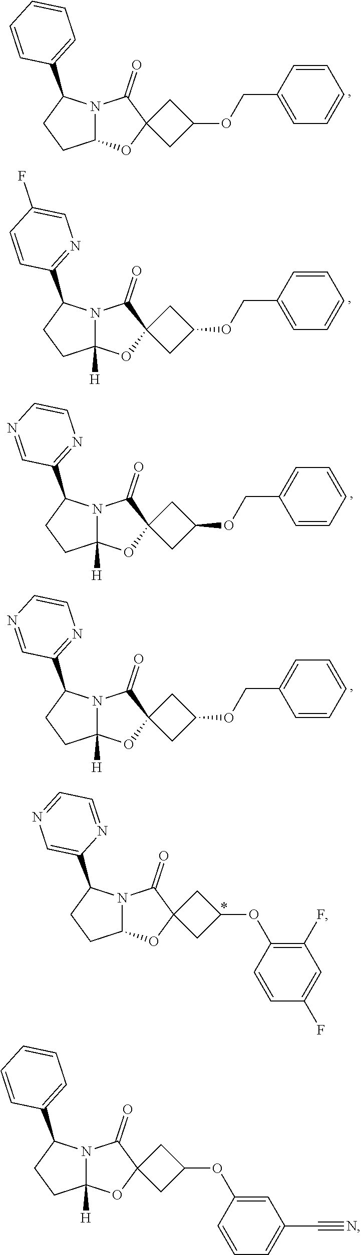

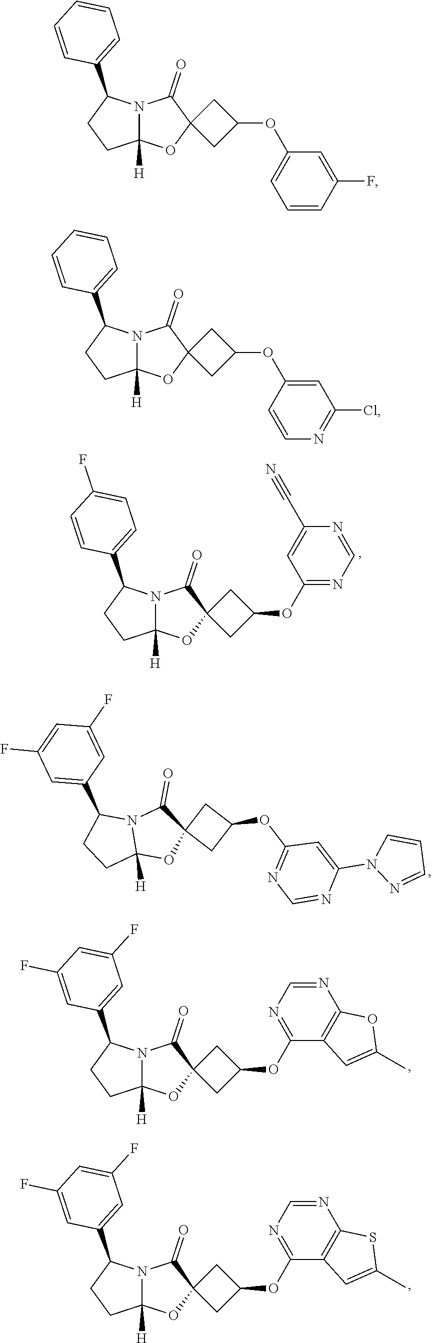

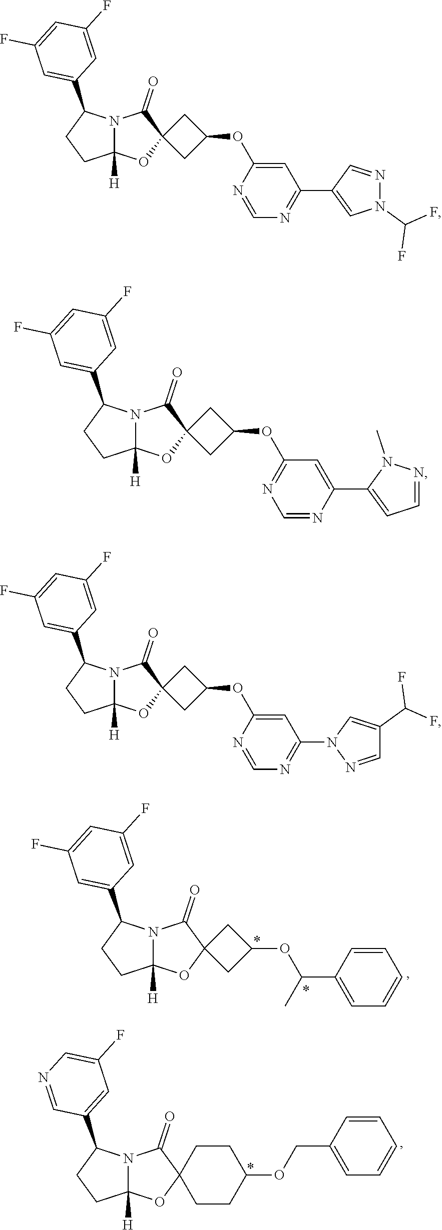 [SMILES: O=C1N2[C@@H](CC[C@H]2c2cc(F)cc(F)c2)O[C@]12C[C@H](Oc1cc(-c3cnn(C(F)F)c3)ncn1)C2 ; Cn1nccc1-c1cc(O[C@H]2C[C@@]3(C2)O[C@@H]2CC[C@@H](c4cc(F)cc(F)c4)N2C3=O)ncn1 ; O=C1N2[C@@H](CC[C@H]2c2cc(F)cc(F)c2)O[C@]12C[C@H](Oc1cc(-n3cc(C(F)F)cn3)ncn1)C2 ; CC(OC1CC2(C1)O[C@@H]1CC[C@@H](c3cc(F)cc(F)c3)N1C2=O)c1ccccc1 ; O=C1N2[C@@H](CC[C@H]2c2cncc(F)c2)OC12CCC(OCc1ccccc1)CC2]

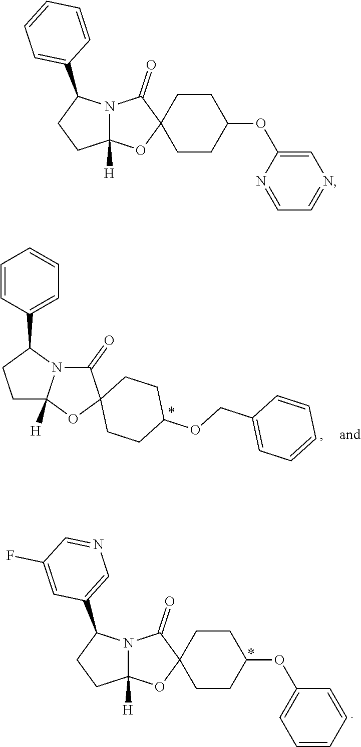 [SMILES: O=C1N2[C@@H](CC[C@H]2c2ccccc2)OC12CCC(Oc1cnccn1)CC2 ; O=C1N2[C@@H](CC[C@H]2c2ccccc2)OC12CCC(OCc1ccccc1)CC2 ; O=C1N2[C@@H](CC[C@H]2c2cncc(F)c2)OC12CCC(Oc1ccccc1)CC2]

24. A method for treating RIPK1 dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis comprising administering to a patient in need thereof a compound, or pharmaceutically acceptable salt thereof, of claim 1.

25. A method of treating amyotrophic lateral sclerosis comprising administering to a patient in need thereof a compound, or pharmaceutically acceptable salt thereof, of claim 1.

26. A pharmaceutical composition comprising a compound of claim 1, or a pharmaceutically acceptable salt thereof and a pharmaceutically acceptable carrier.
